(19) United States
(12) Patent Application Publication
(10) Pub. No.: US 2011/0124626 A1
(43) Pub. Date: May 26, 2011

(54) BENZAZEPINE DERIVATIVES AND THEIR USE AS HISTAMINE H3 ANTAGONISTS

(75) Inventors: Parminder Kaur Pooni, Cambridge (GB); Kevin John Merchant, Cambridge (GB); Catrina Morvern Kerr, Cambridge (GB); Stuart Richard Crosby, Cambridge (GB); Tomohiro Okawa, Osaka (JP); Mitsuru Sasaki, Osaka (JP); Mika Gotou, Osaka (JP); Graham Andrew Showell, Cambridge (GB); Martin Richard Teall, Cambridge (GB)

(73) Assignee: TAKEDA PHARMACEUTICAL COMPANY LIMITED, Osaka-shi, Osaka (JP)

(21) Appl. No.: 13/054,688

(22) PCT Filed: Jul. 17, 2009
(86) PCT No.: PCT/GB2009/001774

§ 371 Date: Jan. 18, 2011

(30) Foreign Application Priority Data
Jul. 18, 2008 (GB) 0813254.0
Mar. 26, 2009 (GB) 0905231.7

(51) Int. Cl. A61K 31/397; C07D 403/12; A61K 31/55; C07D 401/12; C07D 223/16; C07D 413/10; C07D 403/14; A61P 25/28

(52) U.S. Cl. 514/210.18; 540/594; 514/217.01

(57) ABSTRACT

A compound having the formula (1) wherein: R1 is a group selected from C₃₋₈ cycloalkyl, C₁₋₆ alkyl, C₁₋₆ alkylene-C₃₋₈ cycloalkyl, each of which groups may optionally be substituted with C₁₋₆ alkyl, halogen, haloC₁₋₆ alkyl or OR15, or R1 is heterocyclyl, optionally substituted with C₁₋₆ alkyl, haloC₁₋₆ alkyl or OR15; n is 0, 1, 2, 3 or 4, the alkylene group —(CH₂)ₘ— formed thereby being optionally substituted with a group selected from C₁₋₄ alkyl, C₃₋₈ cycloalkyl and arylsulfonyl; A is a group selected from —N(R2)CO—, —CON(R2)-, —OC(O)—, —C(O)O—, —CO—, —C(R2)(OR3)-, —C(═N—O—R3)-, —C(═CR2R3)-, —C₃₋₈ cycloalkylene-, —C(R2)(haloC₁₋₆alkyl)-, C₁₋₄ alkylene and —C(OR3)(haloC₁₋₆alkyl)-; R2 and R3 are each independently selected from H, C₁₋₆ alkyl, and C₃₋₈ cycloalkyl, or, when A is —N(R2)CO— and X is absent, R2 may form, together with the adjacent nitrogen atom and Z, an N-containing heterocyclyl group, which may optionally be substituted; X is absent or is C14 alkylene or C24 alkenylene, each of which may optionally be substituted with one or more C₁₋₄ alkyl groups, OR16, halogen or haloC₁₋₆ alkyl; Z is selected from aryl, heteroaryl, C₃₋₈ cycloalkyl, and heterocyclyl, each of which may optionally be substituted by a group selected from —Y-aryl, heteroaryl, —Y—C₃₋₈ cycloalkyl and —Y-heterocyclyl, or, when X is present, Z may be H, or, when X is absent and A is —C(R2)(OR3)- or —N(R2)CO—, Z may be H, or, when A is —N(R2)CO— and X is absent, Z may form, together with the adjacent nitrogen atom and R2, an N-containing heterocyclyl group which may optionally be substituted, wherein, when A is —CO—, Z is linked to X or A via a carbon atom and wherein, when A is —N(R2)CO— and Z is H, R1 is C₃₋₈ cycloalkyl; and Y represents a bond, C₁₋₆ alkylene, CO, NR14, COC₂₋₆ alkenylene, O, SO₂ or NHCOC₁₋₆ alkylene; wherein said cycloalkyl, aryl, heteroaryl and heterocyclyl groups Z may be optionally substituted by one or more substituents which may be the same or different, and which are selected from halogen, haloC₁₋₆ alkyl, hydroxy, cyano, nitro, ═O, —R4, —CO₂R4, —COR4, —NR5R6, —C₁₋₆ alkyl-NR5R6, —C₃₋₈ cycloalkyl-NR5R6, —CONR12R13, —NR12COR13, —NR5SO₂R6, —OCONR5R6, —NR5CO₂R6, —NR4CONR5R6 or —SO₂NR5R6-SHR8, -alkyl-OR8, —SOR8, —OR9, —SO2R9, —OSO₂R9, -alkyl-SO₂R9, -alkyl-CONHR9, -alkyl-SONHR9, -alkyl-COR10, —CO-alkyl-R10, —O-alkyl-R11 (wherein R4, R5 and R6 independently represent hydrogen, C₁₋₆ alkyl, —C₃₋₈ cycloalkyl, —C₁₋₆ alkylene-C₃₋₈ cycloalkyl, aryl, heterocyclyl or heteroaryl, wherein R8 represents C₁₋₆ alkyl, wherein R9 represents C₁₋₆ alkyl or aryl, wherein R10 represents aryl, wherein R11 represents C₃₋₈ cycloalkyl or aryl, R12, R13, R14, R15 and R16 each independently represent H or C₁₋₆ alkyl, and wherein —NR5R6 and —NR12R13 may represent a nitrogen containing heterocyclyl group); wherein said R4, R5, R6 R8, R9, R11 and R11 groups may be optionally substituted by one or more substituents which may be the same or different, and which are selected from the group consisting of halogen, hydroxy, C₁₋₆ alkyl, C₁₋₆ alkoxy, cyano, amino, ═O or trifluoromethyl; and wherein substituents of Z selected from —Y-aryl, —Y-heteroaryl, —Y—C₃₋₈cycloalkyl and —Y-heterocyclyl may be optionally substituted by one or more substituents selected from ═O, hydroxy, cyano, nitro, halogen, haloC₁₋₆ alkyl and C₁₋₆alkyl; and wherein, when A is C₁₋₄ alkylene, said cycloalkyl, aryl, heteroaryl or heterocyclyl group Z (such as a heterocyclyl group Z) is substituted at least with hydroxy, CF₃, or ═O; and wherein, when A is CON(R2) n is 1; or a pharmaceutically acceptable salt or ester thereof, provided that: when A is —CO—, R1 is CH₃, C₃₋₈ cycloalkyl-substituted C₁₋₆ alkylene or n-butyl, n is 0 and X is —CH₂CH₂—, Z is not N-benzyl substituted 4-piperidinyl, N-(3-fluorobenzyl)-substituted 4-piperidinyl or N-acetyl substituted 4-piperidinyl; when A is —OC(O)—, R1 is cyclobutyl, n is 0 and X is —CH₂CH₂—, Z is not H; when A is —OC(O)—, R1 is n-propyl, n is 0 and X is —CH₂—, Z is not H; and when A is —CO—, R1 is CH₃, n is 0 and X is CH₂, Z is not H.

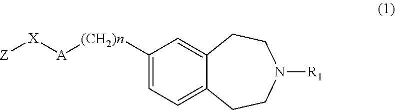

The present invention relates to compounds and their uses, and in particular to compounds having a benzazepine scaffold and their therapeutic use in the treatment or prevention of conditions having an association with the histamine H3 receptor.

The H3 receptor was first identified pharmacologically in 1983 as an autoreceptor that regulates the production of histamine (1). The receptor was later cloned in 1999 (2). It is a constitutively active G protein-coupled receptor that is expressed predominantly in the central nervous system (CNS) and modulates a variety of CNS functions both centrally and peripherally. It is expressed on the presynaptic terminals of CNS neurones and acts as a negative modulator of release of neurotransmitters such as histamine, acetylcholine, norepinephrine, serotonin and dopamine (3). Consequently, the ability of the H3 receptor to regulate the release of a wide range of neurotransmitters has fuelled research into the development of antagonists/inverse agonists which have potential in behavioural and physiological conditions, for example CNS disorders such as narcolepsy, disorders of wakefulness, cognition or attention, pain and in suppression of food intake.

Histaminergic neurones are located in the tuberomammillary nucleus of the posterior hypothalamus and project their axons into brain regions including the hypothalamus, thalamus, cerebral cortex, amygdala, and septum. Activity of histaminergic neurons is closely linked with the sleep/wake cycle and numerous reports in the literature have established that the H3 receptor plays a role in cognition and sleep/wake related processes, based on studies with known H3 receptor antagonists and their effects in animal models (4, 5, 6). H3 antagonist compound A-349821 is currently in preclinical development and has been shown to demonstrate cognition-enhancing effects in the rat (7).

The histaminergic system is one of the targets of leptin signalling in the hypothalamus. Known H3 antagonist clobenpropit increases histamine release in the hypothalamus of mice and has the effect of reducing energy intake in both lean and obese mice (8). The role of the H3 receptor in obesity has been further substantiated through studies with antagonists thioperamide and ciproxifan and more recently with non-imidazole compounds (10).

The non-selective antagonist thioperamide has an antinociceptive effect in a number of acute pain models (11). H3 antagonists have been suggested for the treatment of neuropathic pain (12). In addition GSK207040 and GSK334429 are selective non-imidazole H3 antagonist compounds that display high affinity for both rat and human H3 receptors. Both compounds reduced tactile allodynia in the rat, suggesting H3 antagonists have therapeutic potential in the treatment of neuropathic pain (13).

In an attempt to identify compounds with improved drug-like properties, non-imidazole compounds have been at the forefront of research, for example A-349821 (7) and GSK207040/GSK334429 (13). ABT-239 is currently being investigated for use in attention deficit hyperactivity disorder, Alzheimer's Disease and schizophrenia (14).

WO05/123723, WO06/018260 and WO05/058837 disclose H3 antagonist benzazepine derivatives claimed to be useful in the treatment of neurological and psychiatric disorders. WO05/058328 discloses dopamine D3 receptor benzazepine derivatives claimed to be useful in the treatment of CNS disorders such as schizophrenia and depression. WO02/40471 also discloses benzazepine derivatives useful as modulators of the dopamine D3 receptor. US2003/0158177 discloses melanin-concentrating hormone antagonists claimed to be useful in the treatment of obesity.

There exists a clinical need to generate further classes of H3 antagonist and/or inverse agonist compounds that demonstrate improved drug-like properties (9).

In accordance with a first aspect of the present invention, there is provided a compound having the Formula 1:

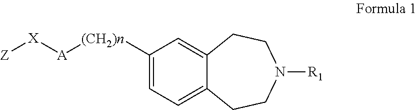

wherein:
R1 is a group selected from C₃₋₄ cycloalkyl, C₁₋₆ alkyl, and C₃₋₄ cycloalkyl-substituted C₁₋₆ alkylene, each of which groups may optionally be substituted with C₁₋₆ alkyl (such as methyl), halogen (such as F), haloC₁₋₆ alkyl (such as CH₂F) or OR15, or R1 is heterocyclyl, optionally substituted with C₁₋₆ alkyl (such as methyl), haloC₁₋₆ alkyl (such as CH₂F) or OR15;
n is 0, 1, 2, 3 or 4, the alkylene group —(CH₂)₆— formed thereby being optionally substituted with a group selected from C₁₋₄ alkyl, C₃₋₄ cycloalkyl and arylsulfonyl;
A is a group selected from —N(R2)CO—, —OC(O)—, —C(O)O—, —CON(R2)-, —CO—, —C(R2)(OR3)-, —C(═N—O—R3)-, —C(═CR2R3)-, —C₃₋₈ cycloalkylene-, —C(R2)(haloC₁₋₆alkyl)-, C₁₋₄ alkylene and —C(OR3)(haloC₁₋₆ alkyl)-;
R2 and R3 are each independently selected from H, C₁₋₆ alkyl (which may be straight- or branched-chain), and C₃₋₄ cycloalkyl, or, when A is —N(R2)CO— and X is absent, R2 may form, together with the adjacent nitrogen atom and Z, an N-containing heterocyclyl group, which may optionally be substituted;
X is absent or is C₁₋₄ alkylene or C₂₋₄ alkenylene, optionally substituted with one or more C₁₋₄ alkyl groups, OR16, halogen (such as F), or haloC₁₋₆allyl (such as CF₃);
Z is selected from aryl, heteroaryl, C₃₋₄ cycloalkyl, and heterocyclyl, each of which may optionally be substituted by a group selected from —Y-aryl, —Y-heteroaryl, —Y—C₃₋₈ cycloalkyl and —Y-heterocyclyl, or, when X is present, Z may be H, or, when X is absent and A is —C(R2)(OR3)- or —N(R2)CO—, Z may be H, or, when A is —N(R2)CO— and X is absent, Z may form, together with the adjacent nitrogen atom and R2, an N-containing heterocyclyl group which may optionally be substituted, wherein, when A is —CO—, Z is linked to X or A via a carbon atom and wherein, when A is —N(R2)CO— and Z is H, R1 is C₃₋₈ cycloalkyl; and
Y represents a bond, C₁₋₆ alkylene, CO, COC₂₋₆ alkenylene, O, SO₂, NR14, or NHCOC₁₋₆ alkylene;
wherein said cycloalkyl, aryl, heteroaryl and heterocyclyl groups Z may be optionally substituted by one or more substituents which may be the same or different, and which are selected from halogen, haloC₁₋₆ alkyl, such as halomethyl, hydroxy, cyano, nitro, ═O, —R4, —CO₂R4, —COR4, —NR5R6, —C₁₋₆ alkyl-NR5R6, —C₃₋₈ cycloalkyl-NR5R6, —CONR12R13, —NR12COR13, —NR5SO₂R6, —OCONR5R6, —NR5CO₂R6, —NR4CONR5R6 or —SO₂NR5R6-SHR8, —C₁₋₆ alkyl-OR8, —SOR8, —OR9, —SO₂R9, —OSO₂R9, —C₁₋₆ alkyl-SO₂R9, —C₁₋₆ alkyl-CONHR9, —C₁₋₆ alkyl-SONHR9, —C₁₋₆ alkyl-COR10, —CO—C₁₋₆ alkyl-R10, —O—C₁₋₆ alkyl-R11 (wherein R4, R5 and R6 independently represent hydrogen, C₁₋₆ alkyl, —C₃₋₈ cycloalkyl, —C₁₋₆ alkylene-C₃₋₈ cycloalkyl, aryl, heterocyclyl or heteroaryl, wherein R8 represents —C₁₋₆ alkyl, wherein R9 represents C₁₋₆ alkyl or aryl, wherein R10 represents aryl, wherein R11 represents C₃₋₈ cycloalkyl or aryl, R12, R13, R14, R15 and R16 each independently represent H or C₁₋₆ alkyl, and wherein —NR5R6 and —NR12R13 may represent a nitrogen containing heterocyclyl group); wherein said R4, R5, R6, R8, R9, R10 and R11 groups may be optionally substituted by one or more substituents which may be the same or different, and which are selected from the group consisting of halogen, hydroxy, C₁₋₆ alkyl, C₁₋₆ alkoxy, cyano, amino, ═O or trifluoromethyl;
wherein substituents of Z selected from —Y-aryl, —Y-heteroaryl, cycloalkyl and —Y-heterocyclyl may be optionally substituted by one or more substituents selected from ═O, hydroxy, cyano, nitro, halogen, haloC₁₋₆ alkyl (such as halomethyl) and C₁₋₆ alkyl;
wherein, when A is C₁₋₄ alkylene, said cycloalkyl, aryl, heteroaryl or heterocyclyl group Z (such as a heterocyclyl group Z) is substituted at least with hydroxy, CF₃ or ═O;
and wherein, when A is CON(R2), n is 1;
or a pharmaceutically acceptable salt or ester thereof,
provided that: when A is —CO—, R1 is CH₃, C₃₋₈ cycloalkyl-substituted C₁₋₆ alkylene or n-butyl, n is 0 and X is —CH₂CH₂—, Z is not N-benzyl substituted 4-piperidinyl, N-(3-fluorobenzyl)-substituted 4-piperidinyl or N-acetyl substituted 4-piperidinyl;
when A is —OC(O)—, R1 is cyclobutyl, n is 0 and X is —CH₂CH₂—, Z is not H;
when A is —OC(O)—, R1 is n-propyl, n is 0 and X is —CH₂—, Z is not H; and
when A is —CO—, R1 is CH₃, n is 0 and X is CH₂, Z is not H.

The compounds of the invention have been found to modulate the histamine H3 receptor. In particular, the compounds possess antagonist or inverse agonist properties at this receptor. Based on the high affinity for the receptor, the compounds may have the potential to display useful selectivity for the H3 receptor. Compounds of the invention where n is at least 1, particularly those in which A is —CON(R2)-, and particularly those in which n is 1, have been found to display blood brain barrier permeability properties rendering them potentially suitable for the treatment of CNS disorders.

The term ‘C_{x-y} alkyl’ as used herein refers to a linear or branched saturated hydrocarbon group containing from x to y carbon atoms. For example, C₁₋₆ alkyl refers to a linear or branched saturated hydrocarbon group containing from 1 to 6 carbon atoms. Examples of C₁₋₆ alkyl groups include methyl, ethyl, n-propyl, isopropyl, n-butyl, isobutyl, sec-butyl, tert butyl, n-pentyl, isopentyl, neopentyl and hexyl.

The term ‘C_{x-y} alkylene’ as used herein refers to a divalent hydrocarbon group obtained by removing one hydrogen atom from ‘C_{x-y} alkyl’ above. Examples of C₁₋₆ alkylene groups include methylene, ethylene and propylene.

The term ‘C_{x-y} alkenyl’ as used herein refers to a linear or branched hydrocarbon group containing one or more carbon-carbon double bonds and having from x to y carbon atoms. Examples of such groups include ethenyl, propenyl, butenyl, pentenyl and hexenyl.

The term ‘C_{x-y} alkenylene’ as used herein refers to a divalent hydrocarbon group obtained by removing one hydrogen atom from ‘C_{x-y} alkenyl’ above. Examples of C₂₋₆ alkenylene groups include vinylene and propenylene.

The term ‘C_{x-y} alkoxy’ as used herein refers to an —O—C_{x-y} alkyl group wherein C_{x-y} alkyl is as defined herein. Examples of such groups include methoxy, ethoxy, propoxy, butoxy, pentoxy and hexoxy.

The term ‘C_{x-y} cycloalkyl’ as used herein refers to a saturated monocyclic hydrocarbon ring of x to y carbon atoms. For example, C₃₋₈ cycloalkyl refers to a saturated monocyclic hydrocarbon ring of 3 to 8 carbon atoms. Examples of C₃₋₈ cycloalkyl groups include cyclopropyl, cyclobutyl, cyclopentyl, cyclohexyl, cycloheptyl and cyclooctyl.

The term ‘C_{x-y} cycloalkylene’ as used herein refers to a divalent hydrocarbon group obtained by removing one hydrogen atom from ‘C_{x-y} cycloalkyl’ above. Examples of C₃₋₈ cycloalkylene groups include cyclopropylene and cyclobutylene.

The term ‘halogen’ as used herein refers to a fluorine, chlorine, bromine or iodine atom, unless otherwise specified.

The term ‘haloC₁₋₆ alkyl’ as used herein refers to a C₁₋₆ alkyl group as defined herein wherein at least one hydrogen atom is replaced with halogen. Examples of such groups include fluoroethyl, trifluoromethyl and trifluoroethyl.

The term ‘aryl’ as used herein refers to a C₆₋₁₂ monocyclic or bicyclic hydrocarbon ring wherein at least one ring is aromatic. Examples of such groups include phenyl, naphthyl and tetrahydronaphthalenyl.

The term ‘heteroaryl’ as used herein refers to a 5-6 membered monocyclic aromatic or a fused 8-10 membered bicyclic aromatic ring which monocyclic or bicyclic ring contains 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such bicyclic aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and imidazopyridyl.

The term ‘heterocyclyl’ refers to a 4-7 membered monocyclic ring or a fused 8-12 membered bicyclic ring which may be saturated or partially unsaturated, which monocyclic or bicyclic ring contains 1 to 4 heteroatoms selected from oxygen, nitrogen, silicon or sulphur. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl and azepanyl. Examples of such bicyclic rings include indolinyl, isoindolinyl, benzopyranyl, quinuclidinyl, 2,3,4,5-tetrahydro-1H-3-benzazepine and tetrahydroisoquinolinyl.

The term ‘N-containing-heterocyclyl’ refers to a ring containing at least one nitrogen atom and selected from among the ‘heterocyclyl’ groups mentioned above. Preferred examples of such rings include pyrrolidinyl, azetidinyl, piperidinyl, piperazinyl, morpholinyl and thiomorpholinyl.

‘Pharmaceutically acceptable salts’ of compounds of Formula I of the present invention include salts with inorganic bases, salts with organic bases, salts with inorganic acids, salts with organic acids and salts with basic or acidic amino acids. Salts with acids may, in particular, be employed in some instances. The compound of Formula I of the present invention may be in either hydrate or non-hydrate form.

‘Pharmaceutically acceptable esters’ of compounds of Formula I are derivatives in which one or more carboxyl (i.e. —C(O)OH) groups of the said compounds are modified by reaction with an alcoholic moiety W—OH so as to yield —C(O)OW groups, wherein W may be C₁₋₁₈ alkyl (e.g. C₁₋₆ alkyl), aryl, heteroaryl, C₃₋₈ cycloalkyl or combinations thereof.

General methods for the preparation of salts and esters are well known to the person skilled in the art. Pharmaceutical acceptability of salts and esters will depend on a variety of factors, including formulation processing characteristics and in vivo behaviour, and the skilled person would readily be able to assess such factors having regard to the present disclosure.

Where compounds of the invention exist in different enantiomeric and/or diastereoisomeric forms (including geometric isomerism about a double bond), these compounds may be prepared as isomeric mixtures or racemates, although the invention relates to all such enantiomers or isomers, whether present in an optically pure form or as mixtures with other isomers. Individual enantiomers or isomers may be obtained by methods known in the art, such as optical resolution of products or intermediates (for example chiral chromatographic separation (e.g. chiral HPLC)), or an enantiomeric synthesis approach. Similarly, where compounds of the invention may exist as alternative tautomeric forms (e.g. keto/enol, amide/imidic acid), the invention relates to the individual tautomers in isolation, and to mixtures of the tautomers in all proportions.

In particular embodiments of the first aspect of the invention, when A is NHCO, R1 is cyclobutyl, ethyl, n-propyl or isobutyl, n is 0 and X is absent, Z is not 1-[[5-chloro-2(2-methylpropoxy)phenyl]methyl]-5-methyl-1H-pyrazol-3-yl, and/or, when A is C₂ alkylene, R1 is CH₃, n is 0 and X is absent, Z is not N-(4-carboxycyclohexyl)-substituted imidazolidinonyl.

In certain embodiments of the first aspect of the invention: R1 is a group selected from C₃₋₈ cycloalkyl, C₁₋₆ alkyl, and C₃₋₈ cycloalkyl-substituted C₁₋₆ alkylene, each of which groups may optionally be substituted with halogen (such as F), haloC₁₋₆ alkyl (such as CF₃) or OR2;

n is 0, 1, 2, 3 or 4, the alkylene group —(CH₂)ₙ formed thereby being optionally substituted with a group selected from C₁₋₄ alkyl and C₃₋₈ cycloalkyl;
A is a group selected from —N(R2)CO—, —OC(O)—, —C(O)O—, —CON(R2)-, —CO—, —C(R2)(OR3)-, —C(═N—O—R3)-, —C(═CR2R3)-, —C₃₋₈ cycloalkylene-, —C(R2)(haloC₁₋₆ alkyl)- and —C(OR3)(haloC₁₋₆ alkyl)-;
R2 and R3 are each independently selected from H, C₁₋₆ alkyl (which may be straight- or branched-chain), and C₃₋₄ cycloalkyl, or, when A is —N(R2)CO— and X is absent, R2 may form, together with the adjacent nitrogen atom and Z, an N-containing heterocyclyl group, which may optionally be substituted;
X is absent or is C₁₋₄ alkylene or C₂₋₄ alkenylene, optionally substituted with one or more C₁₋₄ alkyl groups, OR2, halogen (such as F) or haloC₁₋₆alkyl (such as CF₃);
Z is selected from aryl, heteroaryl, C₃₋₈ cycloalkyl, and heterocyclyl, each of which may optionally be substituted by a group selected from —Y-aryl, —Y-heteroaryl, —Y—C₃₋₈ cycloalkyl and —Y-heterocyclyl, or, when X is present, Z may be H, or, when A is —N(R2)CO— and X is absent, Z may form, together with the adjacent nitrogen atom and R2, an N-containing heterocyclyl group which may optionally be substituted, provided that,
when A is —CO—, Z is linked to X or A via a carbon atom; and
Y represents a bond, C₁₋₆ alkylene, CO, CONH, COC₂₋₆ alkenylene, O, SO₂ or NHCOC₁₋₆ alkylene;
wherein said alkylene, cycloalkyl, aryl, heteroaryl and heterocyclyl groups of Z may be optionally substituted by one or more substituents which may be the same or different, and which are selected from halogen, haloC₁₋₆ alkyl, such as halomethyl, hydroxy, cyano, nitro, ═O, —R4, —CO₂R4, —COR4, —NR5R6, —C₁₋₆ alkyl-NR5R6, —C₃₋₈ cycloalkyl-NR5R6, —CONR5R6, —NR5CR6, —NR5SO₂R6, —OCONR5R6, —NR5CO₂R6, —NR4CONR5R6 or —SO₂NR5R6-SHR8, C₁₋₆ alkyl-OR8, —SOR8, —OR9, —SO₂R9, —OSO₂R9, C₁₋₆ alkyl-SO₂R9, C₁₋₆ alkyl-CONHR9, C₁₋₆ alkyl-SONHR9, C₁₋₆ alkyl-COR10, —CO—C₁₋₆ alkyl-R10, —O—C₁₋₆ alkyl-R11 (wherein R4, R5 and R6 independently represent hydrogen, C₁₋₆ alkyl, —C₃₋₈ cycloalkyl, —C₁₋₆ alkyl-C₃₋₄ cycloalkyl, aryl, heterocyclyl or heteroaryl, wherein R8 represents C₁₋₆ alkyl, wherein R9 represents C₁₋₆ alkyl or aryl, wherein R10 represents aryl, wherein R11 represents C₃₋₈ cycloalkyl or aryl, and wherein —NR5R6 may represent a nitrogen containing heterocyclyl group); wherein said R4, R5, R6, R8, R9, R10 and R11 groups may be optionally substituted by one or more substituents which may be the same or different, and which are selected from the group consisting of halogen, hydroxy, C₁₋₆ alkyl, C₁₋₆ alkoxy, cyano, amino, ═O or trifluoromethyl;
and wherein the provisos to the first aspect apply.

In additional embodiments of the first aspect of the invention, R1 is a group selected from C₃₋₈ cycloalkyl, C₁₋₆ alkyl, and C₃₋₈ cycloalkyl-substituted C₁₋₆ alkylene, each of which groups may optionally be substituted with halogen (such as F), haloC₁₋₆ alkyl (such as CF₃) or OR2;

n is 0, 1, 2, 3 or 4, the alkylene group —(CH₂)₆— formed thereby being optionally substituted with a group selected from C₁₋₄ alkyl and C₃₋₈ cycloalkyl;
A is a group selected from —N(R2)CO—, —OC(O)—, —C(O)O—, —CON(R2)-, —CO—, —C(R2)(OR3)-, —C(═N—O—R3)-, —C(═CR2R3)-, —C₃₋₈ cycloalkylene-, —C(R2)(haloC₁₋₆alkyl)-, C₁₋₄ alkylene and —C(OR3)(haloC₁₋₆alkyl)-;
R2 and R3 are each independently selected from H, C₁₋₆ alkyl (which may be straight- or branched-chain), and C₃₋₈ cycloalkyl, or, when A is —N(R2)CO— and X is absent, R2 may form, together with the adjacent nitrogen atom and Z, an N-containing heterocyclyl group, which may optionally be substituted;
X is absent or is C₁₋₄ alkylene or C₂₋₄ alkenylene, optionally substituted with one or more C₁₋₄ alkyl groups, OR2, halogen (such as F), or haloC₁₋₆ alkyl (such as CF₃);
Z is selected from aryl, heteroaryl, C₃₋₈ cycloalkyl, and heterocyclyl, each of which may optionally be substituted by a group selected from —Y-aryl, —Y-heteroaryl, —Y—C₃₋₈ cycloalkyl and —Y-heterocyclyl, or, when X is present, Z may be H, or, when X is absent and A is —C(R2)(OR3)- or —N(R2)CO—, Z may be H, or, when A is —N(R2)CO— and X is absent, Z may form, together with the adjacent nitrogen atom and R2, an N-containing heterocyclyl group which may optionally be substituted, provided that, when A is —CO—, Z is linked to X or A via a carbon atom; and
Y represents a bond, C₁₋₆ alkylene, CO, CONH, COC₂₋₆ alkenylene, O, SO₂ or NHCOC₁₋₆ alkylene;
wherein said cycloalkyl, aryl, heteroaryl and heterocyclyl groups of Z may be optionally substituted by one or more substituents which may be the same or different, and which are selected from halogen, haloC₁₋₆ alkyl, such as halomethyl, hydroxy, cyano, nitro, ═O, —R4, —CO₂R4, —COR4, —NR5R6, —C₁₋₆ alkyl-NR5R6, —C₃₋₈ cycloalkyl-NR5R6, —CONR5R6, —NR5CR6, —NR5SO₂R6, —OCONR5R6, —NR5CO₂R6, —NR4CONR5R6 or —SO₂NR5R6-SHR8, C₁₋₆ alkyl-OR8, —SOR8, —OR9, —SO₂R9, —OSO₂R9, C₁₋₆ alkyl-SO₂R9, C₁₋₆ alkyl-CONHR9, C₁₋₆ alkyl-SONHR9, C₁₋₆ alkyl-COR10, —CO—C₁₋₆ alkyl-R10, —O—C₁₋₆ alkyl-R11 (wherein R4, R5 and R6 independently represent hydrogen, C₁₋₆ alkyl, —C₃₋₈ cycloalkyl, —C₁₋₆ alkyl-C₃₋₈ cycloalkyl, aryl, heterocyclyl or heteroaryl, wherein R8 represents C₁₋₆ alkyl, wherein R9 represents C₁₋₆ alkyl or aryl, wherein R10 represents aryl, wherein R11 represents C₃₋₈ cycloalkyl or aryl, and wherein —NR5R6 may represent a nitrogen containing heterocyclyl group); wherein said R4, R5, R6, R8, R9, R10 and R11 groups may be optionally substituted by one or more substituents which may be the same or different, and which are selected from the group consisting of halogen, hydroxy, C₁₋₆ alkyl, C₁₋₆ alkoxy, cyano, amino, ═O or trifluoromethyl;
wherein substituents of Z selected from —Y-aryl, —Y-heteroaryl, —Y—C₃₋₈ cycloalkyl and —Y-heterocyclyl may be optionally substituted by one or more substituents selected from ═O, hydroxy, cyano, nitro, halogen, haloC₁₋₆ (alkyl such as halomethyl) and C₁₋₆ alkyl;
and wherein, when A is C₁₋₄ alkylene, said cycloalkyl, aryl, heteroaryl or heterocyclyl group Z (such as a heterocyclyl group Z) is substituted at least with hydroxy;
and wherein the provisos to the first aspect apply.

In certain compounds of the invention in which Z is selected from aryl, heteroaryl, C₃₋₈ cycloalkyl, heterocyclyl, —C₃₋₈ cycloalkyl-Y—C₃₋₈ cycloalkyl, —C₃₋₈ cycloalkyl-Y-aryl, —C₃₋₈ cycloalkyl-Y-heteroaryl, —C₃₋₈ cycloalkyl-Y-heterocyclyl, -aryl-Y—C₃₋₈ cycloalkyl, -aryl-Y-aryl, -aryl-Y-heteroaryl, -aryl-Y-heterocyclyl, -heteroaryl-Y—C₃₋₈ cycloalkyl, -heteroaryl-Y-aryl, -heteroaryl-Y-heteroaryl, -heteroaryl-Y-heterocyclyl, -heterocyclyl-Y—C₃₋₈ cycloalkyl, -heterocyclyl-Y-aryl, -heterocyclyl-Y-heteroaryl, and -heterocyclyl-Y-heterocyclyl, Z may be linked to A or X via a carbon atom (that is, a carbon atom of the group Z). When A is —CO—, such that Z is linked to A or X via a carbon atom, this means that Z is linked to A or X via a carbon atom of Z.

In certain embodiments of the invention, Y represents a bond or C₁₋₆ alkylene (e.g. methylene).

In certain embodiments of the invention, when A is —CO—, n is 0, R1 is C₁₋₆ alkyl (such as CH₃), and X is present (and may be C₁₋₄ alkyl, such as —CH₂CH₂—), Z is not a -heterocyclyl-Y-aryl-group containing a piperidinyl moiety. In particular embodiments within this group, Z is not -heterocyclyl-Y-aryl-.

In particular embodiments, Z may be H when A is —CONH-(or —CON(R2)-) or —NHCO-(or —N(R2)CO—).

In other embodiments, for example when A is —NHCO— (or —N(R2)CO—), and n is 0, Z may be any of the aryl, cycloalkyl, heterocyclyl or heteroaryl-containing moieties defined above, particularly moieties containing a combination of two or more such groups. Such groups of Z are, in certain instances, not substituted with halogen and/or alkoxy (such as butyloxy). In certain instances, when Z comprises said two or more such groups, one or none of the aryl, cycloalkyl, heterocyclyl or heteroaryl groups of Z is further substituted. In certain embodiments, Z is not aryl-Y-heteroaryl, particularly not aryl-CH₂-heteroaryl.

In particular compounds of the invention, A is selected from —N(R2)CO—, —OC(O)—, —C(O)O—, —CON(R2)-, —C(R2)(OR3)-, —C(═N—O—R3)-, —C(═CR2R3)-, —C₃₋₈ cycloalkylene-, CO—, C₁₋₄ alkylene, —C(R2)(haloC₁₋₆ alkyl)- and —C(OR3)(haloC₁₋₆ alkyl)-.

In those embodiments in which A is —N(R2)CO— and X is absent, with R2 forming, together with the adjacent nitrogen atom and Z, an N-containing heterocyclyl group, possible substituents on the N-containing heterocyclyl group include halogen and carbamoyl.

In certain compounds of the invention, R1 is C₁₋₆ alkyl (such as C₁₋₃ alkyl), C₃₋₄ cycloalkyl (such as C₃₋₇ cycloalkyl) or heterocyclyl (preferably tetrahydrofuranyl). Particular C₁₋₆ alkyl or C₃₋₄ cycloalkyl groups of R1 include methyl, ethyl, isopropyl, cyclopropyl, cyclobutyl and cyclopentyl.

Alternatively, R1 may be C₁₋₆ alkyl (such as C₁₋₃ alkyl) substituted with C₃₋₄ cycloalkyl (such as C₃₋₇ cycloalkyl). In such embodiments, R1 may, for example, be cyclopropylethyl or cyclopropylmethyl.

In any event, C₁₋₆ alkyl, C₃₋₄ cycloalkylene-C₁₋₆ alkyl and C₃₋₄ cycloalkyl for R1 may be further substituted with one or more (e.g. 1 to 3) groups selected from hydroxy, C₁₋₆ alkoxy (such as methoxy), C₁₋₆ alkyl (such as methyl), halogen (such as F or Cl) and haloC₁₋₆ alkyl, e.g. halomethyl (such as CH₂F), particularly selected from F and CH₂F.

In particular embodiments of the invention R1 is C₃₋₄ cycloalkyl substituted with hydroxy or methoxy.

In certain embodiments of the invention, n is 0, 1 or 2.

In particular embodiments n is 0.

In particular embodiments n is 1.

In certain embodiments of the invention, A is —N(R2)CO—, —OC(O)—, —CON(R2)-, —CO—, —C(R2)(OR3)-, —C(═N—O—R3)- or —C(═CR2R3)-.

In particular embodiments of the invention, A is —C(R2)(OR3)-, C₁₋₄alkylene, —N(R2)CO—, or —CON(R2)-.

In certain embodiments of the invention, A is —C(R2)(OR3)-.

In certain embodiments of the invention, A is —CON(R2)-.

In particular embodiments of the invention, n is 0 and A is —N(R2)CO— (such as —NHCO—) in particular —N(R2)CO—. In other embodiments, n is 0 and A is —C(R2)(OR3)- (such as —C(R2)OH—).

In other particular embodiments of the invention, n is 1 and A is —N(R2)CO— (such as —NHCO—) or —CON(R2)- (such as —CONH—), in particular —CON(R2)-. In other embodiments, n is 1 and A is —C(R2)(OR3)- (such as —C(R2)OH—).

In certain compounds of the invention, R2 and R3 are each independently H or C₁₋₆ alkyl, such as methyl. In particular embodiments, R2 is H. In particular embodiments, R3 is H.

In certain embodiments of the invention, A is —NHCO—, —N(Me)CO—, —OC(O)—, —CONH—, —CO—, —CH(OH)—, —CH(OMe)-, —C(═N—O-Me)-, —C(═N—O—H)—, —CH₂— or —C(═CH₂)—.

In certain compounds of the invention, where A is —C(R2)(haloC₁₋₆ alkyl)- or —C(OR3)(haloC₁₋₆ alkyl), the group (haloC₁₋₆ alkyl) may be a fluorinated alkyl, such as CF₃.

In certain embodiments of the invention, X is absent or is C₁₋₄ alkylene (e.g., methylene, ethylene) or C₂₋₄ alkenylene (e.g., vinylene), each of which may optionally be substituted with a C₁₋₄ alkyl group (e.g., methyl).

In particular embodiments, X is a C₁₋₄ alkylene group, (preferably straight chain), optionally having one or more (e.g. 1 to 3) methyl or ethyl substituents. In certain compounds, X is methylene or ethylene.

In certain embodiments of the invention, when A is —N(R2)CO— and X is absent, R2 may form, together with the adjacent nitrogen atom and Z, an N-containing heterocyclyl group (e.g., azetidinyl, pyrrolidinyl, piperidinyl, piperazinyl, morpholinyl) which may optionally be substituted by one to three halogen atoms (e.g., F) or carbamoyl groups.

In certain compounds of the present invention, Z is aryl, heteroaryl, C₃₋₈ cycloalkyl or heterocyclyl, each of which may be substituted with one or more (e.g. 1 to 3) substituents selected from C₁₋₆ alkyl (such as methyl, ethyl or isopropyl), C₁₋₆ cycloalkyl (such as cyclobutyl), halogen (such as Cl, Br or F), haloC₁₋₆ alkyl (such as halomethyl (e.g. trifluoromethyl)), cyano, amino, C₁₋₆ alkoxy (such as methoxy), carbonyl (such as C₁₋₆ alkyl-carbonyl (e.g. acetyl), carboxyl, C₁₋₆ alkoxy-carbonyl (e.g. methoxycarbonyl)), amido (such as carbamoyl, C₁₋₆ alkyl-carbamoyl (e.g. methylcarbamoyl)), heterocyclyl-amino (e.g. cyclobutylamino, cyclopropylamino), aryl (such as phenyl), and heteroaryl (such as triazolyl, thiazolyl, pyrazolyl, thiophenyl, pyrrolidinyl, morpholinyl or pyridinyl).

In embodiments in which Z is, or comprises, heteroaryl, said heteroaryl may be selected from thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl, quinolinyl, isoquinolinyl, quinazolinyl, quinoxalinyl, pteridinyl, cinolinyl, phthalazinyl, naphthyridinyl, indolyl, isoindolyl, azaindolyl, indolizinyl, indazolyl, purinyl, pyrrolopyridinyl, fluoropyridinyl, benzofuranyl, isobenzofuranyl, benzothienyl, benzimidazolyl, benzoxazolyl, benzoisoxazolyl, benzothiazolyl, benzoisothiazolyl, benzoxadiazolyl, benzothiadiazolyl and imidazopyridyl. In particular, said heteroaryl may be selected from pyrazolyl, pyridyl, pyrimidyl, pyridazinyl, pyrazinyl, indolyl, indazolyl, benzimidazolyl, benzothiazolyl, imidazopyridyl, imidazolyl, isoxazolyl, thienyl, oxazolyl, thiazolyl, furyl, imidazopyridyl and pyrrolyl groups.

In embodiments in which Z is, or comprises, aryl, said aryl may in particular be a phenyl, naphthyl, or tetrahydronaphthalenyl group, in particular a phenyl group.

In embodiments in which Z is, or comprises, heterocyclyl, said heterocyclyl may be selected from pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl, indolinyl, isoindolinyl, benzopyranyl, quinuclidinyl, 2,3,4,5-tetrahydro-1H-3-benzazepinyl and tetrahydroisoquinolinyl. In particular, said heterocyclyl may be selected from piperidinyl, azetidinyl, tetrahydrofuranyl, pyrrolidinyl, dioxanyl, tetrahydrothiopyranyl, tetrahydrothiophenyl, morpholinyl and tetrahydropyranyl groups.

In embodiments in which Z is, or comprises, C₃₋₈ cycloalkyl, said C₃₋₈ cycloalkyl may in particular be selected from cyclopropyl, cyclobutyl, cyclopentyl, cyclohexyl, cycloheptyl and cyclooctyl groups, more particularly from cyclobutyl, cyclopentyl and cyclohexyl groups.

In particular embodiments, Z is aryl (preferably phenyl), heteroaryl (preferably pyrazolyl, pyridyl, indolyl, indazolyl, benzimidazolyl, benzothiazolyl, imidazopyridyl, imidazolyl, isoxazolyl, thienyl, oxazolyl, thiazolyl, furyl, pyridazinyl, pyrimidyl, pyrazinyl or pyrrolyl), cycloalkyl (preferably cyclobutyl, cyclopentyl or cyclohexyl) or heterocyclyl (preferably piperidinyl, tetrahydrofuranyl, morpholinyl, pyrrolidinyl, tetrahydrothiophenyl, piperazinyl, tetrahydrothiopyranyl, dioxanyl or tetrahydropyranyl),

each of which may optionally be substituted by
(1) a group selected from —Y-aryl, —Y-heteroaryl, Y-heterocyclyl, and —Y—C₃₋₈ cycloalkyl,
wherein Y represents a bond, O, NR14 or C₁₋₆ alkylene (preferably methylene), and said aryl is phenyl, said heteroaryl is selected from triazolyl, thiazolyl, thienyl and pyrazolyl, said heterocyclyl is selected from morpholinyl, tetrahydrofuranyl, tetrahydropyranyl and pyrrolidinyl, and said C₃₋₈ cycloalkyl is selected from cyclobutyl or cyclopropyl; or
(2) one to three substituents selected from
C₁₋₆ alkyl (preferably methyl, ethyl or isopropyl), C₃₋₈ cycloalkyl (preferably cyclobutyl) halogen (preferably F, Cl or Br), haloC₁₋₆ alkyl (preferably trifluoromethyl), cyano, hydroxy, amino, C₁₋₆ alkoxy (preferably methoxy, ethoxy or isopropoxy), C₁₋₆ alkyl-carbonyl (preferably acetyl), carboxyl, C₁₋₆ alkoxy-carbonyl (preferably methoxycarbonyl), carbamoyl, hydroxy-substituted C₁₋₆ alkyl-carbonyl, C₃₋₈ cycloalkyl-carbonyl, C₁₋₆ alkyl-carbamoyl (preferably methylcarbamoyl), C₁₋₆ alkylamino (preferably methylamino), and ═O,
wherein substituents of Z selected from —Y-aryl, —Y-heteroaryl, —Y—C₃₋₈ cycloalkyl and —Y-heterocyclyl may be optionally substituted by one or more substituents selected from ═O, hydroxy, cyano, nitro, halogen, haloC₁₋₆ alkyl (such as halomethyl) and C₁₋₆ alkyl.

In certain embodiments Y is a bond or C₁₋₆ alkylene; in particular embodiments, Y is a bond.

In certain compounds of the present invention, Z is H when X is present.

In certain embodiments of the first aspect of the invention, A is —N(R2)CO— or —CON(R2)-, and n is 0, 1 or 2.

In certain such embodiments, R2 is H. In particular embodiments, A is —CON(R2)-.

In alternative embodiments of the first aspect of the invention, A is —OC(O)— or —C(O)O—, and n is 0, 1 or 2.

In other embodiments of the first aspect of the invention, A is —C(R2)(OR3)- or —CO—, and n is 0, 1 or 2. In certain such embodiments, R2 and/or R3 are H or C₁₋₆ alkyl, such as methyl.

Particular embodiments of the first aspect of the invention include compounds wherein: R1 is C₁₋₆ alkyl (preferably methyl, ethyl, isopropyl or isobutyl), C₃₋₈ cycloalkyl-C₁₋₆ alkylene- (preferably cyclopropylmethyl), or C₃₋₈ cycloalkyl (preferably cyclobutyl or cyclopentyl), each of which may optionally be substituted by hydroxy, C₁₋₆ alkoxy, or one or two halogens (preferably F), or R1 is heterocyclyl (preferably tetrahydrofuranyl), optionally substituted by hydroxy, C₁₋₆ alkoxy, or C₁₋₆ alkyl (such as methyl);

n is 0, 1 or 2;
A is —N(R2)CO—, —OC(O)—, —CON(R2)-, —CO—, —C(R2)(OR3)-, C₁₋₄ alkylene, —C(═N—O—R3)- or —C(═CHR3)-;
R2 and R3 are each independently H or C₁₋₆ alkyl (preferably methyl);
or, when A is —N(R2)CO— and X is absent, R2 may form, together with the adjacent nitrogen atom and Z, an N-containing heterocyclyl group (optionally azetidinyl, pyrrolidinyl, piperidinyl, piperazinyl or morpholinyl, and preferably azetidinyl, pyrrolidinyl, piperidinyl, or morpholinyl) which may optionally be substituted by one to three halogen atoms (preferably F), alkyl carbonyl or carbamoyl groups;
X is absent or is C₁₋₄ alkylene (preferably methylene or ethylene) or C₂₋₄ alkenylene (preferably vinylene), each of which may optionally be substituted with a C₁₋₄ alkyl group (preferably methyl); and
Z is aryl (preferably phenyl), heteroaryl (preferably pyrazolyl, pyridyl (such as 3-pyridyl), indolyl, indazolyl, benzimidazolyl, benzothiazolyl, imidazopyridyl, imidazolyl, isoxazolyl, thienyl, oxazolyl, thiazolyl, furyl, pyridazinyl, pyrimidyl, pyrazinyl or pyrrolyl), C₃₋₈ cycloalkyl (preferably cyclobutyl, cyclopentyl or cyclohexyl) or heterocyclyl (preferably piperidinyl, tetrahydrofuranyl, morpholinyl, pyrrolidinyl, tetrahydrothiophenyl, piperazinyl, tetrahydrothiopyranyl, dioxanyl, or tetrahydropyranyl),
each of which may optionally be substituted by
(1) a group selected from —Y-aryl, —Y-heteroaryl, Y-heterocyclyl, and —Y—C₃₋₈ cycloalkyl,
wherein Y represents a bond, O, NR14, or C₁₋₆ alkylene (preferably methylene), and said aryl is selected from phenyl, said heteroaryl is selected from triazolyl, thiazolyl, thienyl and pyrazolyl, said heterocyclyl is selected from morpholinyl, tetrahydrofuranyl, tetrahydropyranyl and pyrrolidinyl, and said C₃₋₈ cycloalkyl is selected from cyclobutyl and cyclopropyl; or
(2) one to three substituents selected from
C₁₋₆ alkyl (preferably methyl, ethyl or isopropyl), halogen (preferably F, Cl or Br), haloC₁₋₆ alkyl (preferably trifluoromethyl), cyano, hydroxy, amino, C₁₋₆ alkoxy (preferably methoxy, ethoxy or isopropoxy), C₁₋₆ alkyl-carbonyl (preferably acetyl), hydroxy-substituted C₁₋₆ alkyl-carbonyl, C₃₋₈ cycloalkyl-carbonyl, carboxyl, C₁₋₆ alkoxy-carbonyl (preferably methoxycarbonyl), carbamoyl, C₁₋₆ alkyl-carbamoyl (preferably methylcarbamoyl), C₁₋₆ alkylamino (such as methylamino), and ═O; or
Z may be H when X is present, or Z may be H when X is absent and A is —C(R2)(OR3)- or —N(R2)CO—,
wherein substituents of Z selected from —Y-aryl, —Y-heteroaryl, —Y—C₃₋₈cycloalkyl and —Y-heterocyclyl may be optionally substituted by one or more substituents selected from ═O, hydroxy, cyano, nitro, halogen, haloC₁₋₆ alkyl such as halomethyl and C₁₋₆ alkyl,
wherein, when A is C₁₋₄ alkylene, said cycloalkyl, aryl, heteroaryl or heterocyclyl group Z (such as a heterocyclyl group Z) is substituted at least with hydroxy, CF₃ or ═O;
and wherein, when A is CON(R2), n is 1.

Particular embodiments of the first aspect of the invention include compounds wherein: R1 is C₁₋₆ alkyl (preferably methyl, ethyl) or C₃₋₈ cycloalkyl (preferably cyclobutyl or cyclopentyl);

n is 1;

### A is —C(R2)(OR3)-;

R2 and R3 are each independently H or C₁₋₆ alkyl (preferably methyl);
X is absent or is C₁₋₄ alkylene (preferably methylene);
Z is heteroaryl (preferably pyridyl), or heterocyclyl (preferably piperidinyl, or tetrahydropyranyl), each of which may optionally be substituted by one to three substituents selected from C₁₋₆ alkyl (preferably methyl, ethyl or isopropyl), halogen (preferably Cl or Br), haloC₁₋₆ alkyl (preferably trifluoromethyl), cyano, hydroxy, amino, C₁₋₆ alkoxy (preferably methoxy), C₁₋₆ alkyl-carbonyl (preferably acetyl or propionyl), hydroxy-substituted C₁₋₆ alkyl-carbonyl, carboxyl, C₁₋₆ alkoxy-carbonyl (preferably methoxycarbonyl), C₃₋₈ cycloalkyl-carbonyl, carbamoyl, C alkyl-carbamoyl (preferably methylcarbamoyl).

Further embodiments of the first aspect of the invention include compounds wherein: R1 is C₁₋₆ alkyl (preferably methyl or ethyl) or C₃₋₈ cycloalkyl (preferably cyclobutyl or cyclopentyl);

n is 0;
A is C₁₋₄ alkylene (preferably methylene);
R2 and R3 are each independently H or C₁₋₆ alkyl (preferably methyl);
X is absent or is C₁₋₄ alkylene (preferably methylene);
Z is heteroaryl (preferably pyridyl or pyrrolopyridinyl), or heterocyclyl (preferably piperidinyl, pyrrolidinyl or tetrahydropyranyl),
each of which may optionally be substituted by one to three substituents selected from
C₁₋₆ alkyl (preferably methyl, ethyl or isopropyl), halogen (preferably Cl or Br), haloC₁₋₆ alkyl (preferably trifluoromethyl), cyano, hydroxy, amino, C₁₋₆ alkoxy (preferably methoxy), C₁₋₆ alkyl-carbonyl (preferably acetyl or propionyl), hydroxy-substituted C₁₋₆ alkyl-carbonyl, carboxyl, C₁₋₆ alkoxy-carbonyl (preferably methoxycarbonyl), C₃₋₈ cycloalkyl-carbonyl, carbamoyl, C₁₋₆ alkyl-carbamoyl (preferably methylcarbamoyl).
wherein said heteroaryl or heterocyclyl group Z (such as a heterocyclyl group Z) is substituted at least with hydroxy, CF₃ or ═O.

In particular compounds defined according to the preceding two paragraphs, R1 is C₃₋₈ cycloalkyl (preferably cyclobutyl). In certain embodiments, Z is heterocyclyl (such as piperidinyl, pyrrolidinyl or tetrahydropyranyl), which may be substituted with one to three (preferably 1 or 2) of the carbonyl-containing substituents defined in the said paragraphs.

Alternative particular embodiments of the first aspect of the invention include compounds wherein: R1 is C₁₋₆ alkyl (preferably methyl or ethyl), C₃₋₈ cycloalkyl (preferably cyclobutyl or cyclopentyl), optionally substituted with halogen (such as F) or C₁₋₆ alkoxy (such as methoxy), or R1 is heterocyclyl (preferably tetrahydrofuranyl), optionally substituted with C₁₋₆ alkyl;

n is 1;

### A is —CON(R2)- or —N(R2)CO—;

R2 is selected from H and C₁₋₆ alkyl (preferably methyl or isobutyl);
X is absent or is C₁₋₄ alkylene (preferably methylene, ethylene, propylene, isopropylene, t-butylene or isobutylene), which may be optionally substituted with one or more C₁₋₄ alkyl (such as methyl) or hydroxy groups;
Z is aryl (preferably phenyl), heteroaryl (preferably pyrazolyl, pyridyl (such as 3-pyridyl), pyrrolyl, isoxazolyl, pyridazinyl, pyrimidyl, pyrazinyl, thiazolyl, oxazolyl or furyl), C₃₋₈ cycloalkyl (preferably cyclohexyl) or heterocyclyl (preferably piperidinyl, tetrahydrofuranyl, morpholinyl, pyrrolidinyl, tetrahydrothiophenyl, tetrahydropyridyl, piperazinyl, tetrahydrothiopyranyl, dioxanyl or tetrahydropyranyl),
each of which may optionally be substituted by
(1) a group selected from —Y-aryl, —Y-heteroaryl, Y-heterocyclyl, and —Y—C₃₋₈ cycloalkyl,
wherein Y represents a bond, O, NR14 (such as NH), or C₁₋₆ alkylene (preferably methylene), and said aryl is selected from phenyl, said heteroaryl is selected from triazolyl, thiazolyl, thienyl and pyrazolyl, said heterocyclyl is selected from morpholinyl, tetrahydrofuranyl, tetrahydropyranyl and pyrrolidinyl, and said C₃₋₈ cycloalkyl is selected from cyclobutyl and cyclopropyl; or
(2) one to three substituents selected from
C₁₋₆ alkyl (preferably methyl, ethyl or isopropyl), halogen (preferably F, Cl or Br), haloC₁₋₆ alkyl (preferably trifluoromethyl), cyano, amino, C₁₋₆ alkylamino (such as methylamino), N,N—C₁₋₆ dialkylamino (such as hydroxpropyl(methyl)amino) C₁₋₆ alkoxy (preferably methoxy, ethoxy or isopropoxy), C₁₋₆ alkyl-carbonyl (preferably acetyl), carboxyl, C₁₋₆ alkoxy-carbonyl (preferably methoxycarbonyl), carbamoyl, C₁₋₆ alkyl-carbamoyl (preferably methylcarbamoyl), hydroxy C₁₋₆ alkyl and ═O; or
Z may be H when X is present,
wherein substituents of Z selected from —Y-aryl, —Y-heteroaryl, cycloalkyl and —Y-heterocyclyl may be optionally substituted by one or more substituents selected from ═O, hydroxy, cyano, nitro, halogen (such as F), haloC₁₋₆ alkyl (such as halomethyl) and C₁₋₆ alkyl.

In particular compounds defined according to the preceding paragraph, R1 is C₃₋₈ cycloalkyl (preferably cyclobutyl). In certain compounds, A is —CON(R2)-. In certain embodiments, Z is heteroaryl (such as pyridazinyl or pyridyl), which may be substituted with one to three (preferably 1 or 2) substituents selected from —Y-heterocyclyl (such as morpholinyl or pyrrolidinyl), C₁₋₆ alkoxy (such as methoxy) and C₁₋₆ alkylamino (such as methylamino).

In accordance with a second aspect of the invention, there is provided a pharmaceutical composition comprising a compound according to the first aspect of the invention, together with one or more pharmaceutically acceptable excipients.

Pharmaceutical compositions of this invention comprise any of the compounds of the first aspect of the present invention, or pharmaceutically acceptable salts and esters thereof, with any pharmaceutically acceptable carrier, adjuvant or vehicle. Pharmaceutically acceptable carriers, adjuvants and vehicles that may be used in the pharmaceutical compositions of this invention are those conventionally employed in the field of pharmaceutical formulation, and include, but are not limited to, sugars, sugar alcohols, starches, ion exchangers, alumina, aluminium stearate, lecithin, serum proteins, such as human serum albumin, buffer substances such as phosphates, glycerine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, rectally, nasally, buccally, vaginally or via an implanted reservoir. Oral administration is preferred. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as that described in Ph. Helv, or a similar alcohol.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

The compounds of the present invention may be administered in a dose of around 1 to around 20,000 μg/kg per dose, depending on the condition to be treated or prevented, and the characteristics of the subject being administered with the compound. In many instances, the dose may be around 1 to around 1500 μg/kg per dose. The dosing regimen for a given compound could readily be determined by the skilled person having access to this disclosure.

In one particular embodiment, the pharmaceutical composition of the invention additionally comprises one or more additional active pharmaceutical ingredients. These additional active ingredients may be agents known to the skilled person to be useful in the treatment or prevention of the diseases mentioned in the present disclosure.

In a third aspect, the present invention provides a compound according to the first aspect of the invention, or a composition according to the second aspect, for use in therapy.

In a fourth aspect, the invention provides a compound according to the first aspect of the invention, or a composition according to the second aspect, for use in the treatment or prevention of a condition whose development or symptoms are linked to histamine H3 receptor activity, wherein the provisos to the first aspect do not apply.

In certain embodiments of the fourth aspect, the first, second, third, and/or fourth provisos to the first aspect do apply.

A number of conditions whose development or symptoms are linked to histamine H3 receptor activity are known to the skilled person.

In a fifth aspect, the invention also provides a method of treatment or prevention of a condition whose development or symptoms are linked to histamine H3 receptor activity, the method comprising the administration, to a subject in need of such treatment or prevention, of a therapeutically effective amount of a compound according to the first aspect of the invention, or a composition according to the second aspect, wherein the provisos to the first aspect do not apply.

In certain embodiments of the fifth aspect, the first, second, third, and/or fourth provisos to the first aspect do apply.

A compound according to the fourth aspect, or a method according to the fifth aspect, wherein the condition is a disorder of the central nervous system.

In certain embodiments, the condition to be treated may be selected from sleep disorders (such as narcolepsy), cognitive disorders (such as dementia), attentional disorders (such as attention deficit hyperactivity disorder), neurodegenerative disorders (such as AD), schizophrenia, epilepsy, pain (such as neuropathic pain) and obesity.

In preferred embodiments the condition may be selected from schizophrenia, Alzheimer's Disease (AD) and dementia. In an alternative embodiment, the condition may be selected from narcolepsy, pain and obesity.

In particular embodiments, the condition may be selected from narcolepsy, neuropathic pain and obesity.

In a sixth aspect, the present invention provides an intermediate compound having the formula:

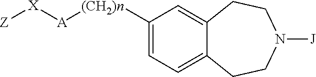

wherein n, A, X and Z have the same meaning as in Formula 1 above, or Z—X-A- together represents C₁₋₆ alkylsulfonyloxy, nitro, halogen (such as Br), carbaldehyde O—C₁₋₆ alkyl oxime, amino, amino attached to an amino protecting group or arylsulfonyl, and wherein J is an amino protecting group or H, provided that Z is linked to X or A via a carbon atom when Z contains a piperazinyl moiety, and provided that:
when A is —OC(O)—, J is H, n is 0 and X is —CH₂CH₂—, Z is not H;
when A is —OC(O)—, J is tert-butoxycarbonyl, n is 0 and X is —CH₂—, Z is not H;
when A is —NHCO—, J is tert-butoxycarbonyl, n is 0 and X is -isopropyl, Z is not H; and
when A is —NHCO—, J is tert-butoxycarbonyl or H, n is 0 and X is —CH₂— or —CH₂CH₂—, Z is not pyrrolidin-2-yl substituted with oxo, phenylpropyl and acetic acid substituents.

In particular embodiments of the sixth aspect, A, X znd Z have the same meaning as in Formula I above. In certain embodiments of the sixth aspect, Z is linked to X or A via a carbon atom (i.e. regardless of whether Z contains a piperazinyl moiety).

In certain embodiments of the sixth aspect, when A is —NHCO, J is H, n is 0 and X is absent, Z is not 1-[[5-chloro-2(2-methylpropoxy)phenyl]methyl]-5-methyl-1H-pyrazol-3-yl.

A preferred amino protecting group is tert-butoxycarbonyl (Boc), although many other protecting groups will be known to those skilled in the art. The methods of addition and removal of such protecting groups are those which would conventionally be used in relation to the particular molecule-type or group being protected, for example the methods described in standard works of reference in synthetic methodology, such as Kocienski (2004) Protecting Groups. 4th Edn. Georg Thieme Verlag.

In a seventh aspect, the present invention also provides an intermediate compound having the formula:

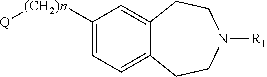

wherein n and R1 have the same meaning as in Formula I above, and wherein Q is selected from cyano, amino, amino attached to an amino protecting group (such as t-butyloxycarbonyl), arylsulfonyl (such as phenylsulfonyl) and halogen (such as Br).

In a related eighth aspect, the invention also provides the use of an intermediate compound according to the sixth or seventh aspects in the synthesis of a compound according to the first aspect, wherein the provisos specified in the sixth aspect do not apply. In certain embodiments of the eighth aspect, one or more of the provisos specified in the sixth aspect do apply.

It will be appreciated by the skilled person that, following removal of the protecting group (where present) on the intermediate, the nitrogen of the azepine ring becomes available for nucleophilic attack on a suitable R1-containing reagent (for example, in an alkylation or reductive amination reaction).

In a ninth aspect, the present invention provides the use of a compound according to the first aspect of the invention in the preparation of a medicament for the treatment or prevention of a condition whose development or symptoms are linked to histamine H3 receptor activity, wherein the provisos to the first aspect do not apply.

Exemplary conditions of relevance to the ninth aspect are mentioned above.

In a tenth aspect, the present invention also provides a method of synthesis of a compound according to the first aspect, wherein A is —N(R2)CO—, the method comprising the reaction of an intermediate having the formula:

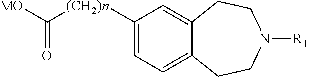

with an amine (Z—X)(R2)NH in the presence of a catalyst, wherein n, Z, X, R1 and R2 have the same meaning as given in relation to the first aspect, and wherein M represents H or a monovalent metal cation.

In certain embodiments of the tenth aspect, M is a monovalent metal cation, such as Li. The catalyst may be thionyl chloride, such that the reaction proceeds via the creation of an acyl chloride intermediate. The acyl chloride may, if necessary, be isolated before introduction of (Z—X)(R2)NH.

In an eleventh aspect, the present invention provides a method of synthesis of a compound according to the first aspect, wherein A is —CO— or —C(R2)(OR3)- and X is present, the method comprising the reaction of a protected intermediate:

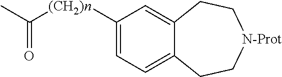

with an aldehyde Z—CHO in the presence of a catalyst, followed by deprotection of the protected amine and substitution thereof with R1 and, optionally, by catalytic hydrogenation, wherein n, Z, X, R1, R2 and R3 have the same meaning as given in relation to the first aspect, and wherein Prot represents an amine protecting group.

The catalyst may be, for example, 1,8-Diazabicyclo[5.4.0]undec-7-ene (DBU). In compounds in which X is alkenylene, the catalytic hydrogenation step may be omitted. Such a step (e.g. using H₂ gas in the presence of Pt(IV)O₂) saturates the double bond which results from the reaction between Z—CHO and the protected intermediate. Suitable amine protecting groups are described above.

The invention will now be described in more detail by way of example only.

### 1. SYNTHETIC METHODOLOGIES

The methods used for synthesis of the compounds of the invention are illustrated by the general schemes below and the preparative examples that follow. All compounds and intermediates were characterised at least by liquid chromatography-mass spectroscopy (LCMS). The starting materials and reagents used in preparing these compounds are available from commercial suppliers. These general schemes are merely illustrative of methods by which the compounds of this invention can be synthesised, and various modifications to these schemes can be made and will be suggested to one skilled in the art having referred to this disclosure.

Nuclear magnetic resonance (NMR) spectra were recorded at 400 MHz,; the chemical shifts (5) are reported in parts per million.

Mass spectra were recorded using an LCMS system (ZQ mass spec detector).

Compounds were purified using normal phase chromatography on silica or alumina, or by reverse phase chromatographic methods.

Room temperature in the following schemes means the temperature ranging from 20° C. to 25° C.

The desired compounds of Formula 1 can be prepared as outlined in Schemes 1-16, as follows:

### LIST OF ABBREVIATIONS

### Ac Acetyl

### AcOH Acetic Acid

### (Boc)₂O Boc Anhydride

### DCM Dichloromethane

### DMSO Dimethyl Sulfoxide

### DMF Dimethyl Formamide

### EtOAc Ethyl Acetate

### EtOH Ethanol

### HCl Hydrochloric Acid

H₂SO₄ Sulfuric acid

### IPE Di-isopropyl Ether

### KOH Potassium Hydroxide

### LCMS Liquid Chromatography Mass Spectrum

### MgSO₄ Magnesium Sulfate

### MS Mass Spectrum

### MeOD Deuterated Methanol

### MeOH Methanol

### Min Minute

NaOH Sodium hydroxide

### NMR Nuclear Magnetic Resonance

### RT Room Temperature

### THF Tetrahydrofuran

### TLC Thin Layer Chromatography

In the following schemes, Rₐ represents R1, R_{b} and R_{c} independently represent R2 or R3, or R_{b} and R_{c} represent X—Z, and R_{d} represents X—Z, wherein R1, R2, R3, X and Z are as defined above.

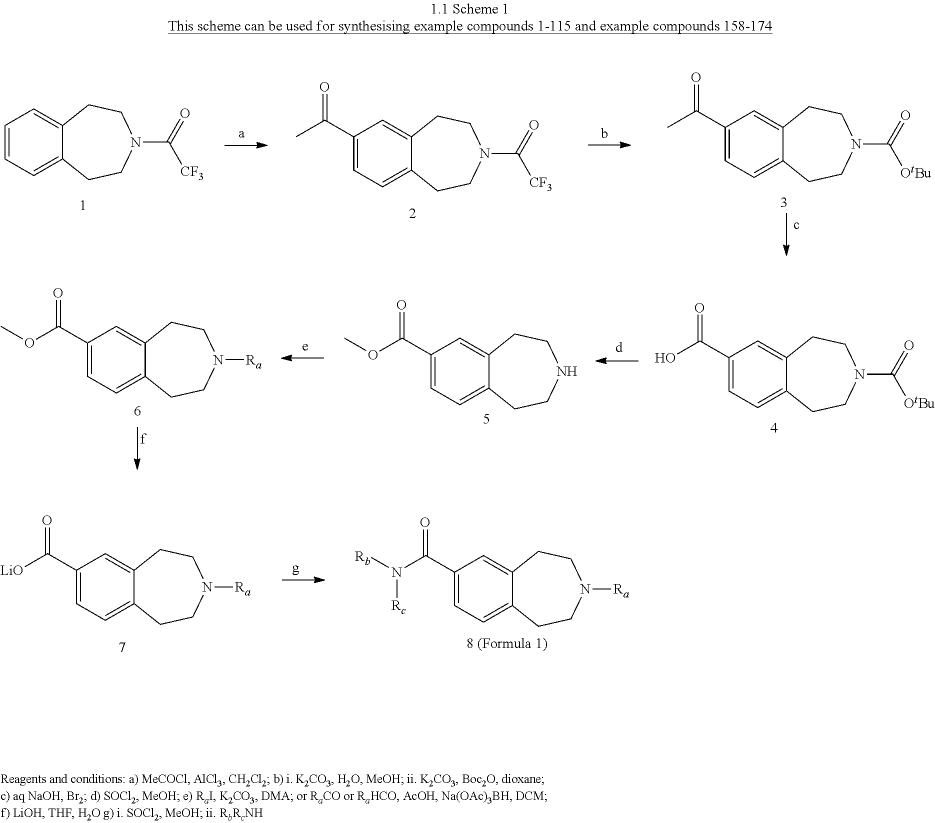

### 1.1.1 The Benzazepine Intermediate

(1) can be prepared by methods outlined in WO 2005/058328 and WO 2005/094834. The alkanoyl benzazepine (2) can be prepared from the corresponding (1) by Friedel-Crafts acylation as outlined in US 2005/20616. Removal of the trifluoroacetyl group of (2) under basic conditions followed by protection of the benzazepine nitrogen as a t-butyl carbamate using standard conditions well known in the art (for example, Bioorg. Med. Chem. 13 (2005) 1901-1911) gave intermediate (3). Modification of the acyl group using standard literature conditions (for example US2003/207863) gave the carboxylic acid intermediate (4). The carboxylic acid of (4) can be converted to a methyl ester using well known conditions. These conditions may also remove the t-butyl oxy carbonyl protecting group. The t-butyl oxy carbonyl protecting group can also be removed using other standard conditions well known in the art such as treatment with trifluoroacetic acid. Alkylation of the benzazepine nitrogen can be done using well known standard conditions of reductive amination or by use of alkyl halides. Further modifications using standard conditions well known in the art for saponification of the ester, conversion of the resulting acid into the acid chloride with subsequent amide formation furnishes compounds of formula 1.

### 1.1.2 Intermediate 2

To a solution of 1-(4,5-dihydro-1H-benzo[d]azepin-3(2H)-yl)-2,2,2-trifluoroethanone (15.5 g, 63.7 mmol) (1) and acetyl chloride (10 eq, 50 g, 637 mmol) in dichloromethane (300 ml) was added aluminium chloride (34 g, 255 mmol) portion-wise. The reaction was allowed to stir at room temperature for 1 hour. The reaction was quenched by pouring into a mixture of ice and saturated sodium hydrogen carbonate solution. Further solid sodium hydrogen carbonate was then added until the aqueous solution became basic. The reaction mixture was then filtered through celite and diluted with dichloromethane. The combined organics were then dried over sodium sulphate and concentrated in vacuo to give 1-(7-acetyl-4,5-dihydro-1H-benzo[d]azepin-3(2H)-yl)-2,2,2-trifluoroethanone (2) as a dark pink solid in 60 mmol.

MS ES⁺ : 286

1H NMR (400 MHz, DMSO-d6): δ 7.72-7.82 (m, 2H), 7.29-7.39 (m, 1H), 3.72 (br. s., 4H), 3.02-3.16 (m, 4H), 2.56 (s, 3H)

### 1.1.3 Intermediate 3

To a suspension of 1-(7-acetyl-4,5-dihydro-1H-benzo[d]azepin-3(2H)-yl)-2,2,2-trifluoroethanone (2) (10 g, 35 mmol) in methanol (496 ml) was added water (164 ml) and saturated potassium carbonate solution (164 ml) and the reaction allowed to stir at room temperature for 16 hours. The reaction mixture was concentrated in vacuo and the residue partitioned between ethyl acetate and water. The aqueous layer was washed with ethyl acetate and the combined organics washed with brine and dried over magnesium sulphate. The residue was azeotroped with toluene and the material used directly.

MS ES⁺ : 190

1H NMR (400 MHz, DMSO-d6): δ 7.64-7.70 (m, 2H), 7.20-7.26 (m, 1H), 2.86-2.93 (m, 4H), 2.73-2.80 (m, 4H), 2.53 (s, 3H)).

To a solution of 1-(2,3,4,5-tetrahydro-1H-benzo[d]azepin-7-yl)ethanone (10.44 g, 35 mmol) in dioxane (93 ml) and water (35 ml) was added Boc anhydride (7.64 g, 35 mmol) and potassium carbonate (4.84 g, 35 mmol) and the reaction allowed to stir at room temperature for 48 hours. The reaction mixture was concentrated in vacuo and partitioned between ethyl acetate and aqueous saturated sodium hydrogen carbonate solution. The combined organics were washed with brine and dried over magnesium sulphate. The crude oil was crystallised over night to give tert-butyl 7-acetyl-4,5-dihydro-1H-benzo[d]azepine-3(2H)-carboxylate (3) as a cream solid in 16.8 mmol.

MS ES⁺ : 189, ES: 216

1H NMR (400 MHz, DMSO-d6) δ 7.66-7.74 (m, 2H), 7.21-7.30 (m, 1H), 3.37-3.48 (m, 4H), 2.83-2.93 (m, 4H), 2.50 (s, 3H), 1.36 (s, 9H)

### 1.1.4 Intermediate 4

To a solution of tert-butyl 7-acetyl-4,5-dihydro-1H-benzo[d]azepine-3(2H)-carboxylate (3) (2 g, 6.9 mmol) in dioxane (23 ml) was added an aqueous solution of sodium hydroxide (2.2 g, 55.2 mmol in 32 ml of water) drop wise. The reaction mixture was then cooled to 0° C. and bromine (1.06 ml, 20.7 mmol) added drop wise. The reaction was allowed to stir at 0° C. for 1 hour. The reaction was carefully quenched with acetone and the reaction reduced in vacuo. The remaining aqueous layer was washed with ethyl acetate and then acidified with 5N hydrochloride acid. The aqueous layer was then extracted with ethyl acetate and the combined organics washed with brine and dried over magnesium sulphate to give 3(tert-butoxycarbonyl)-2,3,4,5-tetrahydro-1H-benzo[d]azepine-7-carboxylic acid (4) as a pale yellow solid in quantitative yield.

MS ES⁺ : 192

1H NMR (400 MHz, DMSO-d6) δ 7.60-7.80 (m, 2H), 7.16-7.32 (m, 1H), 3.41-3.52 (m, 4H), 2.84-2.97 (m, 4H), 1.34-1.47 (m, 9H)

### 1.1.5 Intermediate 5

Thionyl chloride (60 mL, 825 mmol) was added dropwise to a stirred solution of methanol (IL) at −20° C. and the reaction warmed to room temperature. To this solution 3-(tert-butoxycarbonyl)-2,3,4,5-tetrahydro-1H-benzo[d]azepine-7-carboxylic acid 25 g, 85.8 mmol) (4) was added portion-wise to give an orange solution. The reaction mixture was concentrated in vacuo and azeotroped with toluene to give methyl 2,3,4,5-tetrahydro-1H-benzo[d]azepine-7-carboxylate (5) as a pale brown solid in quantitative yield.

MS ES⁺ : 206 1H NMR (400 MHz, DMSO-d6): δ 9.46 (br. s., 2H), 7.71-7.87 (m, 2H), 7.32-7.42 (m, 1H), 3.85 (s, 3H), 3.19 (br. s., 8H)

### 1.1.6 Synthesis of Intermediate 6

### Method A

### Alkylation:

To a solution of methyl 2,3,4,5-tetrahydro-1H-benzo[d]azepine-7-carboxylate (5) (7.58 g, 31.4 mmol) in dimethylacetamide (75 mL) was added potassium carbonate (10.8 g, 78.5 mmol) and iodoethane (2.63 mL, 32.9 mmol) and the reaction stirred at room temperature for 18 hours. The reaction mixture was diluted with ethyl acetate and washed with saturated aqueous potassium carbonate and brine, dried over magnesium sulphate and reduced in vacuo. Purification by silica chromatography using methanol/dichloromethane mixtures with added ammonia afforded methyl 3-ethyl-2,3,4,5-tetrahydro-1H-benzo[d]azepine-7-carboxylate (6) in 12.9 mmol.

MS ES⁺ : 234 ¹H NMR (400 MHz, DMSO-d₆): δ 7.66-7.75 (m, 2H), 7.21-7.32 (m, 1H), 3.83 (s, 3H), 2.87-2.97 (m, 4H), 2.43-2.60 (m, 6H), 0.97-1.05 (m, 3H)

### Method B

### Reductive Amination:

To a solution of methyl 2,3,4,5-tetrahydro-1H-benzo[d]azepine-7-carboxylate (5) (20 g, 82.7 mmol) in dichloromethane (200 mL) was added cyclobutanone (9.27 mL, 124.1 mmol), acetic acid (0.5 mL), sodium triacetoxyborohydride (26.3 g, 124.1 mmol) and triethylamine (11.5 mL, 82.7 mmol). The reaction was stirred for 18 hours and the reaction mixture concentrated in vacuo. Aqueous sodium hydroxide was added and the aqueous phase extracted into dichloromethane (2×100 mL). The combined organics were dried over magnesium sulphate and concentrated in vacuo to give methyl 3-cyclobutyl-2,3,4,5-tetrahydro-1H-benzo[d]azepine-7-carboxylate (6) as a brown solid in quantitative yield.

MS ES⁺ : 260

¹H NMR (400 MHz, DMSO-d₆): δ 7.67-7.74 (m, 2H), 7.23-7.29 (m, 1H), 3.83 (s, 3H), 2.86-2.95 (m, 4H), 2.71-2.82 (m, 1H), 2.35 (br. s., 4H), 1.95-2.05 (m, 2H), 1.72-1.84 (m, 2H), 1.51-1.66 (m, 2H)

### 1.1.7 Synthesis of Intermediate 7

To a solution of methyl 3-cyclobutyl-2,3,4,5-tetrahydro-1H-benzo[d]azepine-7-carboxylate (6) (22.03 g, 84.9 mmol) in tetrahydrofuran (300 mL) and water (100 mL) was added lithium hydroxide (2.44 g, 101.9 mmol) and the reaction refluxed for 18 hours. The reaction mixture was concentrated in vacuo and azeotroped with toluene (×3) to give the lithium salt of 3-cyclobutyl-2,3,4,5-tetrahydro-1H-benzo[d]azepine-7-carboxylic acid (7) as a pale brown solid in quantitative yield.

MS ES⁺ : 246

¹H NMR (400 MHz, DMSO-d₆): δ 7.60-7.70 (m, 2H), 6.95-7.05 (m, 1H), 2.81 (br. s., 4H), 2.69-2.77 (m, 1H), 2.33 (br. s., 4H), 1.95-2.05 (m, 2H), 1.71-1.84 (m, 2H), 1.51-1.65 (m, 2H)

### 1.1.8 Synthesis of Compound 8 (Formula 1)/Example 1

The lithium salt of 3-cyclobutyl-2,3,4,5-tetrahydro-1H-benzo[d]azepine-7-carboxylic acid (7)(0.502 g, 2 mmol) was stirred in thionyl chloride (7.30 mL, 100 mmol) for 60 minutes and then concentrated in vacuo and azeotroped with toluene to give the acid chloride as a brown solid. This material was suspended in dichloromethane (10 mL) and to this was added C-(2-methyl-2H-pyrazol-3-yl)-methylamine (0.22 g, 2 mmol) and triethylamine (0.28 mL, 8 mmol) and the reaction stirred for 18 hours. The reaction mixture was concentrated in vacuo and loaded onto an SCX-2 cartridge (20 g) eluting firstly with methanol (10 mL x3) and then 2M ammonia/methanol. Fractions corresponding to the product were combined and concentrated in vacuo and then purified by silica chromatography using methanol/dichloromethane mixtures with added ammonia to give 3-cyclobutyl-N-((1-methyl-1H-pyrazol-5-yl)methyl)-2,3,4,5-tetrahydro-1H-benzo[d]azepine-7-carboxamide (8) in 0.9 mmol.

¹H NMR (400 MHz, DMSO-d₆): δ 8.80-8.91 (m, 1H), 7.57-7.66 (m, 2H), 7.26-7.33 (m, 1H), 7.16-7.23 (m, 1H), 6.11-6.18 (m, 1H), 4.45-4.53 (m, 2H), 3.81 (s, 3H), 2.81-2.94 (m, 4H), 2.69-2.80 (m, 1H), 2.34 (br. s., 4H), 1.95-2.06 (m, 2H), 1.71-1.84 (m, 2H), 1.49-1.65 (m, 2H)

Compounds of Formula 1 can also prepared as illustrated in scheme 2 using standard conditions well known in the art.

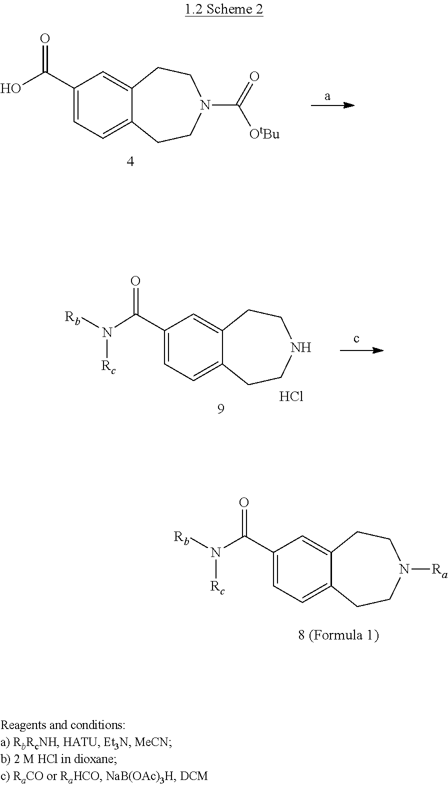

### 1.2.1 Synthesis of Intermediate 9

To a solution of 3(tert-butoxycarbonyl)-2,3,4,5-tetrahydro-1H-benzo[d]azepine-7-carboxylic acid (4.86 g, 16.67 mmol) (4) in acetonitrile (20 mL) was added triethylamine (5.5 mL, 40.011=01), benzylamine (2 mL, 18.34 mmol) and 2-(1H-7-Azabenzotriazol-1-yl)-1,1,3,3-tetramethyluronium hexafluorophosphate (HATU) (7.61 g, 20 mmol) and the reaction stirred for 16 hours. The reaction mixture was concentrated in vacuo, washed with brine and extracted into ethyl acetate (3×50 mL) and the combined organics dried over magnesium sulphate. The organics were concentrated in vacuo and the crude material purified by silica chromatography using methanol/dichloromethane mixtures with added ammonia to give tert-butyl 7-(benzylcarbamoyl)-4,5-dihydro-1H-benzo[d]azepine-3(2H)-carboxylate as a yellow solid in 15.7 mmol.

MS ES⁺ : 381, 324

¹H NMR (400 MHz, DMSO-d₆): δ 8.98-9.05 (m, 1H), 7.70-7.78 (m, 2H), 7.35-7.42 (m, 4H), 7.27-7.34 (m, 2H), 4.50-4.56 (m, 2H), 3.50-3.57 (m, 4H), 2.92-3.00 (m, 4H), 1.48 (s, 9H)

Tert-butyl 7-(benzylcarbamoyl)-4,5-dihydro-1H-benzo[d]azepine-3(2H)-carboxylate (5.95 g, 15.64 mmol) was stirred in refluxing saturated methanolic HCl for 2 hours Reaction mixture was concentrated in vacuo to give N-benzyl-2,3,4,5-tetrahydro-1H-benzo[d]azepine-7-carboxamide hydrochloride (9) as a pale yellow solid in quantitative yield.

MS ES⁺ : 281

¹H NMR (400 MHz, DMSO-d₆): δ 9.27 (br. s., 1H), 8.93-9.06 (m, 1H), 7.69-7.78 (m, 2H), 7.17-7.39 (m, 6H), 4.43-4.50 (m, 2H), 3.61-3.88 (m, 8H)

### 1.2.2 Synthesis of Compound 8 (Formula 1)/Example 115

To a solution of N-benzyl-2,3,4,5-tetrahydro-1H-benzo[d]azepine-7-carboxamide hydrochloride (9) (0.28 g, 1 mmol) in dichloromethane (5 mL) was added 3-methylbutanal (0.18 mL, 2 mmol) and acetic acid (1 drop). The reaction was stirred at room temperature for 15 mins prior to the addition of sodium triacetoxyborohydride (0.42 g, 2 mmol). After 1 hour the crude material was loaded onto an SCX-2 cartridge eluting with methanol and then 2M ammonia/methanol. Fractions corresponding to the product were combined and concentrated in vacuo and then purified by preparative HPLC to give N-benzyl-3-isobutyl-2,3,4,5-tetrahydro-1H-benzo[d]azepine-7-carboxamide (8) in 0.72 mmol.

MS ES⁺ : 337

¹H NMR (400 MHz, CDCl₃): δ 7.49-7.57 (m, 2H), 7.28-7.40 (m, 5H), 7.11-7.16 (m, 1H), 4.63-4.68 (m, 2H), 2.91-2.98 (m, 4H), 2.55-2.64 (m, 4H), 2.16-2.22 (m, 2H), 1.74-1.86 (m, 1H), 0.89-0.96 (m, 6H)

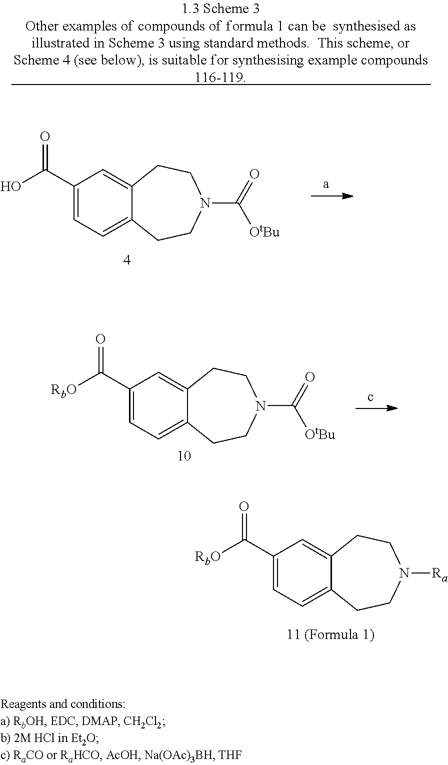

### 1.3.1 Synthesis of Intermediate 10

To a solution of (1-benzylpiperidin-4-yl)methanol (0.211 g, 1 mmol) in dichloromethane (15 mL) was added 3(tert-butoxycarbonyl)-2,3,4,5-tetrahydro-1H-benzo[d]azepine-7-carboxylic acid (4) (0.3 g, 1 mmol), N-(3-dimethylaminopropyl)-N-ethylcarbodiimide (EDC) (575 mg, 3 mmol), 4-dimethylaminopyridine (DMAP) (catalytic) and triethylamine (0.42 mL, 3 mmol) and the reaction stirred for 18 hours. Reaction mixture was concentrated in vacuo and partitioned between ethyl acetate and 5% aqueous citric acid/brine. The organics were washed with saturated aqueous sodium bicarbonate and brine, dried over magnesium sulphate and concentrated in vacuo. Material was purified by silica chromatography using ethyl acetate/petrol mixtures to give 7-(1-benzylpiperidin-4-yl)methyl 3-tert-butyl 4,5-dihydro-1H-benzo[d]azepine-3,7(2H)-dicarboxylate (10) in 0.45 mmol.

MS ES⁺ : 479

¹H NMR (400 MHz, DMSO-d₆): δ 7.70-7.76 (m, 2H), 7.21-7.34 (m, 6H), 4.10-4.15 (m, 2H), 3.42-3.49 (m, 6H), 2.88-2.95 (m, 4H), 2.79-2.86 (m, 2H), 1.89-1.97 (m, 2H), 1.66-1.78 (m, 3H), 1.39 (s, 9H), 1.22-1.35 (m, 2H)

### 1.3.2 Synthesis of Compound II (Formula 1)/Example 116

To a solution of give 7-(1-benzylpiperidin-4-yl)methyl 3-tert-butyl 4,5-dihydro-1H-benzo[d]azepine-3,7(2H)-dicarboxylate (10) (0.214 g, 0.45 mmol) in diethyl ether (2 mL) was added 2M HCl in diethyl ether (2 mL) to give a white solid. Methanol was added to give a colourless solution that slowly precipitated over 1 hour. Reaction mixture was concentrated in vacuo to give (1-benzylpiperidin-4-yl)methyl 2,3,4,5-tetrahydro-1H-benzo[d]azepine-7-carboxylate hydrochloride in 0.39 mmol. This was used without further purification in the next step.

MS ES⁺ : 379

To a partial suspension of (1-benzylpiperidin-4-yl)methyl 2,3,4,5-tetrahydro-1H-benzo[d]azepine-7-carboxylate hydrochloride (0.162 g, 0.39 mmol) in tetrahydrofuran (8 mL) was added acetaldehyde (0.12 mL, 2.14 mmol), acetic acid (0.05 mL, 0.86 mmol), 4 Å molecular sieves and sodium triacetoxyborohydride (0.146 g, 0.69 mmol) and the reaction mixture stirred at room temperature for 1 hour. The reaction was filtered and concentrated in vacuo and the residue partitioned between ethyl acetate and saturated aqueous sodium bicarbonate and the organics washed with brine, dried over magnesium sulphate and reduced in vacuo. Purification by silica chromatography using methanol/dichloromethane mixtures afforded (1-benzylpiperidin-4-yl)methyl 3-ethyl-2,3,4,5-tetrahydro-1H-benzo[d]azepine-7-carboxylate (11) in 0.32 mmol.

MS ES⁺ : 407

¹H NMR (400 MHz, DMSO-d₆): δ 7.60-7.67 (m, 2H), 7.13-7.29 (m, 6H), 4.01-4.08 (m, 2H), 3.38 (s, 2H), 2.81-2.89 (m, 4H), 2.72-2.79 (m, 2H), 2.36-2.50 (m, 6H), 1.81-1.91 (m, 2H), 1.59-1.70 (m, 3H), 1.15-1.29 (m, 2H), 0.90-0.97 (m, 3H)

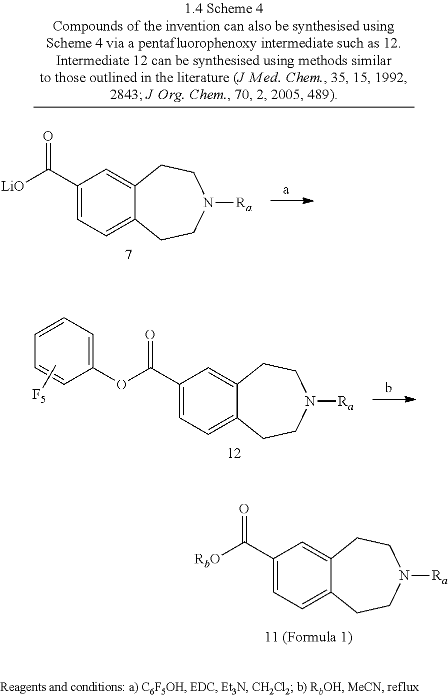

### 1.4.1 Synthesis of Intermediate 12

A suspension of the lithio-derivative of 3-cyclobutyl-2,3,4,5-tetrahydro-1H-benzo[d]azepine-7-carboxylic acid (7) (1.08 g, 4.401=01) in dichloromethane (30 mL) was treated with pentafluorophenol (0.81 g, 4.4 mmol), triethylamine (2.2 mL, 15.4 mmol) and N-(3-dimethylaminopropyl)-N-ethylcarbodiimide (EDC) (1.01 g, 5.28 mmol). The reaction was stirred for 18 hours at room temperature after which the reaction was concentrated in vacuo, washed with water and extracted into dichloromethane and dried over magnesium sulphate. The solvents were removed in vacuo and the residue was purified by silica chromatography using ethyl acetate/methanol mixtures to give perfluorophenyl 3-cyclobutyl-2,3,4,5-tetrahydro-1H-benzo[d]azepine-7-carboxylate (12) in 1.94 mmol.

¹H NMR (400 MHz, MeOD): δ 7.94-7.99 (m, 2H), 7.35-7.39 (m, 1H), 3.03-3.09 (m, 4H), 2.85-2.93 (m, 1H), 2.48-2.61 (m, 4H), 2.08-2.17 (m, 2H), 1.91-2.01 (m, 2H), 1.65-1.77 (m, 2H)

### 1.4.2 Synthesis of Compound II (Formula 1)/Example 119

To an acetonitrile (10 mL) solution of perfluorophenyl 3-cyclobutyl-2,3,4,5-tetrahydro-1H-benzo[d]azepine-7-carboxylate (12) (0.087 g, 0.20 mmol) was added (4-methoxyphenyl)methanol (0.028 g, 0.20 mmol) and the reaction refluxed for 48 hours. The reaction mixture was concentrated in vacuo and purified by silica chromatography using ethyl acetate/methanol mixtures to afford 4-methoxybenzyl 3-cyclobutyl-2,3,4,5-tetrahydro-1H-benzo[d]azepine-7-carboxylate (11) in 6.0 mmol.

¹H NMR (400 MHz, CDCl₃) δ 7.81-7.87 (m, 1H), 7.80 (s, 1H), 7.35-7.41 (m, 2H), 7.13-7.19 (m, 1H), 6.88-6.94 (m, 2H), 5.28 (s, 2H), 3.82 (s, 3H), 3.08 (br. s., 4H), 2.90-3.01 (m, 1H), 2.67 (br. s., 4H), 2.07-2.24 (m, 4H), 1.56-1.81 (m, 2H)

MS ES⁺ : 366

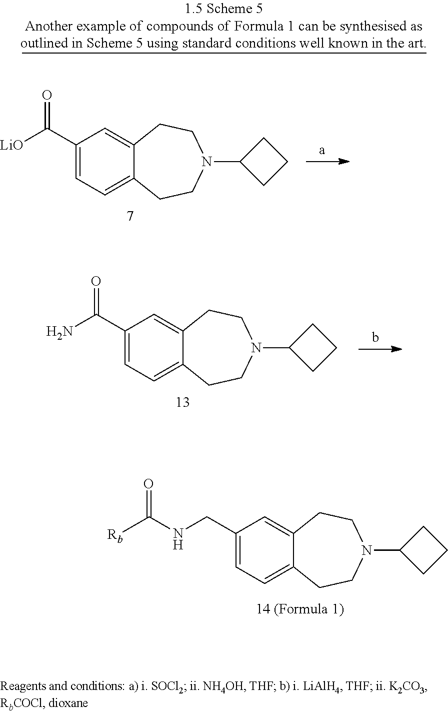

### 1.5.1 Intermediate 13

A 100 ml round-bottomed flask was charged with 3-cyclobutyl-2,3,4,5-tetrahydro-1H-benzo[d]azepine-7-carbonyl chloride (2 g, 7.58 mmol) (prepared from intermediate 7) in THF, to give an orange suspension. Concentrated ammonium hydroxide was added and the reaction mixture was stirred for one hour. The reaction mixture was extracted with dichloromethane and dried and evaporated to yield 3-cyclobutyl-2,3,4,5-tetrahydro-1H-benzo[d]azepine-7-carboxamide (6.14 mmol)

MS ES⁺ : 245

¹H NMR (400 MHz, MeOD): δ 7.61 (m, 2H), 7.15-7.21 (m, 1H), 2.96 (br. s., 4H), 2.77-2.86 (m, 1H), 2.47 (br. s., 4H), 2.03-2.14 (m, 2H), 1.86-1.98 (m, 2H), 1.60-1.76 (m, 2H).

### 1.5.2 Synthesis of Compound 14 (Formula 1)/Example 120

A 100 ml round-bottomed flask was charged with 3-cyclobutyl-2,3,4,5-tetrahydro-1H-benzo[d]azepine-7-carboxamide (2 g, 8.19 mmol) in tetrahydrofuran (20 mL), to give a tan suspension. Lithium aluminium hydride (16.37 ml, 16.37 mmol) was added and the reaction was stirred at reflux for 2 hours. The reaction was cooled and carefully quenched with saturated sodium sulphate. The reaction was dried with magnesium sulphate, filtered through celite and evaporated. The residue was taken up into dioxane and saturated potassium carbonate was added followed by nicotinoyl chloride (1.159 g, 8.19 mmol). The reaction was stirred for 2 hours before extracting into dichloromethane (×3) and drying and evaporating. The residue was purified by Biotage SNAP 50 g eluting with ammonia methanol dichloromethane to yield N-((3-cyclobutyl-2,3,4,5-tetrahydro-1H-benzo[d]azepin-7-yl)methyl)nicotinamide (0.626 mmol,).

MS ES⁺ : 336

¹H NMR (400 MHz, MeOD): δ 8.96-9.02 (m, 1H), 8.65-8.71 (m, 1H), 8.21-8.30 (m, 1H), 7.50-7.58 (m, 1H), 7.02-7.14 (m, 3H), 4.53 (s, 2H), 2.87-2.96 (m, 4H), 2.76-2.86 (m, 1H), 2.36-2.53 (m, 4H), 2.05-2.14 (m, 2H), 1.86-1.99 (m, 2H), 1.61-1.78 (m, 2H)

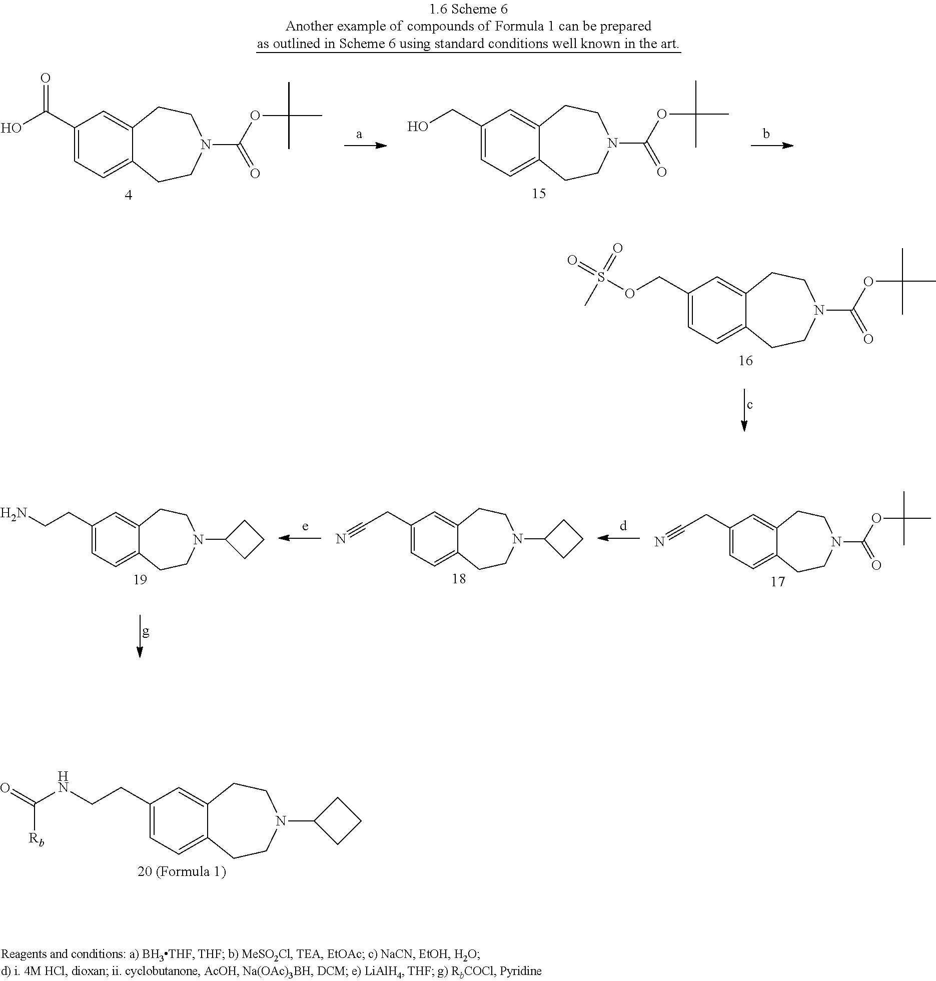

### 1.6.1 Intermediate 15

A 500 ml round-bottomed flask was charged with 3-(tert-butoxycarbonyl)-2,3,4,5-tetrahydro-1H-benzo[d]azepine-7-carboxylic acid (5.83 g, 20 mmol) in tetrahydrofuran (100 ml), to give a colourless solution. Borane tetrahydrofuran complex (30.4 ml, 30.4 mmol) was added drop wise at 0° C. The solution was stirred at 0° C. for one hour and then stirred at ambient temperature for 16 hours. The reaction was quenched by the addition of 100 mL of saturated bicarbonate solution and the THF was removed by evaporation. The residue was taken up into ethyl acetate and washed with water (×2). The organic layer was evaporated and the residue was purified by Biotage SNAP 50 g column using ethyl acetate petrol 1:1 to yield a colourless oil tert-butyl 7-(hydroxymethyl)-4,5-dihydro-1H-benzo[d]azepine-3(2H)-carboxylate (15.14 mmol).

ES⁺ 278 (M+H)

¹H NMR (400 MHz, MeOD): δ 7.11 (s, 3H), 4.54 (s, 2H), 3.47-3.58 (m, 4H), 2.85-2.92 (m, 4H), 1.47 (s, 9H)

### 1.6.2 Intermediate 16

A 100 ml round bottom flask was charged with tert-butyl 7-(hydroxymethyl)-4,5-dihydro-1H-benzo[d]azepine-3(2H)-carboxylate (4.2 g, 15.14 mmol) and triethylamine (2 ml, 15.14 mmol) in ethyl acetate (50 ml) to give a colourless solution. Methanesulfonyl chloride (1.3 ml, 16.7 mmol) was added and the reaction was stirred for 16 hours. The reaction mixture was diluted with ethyl acetate and washed with saturated bicarbonate, dried, filtered and evaporated. The residue was purified by Biotage SNAP 50 g column ethyl acetate/petrol 1:1 to yield tert-butyl 7-((methylsulfonyloxy)methyl)-4,5-dihydro-1H-benzo[d]azepine-3(2H)-carboxylate (11.25 mmol,).

MS ES⁺ : 378

### 1.6.3 Intermediate 17

A 100 ml round-bottomed flask was charged with cyanosodium (0.551 g, 11.25 mmol) and tert-butyl 7-((methylsulfonyloxy)methyl)-4,5-dihydro-1H-benzo[d]azepine-3(2H)-carboxylate (4 g, 11.25 mmol) in Water (2.500 ml) and Ethanol (10 ml) to give a colourless solution. The reaction was heated to reflux for 2 hours. After an aqueous workup and purification by Biotage (20% EtOAc/Petrol) tert-butyl 7-(cyanomethyl)-4,5-dihydro-1H-benzo[d]azepine-3(2H)-carboxylate (2.79 mmol,) was obtained.

MS ES⁺ : 213

¹H NMR (400 MHz, MeOD): δ 7.06-7.16 (m, 3H), 3.78 (s, 2H), 3.50 (br. s., 4H), 2.79-2.93 (m, 4H), 1.45 (s, 9H)

### 1.6.4 Intermediate 18

tert-Butyl 7-(cyanomethyl)-4,5-dihydro-1H-benzo[d]azepine-3(2H)-carboxylate (Intermediate 17) (2.5 mmol) was stirred in 4M HCl in dioxane for 2 hours and then the solvent was removed by evaporation.

MS ES⁺ : 187

To this residue was added sodium triacetoxyborohydride (0.795 g, 3.75 mmol), acetic acid (0.215 ml, 3.75 mmol) and cyclobutanone (0.263 g, 3.75 mmol) in dichloromethane (10 ml) to give a colourless solution. The reaction was stirred for 1 hour. The reaction mixture was diluted with 5% aqueous sodium hydroxide and extracted with dichloromethane (×3) dried and evaporated to yield an oil, triturated with ether to yield a white solid 2-(3-cyclobutyl-2,3,4,5-tetrahydro-1H-benzo[d]azepin-7-yl)acetonitrile (2.351 mmol) which was used in the next step without further purification.

### 1.6.5 Intermediate 19

A 25 ml round-bottomed flask was charged with lithium aluminium hydride (0.134 g, 3.53 mmol) in tetrahydrofuran (10 ml), to give a colourless solution. Lithium aluminium hydride (0.134 g, 3.53 mmol) in tetrahydrofuran (3.5 mL) was added. The reaction was refluxed for 12 hours and then cooled and quenched with saturated sodium sulphate. The reaction mixture was dried, filtered and evaporated and used without further purification in the next step 2-(3-cyclobutyl-2,3,4,5-tetrahydro-1H-benzo[d]azepin-7-yl)ethanamine (1.179 mmol).

### 1.6.6 Compound 20 (Formula 1)/Example 121

A 25 ml round-bottomed flask was charged with 2-(3-cyclobutyl-2,3,4,5-tetrahydro-1H-benzo[d]azepin-7-yl)ethanamine (0.288 g, 1.179 mmol) and nicotinoyl chloride (0.250 g, 1.769 mmol) in pyridine (10 ml) to give a colourless solution. The reaction was stirred for one hour and then azeotroped with toluene. The residue was taken up into ethyl acetate and washed with 1M sodium hydroxide solution. The organic layer was dried and the residue was purified using 10% methanol in dichloromethane with 0.2% ammonium hydroxide to yield crude product which was purified using preparative HPLC to give N-(2-(3-cyclobutyl-2,3,4,5-tetrahydro-1H-benzo[d]azepin-7-yl)ethyl)nicotinamide (0.086 mmol).

MS ES⁺ : 350

¹H NMR (400 MHz, MeOD): δ 8.87-8.91 (m, 1H), 8.64-8.69 (m, 1H), 8.15-8.20 (m, 1H), 7.50-7.56 (m, 1H), 6.97-7.05 (m, 3H), 3.55-3.62 (m, 2H), 2.77-2.96 (m, 7H), 2.44 (br. s., 4H), 2.05-2.14 (m, 2H), 1.86-1.99 (m, 2H), 1.61-1.77 (m, 2H)

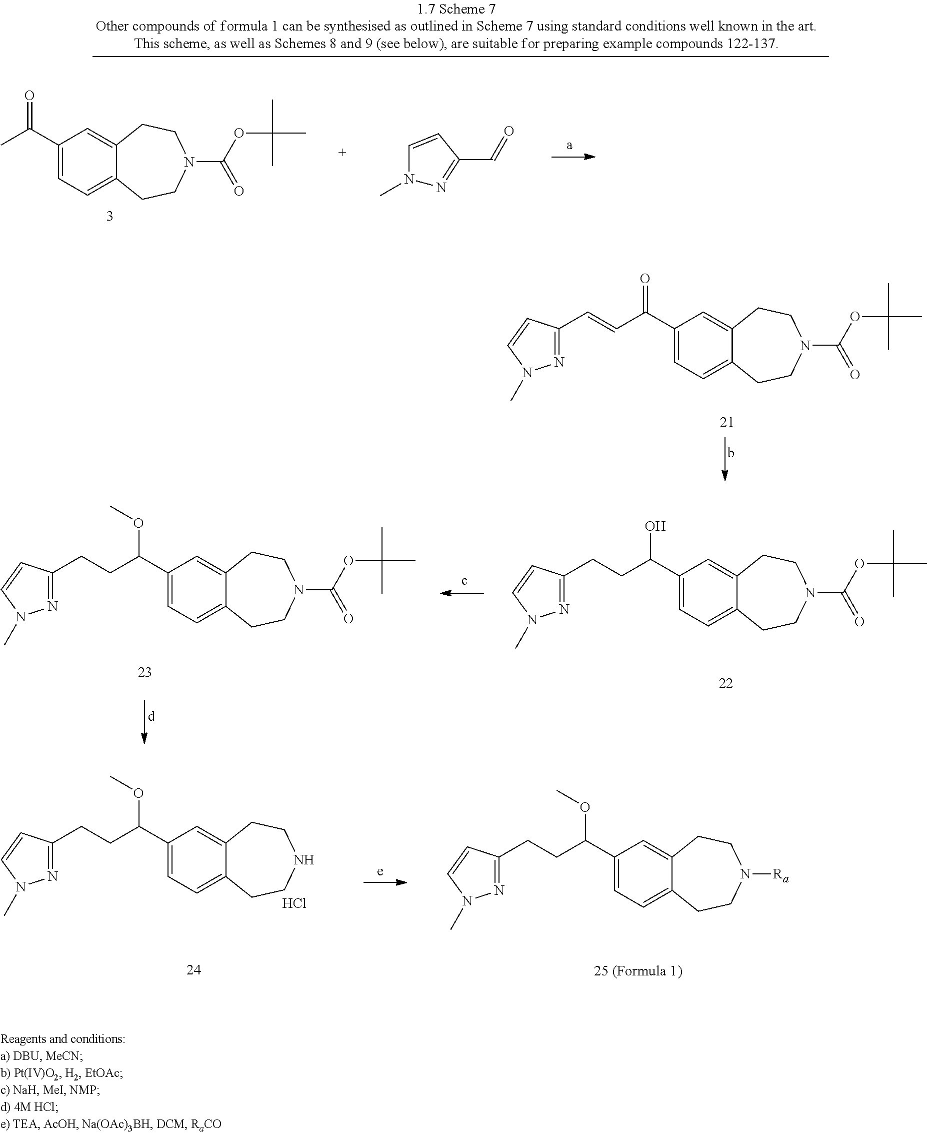

### 1.7.1 Intermediate 21

A 25 ml round-bottomed flask was charged with lithium chloride (0.366 g, 8.64 mmol), 1-methyl-1H-pyrazole-3-carbaldehyde (0.951 g, 8.64 mmol), and tert-butyl 7-acetyl-4,5-dihydro-1H-benzo[d]azepine-3(2H)-carboxylate (2.5 g, 8.64 mmol) in acetonitrile (10 ml) to give a colourless solution. DBU (1.302 ml, 8.64 mmol) was added and 4 A molecular sieves. The reaction was stirred for one hour. The solvent was evaporated and the residue was taken up into ethyl acetate and washed with 5% citric acid (×2), saturated sodium bicarbonate, and dried and evaporated. The residue was purified by Biotage 50 g SNAP column using 50% EtOAc/Petrol to yield pure (E)-tert-butyl 7-(3-(1-methyl-1H-pyrazol-3-yl)acryloyl)-4,5-dihydro-1H-benzo[d]azepine-3(2H)-carboxylate (6.82 mmol)

MS ES⁺ : 326

¹H NMR (400 MHz, MeOD): δ 7.80-7.85 (m, 2H), 7.64-7.66 (m, 2H), 7.60-7.63 (m, 1H), 7.27-7.32 (m, 1H), 6.75-6.78 (m, 1H), 3.92 (s, 3H), 3.56 (br. s., 4H), 2.95-3.02 (m, 4H), 1.44 (s, 9H)

### 1.7.2 Intermediate 22

A 100 ml round-bottomed flask was charged with (E)-tert-butyl 7-(3-(1-methyl-1H-pyrazol-3-yl)acryloyl)-4,5-dihydro-1H-benzo[d]azepine-3(2H)-carboxylate (2.6 g, 6.82 mmol) and platinum (IV) oxide (0.077 g, 0.341 mmol) in ethyl acetate (50 ml) to give a black suspension. The reaction was hydrogenated at atmospheric pressure monitoring by LCMS. The reaction was filtered through celite and evaporated to yield an oil, purified by Biotage 50 g using ethyl acetate to elute product tert-butyl 7-(1-hydroxy-3-(1-methyl-1H-pyrazol-3-yl)propyl)-4,5-dihydro-1H-benzo[d]azepine-3(2H)-carboxylate (6.23 mmol).

MS ES⁺ : 408

¹H NMR (400 MHz, MeOD): δ 7.41-7.45 (m, 1H), 7.08-7.25 (m, 3H), 5.97-6.10 (m, 1H), 4.52-4.67 (m, 1H), 3.80 (s, 3H), 3.44-3.65 (m, 4H), 2.82-2.99 (m, 4H), 2.51-2.74 (m, 2H), 1.87-2.13 (m, 2H), 1.47 (s, 9H)

### 1.7.3 Intermediate 23

A 50 ml round-bottomed flask was charged with tert-butyl 7-(1-hydroxy-3-(1-methyl-1H-pyrazol-3-yl)propyl)-4,5-dihydro-1H-benzo[d]azepine-3(2H)-carboxylate (1 g, 2.59 mmol) in N-methylpyrrolidone (NMP), to give a colourless solution. Sodium hydride (0.125 g, 3.11 mmol) was added and the reaction was stirred for one hour and then iodomethane was added to the mixture and the reaction was stirred for 16 hours. The reaction was diluted with water and extracted with ethyl acetate. The organic layer was washed with water (×3) dried and evaporated. The residue was purified by Biotage SNAP 50 g using ethyl acetate petrol (1:1) to yield tert-butyl 7-(1-methoxy-3-(1-methyl-1H-pyrazol-3-yl)propyl)-4,5-dihydro-1H-benzo[d]azepine-3(2H)-carboxylate (1.502 mmol).

MS ES⁺ : 422

¹H NMR (400 MHz, MeOD): δ 7.41-7.45 (m, 1H), 7.10-7.15 (m, 1H), 7.03-7.07 (m, 2H), 6.01-6.06 (m, 1H), 4.07-4.13 (m, 1H), 3.80 (s, 3H), 3.54 (br. s., 4H), 3.17 (s, 3H), 2.86-2.92 (m, 4H), 2.51-2.70 (m, 2H), 2.00-2.12 (m, 1H), 1.82-1.95 (m, 1H), 1.46 (s, 9H)

### 1.7.4 Intermediate 24

A 25 ml round-bottomed flask was charged with tert-butyl 7-(1-methoxy-3-(1-methyl-1H-pyrazol-3-yl)propyl)-4,5-dihydro-1H-benzo[d]azepine-3(2H)-carboxylate (0.65 g, 1.627 mmol) in 4M HCl, to give a colourless solution. The reaction was stirred for two hours and then evaporated to yield 7-(1-methoxy-3-(1-methyl-1H-pyrazol-3-yl)propyl)-2,3,4,5-tetrahydro-1H-benzo[d]azepinium chloride (1.548 mmol). This material was used without further purification.

MS ES⁺ : 268

### 1.7.5 Synthesis of Compound 25 (Formula 1)/Example 136

A 50 ml round-bottomed flask was charged with 7-(1-methoxy-3-(1-methyl-1H-pyrazol-3-yl)propyl)-2,3,4,5-tetrahydro-1H-benzo[d]azepinium chloride (0.520 g, 1.548 mmol), cyclobutanone (0.217 g, 3.10 mmol), and triethylamine (0.216 ml, 1.548 mmol) in dichloromethane (25 mL) to give a colourless solution. Sodium triacetoxyborohydride (0.656 g, 3.10 mmol) and acetic acid (0.886 ml, 15.48 mmol) were added and the reaction was stirred for 16 hours before diluting with dichloromethane and washing with 5% sodium hydroxide solution. The organic phase was dried and evaporated and the residue was purified by Biotage SNAP 50 g eluting with 2% ammonia methanol/dichloromethane (1-20%) to yield 3-cyclobutyl-7-(1-methoxy-3-(1-methyl-1H-pyrazol-3-yl)propyl)-2,3,4,5-tetrahydro-1H-benzo[d]azepinium chloride after treatment with ethereal HCl.

MS ES⁺ : 322

¹H NMR (400 MHz, MeOD): δ 7.85-7.90 (m, 1H), 7.09-7.20 (m, 3H), 6.35-6.41 (m, 1H), 4.09-4.15 (m, 1H), 3.96 (s, 3H), 3.56-3.70 (m, 4H), 3.27-3.39 (m, 2H), 3.12 (s, 3H), 2.97-3.08 (m, 2H), 2.67-2.84 (m, 4H), 2.26-2.45 (m, 4H), 1.67-2.10 (m, 3H)

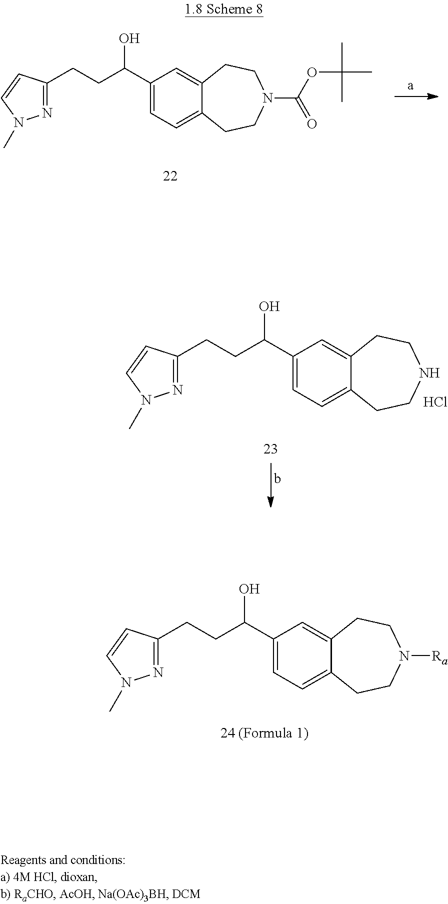

### 1.8.1 Intermediate 23

A 25 ml round-bottomed flask was charged with tert-butyl 7-(1-hydroxy-3-(1-methyl-1H-pyrazol-3-yl)propyl)-4,5-dihydro-1H-benzo[d]azepine-3(2H)-carboxylate (1.7 g, 4.41 mmol) in 4M HCl, to give a colourless solution. The reaction was stirred for two hours and then evaporated to yield 7-(1-hydroxy-3-(1-methyl-1H-pyrazol-3-yl)propyl)-2,3,4,5-tetrahydro-1H-benzo[d]azepinium chloride (4.35 mmol).

MS ES⁺: 268 M−18

### 1.7.5 Synthesis of Compound 24 (Formula 1)/Example 135

A 100 ml round-bottomed flask was charged with acetic acid (2.490 ml, 43.5 mmol), sodium triacetoxyborohydride (1.844 g, 8.70 mmol), 7-(1-hydroxy-3-(1-methyl-1H-pyrazol-3-yl)propyl)-2,3,4,5-tetrahydro-1H-benzo[d]azepinium chloride (1.4 g, 4.35 mmol) and triethylamine (0.606 ml, 4.35 mmol) in DCM (50 ml) to give a colourless solution. Cyclobutanone (0.610 g, 8.70 mmol) was added. The reaction was stirred for 16 hours and then the reaction mixture was washed with 5% NaOH solution and dried and evaporated. The residue was purified by Biotage SNAP 100 g using 10% Methanol/DCM with 2% ammonia to yield 1-(3-cyclobutyl-2,3,4,5-tetrahydro-1H-benzo[d]azepin-7-yl)-3-(1-methyl-1H-pyrazol-3-yl)propan-1-ol (2.504 mmol)

MS ES⁺ : 323

¹H NMR (400 MHz, MeOD): δ 7.40-7.46 (m, 1H), 7.03-7.13 (m, 3H), 6.02-6.10 (m, 1H), 4.53-4.62 (m, 1H), 3.80 (s, 3H), 2.77-2.97 (m, 5H), 2.34-2.70 (m, 6H), 1.87-2.15 (m, 6H), 1.59-1.78 (m, 2H)

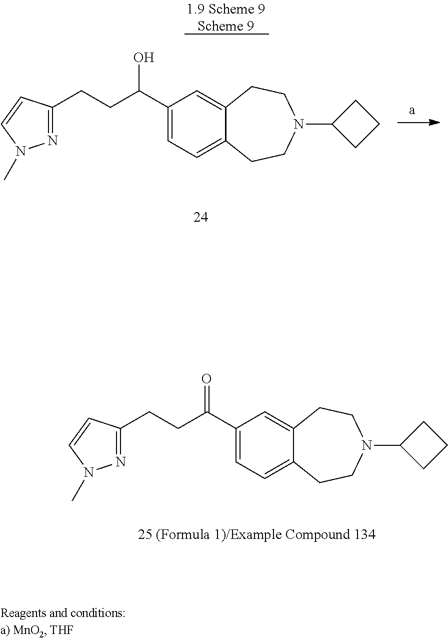

### 1.9.1 Synthesis of Compound 25 (Formula 1)/Example 134

A 100 ml round-bottomed flask was charged with 1-(3-cyclobutyl-2,3,4,5-tetrahydro-1H-benzo[d]azepin-7-yl)-3-(1-methyl-1H-pyrazol-3-yl)propan-1-ol (0.5 g, 1.473 mmol) and manganese dioxide (1.280 g, 14.73 mmol) in tetrahydrofuran (50 ml) to give a black suspension. The reaction was heated to reflux for 2 hours, and then filtered and evaporated and purified by biotage SNAP 100 g eluting with 10% Methanol in dichloromethane with ammonia to yield 1-(3-cyclobutyl-2,3,4,5-tetrahydro-1H-benzo[d]azepin-7-yl)-3-(1-methyl-1H-pyrazol-3-yl)propan-1-one (0.504 mmol).

MS ES⁺ : 338

¹H NMR (400 MHz, CDCl₃): δ 7.71-7.78 (m, 2H), 7.23-7.26 (m, 1H), 7.15-7.20 (m, 1H), 6.05-6.11 (m, 1H), 3.88 (s, 3H), 3.30-3.39 (m, 2H), 3.03-3.12 (m, 2H), 2.97 (br. s., 4H), 2.72-2.85 (m, 1H), 2.45 (br. s., 4H), 2.03-2.15 (m, 2H), 1.84-1.99 (m, 2H), 1.55-1.79 (m, 2H).

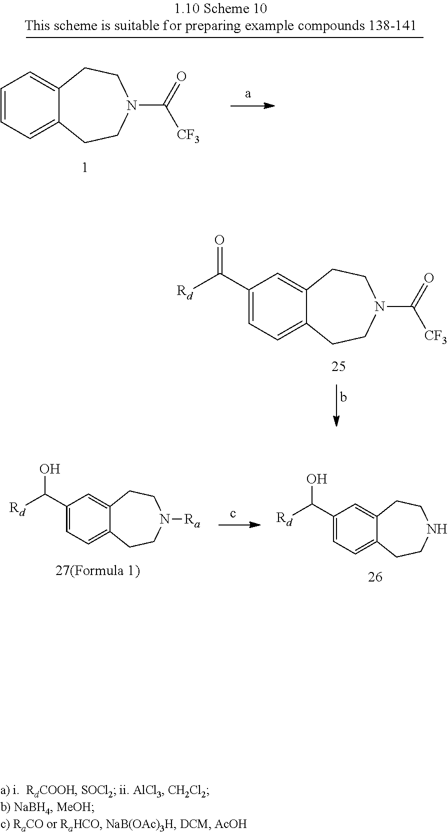

### 1.10.1 Intermediate 25

A round bottomed flask was charged with thionyl chloride (7.3 ml, 100 mmol) and 1-acetylpiperidine-4-carboxylic acid (1.7 g, 10.00 mmol) to give a colourless solution. After about 15 minutes crystals formed. The reaction was diluted with petrol and the reaction was filtered and washed with petrol. The crystals were dried and evaporated and 1-(4,5-dihydro-1H-benzo[d]azepin-3(2H)-yl)-2,2,2-trifluoroethanone (2.4 g, 10 mmol) was added and the mixture was dissolved in dichloromethane (20 mL). Aluminum trichloride (4 g, 30 mmol) was added cautiously, portion wise, and the mixture was stirred for two hours before pouring onto ice and extracting with ethyl acetate. The organic extract was washed with brine and dried and evaporated. The residue was purified by column chromatography on silica using 10% methanol in dichloromethane to yield 1-(7-(1-acetylpiperidine-4-carbonyl)-4,5-dihydro-1H-benzo[d]azepin-3(2H)-yl)-2,2,2-trifluoroethanone (2.6 g, 6.6 mmol, 65.6% yield) as an oil.

¹H NMR (400 MHz, MeOD-d4) δ 7.77-7.95 (m, 2H), 7.32-7.45 (m, 1H), 4.47-4.58 (m, 1H), 3.95-4.09 (m, 1H), 3.65-3.91 (m, 5H), 3.35-3.40 (m, 1H), 3.06-3.22 (m, 4H), 2.83-3.02 (m, 1H), 2.09-2.20 (m, 3H), 1.84-2.02 (m, 2H), 1.48-1.79 (m, 2H)

MS ES⁺ : 397

### 1.10.2 Intermediate 26

A flask was charged with 1-(7-(1-acetylpiperidine-4-carbonyl)-4,5-dihydro-1H-benzo[d]azepin-3(2H)-yl)-2,2,2-trifluoroethanone (2.6 g, 6.6 mmol) and sodium borohydride (0.76 g, 20 mmol) in methanol (20 mL) to give a colourless solution. The reaction was stirred for 16 h. The solvent was removed and the product was used crude in the next step

### 1.10.3 Compound 27 (Formula 1)/Example 138

A flask was charged with 1-(4-(hydroxy(2,3,4,5-tetrahydro-1H-benzo[d]azepin-7-yl)methyl)piperidin-1-yl)ethanone) (2.0 g, 6.6 mmol), cyclobutanone (0.25 g, 13.2 mmol) and triethylamine (0.69 g, 6.6 mmol) in dichloromethane (20 mL) to give a colourless solution. Sodium triacetoxy borohydride (2.1 g, 9.9 mmol) was added and the reaction was stirred for two hours before diluting with 2M sodium hydroxide and extracting with dichloromethane. The organic layer was dried and evaporated and purified by silica chromatography, eluting with 10% methanol in dichloromethane to yield 1-(4-((3-cyclobutyl-2,3,4,5-tetrahydro-1H-benzo[d]azepin-7-yl)(hydroxy)methyl)piperidin-1-yl)ethanone (0.9 g, 2.6 mmol, 40% yield) as a white solid after trituration with ether.

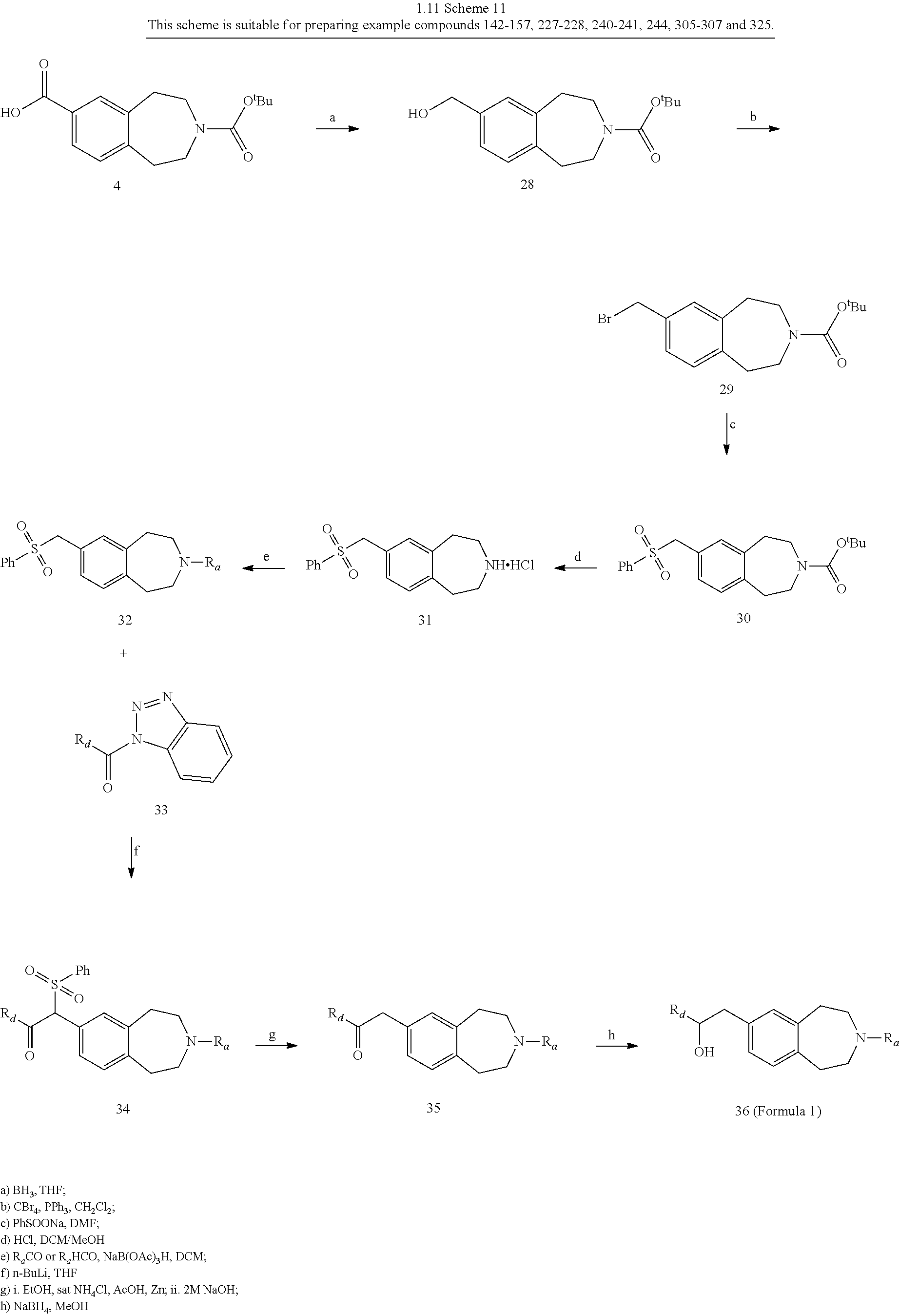

### 1.11.1 Intermediate 4

Made using a previous described method (see 1.1.4).

### 1.11.2 Intermediate 28

3-(tert-butoxycarbonyl)-2,3,4,5-tetrahydro-1H-benzo[d]azepine-carboxylic acid (10 g, 34.32 mmol) was added portionwise to borane 1M in tetrahydrofuran (51.5 ml, 51.49 mmol) and resultant mixture stirred for 16 hr at room temperature under nitrogen. Sodium bicarbonate was added slowly until bubbling ceased. Reaction was then extracted with EtOAc and organic phase dried over magnesium sulphate, filtered and concentrated in vacuo to yield tert-butyl 7-(hydroxymethyl)-4,5-dihydro-1H-benzo[d]azepine-3(2H)-carboxylate as a colourless oil (9.34 g, 33.67 mmol, 98%).

¹H NMR (400 MHz, MeOD) δ ppm 1.49 (s, 9H) 2.84-2.96 (m, 4H) 3.48-3.62 (m, 4H) 4.50-4.59 (m, 2H) 7.12 (s, 3H)

MS ES⁻ 276

### 1.11.3 Intermediate 29

Tert-butyl 7-(hydroxymethyl)-4,5-dihydro-1H-benzo[d]azepine-3(2H)-carboxylate (19.85 g, 71.57 mmol) and triphenylphosphine (18.77 g, 71.57 mmol) in dichloromethane (60 mL) were cooled to 0° C. and a solution of carbon tetrabromide (23.73 g, 71.57 mmol) in dichloromethane (60 mL) was added via a dropping funnel over 1 h. Upon complete addition, the mixture was warmed to RT and stirred overnight (LCMS indicated mostly product after this time). The reaction mixture was poured into petrol (500 mL) and filtered through Celite. The residual solid remaining in the flask was triturated with petrol (3×200 mL) and filtered as before. The filtrate was concentrated and dichloromethane and silica gel were added. After concentration to dryness, the product was purified by dry flash chromatography on silica gel, eluting with 0-30% EtOAc/petrol step-gradient (product eluted at 10-20% EtOAc) to give 4 as an oil which crystallised to tert-butyl 7-(bromomethyl)-4,5-dihydro-1H-benzo[d]azepine-3(2H)-carboxylate as a white solid upon scratching/seeding/standing (16.24 g, 47.73 mmol, 67%).

¹H NMR (400 MHz, MeOD) δ ppm 1.48 (s, 9H) 2.85-2.98 (m, 4H) 3.48-3.64 (m, 4H) 4.48-4.59 (m, 2H) 7.08-7.25 (m, 3H)

MS ES⁺ 340 342

### 1.11.4 Intermediate 30

Tert-butyl 7-(bromomethyl)-4,5-dihydro-1H-benzo[d]azepine-3(2H)-carboxylate (10.00 g, 29.4 mmol) was suspended in DMF (30 ml) and sodium benzenesulfinate (7.24 g, 44.1 mmol) was added. The reaction was stirred for 16 hr at room temperature.

The reaction mixture was diluted with EtOAc (50 mL) and washed with brine (5×50 mL). The organic phase was dried over magnesium sulphate, filtered and concentrated in vacuo to yield 12.097 g of white powder. This was washed with diethyl ether and filtered to yield tert-butyl 7-(phenylsulfonylmethyl)-4,5-dihydro-1H-benzo[d]azepine-3(2H)-carboxylate (8.75 g, 21.79 mmol, 74%).

¹H NMR (400 MHz, MeOD) δ ppm 1.49 (s, 9H) 2.72-2.84 (m, 2H) 2.85-2.94 (m, 2H) 3.41-3.61 (m, 4H) 4.44 (s, 2H) 6.75-7.00 (m, 2H) 7.05 (s, 1H) 7.56 (d, J=7.33 Hz, 2H) 7.68 (d, J=7.58 Hz, 3H)

MS ES⁺ 402

### 1.11.5 Intermediate 31

Tert-butyl 7-(phenylsulfonylmethyl)-4,5-dihydro-1H-benzo[d]azepine-3(2H)-carboxylate (8.75 g, 21.79 mmol) was dissolved in 10 ml of DCM/MeOH (9:1) and 4M HCl in 1,4-Dioxane (25 ml, 25 mmol) was added and mixture stirred for 1 hour at room temperature. Mixture was concentrated in vacuo to dryness to yield 7-(phenylsulfonylmethyl)-2,3,4,5-tetrahydro-1H-benzo[d]azepine (6.781 g, 22.50 mmol, 100%) as a white solid.

¹H NMR (400 MHz, MeOD) δ ppm 3.02-3.11 (m, 2H) 3.12-3.19 (m, 2H) 3.23-3.30 (m, 4H) 4.49 (s, 2H) 6.97-7.08 (m, 2H) 7.13-7.20 (m, 1H) 7.53-7.63 (m, 2H) 7.66-7.78 (m, 3H)

MS ES⁺ 302

### 1.11.6 Intermediate 32

7-(phenylsulfonylmethyl)-2,3,4,5-tetrahydro-1H-benzo[d]azepine (6.78 g, 22.49 mmol) was suspended in CH₂Cl₂ (20 mL) and triethylamine (3.43 mL, 24.74 mmol) and cyclobutanone (2.52 mL, 33.70 mmol) were added. The mixture was stirred under nitrogen and sodium triacetoxyhydroborate (7.15 g, 33.70 mmol) and acetic acid (1.93 mL, 33.70 mmol) were added. The mixture was stirred at room temperature for 16 hrs. The mixture was quenched with NaOH (2M, aq, 50 mL) and the phases separated. The aqueous phase was extracted with DCM (3×10 mL) and the organic layers combined, dried over magnesium sulphate, filtered and concentrated in vacuo to yield a colourless oil. The oil was triturated with diethyl ether to yield 3-cyclobutyl-7-(phenylsulfonylmethyl)-2,3,4,5-tetrahydro-1H-benzo[d]azepine (5.361 g, 15.08 mmol, 67%) as a white solid.

¹H NMR (400 MHz, MeOD) δ ppm 1.59-1.83 (m, 2H) 1.87-2.02 (m, 2H) 2.05-2.19 (m, 2H) 2.23-2.61 (m, 4H) 2.74-2.97 (m, 5H) 4.44 (s, 2H) 6.78-6.85 (m, 1H) 6.87-6.94 (m, 1H) 6.98-7.07 (m, 1H) 7.49-7.59 (m, 2H) 7.66 (s, 3H)

MS ES⁺ 356

### 1.11.7 Intermediate 33

A 20 mL microwave vial was charged with 1-(methylsulfonyl)-1H-benzo[d][1,2,3]triazole (0.986 g, 5.00 mmol) and 2-(1-propionylpiperidin-4-yl)acetic acid (0.996 g, 5 mmol) in THF (10 ml) to give a colourless solution. Triethylamine (0.976 mL, 7.00 mmol) was added and the reaction was microwaved at 130° C. for 20 minutes. TLC 1:1 EtOAc/Petrol showed complete reaction. The solvent was evaporated and the residue was purified by silica chromatography using ethyl acetate/petrol 0-100% to yield 1-(4-(2-(1H-benzo[d][1,2,3]triazol-1-yl)-2-oxoethyl)piperidin-1-yl)propan-1-one (1.1 g, 3.66 mmol, 73.2% yield)

¹H NMR (400 MHz, CDCl₃) δ 8.28-8.35 (m, 1H), 8.12-8.19 (m, 1H), 7.66-7.73 (m, 1H), 7.50-7.58 (m, 1H), 3.55-5.00 (bm, 2H), 3.37-3.44 (m, 2H), 2.50-3.33 (bm, 2H), 2.32-2.44 (m, 3H), 1.89-2.00 (m, 2H), 1.31-1.45 (m, 2H), 1.13-1.22 (m, 3H)

### 1.11.8 Intermediate 34

3-Cyclobutyl-7-(phenylsulfonylmethyl)-2,3,4,5-tetrahydro-1H-benzo[d]azepine (1.422 g, 4 mmol) was dissolved in THF (30 mL) at 0° C. and n-butyllithium (5.00 mL of 1.6M, 8.00 mmol) was added dropwise. The reaction was warmed to room temperature for one hour and then cooled to −78° C. 1-(4-(2-(1H-Benzo[d][1,2,3]triazol-1-yl)-2-oxoethyl)piperidin-1-yl)propan-1-one (1.201 g, 4.00 mmol) in THF (5 mL) was added dropwise. The reaction was allowed to warm to room temperature over 16 hours. The reaction was quenched with saturated ammonium chloride, diluted with DCM and basified with sodium carbonate solution. The aqueous layer was extracted (×3) with dichloromethane, dried and evaporated. The residue was purified by KPNH silica cartridge to yield 1-(4-(3-(3-Cyclobutyl-2,3,4,5-tetrahydro-1H-benzo[d]azepin-7-yl)-2-oxo-3-(phenylsulfonyl)propyl)piperidin-1-yl)propan-1-one (1.6 g, 2.98 mmol, 74.5% yield)

¹H NMR (400 MHz, MeOD) δ 7.59-7.72 (m, 3H), 7.47-7.55 (m, 2H), 6.96-7.13 (m, 3H), 4.41 (br. s., 1H), 3.86 (br. s., 1H), 2.98-3.11 (m, 1H), 2.76-2.98 (m, 5H), 2.32-2.70 (m, 8H), 1.84-2.18 (m, 5H), 1.51-1.81 (m, 4H), 0.82-1.44 (m, 7H)

### 1.11.9 Intermediate 35

1-(4-(3-(3-Cyclobutyl-2,3,4,5-tetrahydro-1H-benzo[d]azepin-7-yl)-2-oxo-3-(phenylsulfonyl)propyl)piperidin-1-yl)propan-1-one (1.6 g, 2.98 mmol) was dissolved in ethanol (50 mL) and sat. ammonium chloride and acetic acid (3.41 mL, 59.6 mmol) were added followed by zinc (1.950 g, 29.8 mmol) and the reaction was refluxed for 1.5 h. LCMS showed completion of reaction, and the reaction mixture was cooled, basified with 2M NaOH and extracted with dichloromethane (×3), dried and evaporated. The residue was purified by KPNH cartridge (55 g) eluting with ethyl acetate petrol 0-100% to yield 1-(4-(3-(3-Cyclobutyl-2,3,4,5-tetrahydro-1H-benzo[d]azepin-7-yl)-2-oxopropyl)piperidin-1-yl)propan-1-one (1.1 g, 2.77 mmol, 93% yield)

¹H NMR (400 MHz, MeOD) δ 7.02-7.12 (m, 1H), 6.97 (br. s., 2H), 4.39-4.52 (m, 1H), 3.82-3.94 (m, 1H), 3.67 (s, 2H), 3.00-3.13 (m, 1H), 2.77-3.00 (m, 5H), 2.31-2.69 (m, 9H), 1.87-2.19 (m, 5H), 1.59-1.81 (m, 4H), 0.90-1.15 (m, 5H)

MS ES⁺ 397

### 1.11.10 Compound 36 (Formula 1)/Example 147

1-(4-(3-(3-Cyclobutyl-2,3,4,5-tetrahydro-1H-benzo[d]azepin-7-yl)-2-oxopropyl)piperidin-1-yl)propan-1-one (1.1 g, 2.77 mmol) was dissolved in methanol (30 mL) and sodium borohydride (0.315 g, 8.32 mmol) was added to the stirred solution. The reaction was stirred for an hour before quenching with 2M HCl (10 mL) and evaporating. The residue was taken up into dichloromethane and basified with 2M NaOH and extracted with more dichloromethane. The extract was evaporated and the residue was purified by KPNH silica using ethyl acetate/petrol to yield a solid which was recrystallised from ethyl acetate heptane to yield 1444343-Cyclobutyl-2,3,4,5-tetrahydro-1H-benzo[d]azepin-7-yl)-2-hydroxypropyl)piperidin-1-yl)propan-1-one (0.739 g, 1.854 mmol, 66.8% yield) as a first crop.

¹H NMR (400 MHz, MeOD) δ 6.90-7.07 (m, 3H), 4.42-4.56 (m, 1H), 3.82-3.98 (m, 2H), 2.98-3.12 (m, 1H), 2.79-2.98 (m, 5H), 2.31-2.77 (m, 9H), 2.05-2.19 (m, 2H), 1.89-2.05 (m, 2H), 1.58-1.89 (m, 5H), 1.27-1.49 (m, 2H), 0.86-1.23 (m, 5H)

MS ES⁺ 399

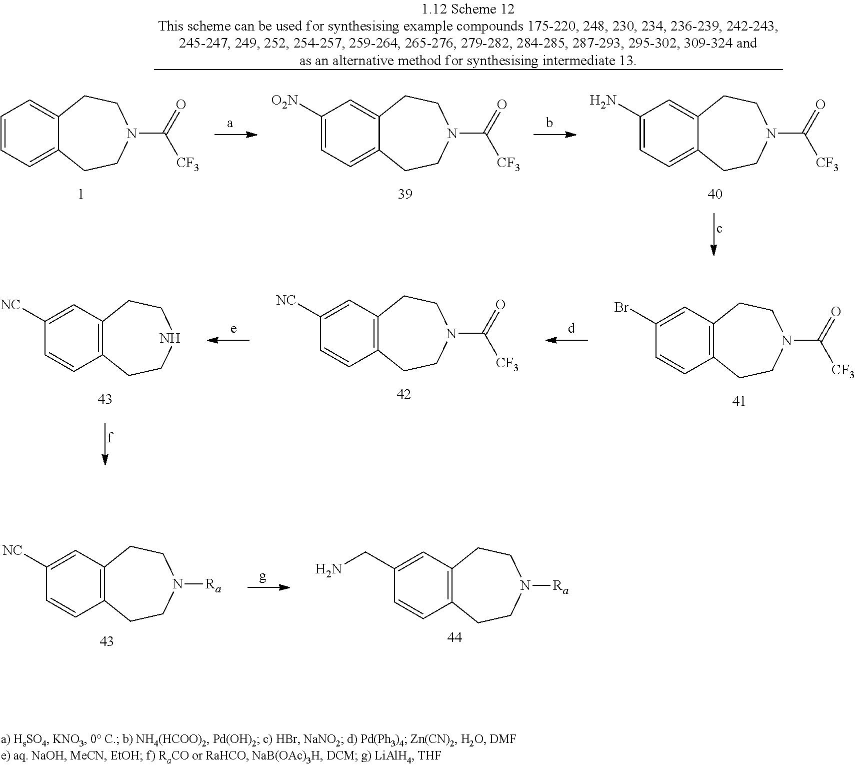

### 1.12.1 Intermediate 39

1-(4,5-Dihydro-1H-benzo[d]azepin-3(2H)-yl)-2,2,2-trifluoroethanone (100 g, 411 mmol) was dissolved in sulfuric acid (400 ml) and the solution was cooled in an ice-bath. Potassium nitrate (45.7 g, 452 mmol) was added portion-wise over 20 minutes (no exotherm observed, solution turned pale yellow). The solution was stirred for a further 10 minutes after which time the reaction was complete by TLC (30% ethyl acetate in petrol, Potassium permanganate staining). The reaction mixture was slowly poured into 2×2 litre beakers full of ice with stirring. A thick white ppt. formed, and once about half of the product solution had been added, about 400 ml of DCM was added to the beakers to dissolve this. A further 200 ml of DCM was added to the beakers and the ice-cold layers were separated and the aqueous layer was extracted with DCM (1×200 ml). The combined organic layers were washed with brine (500 ml) and dried (MgSO4) and the solvent was removed under reduced pressure. Ethyl acetate (200 ml) was added to the resulting oil, and the product was left to crystallise overnight and then filtered to give 2,2,2-trifluoro-1-(7-nitro-4,5-dihydro-1H-benzo[d]azepin-3(2H)-yl)ethanone as a white solid, 50 g, 42%.

¹H NMR (400 MHz, CDCl₃) δ 7.95-8.16 (m, 2H), 7.24-7.47 (m, 1H), 3.62-4.01 (m, 4H), 2.95-3.32 (m, 4H)

### 1.12.2 Intermediate 40

2,2,2-Trifluoro-1-(7-nitro-4,5-dihydro-1H-benzo[d]azepin-3(2H)-yl)ethanone (50 g, 174 mmol) was dissolved in ethanol (1 litre) and ammonium formate (110 g, 10 eq.) was added, followed by Pd(OH)₂ (20% on carbon, wet, 2 g). The mixture was stirred vigorously under nitrogen and the reaction was initiated by warming in a hot water bath for 5 minutes. The reaction mixture began to effervesce quite vigorously. After 30 minutes the reaction was complete by tlc (30% EtOAc, petrol) and the mixture was filtered through celite with ethanol washing. The ethanol was then removed under reduced pressure and a water/ethyl acetate work-up was carried out and the combined organic layers were dried (MgSO₄) and the solvent removed to give an off-white solid which was triturated with ether to give 1-(7-Amino-4,5-dihydro-1H-benzo[d]azepin-3(2H)-yl)-2,2,2-trifluoroethanone as a white solid, 40 g, 89%.

1H NMR (400 MHz, CDCl₃) δ 6.82-7.09 (m, 1H), 6.44-6.61 (m, 2H), 3.59-3.84 (m, 4H), 2.66-2.99 (m, 4H).

### 1.12.3 Intermediate 41

1-(7-Amino-4,5-dihydro-1H-benzo[d]azepin-3(2H)-yl)-2,2,2-trifluoroethanone (70.3 g, 272 mmol) was suspended in hydrobromic acid (210 ml), and the mixture was cooled in a brine ice-bath and a solution of sodium nitrite in water (20 ml) was added drop-wise over 20 minutes to give a bright yellow thick suspension. Meanwhile 1-(7-amino-4,5-dihydro-1H-benzo[d]azepin-3(2H)-yl)-2,2,2-trifluoroethanone (70.3 g, 272 mmol) was dissolved in hydrobromic acid (210 ml) and the solution was heated to 85° C. in a 2 litre 2 neck flask with condenser. The suspension of diazonium salt was then added by pouring portion-wise over 20 minutes through a funnel—effervescence was observed on each addition. The residual diazonium salt was washed out with about 20 ml of hydrobromic acid and the solution was stirred for a further 1 hour at 85° C. After this time the TLC (30% ethyl acetate/petrol, Potassium permanganate staining, no uv) of an aliquot quenched in water and extracted with DCM showed complete conversion of the starting material to the desired product (top spot, and some of the phenol which shows up brightly with Potassium permanganate staining. The 1H NMR spectrum showed about 70% product+one other impurity, probably the phenol. The mixture was cooled to room temperature and diluted with water (about 600 ml) and the mixture was extracted with DCM (2×500 ml), and the combined organic layers washed with saturated NaHCO₃ (400 ml), dried (MgSO4) and the solvent was removed under reduced pressure to give a brown oil. This material was purified by dry flash column chromatography, eluting with 10-30% ethyl acetate in petrol to give 1-(7-Bromo-4,5-dihydro-1H-benzo[d]azepin-3(2H)-yl)-2,2,2-trifluoroethanone as a white crystalline solid, (40 g, 56%).

¹H NMR (400 MHz, CDCl₃) δ 6.85-7.07 (m, 1H), 6.40-6.61 (m, 2H), 3.62-3.87 (m, 4H), 2.69-3.05 (m, 4H).

### 1.12.4 Intermediate 42

1-(7-Bromo-4,5-dihydro-1H-benzo[d]azepin-3(2H)-yl)-2,2,2-trifluoroethanone (20 g, 62.1 mmol) was dissolved in DMF (150 ml) and water (0.50 ml) and tetrakis(triphenylphosphine) palladium (0) (0.789 g, 0.683 mmol) and dicyanozinc (5.04 g, 43.5 mmol) were added and the solution was heated under reflux under nitrogen for 3 hours. After this time there was still some starting material by tlc (30% EtOAc/petrol, Potassium permanganate staining, no uv) so another 500 mg of tetrakis(triphenylphosphine) palladium (0) was added and the solution was heated for a further hour after which time the reaction had gone to completion. Most of the DMF was removed under reduced pressure and saturated brine and ethyl acetate were added and the layers were separated. The organic layer was washed with brine (3×100 ml), dried (MgSO4) and the solvent was removed to give a dark grey-brown oil. This crude product was purified by dry flash column chromatography, eluting with 30% ethyl acetate in petrol to give the product as a colourless oil which crystallised on standing to give 3-(2,2,2-Trifluoroacetyl)-2,3,4,5-tetrahydro-1H-benzo[d]azepine-7-carbonitrile as a white solid, 12.8 g, 77%

¹H NMR (400 MHz, CDCl₃) δ 7.50 (s, 2H), 7.28 (s, 1H), 3.57-3.93 (m, 4H), 2.82-3.30 (m, 4H).

### 1.12.5 Intermediate 43

3-(2,2,2-Trifluoroacetyl)-2,3,4,5-tetrahydro-1H-benzo[d]azepine-7-carbonitrile (2 g, 7.46 mmol) was dissolved in ethanol (50 ml) and acetonitrile (10.0 ml) and 2 M aqueous sodium hydroxide (7.5 ml, 14.91 mmol) was added and the solution was stirred for 30 minutes, after which time the reaction had gone to completion by TLC (30% EtOAc/petrol). The solvent was removed under reduced pressure and water (50 ml) and DCM (100 ml) were added and the layers were separated and the organic layer was washed with brine (50 ml) and dried (MgSO₄) to give 3-cyclobutyl-2,3,4,5-tetrahydro-1H-benzo[d]azepine-7-carbonitrile as a colourless oil, 1.1 g, 86%.

¹H NMR (400 MHz, CDCl₃) δ 7.32-7.48 (m, 2H), 7.13-7.24 (m, 1H), 2.80-3.10 (m, 8H).

### 1.12.6 Intermediate 44

3-Cyclobutyl-2,3,4,5-tetrahydro-1H-benzo[d]azepine-7-carbonitrile (8.1 g, 35.8 mmol) was dissolved in THF (50 ml) and the solution was added drop-wise over 20 minutes to a stirred solution of 1.0 M LiAlH₄ in THF (35.8 ml, 35.8 mmol) which was warmed to about 30° C. in a water bath. After 1 hour the reaction was complete by tlc of an aliquot quenched with water and diluted with ethyl acetate (10% MeOH/DCM+NH₃, ninhydrin). Hence the solution was cooled in an ice-bath and quenched by drop-wise addition of saturated Na₂SO₄ and then diluted with ethyl acetate (200 ml) and dried (MgSO₄) and the solution was filtered through celite and the solvent removed to give (3-Cyclobutyl-2,3,4,5-tetrahydro-1H-benzo[d]azepin-7-yl)methanamine as a waxy off-white solid, 6.96 g, 84%.

MS ES+ 231

¹H NMR (400 MHz, CDCl₃) δ 6.95 (s, 3H), 3.71 (s, 2H), 2.75-2.91 (m, 4H), 2.59-2.75 (m, 1H), 2.16-2.51 (m, 4H), 1.89-2.06 (m, 2H), 1.68-1.88 (m, 2H), 1.42-1.67 (m, 2H).

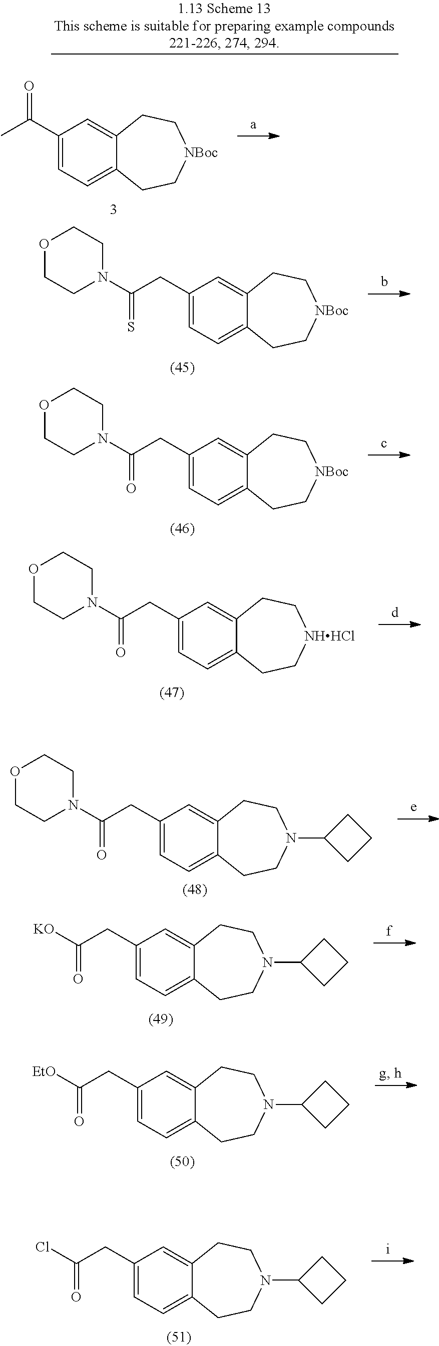

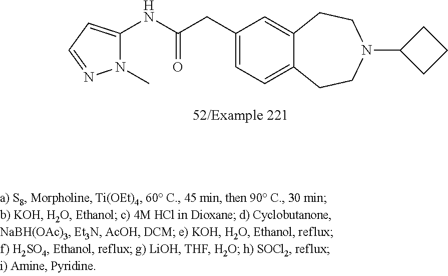

### 2-(3-cyclobutyl-2,3,4,5-tetrahydro-1H-benzo[d]azepin-7-yl)-N-(1-methyl-1H-pyrazol-5-yl)acetamide (example 221)

### 1.13.1 Intermediate 45

To a slurry of tert-butyl 7-acetyl-4,5-dihydro-1H-benzo[d]azepine-3(2H)-carboxylate (20.00 g, 69.12 mmol) in morpholine (9.1 mL, 9.03 g, 87.12 mmol) at RT was added sulphur powder (3.32 g, 103.67 mmol). The brown mixture was stirred for 5 min and then titanium(IV) ethoxide (29.0 mL, 31.53 g, 138.24 mmol) was added. The reaction was heated to 60° C. for 45 min and then to 90° C. for 30 min. LCMS indicated complete reaction, EtOAc (100 mL) and brine (50 mL) were added, the mixture was stirred at RT for 30 min and then filtered through celite and the solid washed with EtOAc. The filtrate was washed with brine (2×50 mL), dried (MgSO₄), filtered and concentrated. Purified on Biotage silica gel column, eluting with 10-50% EtOAc/petrol gradient to afford tert-butyl 7-(2-morpholino-2-thioxoethyl)-4,5-dihydro-1H-benzo[d]azepine-3(2H)-carboxylate as a brown gum (18.60 g, 47.63 mmol, 69%).

¹H NMR (400 MHz, MeOD) δ 7.05-7.21 (m, 3H), 4.26-4.38 (m, 4H), 3.64-3.81 (m, 4H), 3.45-3.64 (m, 4H), 3.38-3.46 (m, 2H), 2.78-3.01 (m, 4H), 1.40-1.58 (m, 9H)

MS ES⁺ 391

### 1.13.2 Intermediate 46

To a solution of tert-butyl 7-(2-morpholino-2-thioxoethyl)-4,5-dihydro-1H-benzo[d]azepine-3(2H)-carboxylate (45) (18.60 g, 47.63 mmol) in ethanol (200 mL) was added KOH (1M, aq., 95.3 mL, 95.30 mmol). The mixture was stirred overnight at RT and then further KOH (1M, aq., 47.6 mL, 47.60 mmol) was added and the reaction stirred for a further 20 h at RT. The reaction was poured into water (50 mL) and DCM (150 mL) was added. The phases were separated and the aqueous phase extracted with DCM (3×100 mL). The combined organic phases were washed with brine (2×100 mL), dried (MgSO₄), filtered and concentrated. Purified on silica gel, eluting with 20-40-60-80-100% EtOAc/petrol stepwise gradient to yield tert-butyl 7-(2-morpholino-2-oxoethyl)-4,5-dihydro-1H-benzo[d]azepine-3(2H)-carboxylate as a light yellow solid (13.04 g, 34.82 mmol, 73%).

¹H NMR (400 MHz, DMSO δ 7.03-7.12 (m, 1H), 6.92-7.03 (m, 2H), 3.64 (s, 2H), 3.38-3.57 (m, 12H), 2.81 (d, J=3.54 Hz, 4H), 1.33-1.52 (m, 9H)

MS ES⁺ 375

### 1.13.3 Intermediate 47

A solution of tert-butyl 7-(2-morpholino-2-oxoethyl)-4,5-dihydro-1H-benzo[d]azepine-3(2H)-carboxylate (13.00 g, 34.72 mmol) in dioxane (100 mL) was treated with HCl in dioxane (4M, 40 mL). The reaction was treated with further HCl in dioxane (4M, 25 mL) and allowed to stand over the weekend. Removal of the solvent under reduced pressure afforded the product as the HCl salt 1-morpholino-2-(2,3,4,5-tetrahydro-1H-benzo[d]azepin-7-yl)ethanone hydrochloride (47) in quantitative yield, which was used immediately in the next reaction.

¹H NMR (400 MHz, DMSO-d₆) δ 9.03-9.25 (m, 2H), 7.09-7.17 (m, 1H), 6.98-7.09 (m, 2H), 3.63-3.69 (m, 2H), 3.38-3.55 (m, 8H), 2.98-3.22 (m, 8H)

MS ES⁺ 275

### 1.13.4 Intermediate 48

To a solution of 1-morpholino-2-(2,3,4,5-tetrahydro-1H-benzo[d]azepin-7-yl)ethanone hydrochloride (assume 34.72 mmol) in DCM (100 mL) was added triethylamine (5.32 mL, 3.86 g, 38.19 mmol) and cyclobutanone (3.9 mL, 3.65 g, 52.08 mmol). The mixture was stirred for 5 min and then sodium triacetoxyborohydride (11.04 g, 52.08 mmol) was added, followed by AcOH (1 mL). The mixture was stirred at RT for 3 h, LCMS indicated complete and the reaction mixture was poured into NaOH (2M, aq., 100 mL). The phases were separated and the aqueous phase was extracted with DCM (3×50 mL). The combined organic phases washed with brine (2×50 mL), dried (MgSO₄), filtered and concentrated. Purified by Biotage silica gel column, eluting with 1-10% of 2% NH₃/methanol in DCM. Pure fractions concentrated and product crystallised by addition of ether to yield 2-(3-cyclobutyl-2,3,4,5-tetrahydro-1H-benzo[d]azepin-7-yl)-1-morpholinoethanone (9.21 g, 28.04 mmol, 81%).

¹H NMR (400 MHz, DMSO) δ 6.99-7.07 (m, 1H), 6.90-6.99 (m, 2H), 3.63 (s, 2H), 3.41-3.56 (m, 8H), 2.65-2.85 (m, 5H), 2.32 (br. s., 4H), 1.92-2.08 (m, 2H), 1.69-1.85 (m, 2H), 1.49-1.67 (m, 2H)

MS ES⁺ 329.

### 1.13.5 Intermediate 49

A mixture of 2-(3-cyclobutyl-2,3,4,5-tetrahydro-1H-benzo[d]azepin-7-yl)-1-morpholinoethanone (5.16 g, 15.72 mmol) and KOH (4M, aq., 19.7 mL, 78.60 mmol) in ethanol (100 mL) was heated at reflux for 6 h, cooled to RT and allowed to stir over the weekend and then heated at reflux for a further 7 h. The reaction mixture was concentrated and used directly in the esterification reaction.

To a solution of potassium 2-(3-cyclobutyl-2,3,4,5-tetrahydro-1H-benzo[d]azepin-7-yl)acetate (49) (assume 17.24 mmol) in ethanol (200 mL) was added H₂SO₄ (conc., 30 mL). The mixture was heated at reflux and ethanol/water distilled off. Further ethanol was added as necessary, as well as further H₂SO₄ (10 mL). When complete by LCMS, the reaction mixture was poured onto ice and DCM (100 mL) was added. Solid Na₂CO₃ was added to neutralise the acid and raise the pH to ˜10. The solid was filtered off, washed with DCM (200 mL) and the filtrate phases were separated. The aqueous phase was extracted with DCM (3×100 mL) and the combined organic phases were dried (MgSO₄), filtered and concentrated. Purification by Biotage silica gel column, eluting with 0-10% of 2% NH₃/MeOH in DCM afforded the ethyl 2-(3-cyclobutyl-2,3,4,5-tetrahydro-1H-benzo[d]azepin-7-yl)acetate as a yellow oil (4.28 g, 14.89 mmol, 86% (2 steps)).

¹H NMR (400 MHz, MeOD) δ 6.95-7.14 (m, 3H), 4.07-4.24 (m, 2H), 3.57 (s, 2H), 2.80-3.01 (m, 5H), 2.50 (br. s., 4H), 2.06-2.22 (m, 2H), 1.89-2.03 (m, 2H), 1.61-1.82 (m, 2H), 1.17-1.33 (m, 3H)

MS ES⁺ 288.

### 1.13.6 Compound 52/(Example 221)

Ethyl 2-(3-cyclobutyl-2,3,4,5-tetrahydro-1H-benzo[d]azepin-7-yl)acetate (1.4 g, 4.87 mmol) was hydrolysed with lithium hydroxide (0.117 g, 4.87 mmol) in THF water 5:1 at reflux for 16 hours. The reaction mixture was evaporated and azeotroped with toluene (×3) to yield crude acid. This was then converted to the acid chloride by heating to reflux with excess thionyl chloride. The solvents were removed and the residue was azeotroped with toluene (×3) to yield 2-(3-cyclobutyl-2,3,4,5-tetrahydro-1H-benzo[d]azepin-7-yl)acetyl chloride (51) which was used unpurified in the next step.

2-(3-Cyclobutyl-2,3,4,5-tetrahydro-1H-benzo[d]azepin-7-yl)acetyl chloride (51) (0.895 g, 3.2 mmol) was dissolved in pyridine (3 mL) and 1-methyl-1Hpyrazol-5-ylamine was added and stirred together for 65 hours. The solvent was removed by evaporation and the residue was partitioned between ethyl acetate and 2M NaOH. The organic extract was dried and evaporated and the residue was purified by silica chromatography using 0-10% Methanol DCM with ammonia to yield 2-(3-cyclobutyl-2,3,4,5-tetrahydro-1H-benzo[d]azepin-7-yl)-N-(1-methyl-1H-pyrazol-5-yl)acetamide (0.45 g, 41%)

¹H NMR (400 MHz, MeOD) δ 7.33-7.44 (m, 1H), 7.07-7.19 (m, 3H), 6.18-6.26 (m, 1H), 3.60-3.74 (m, 5H), 2.82-3.01 (m, 5H), 2.52 (br. s., 4H), 2.07-2.21 (m, 2H), 1.87-2.04 (m, 2H), 1.59-1.83 (m, 2H)

MS ES⁺ 339

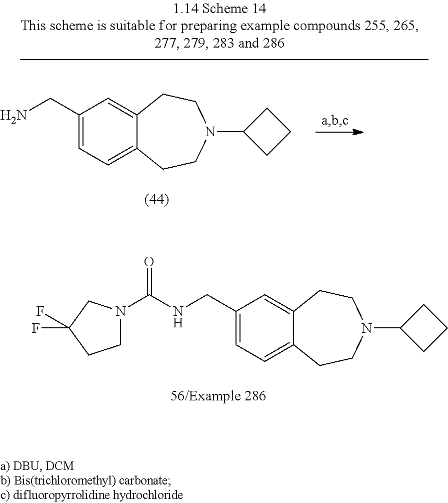

### 1.14.1 Compound 56/(Example 286)

Bis(trichloromethyl) carbonate (587 mg, 1.978 mmol) was dissolved in 20 ml dichloromethane and the solution was cooled in an ice-bath. (3-Cyclobutyl-2,3,4,5-tetrahydro-1H-benzo[d]azepin-7-yl)methanamine dihydrochloride (500 mg, 1.649 mmol) was suspended in 20 ml DCM and DBU (1 g, 6.6 mmol) was added to give a colourless solution which was added drop-wise to the bis(trichloromethyl)carbonate solution. After 10 minutes 3,3-difluoropyrrolidine hydrochloride (473 mg, 3.30 mmol) was added and the solution was warmed to room temperature and stirred for one hour and then heated to reflux for 30 minutes. The reaction mixture was washed with saturated sodium carbonate solution and the organic extract was dried and evaporated. The residue was purified by silica chromatography eluting with 0-12% methanol/dichloromethane with ammonia to give a colourless oil which was converted into the hydrochloride salt by dissolving in ethanol and adding 2M hydrogen chloride in ether. The product was crystallised using tert-butylmethylether and ethanol to give N-((3-cyclobutyl-2,3,4,5-tetrahydro-1H-benzo[d]azepin-7-yl)methyl)-3,3-difluoropyrrolidine-1-carboxamide Hydrochloride as a white solid white.

¹H NMR (400 MHz, METHANOL-d₄) δ 7.07 (s, 3H), 4.21 (s, 2H), 3.42-3.67 (m, 7H), 3.06-3.17 (m, 2H), 2.90-3.04 (m, 2H), 2.61-2.76 (m, 2H), 2.12-2.39 (m, 6H), 1.60-1.91 (m, 2H)

ES+ 364

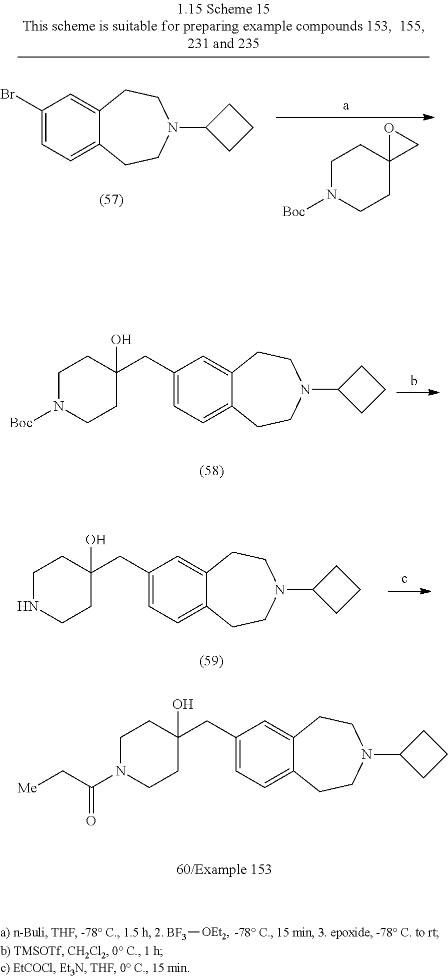

### 1.15.1 Intermediate 57

Acetic acid (2 mL) was added to a solution of 7-bromo-2,3,4,5-tetrahydro-1H-benzo[d]azepine (3.73 g, 16.50 mmol) and cyclobutanone (6.16 mL, 82 mmol) in dichloromethane (38 mL) at 0° C. The mixture was stirred at 0° C. for 1 hour. Sodium triacetoxyborohydride (10.49 g, 49.5 mmol) was added at 0° C. and the reaction mixture was slowly allowed to warm to 20° C. and stirred for 16 hours. To the reaction mixture was added 2N NaOH (aq.) (200 mL) and the mixture stirred for 20 minutes. The product was extracted with dichloromethane (3×150 mL), and the solvent removed under reduced pressure. The residue was purified by a strong cation exchange cartridge (50 g), loading with dichloromethane, and washing with methanol and eluting with 2M ammonia in methanol. The eluted solvent was removed under reduced pressure to give 7-bromo-3-cyclobutyl-2,3,4,5-tetrahydro-1H-benzo[d]azepine (4.47 g, >90%) as a white solid.

MS ES⁺ 280, 282

¹H NMR (400 MHz, DMSO-d₆) δ 7.29-7.35 (m, 1H), 7.23-7.29 (m, 1H), 7.03-7.09 (m, 1H), 2.69-2.85 (m, 5H), 2.32 (br. s., 4H), 1.93-2.05 (m, 2H), 1.68-1.82 (m, 2H), 1.47-1.65 (m, 2H)

### 1.15.2 Intermediate 58

A solution of n-butyllithium in n-hexane (1.6 M, 3.35 mL) was added to a solution of 7-bromo-3-cyclobutyl-2,3,4,5-tetrahydro-1H-benzo[d]azepine (1.5 g) in tetrahydrofuran (20 mL) at −78° C. The resulting mixture was stirred at −78° C. for 1.5 hours. Boron trifluoride etherate (0.68 mL) was added at −78° C. The resulting mixture was stirred at −78° C. for 15 minutes. A solution of tert-butyl 1-oxa-6-azaspiro[2.5]octane-6-carboxylate (1.14 g) in tetrahydrofuran (10 mL) was added at −78° C. The mixture was stirred at −78° C. for 2 hours and then warmed to room temperature for 16 hours. Saturated ammonium chloride aqueous solution (5.0 mL) was added at 0° C. The reaction mixture was partitioned between ethyl acetate and 2N NaOH (aq.). The organic layer was washed with brine and dried (Na₂SO₄), and the solvent was removed under reduced pressure. The residue was purified on basic silica eluting with 15-50% ethyl acetate in petrol) to give tert-butyl 4-((3-cyclobutyl-2,3,4,5-tetrahydro-1H-benzo[d]azepin-7-yl)methyl)-4-hydroxypiperidine-1-carboxylate (1.11 g, 50%) as white solid.

¹H NMR (400 MHz, DMSO-d₆) δ ppm 1.39-1.49 (m, 13H) 1.58-1.76 (m, 2H) 1.81-1.94 (m, 2H) 2.04-2.16 (m, 2H) 2.34-2.52 (m, 4H) 2.67-2.73 (m, 2H) 2.75-2.95 (m, 5H) 3.01-3.28 (m, 2H) 3.65-3.82 (m, 2H) 4.41 (s, 1H) 6.96-7.13 (m, 3H)

### 1.15.3 Intermediate 59

Trimethylsilyl trifluoromethane sulfonate (0.286 mL) was added to a solution of tert-butyl 4-((3-cyclobutyl-2,3,4,5-tetrahydro-1H-benzo[d]azepin-7-yl)methyl)-4-hydroxypiperidine-1-carboxylate (328 mg) in dichloromethane (10 mL) at 0° C. The mixture was stirred at 0° C. for 1 hour. The reaction mixture was partitioned between dichloromethane and 2N NaOH (aq.). The organic layer was washed with brine and dried (Na₂SO₄), and the solvent was removed under reduced pressure to give 4-((3-cyclobutyl-2,3,4,5-tetrahydro-1H-benzo[d]azepin-7-yl)methyl)piperidin-4-ol (231 mg, 93%) as pale yellow foam.

¹H NMR (400 MHz, DMSO-d₆) δ ppm 1.11-1.38 (m, 4H) 1.41-1.61 (m, 2H) 1.64-1.79 (m, 2H) 1.88-2.00 (m, 2H) 2.20-2.35 (m, 4H) 2.47-2.78 (m, 12H) 3.93 (s, 1H) 6.79-6.96 (m, 3H)

### 1.15.4 Compound 60/Example 153

Propionyl chloride (70 μL) was added to a solution of 4-((3-cyclobutyl-2,3,4,5-tetrahydro-1H-benzo[d]azepin-7-yl)methyl)piperidin-4-ol (230 mg) and triethylamine (150 μL) in THF (7.5 mL) at 0° C. The mixture was stirred at 0° C. for 15 minutes. The reaction mixture was partitioned between ethyl acetate and 2N NaOH (aq.). The organic layer was washed with brine and dried (Na₂SO₄), and the solvent was removed under reduced pressure. The residue was purified with basic silica, eluting with 25-100% ethyl acetate in petrol) and by recrystallisation from ethyl acetate to give 1-(4-((3-cyclobutyl-2,3,4,5-tetrahydro-1H-benzo[d]azepin-7-yl)methyl)-4-hydroxypiperidin-1-yl)propan-1-one (207 mg, 76%) as white crystals.

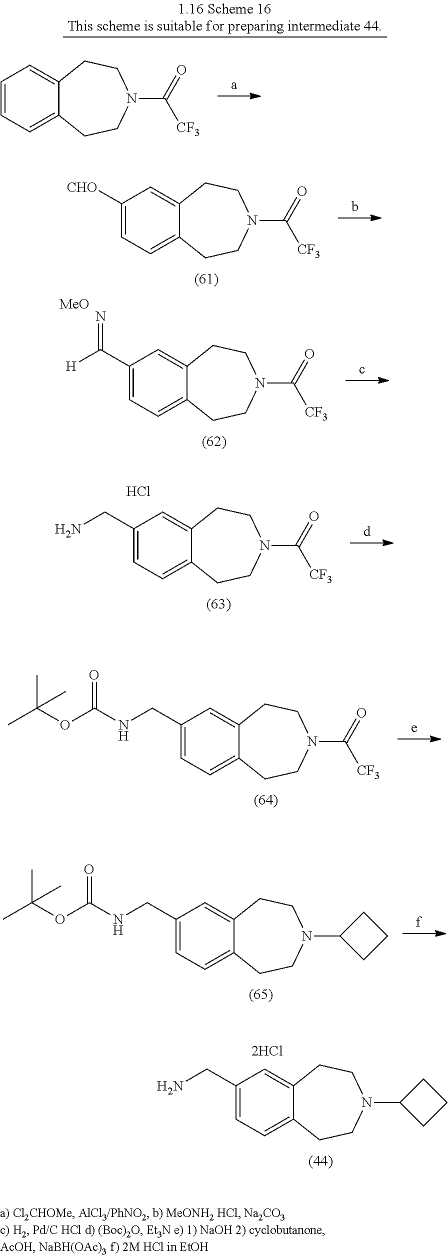

### 1.16.1 Intermediate 61

To a mixture of compound 3-(trifluoroacetyl)-2,3,4,5-tetrahydro-1H-3-benzazepine (24.3 g, 0.10 mol) and PhNO₂ (24 mL), was added AlCl₃ (26.7 g, 0.20 mol) at 5° C. (internal temperature) in one portion and stirred for 15 min. To the resulting mixture, was added a solution of Cl₂CHOCH₃ (34.5 g, 0.30 mol) in PhNO₂ (24 mL) dropwise at 5° C. over 50 min and the mixture was stirred at r.t. for 8 hr. The reaction mixture was diluted with AcOEt (100 mL) and poured onto ice (150 g) carefully. The mixture was extracted with AcOEt (100 mL×2) and was washed with water (50 mL×2). The combined organic layers were washed with brine (200 mL), dried over MgSO₄ and concentrated. The residue was purified by column chromatography on SiO₂ (350 g) (AcOEt/hexane=1/20 to 3/7) to give crude solid (25.0 g). The obtained solid was dissolved in IPE (30 mL) and hexane (90 mL) was added dropwise to the solution with stirring at 50° C. The mixture was cooled to r.t. and was stirred for 30 min. The deposited precipitate was filtered and was washed (AcOEt/hexane=1/5, 50 mL) to give 3-(Trifluoroacetyl)-2,3,4,5-tetrahydro-1H-3-benzazepine-7-carbaldehyde as pale yellow powder (20.3 g, 74.8%).

¹H-NMR (300 MHz, CDCl₃) δ: 3.05-3.10 (4H, m), 3.72-3.82 (4H, m), 7.31-7.72 (2H, m), 9.981 (1H, s). MS (POS/ESI), m/z 272.00 (M+1)⁺.

### 1.16.2 Intermediate 62

To a solution of Na₂CO₃ (6.36 g, 0.060 mol) in water (140 mL), was added MeONH₂HCl (10.0 g, 0.120 mol) portionwise at 5° C. (internal temperature) and stirred for 30 min. To the mixture, was added a solution of 3-(Trifluoroacetyl)-2,3,4,5-tetrahydro-1H-3-benzazepine-7-carbaldehyde 1 (27.1 g, 0.100 mol) in THF (140 mL) dropwise at 5° C. and the mixture was stirred at room temperature for 2 h. The reaction mixture was diluted with AcOEt (280 mL) and undissolved material was filtered. The separated aqueous layer was extracted with AcOEt (140 mL) and organic layers were combined and washed with brine (140 mL), and then dried over MgSO₄. The solvent was evaporated under reduced pressure to afford yellow oil (31 g) which was dissolved in IPE (62 mL) and then hexane (124 m) was added dropwise with stirring. The precipitate appeared was collected by filtration and washed with IPE-hexane (1:2, 50 mL), and then was dried under reduced pressure to afford 3-(Trifluoroacetyl)-2,3,4,5-tetrahydro-1H-3-benzazepine-7-carbaldehyde O-methyloxime as pale yellow powder (23.0 g, 76.6%).

¹H-NMR (400 MHz, CDCl₃) δ: 2.97-3.02 (4H, m), 3.68-3.71 (2H, m), 3.76-3.78 (2H, m), 3.97 (3H, s), 7.13-7.18 (1H, m), 7.33-7.36 (1H, m), 7.41-7.44 (1H, m), 8.03 (1H, s). MS (POS/ESI), m/z 300.98M⁺.

### 1.16.3 Intermediate 63

To a solution of 3-(Trifluoroacetyl)-2,3,4,5-tetrahydro-1H-3-benzazepine-7-carbaldehyde O-methyloxime 2 (21.0 g, 0.070 mol) in MeOH (420 mL) and aqueous 12 N HCl (5.3 mL, 175 mmol), was added 10% Pd/C (wet 50%, 2.1 g) and the mixture was hydrogenated under an atmospheric pressure at room temperature for 1 hr. The catalyst was removed by filtration and the filtrate was concentrated under reduced pressure. The resulting solid was treated with IPE (200 mL) and was collected by filtration, and then was dried under reduced pressure to afford 1-[3-(Trifluoroacetyl)-2,3,4,5-tetrahydro-1H-3-benzazepin-7-yl]methanamine hydrochloride (20.1 g, 92.8%) as white solid.

¹H-NMR (400 MHz, DMSO-d₆) δ: 2.96-3.02 (4H, m), 3.66-3.71 (4H, m), 3.96 (2H, s), 7.21-7.30 (3H, m), 8.33 (3H, brs).

### 1.16.4 Intermediate 64

To a solution of 1-[3-(Trifluoroacetyl)-2,3,4,5-tetrahydro-1H-3-benzazepin-7-yl]methanamine hydrochloride (18.5 g, 60 mmol) in THF (90 mL) and water (82 mL), was added (Boc)2O (13.1 g, 60 mmol) in one portion at 5° C. (internal temperature), and then aqueous 8N NaOH (7.5 mL, 60 mL) solution dropwise at the same temperature. The mixture was stirred at room temperature for 1 h. The reaction mixture was extracted with AcOEt (90 mL×2) and combined organic layers were washed with brine (90 mL), and then dried over MgSO₄. The solvent was evaporated under reduced pressure to give light brown syrup, which was treated with hexane (70 mL) to afford white precipitate. The obtained precipitate was collected by filtration and washed with hexane (20 mL), and then was dried under reduced pressure to give tert-Butyl {[3-(trifluoroacetyl)-2,3,4,5-tetrahydro-1H-3-benzazepin-7-yl]methyl}carbamate (21.0 g, 94%) as white powder.

¹H-NMR (400 MHz, CDCl₃) δ: 1.46 (9H, s), 2.94-2.99 (4H, m), 3.67-3.69 (2H, m), 3.74 3.78 (2H, m), 4.27-4.29 (2H, m), 4.83 (1H, brs), 7.06-7.14 (3H, m).

### 1.16.5 Intermediate 65

To a solution of tert-Butyl {[3-(trifluoroacetyl)-2,3,4,5-tetrahydro-1H-3-benzazepin-7-yl]methyl}carbamate (16.8 g, 45.0 mmol) in MeOH (170 mL), was added aqueous 8N NaOH solution (6.2 mL, 49.5 mmol) at 5° C. (internal temperature) and the mixture was stirred at room temperature for 1 h. To the resulting mixture, were added AcOH (3.9 mL, 67.5 mmol), cyclobutanone (4.7 g, 67.5 mmol), and NaBH(OAc)₃ (14.3 g, 67.5 mmol) at 5° C. and the mixture was stirred at room temperature for 3 h. To the mixture, were added cyclobutanone (4.7 g, 67.5 mmol) and NaBH(OAc)₃ (14.3 g, 67.5 mmol) again and the mixture was stirred at room temperature for 1 h. The reaction mixture was concentrated under reduced pressure and the residue was treated with water (150 mL). The aqueous mixture was made basic (pH=9) with aqueous NaOH solution under cooling and was extracted with AcOEt (150 mL×2). The combined organic layers were washed with brine (150 mL) and dried over MgSO₄. The solution was subjected to short silica-gel pad (40 g) and the solvent was evaporated under reduced pressure. The obtained solid was treated with hexane-IPE (1:1, 100 mL) and was collected by filtration. The solid was washed with hexane (10 mL) and was dried under reduced pressure to afford tert-Butyl [(3-cyclobutyl-2,3,4,5-tetrahydro-1H-3-benzazepin-7-yl)methyl]carbamate (12.4 g, 83.3%) as white solid.

¹H-NMR (400 MHz, CDCl₃) δ: 1.55-1.75 (2H, m), 1.85-1.97 (2H, m), 2.03-2.12 (2H, m), 2.35-2.50 (4H, m), 2.72-2.81 (1H, m), 2.87-2.94 (4H, m), 4.25-4.27 (2H, m), 4.78 (1H, brs), 7.01-7.07 (3H, m). MS (POS/ESI), m/z 331.22 (M+1)⁺.

Deprotection of the benzazepine nitrogen of intermediate 64 (step (e)(1) of Scheme 17) leads to tert-butyl (2,3,4,5-tetrahydro-1H-benzo[d]azepin-7-yl)methylcarbamate. ¹H-NMR (400 MHz, CDCl₃) δ 1.55-1.75 (2H, m), 1.85-1.97 (2H, m), 2.03-2.12 (2H, m), 2.35-2.50 (4H, m), 2.72-2.81 (1H, m), 2.87-2.94 (4H, m), 4.25-4.27 (2H, m), 4.78 (1H, brs), 7.01-7.07 (3H, m).

### 1.16.6 Intermediate 44

A mixture of tert-Butyl [(3-cyclobutyl-2,3,4,5-tetrahydro-1H-3-benzazepin-7-yl)methyl]carbamate (11.6 g, 35.0 mmol) and 2M ethanolic HCl solution (87.5 mL, 175 mmol) was warmed at 50° C. for 30 min. The reaction mixture was cooled in an iced water bath and treated with IPE (100 mL). The deposited precipitate was collected by filtration and was washed with IPE (20 mL), and then was dried under reduced pressure to afford 1-(3-Cyclobutyl-2,3,4,5-tetrahydro-1H-3-benzazepin-7-yl)methanamine dihydrochloride (9.5 g, 90.0%) as white powder.

¹H-NMR (400 MHz, DMSO-d₆) δ: 1.58-1.74 (2H, m), 2.15-2.17 (2H, m), 2.49-2.54 (2H, m), 2.68-2.71 (2H, m), 2.94-3.00 (2H, m), 3.50-3.52 (4H, m), 3.64-3.66 (1H, m), 7.23-7.26 (1H, m), 7.33-7.34 (2H, m), 8.56 (3H, brs), 11.94 (1H, brs). MS (POS/ESI), m/z 231.15 (M+1)⁺.

### 2. EXAMPLE COMPOUNDS

### 2.1 Example 1

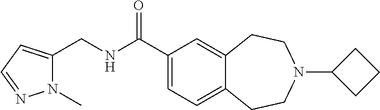

### 3-Cyclobutyl-N-((1-methyl-1H-pyrazol-5-yl)methyl)-2,3,4,5-tetrahydro-1H-benzo[d]azepine-7-carboxamide

¹H NMR (400 MHz, DMSO-d6) δ 8.79-8.96 (m, 1H), 7.56-7.68 (m, 2H), 7.27-7.34 (m, 1H), 7.15-7.25 (m, 1H), 6.11-6.20 (m, 1H), 4.43-4.55 (m, 2H), 3.35 (s, 3H), 2.81-2.96 (m, 4H), 2.69-2.80 (m, 1H), 2.34 (br. s., 4H), 1.91-2.08 (m, 2H), 1.70-1.85 (m, 2H), 1.47-1.67 (m, 2H)

Mass Spec: ES⁺ 339

### 2.2 Example 2

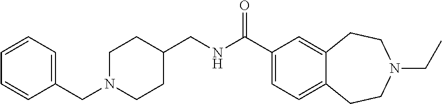

### N-((1-Benzylpiperidin-4-yl)methyl)-3-ethyl-2,3,4,5-tetrahydro-1H-benzo[d]azepine-7-carboxamide

¹H NMR (400 MHz, CDCl₃) δ 7.38-7.53 (m, 2H), 7.17-7.35 (m, 6H), 7.03-7.17 (m, 1H), 6.13 (t, J=5.43 Hz, 1H), 3.46 (s, 2H), 3.31 (t, J=6.32 Hz, 2H), 2.79-3.03 (m, 5H), 2.47-2.73 (m, 5H), 1.85-2.01 (m, 2H), 1.51-1.76 (m, 3H), 1.16-1.42 (m, 3H), 1.01-1.11 (m, 3H)

Mass Spec: ES⁺ 406

### 2.3 Example 3

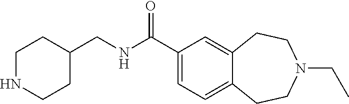

### 3-Ethyl-N-(piperidin-4-ylmethyl)-2,3,4,5-tetrahydro-1H-benzo[d]azepine-7-carboxamide Hydrochloride

¹H NMR (400 MHz, MeOD) δ 7.69-7.78 (m, 2H), 7.33-7.41 (m, 1H), 3.76-3.86 (m, 2H), 3.27-3.49 (m, 9H), 3.14-3.26 (m, 2H), 2.94-3.13 (m, 4H), 1.93-2.09 (m, 3H), 1.38-1.59 (m, 5H)

Mass Spec: ES⁺ 316

### 2.4 Example 4

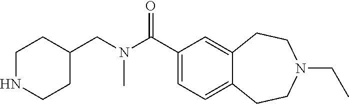

### 3-Ethyl-N-methyl-N-(piperidin-4-ylmethyl)-2,3,4,5-tetrahydro-1H-benzo[d]azepine-7-carboxamide

¹H NMR (400 MHz, MeOD) δ 7.08-7.28 (m, 3H), 3.43-3.53 (m, 1H), 3.23-3.31 (m, 1H), 3.11-3.22 (m, 1H), 3.08 (br. s., 1H), 3.01 (br. s., 4H), 2.49-2.76 (m, 8H), 1.95-2.09 (m, 1H), 1.74-1.91 (m, 3H), 1.53-1.62 (m, 1H), 1.25-1.42 (m, 2H), 1.10-1.19 (m, 3H), 0.82-0.97 (m, 1H)

Mass Spec: ES⁺ 330

### 2.5 Example 5

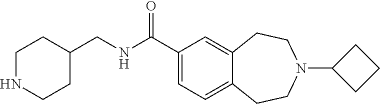

### 3-Cyclobutyl-N-(piperidin-4-ylmethyl)-2,3,4,5-tetrahydro-1H-benzo[d]azepine-7-carboxamide

¹H NMR (400 MHz, D₂O) δ 7.43-7.60 (m, 2H), 7.19-7.34 (m, 1H), 3.37-3.46 (m, 1H), 3.24-3.37 (m, 4H), 3.06-3.21 (m, 1H), 2.88-3.06 (m, 7H), 2.73 (br. s., 4H), 2.08-2.22 (m, 1H), 1.87-2.07 (m, 5H), 1.56-1.80 (m, 2H), 1.35-1.53 (m, 2H)

Mass Spec: ES⁺ 342

### 2.6 Example 6

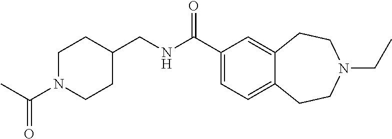

### N-((1-Acetylpiperidin-4-yl)methyl)-3-ethyl-2,3,4,5-tetrahydro-1H-benzo[d]azepine-7-carboxamide

¹H NMR (400 MHz, MeOD) δ 7.41-7.67 (m, 2H), 7.00-7.34 (m, 1H), 4.34-4.66 (m, 1H), 3.82-4.04 (m, 1H), 2.92-3.18 (m, 7H), 2.48-2.84 (m, 8H), 2.09 (s, 3H), 1.67-2.00 (m, 3H), 1.01-1.37 (m, 5H)

Mass Spec: ES⁺ 358

### 2.7 Example 7

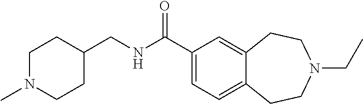

### 3-Ethyl-N-((1-methylpiperidin-4-yl)methyl)-2,3,4,5-tetrahydro-1H-benzo[d]azepine-7-carboxamide

1H NMR (400 MHz, MeOD) δ 7.52-7.62 (m, 2H), 7.14-7.28 (m, 1H), 3.24-3.28 (m, 1H), 2.94-3.10 (m, 4H), 2.79-2.94 (m, 2H), 2.46-2.79 (m, 6H), 2.17-2.37 (m, 3H), 1.92-2.14 (m, 2H), 1.51-1.89 (m, 4H), 1.23-1.44 (m, 2H), 1.01-1.22 (m, 3H)

Mass Spec: ES⁺ 330

### 2.8 Example 8

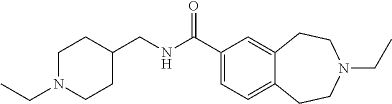

### 3-Ethyl-N-((1-ethylpiperidin-4-yl)methyl)-2,3,4,5-tetrahydro-1H-benzo[d]azepine-7-carboxamide

¹H NMR (400 MHz, MeOD) δ 7.52-7.62 (m, 2H), 7.15-7.24 (m, 1H), 3.25-3.29 (m, 2H), 2.90-3.10 (m, 6H), 2.53-2.82 (m, 6H), 2.38-2.45, 2H), 1.87-2.08 (m, 2H), 1.59-1.85 (m, 3H), 1.23-1.45 (m, 2H), 0.99-1.23 (m, 6H)

Mass Spec: ES⁺ 344

### 2.9 Example 9

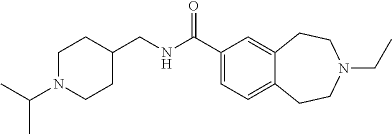

### 3-Ethyl-N-((1-isopropylpiperidin-4-yl)methyl)-2,3,4,5-tetrahydro-1H-benzo[d]azepine-7-carboxamide

¹H NMR (400 MHz, MeOD) δ 7.51-7.63 (m, 2H), 7.16-7.25 (m, 1H), 3.26 (d, J=6.57 Hz, 2H), 2.84-3.12 (m, 6H), 2.48-2.80 (m, 7H), 2.07-2.30 (m, 2H), 1.56-1.87 (m, 3H), 1.22-1.45 (m, 2H), 0.97-1.21 (m, 9H)

Mass Spec: ES⁺ 358

### 2.10 Example 10

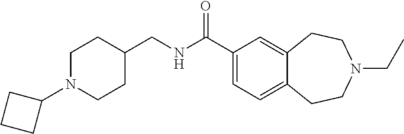

### N-((1-Cyclobutylpiperidin-4-yl)methyl)-3-ethyl-2,3,4,5-tetrahydro-1H-benzo[d]azepine-7-carboxamide

¹H NMR (400 MHz, MeOD) δ 7.51-7.63 (m, 2H), 7.16-7.24 (m, 1H), 3.24-3.29 (m, 2H), 2.88-3.10 (m, 6H), 2.51-2.88 (m, 7H), 1.99-2.16 (m, 2H), 1.59-2.00 (m, 9H), 1.22-1.45 (m, 2H), 1.05-1.21 (m, 3H)

Mass Spec: ES⁺ 370

### 2.11 Example 11

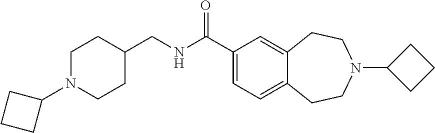

### 3-Cyclobutyl-N-((1-cyclobutylpiperidin-4-yl)methyl)-2,3,4,5-tetrahydro-1H-benzo[d]azepine-7-carboxamide

¹H NMR (400 MHz, MeOD) δ 7.45-7.68 (m, 2H), 7.14-7.28 (m, 1H), 3.25-3.29 (m, 2H), 2.98 (br. s., 6H), 2.78-2.93 (m, 2H), 2.51 (br. s., 4H), 2.03-2.19 (m, 4H), 1.86-2.03 (m, 6H), 1.61-1.86 (m, 7H), 1.25-1.46 (m, 2H)

Mass Spec: ES⁺ 396

### 2.12 Example 12

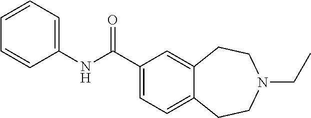

### 3-Ethyl-N-phenyl-2,3,4,5-tetrahydro-1H-benzo[d]azepine-7-carboxamide

¹H NMR (400 MHz, DMSO-d₆) δ 10.12 (s, 1H), 7.76 (d, J=7.83 Hz, 2H), 7.65-7.73 (m, 2H), 7.29-7.41 (m, 2H), 7.21-7.29 (m, 1H), 7.00-7.17 (m, 1H), 2.83-3.02 (m, 4H), 2.41-2.67 (m, 8H), 0.95-1.08 (m, 3H)

Mass Spec: ES⁺ 295

### 2.13 Example 13

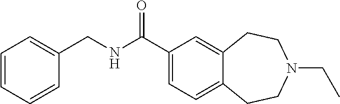

### N-Benzyl-3-ethyl-2,3,4,5-tetrahydro-1H-benzo[d]azepine-7-carboxamide

¹H NMR (400 MHz, MeOD) δ 7.53-7.69 (m, 2H), 7.15-7.44 (m, 6H), 4.49-4.65 (m, 2H), 2.90-3.08 (m, 4H), 2.47-2.79 (m, 6H), 1.04-1.20 (m, 3H)

Mass Spec: ES⁺ 309

### 2.14 Example 14

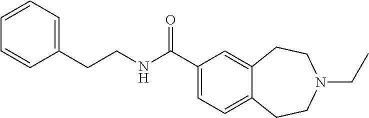

### 3-Ethyl-N-phenethyl-2,3,4,5-tetrahydro-1H-benzo[d]azepine-7-carboxamide

¹H NMR (400 MHz, MeOD) δ 7.45-7.64 (m, 2H), 7.03-7.36 (m, 6H), 3.48-3.65 (m, 2H), 2.83-3.07 (m, 6H), 2.45-2.75 (m, 6H), 0.98-1.29 (m, 3H)

Mass Spec: ES⁺ 323

### 2.15 Example 15

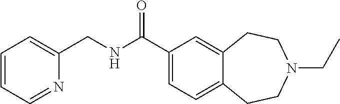

### 3-Ethyl-N-(pyridin-2-ylmethyl)-2,3,4,5-tetrahydro-1H-benzo[d]azepine-7-carboxamide

¹H NMR (400 MHz, MeOD) δ 8.49 (d, 1H), 7.76-7.85 (m, 1H), 7.63-7.69 (m, 2H), 7.39-7.42, 1H), 7.27-7.35 (m, 1H), 7.19-7.27 (m, 1H), 4.68 (s, 2H), 2.92-3.13 (m, 4H), 2.45-2.81 (m, 6H), 1.05-1.22 (m, 3H)

Mass Spec: ES⁺ 310

### 2.16 Example 16

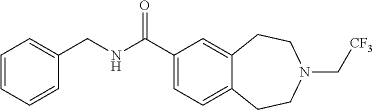

### N-Benzyl-3-(2,2,2-trifluoroethyl)-2,3,4,5-tetrahydro-1H-benzo[d]azepine-7-carboxamide

1H NMR (400 MHz, CDCl₃) δ 7.49-7.65 (m, 2H), 7.24-7.46 (m, 5H), 7.10-7.20 (m, 1H), 6.48 (br. s., 1H), 4.35-4.92 (m, 2H), 3.11-3.27 (m, 2H), 2.82-3.03 (m, 8H)

Mass Spec: ES⁺ 363

### 2.17 Example 17

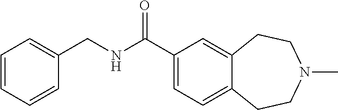

### N-Benzyl-3-methyl-2,3,4,5-tetrahydro-1H-benzo[d]azepine-7-carboxamide

¹H NMR (400 MHz, CDCl₃) δ 7.49-7.66 (m, 2H), 7.24-7.45 (m, 5H), 7.01-7.21 (m, 1H), 6.51 (br. s., 1H), 4.64 (d, J=5.56 Hz, 2H), 2.97 (br. s., 4H), 2.47-2.70 (m, 4H), 2.36 (s, 3H)

Mass Spec: ES⁺ 295

### 2.18 Example 18

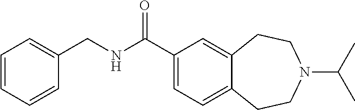

### N-Benzyl-3-isopropyl-2,3,4,5-tetrahydro-1H-benzo[d]azepine-7-carboxamide

¹H NMR (400 MHz, CDCl₃) δ 7.48-7.60 (m, 2H), 7.28-7.41 (m, 5H), 7.12-7.18 (m, 1H), 6.37 (br. s., 1H), 4.57-4.73 (m, 2H), 2.85-3.06 (m, 5H), 2.53-2.74 (m, 4H), 0.90-1.11 (m, 6H)

Mass Spec: ES⁺ 323

### 2.19 Example 19

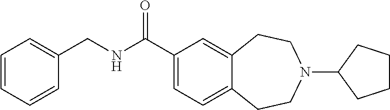

### N-Benzyl-3-cyclopentyl-2,3,4,5-tetrahydro-1H-benzo[d]azepine-7-carboxamide

¹H NMR (400 MHz, CDCl₃) δ 7.47-7.60 (m, 2H), 7.28-7.44 (m, 5H), 7.10-7.18 (m, 1H), 6.33 (br. s., 1H), 4.49-4.80 (m, 2H), 2.91-3.04 (m, 4H), 2.79-2.91 (m, 1H), 2.62-2.77 (m, 4H), 1.80-1.93 (m, 2H), 1.61-1.76 (m, 2H), 1.39-1.62 (m, 4H)

Mass Spec: ES⁺ 349

### 2.20 Example 20

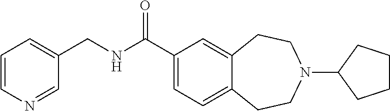

### 3-Cyclopentyl-N-(pyridin-3-ylmethyl)-2,3,4,5-tetrahydro-1H-benzo[d]azepine-7-carboxamide

¹H NMR (400 MHz, MeOD) δ 8.52-8.58 (m, 1H), 8.41-8.49 (m, 1H), 7.82-7.89 (m, 1H), 7.67-7.76 (m, 2H), 7.38-7.46 (m, 1H), 7.29-7.36 (m, 1H), 4.60 (s, 2H), 3.57 (br. s., 1H), 3.15-3.27 (m, 8H), 2.08-2.22 (m, 2H), 1.61-1.90 (m, 6H)

Mass Spec: ES⁺ 350

### 2.21 Example 21

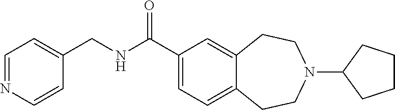

### 3-Cyclopentyl-N-(pyridin-4-ylmethyl)-2,3,4,5-tetrahydro-1H-benzo[d]azepine-7-carboxamide

¹H NMR (400 MHz, DMSO-d₆) δ 8.80-8.96 (m, 1H), 8.44-8.56 (m, 2H), 7.57-7.70 (m, 2H), 7.24-7.35 (m, 2H), 7.15-7.24 (m, 1H), 4.39-4.55 (m, 2H), 2.90 (br. s., 4H), 2.64 (br. s., 4H), 1.69-1.88 (m, 2H), 1.28-1.69 (m, 6H)

Mass Spec: ES⁺ 315

### 2.22 Example 22

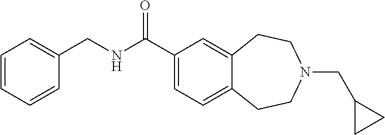

### N-Benzyl-3-(cyclopropylmethyl)-2,3,4,5-tetrahydro-1H-benzo[d]azepine-7-carboxamide

¹H NMR (400 MHz, CDCl₃) δ 8.41 (s, 1H), 7.44-7.57 (m, 2H), 7.19-7.32 (m, 3H), 7.18 (s, 1H), 7.04-7.13 (m, 1H), 6.37-6.53 (m, 1H), 4.47-4.63 (m, 2H), 2.98-3.30 (m, 8H), 2.63-2.85 (m, 2H), 0.85-1.10 (m, 1H), 0.55-0.66 (m, 2H), 0.12-0.30 (m, 2H)

Mass Spec: ES⁺ 335

### 2.23 Example 23

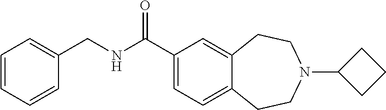

### N-Benzyl-3-cyclobutyl-2,3,4,5-tetrahydro-1H-benzo[d]azepine-7-carboxamide

¹H NMR (400 MHz, MeOD) δ 7.60-7.65 (m, 2H), 7.29-7.38 (m, 4H), 7.17-7.28 (m, 2H), 4.46-4.67 (m, 2H), 2.90-3.10 (m, 4H), 2.75-2.91 (m, 1H), 2.49 (br. s., 4H), 2.02-2.21 (m, 2H), 1.85-2.03 (m, 2H), 1.58-1.82 (m, 2H)

Mass Spec: ES⁺ 335

### 2.24 Example 24

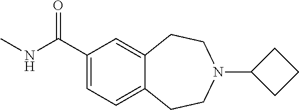

### 3-Cyclobutyl-N-methyl-2,3,4,5-tetrahydro-1H-benzo[d]azepine-7-carboxamide

¹H NMR (400 MHz, MeOD) δ 7.54-7.59 (m, 2H), 7.17-7.22 (m, 1H), 2.94-3.04 (m, 4H), 2.91 (s, 3H), 2.80-2.88 (m, 1H), 2.49 (br. s., 4H), 2.06-2.16 (m, 2H), 1.88-2.01 (m, 2H), 1.62-1.79 (m, 2H)

Mass Spec: ES⁺ 259

### 2.25 Example 25

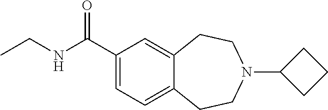

### 3-Cyclobutyl-N-ethyl-2,3,4,5-tetrahydro-1H-benzo[d]azepine-7-carboxamide

¹H NMR (400 MHz, MeOD) δ 7.58 (m, 2H), 7.09-7.27 (m, 1H), 3.36-3.49 (m, 2H), 2.93-3.08 (m, 4H), 2.74-2.92 (m, 1H), 2.50 (br. s., 4H), 2.02-2.22 (m, 2H), 1.83-2.05 (m, 2H), 1.60-1.82 (m, 2H), 1.12-1.36 (m, 3H)

Mass Spec: ES⁺ 273

### 2.26 Example 26

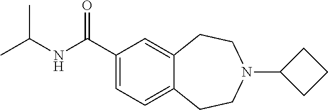

### 3-Cyclobutyl-N-isopropyl-2,3,4,5-tetrahydro-1H-benzo[d]azepine-7-carboxamide

¹H NMR (400 MHz, MeOD) δ 7.57 (d, 2H), 7.07-7.29 (m, 1H), 4.07-4.29 (m, 1H), 2.99 (br. s., 4H), 2.78-2.91 (m, 1H), 2.49 (br. s., 4H), 2.03-2.21 (m, 2H), 1.86-2.03 (m, 2H), 1.56-1.82 (m, 2H), 1.15-1.36 (m, 6H)

Mass Spec: ES⁺ 287

### 2.27 Example 27

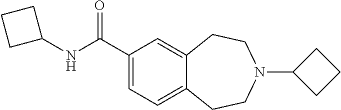

### N,3-Dicyclobutyl-2,3,4,5-tetrahydro-1H-benzo[d]azepine-7-carboxamide

¹H NMR (400 MHz, MeOD) δ 7.46-7.68 (m, 2H), 7.09-7.29 (m, 1H), 4.40-4.60 (m, 1H), 2.91-3.06 (m, 4H), 2.77-2.91 (m, 1H), 2.39-2.61 (m, 4H), 2.28-2.38 (m, 2H), 2.03-2.19 (m, 4H), 1.86-2.02 (m, 2H), 1.60-1.84 (m, 4H)

Mass Spec: ES⁺ 299

### 2.28 Example 28

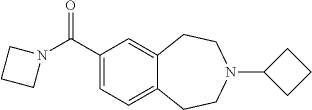

### Azetidin-1-yl(3-cyclobutyl-2,3,4,5-tetrahydro-1H-benzo[d]azepin-7-yl)methanone

¹H NMR (400 MHz, DMSO-d₆) δ 7.29-7.41 (m, 2H), 7.12-7.22 (m, 1H), 4.18-4.37 (m, 2H), 3.93-4.13 (m, 2H), 2.81-2.94 (m, 4H), 2.68-2.82 (m, 1H), 2.34 (br. s., 4H), 2.18-2.29 (m, 2H), 2.01 (q, J=7.8 Hz, 2H), 1.70-1.86 (m, 2H), 1.49-1.67 (m, 2H)

Mass Spec: ES⁺ 285

### 2.29 Example 29

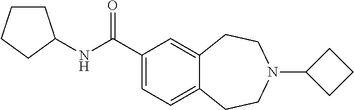

### 3-Cyclobutyl-N-cyclopentyl-2,3,4,5-tetrahydro-1H-benzo[d]azepine-7-carboxamide

¹H NMR (400 MHz, MeOD) δ 7.44-7.65 (m, 2H), 7.11-7.26 (m, 1H), 4.20-4.41 (m, 1H), 2.90-3.05 (m, 4H), 2.76-2.91 (m, 1H), 2.35-2.61 (m, 4H), 1.48-2.20 (m, 14H)

Mass Spec: ES⁺ 313

### 2.30 Example 30

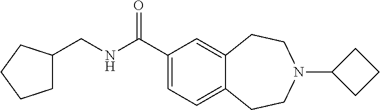

### 3-Cyclobutyl-N-(cyclopentylmethyl)-2,3,4,5-tetrahydro-1H-benzo[d]azepine-7-carboxamide

¹H NMR (400 MHz, DMSO-d₆) δ 8.21-8.48 (m, 1H), 7.47-7.71 (m, 2H), 7.07-7.30 (m, 1H), 3.11-3.22 (m, 2H), 2.68-2.98 (m, 4H), 2.22-2.45 (m, 4H), 2.07-2.21 (m, 1H), 1.95-2.07 (m, 2H), 1.72-1.89 (m, 2H), 1.42-1.71 (m, 9H), 1.17-1.31 (m, 2H)

Mass Spec: ES⁺ 327

### 2.31 Example 31

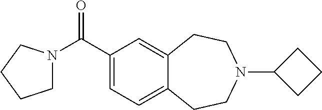

### (3-Cyclobutyl-2,3,4,5-tetrahydro-1H-benzo[d]azepin-7-yl)(pyrrolidin-1-yl)methanone

¹H NMR (400 MHz, DMSO-d₆) δ 7.04-7.42 (m, 3H), 3.39 (br. s., 4H), 2.81-3.13 (m, 4H), 2.47-2.52 (br. s., 4H), 2.10 (br. s., 2H), 1.75-1.96 (m, 6H), 1.53-1.74 (m, 2H)

Mass Spec: ES⁺ 299

### 2.32 Example 32

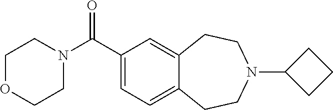

### (3-Cyclobutyl-2,3,4,5-tetrahydro-1H-benzo[d]azepin-7-yl)(morpholino)methanone

¹H NMR (400 MHz, MeOD) δ 7.06-7.35 (m, 3H), 3.54-3.85 (m, 6H), 3.40-3.53 (m, 2H), 2.97-3.16 (m, 5H), 2.58-2.83 (m, 4H), 2.13-2.25 (m, 2H), 1.98-2.13 (m, 2H), 1.64-1.86 (m, 2H)

Mass Spec: ES⁺ 315

### 2.33 Example 33

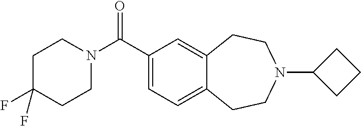

### (3-Cyclobutyl-2,3,4,5-tetrahydro-1H-benzo[d]azepin-7-yl)(4,4-difluoropiperidin-1-yl)methanone

1H NMR (400 MHz, DMSO-d6) δ 7.04-7.29 (m, 3H), 3.57 (br. s., 4H), 2.88 (br. s., 6H), 2.40 (br. s., 3H), 2.02 (br. s., 6H), 1.71-1.92 (m, 2H), 1.50-1.68 (m, 2H)

Mass Spec: ES⁺ 349

### 2.34 Example 34

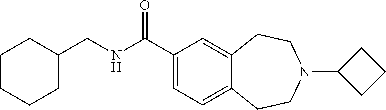

### 3-Cyclobutyl-N-(cyclohexylmethyl)-2,3,4,5-tetrahydro-1H-benzo[d]azepine-7-carboxamide

1H NMR (400 MHz, CDCl₃) δ 7.52-7.62 (m, 3H), 7.17-7.23 (m, 1H), 6.24-6.35 (m, 1H), 3.72-3.84 (m, 2H), 3.53-3.65 (m, 2H), 3.27-3.41 (m, 3H), 2.92-3.06 (m, 2H), 2.58-2.73 (m, 2H), 2.42-2.57 (m, 2H), 2.22-2.35 (m, 2H), 1.88-2.00 (m, 1H), 1.65-1.84 (m, 6H), 1.54-1.65 (m, 1H), 1.12-1.33 (m, 2H), 0.94-1.08 (m, 2H)

Mass Spec: ES⁺ 341

### 2.35 Example 35

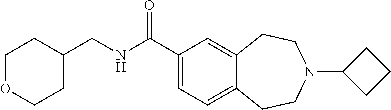

### 3-Cyclobutyl-N-((tetrahydro-2H-pyran-4-yl)methyl)-2,3,4,5-tetrahydro-1H-benzo[d]azepine-7-carboxamide

¹H NMR (400 MHz, DMSO-d6) δ 8.18-8.27 (m, 1H), 7.53-7.63 (m, 2H), 7.12-7.21 (m, 1H), 3.78-3.89 (m, 2H), 3.22-3.31 (m, 2H), 3.11-3.17 (m, 2H), 2.75-2.92 (m, 5H), 2.37 (br. s., 4H), 1.95-2.06 (m, 2H), 1.72-1.86 (m, 3H), 1.51-1.68 (m, 4H), 1.10-1.28 (m, 2H)

Mass Spec: ES⁺ 350

### 2.36 Example 36

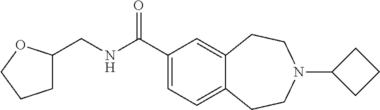

### 3-cyclobutyl-N-((tetrahydrofuran-2-yl)methyl)-2,3,4,5-tetrahydro-1H-benzo[d]azepine-7-carboxamide

¹H NMR (400 MHz, MeOD) δ 7.49-7.77 (m, 2H), 7.13-7.39 (m, 1H), 4.00-4.18 (m, 1H), 3.84-3.95 (m, 1H), 3.71-3.82 (m, 1H), 3.36-3.53 (m, 2H), 2.93-3.13 (m, 5H), 2.66 (br. s., 4H), 2.11-2.26 (m, 2H), 1.84-2.11 (m, 5H), 1.59-1.84 (m, 3H)

Mass Spec: ES⁺ 329

### 2.37 Example 37

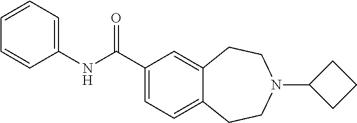

### 3-Cyclobutyl-N-phenyl-2,3,4,5-tetrahydro-1H-benzo[d]azepine-7-carboxamide

¹H NMR (400 MHz, DMSO-d6) δ 10.13 (s, 1H), 7.74-7.81 (m, 2H), 7.68-7.73 (m, 2H), 7.31-7.38 (m, 2H), 7.23-7.29 (m, 1H), 7.06-7.13 (m, 1H), 2.86-2.98 (m, 4H), 2.72-2.83 (m, 1H), 2.38 (br. s., 4H), 1.96-2.07 (m, 2H), 1.73-1.87 (m, 2H), 1.51-1.68 (m, 2H)

Mass Spec: ES⁺ 321

### 2.38 Example 38

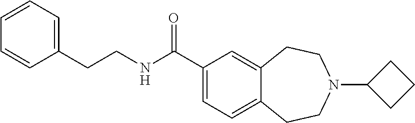

### 3-Cyclobutyl-N-phenethyl-2,3,4,5-tetrahydro-1H-benzo[d]azepine-7-carboxamide

¹H NMR (400 MHz, MeOD) δ 7.45-7.59 (m, 2H), 7.13-7.34 (m, 6H), 3.52-3.63 (m, 2H), 2.74-3.04 (m, 7H), 2.47 (br. s., 4H), 2.03-2.17 (m, 2H), 1.85-2.02 (m, 2H), 1.60-1.81 (m, 2H)

Mass Spec: ES⁺ 349

### 2.39 Example 39

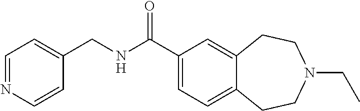

### 3-Ethyl-N-(pyridin-4-ylmethyl)-2,3,4,5-tetrahydro-1H-benzo[d]azepine-7-carboxamide

¹H NMR (400 MHz, MeOD) δ 8.44-8.51 (m, 2H), 7.62-7.70 (m, 2H), 7.36-7.44 (m, 2H), 7.20-7.29 (m, 1H), 4.62 (s, 2H), 3.03 (br. s., 4H), 2.56-2.80 (m, 6H), 1.15 (m, 3H)

Mass Spec: ES⁺ 310

### 2.40 Example 40

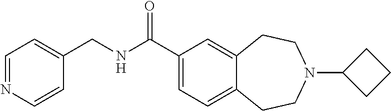

### 3-Cyclobutyl-N-(pyridin-4-ylmethyl)-2,3,4,5-tetrahydro-1H-benzo[d]azepine-7-carboxamide

¹H NMR (400 MHz, MeOD) δ 8.40-8.50 (m, 2H), 7.58-7.69 (m, 2H), 7.34-7.45 (m, 2H), 7.17-7.30 (m, 1H), 4.61 (s, 2H), 2.99 (br. s., 4H), 2.77-2.90 (m, 1H), 2.48 (br. s., 4H), 2.05-2.17 (m, 2H), 1.85-2.02 (m, 2H), 1.60-1.81 (m, 2H)

Mass Spec: ES⁺ 336

### 2.41 Example 41

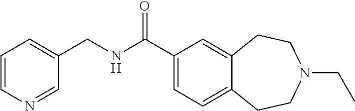

### 3-Ethyl-N-(pyridin-3-ylmethyl)-2,3,4,5-tetrahydro-1H-benzo[d]azepine-7-carboxamide

¹H NMR (400 MHz, MeOD) δ 8.54-8.60 (m, 1H), 8.42-8.49 (m, 1H), 7.82-7.92 (m, 1H), 7.59-7.68 (m, 2H), 7.39-7.48 (m, 1H), 7.19-7.29 (m, 1H), 4.62 (s, 2H), 2.96-3.10 (m, 4H), 2.52-2.78 (m, 6H), 1.07-1.23 (m, 3H)

Mass Spec: ES⁺ 310

### 2.42 Example 42

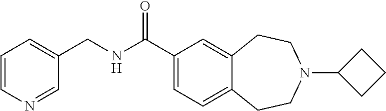

### 3-Cyclobutyl-N-(pyridin-3-ylmethyl)-2,3,4,5-tetrahydro-1H-benzo[d]azepine-7-carboxamide

¹H NMR (400 MHz, MeOD) δ 8.55 (s, 1H), 8.37-8.50 (m, 1H), 7.78-7.94 (m, 1H), 7.56-7.67 (m, 2H), 7.35-7.50 (m, 1H), 7.13-7.31 (m, 1H), 4.59 (s, 2H), 2.99 (br. s., 4H), 2.77-2.91 (m, 1H), 2.50 (br. s., 4H), 2.05-2.18 (m, 2H), 1.85-2.03 (m, 2H), 1.60-1.81 (m, 2H)

Mass Spec: ES⁺ 336

### 2.43 Example 43

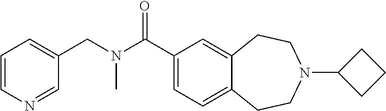

### 3-cyclobutyl-N-methyl-N-(pyridin-3-ylmethyl)-2,3,4,5-tetrahydro-1H-benzo[d]azepine-7-carboxamide

¹H NMR (400 MHz, MeOD) δ 8.93 (br. s., 1H), 8.78-8.86 (m, 1H), 8.62-8.72 (m, 1H), 8.05-8.18 (m, 1H), 7.29-7.47 (m, 3H), 4.94 (br. s., 2H), 3.64-3.79 (m, 4H), 3.06-3.22 (m, 5H), 2.77-2.90 (m, 2H), 2.31-2.44 (m, 4H), 1.75-1.99 (m, 2H)

Mass Spec: ES⁺ 350

### 2.44 Example 44

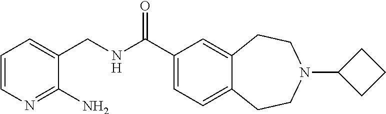

### N-((2-Aminopyridin-3-yl)methyl)-3-cyclobutyl-2,3,4,5-tetrahydro-1H-benzo[d]azepine-7-carboxamide

^{1H} NMR (400 MHz, MeOD) δ 7.77-7.91 (m, 1H), 7.53-7.65 (m, 2H), 7.40-7.52 (m, 1H), 7.15-7.29 (m, 1H), 6.53-6.72 (m, 1H), 4.44 (s, 2H), 2.98 (br. s., 4H), 2.77-2.92 (m, 1H), 2.50 (br. s., 4H), 2.04-2.20 (m, 2H), 1.84-2.03 (m, 2H), 1.57-1.82 (m, 2H)

Mass Spec: ES⁺ 351

### 2.45 Example 45

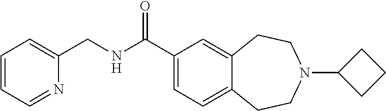

### 3-Cyclobutyl-N-(pyridin-2-ylmethyl)-2,3,4,5-tetrahydro-1H-benzo[d]azepine-7-carboxamide

¹H NMR (400 MHz, MeOD) δ 8.36-8.55 (m, 1H), 7.73-7.87 (m, 1H), 7.55-7.72 (m, 2H), 7.36-7.51 (m, 1H), 7.26-7.36 (m, 1H), 7.16-7.26 (m, 1H), 4.68 (s, 2H), 2.99 (br. s., 4H), 2.76-2.90 (m, 1H), 2.49 (br. s., 4H), 2.05-2.18 (m, 2H), 1.86-2.00 (m, 2H), 1.59-1.82 (m, 2H)

Mass Spec: ES⁺ 336

### 2.46 Example 46

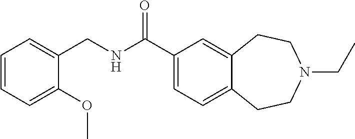

### 3-Ethyl-N-(2-methoxybenzyl)-2,3,4,5-tetrahydro-1H-benzo[d]azepine-7-carboxamide

¹H NMR (400 MHz, MeOD) δ 7.56-7.64 (m, 2H), 7.16-7.27 (m, 3H), 6.93-6.99 (m, 1H), 6.86-6.93 (m, 1H), 4.56 (s, 2H), 3.86 (s, 3H), 2.93-3.06 (m, 4H), 2.52-2.75 (m, 6H), 1.07-1.19 (m, 3H)

Mass Spec: ES⁺ 339

### 2.47 Example 47

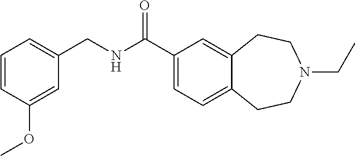

### 3-Ethyl-N-(3-methoxybenzyl)-2,3,4,5-tetrahydro-1H-benzo[d]azepine-7-carboxamide

¹H NMR (400 MHz, MeOD) δ 7.56-7.65 (m, 2H), 7.18-7.26 (m, 2H), 6.88-6.95 (m, 2H), 6.77-6.85 (m, 1H), 4.55 (s, 2H), 3.79 (s, 3H), 2.89-3.12 (m, 4H), 2.49-2.79 (m, 6H), 1.03-1.27 (m, 3H)

Mass Spec: ES⁺ 339

### 2.48 Example 48

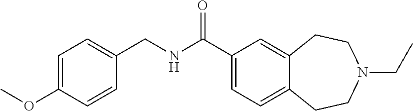

### 3-Ethyl-N-(4-methoxybenzyl)-2,3,4,5-tetrahydro-1H-benzo[d]azepine-7-carboxamide

¹H NMR (400 MHz, MeOD) δ 7.54-7.64 (m, 2H), 7.58 (none, 211), 7.21-7.31 (m, 2H), 7.13-7.20 (m, 1H), 6.81-6.92 (m, 2H), 4.48 (s, 2H), 3.75 (s, 3H), 2.89-3.05 (m, 4H), 2.48-2.73 (m, 6H), 1.03-1.19 (m, 3H)

Mass Spec: ES⁺ 336

### 2.49 Example 49

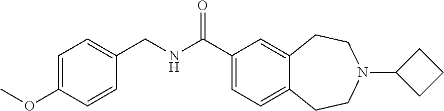

### 3-Cyclobutyl-N-(4-methoxybenzyl)-2,3,4,5-tetrahydro-1H-benzo[d]azepine-7-carboxamide

¹H NMR (400 MHz, CDCl₃) δ 7.47-7.56 (m, 2H), 7.24-7.32 (m, 2H), 7.09-7.18 (m, 1H), 6.85-6.93 (m, 2H), 6.29 (br. s., 1H), 4.53-4.63 (m, 2H), 3.81 (s, 3H), 2.95 (br. s., 4H), 2.78 (t, J=7.71 Hz, 1H), 2.44 (br. s., 4H), 2.02-2.15 (m, 2H), 1.83-1.98 (m, 2H), 1.57-1.77 (m, 2H)

Mass Spec: ES⁺ 356

### 2.50 Example 50

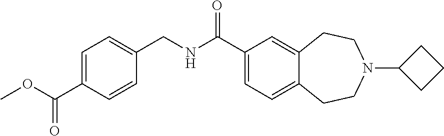

### Methyl 4-((3-cyclobutyl-2,3,4,5-tetrahydro-1H-benzo[d]azepine-7-carboxamido)methyl)benzoate

¹H NMR (400 MHz, CDCl₃) δ 7.97-8.08 (m, 2H), 7.49-7.62 (m, 2H), 7.38-7.47 (m, 2H), 7.11-7.21 (m, 1H), 6.25-6.59 (m, 1H), 4.63-4.79 (m, 2H), 3.92 (s, 3H), 2.97 (br. s., 4H), 2.72-2.85 (m, 1H), 2.45 (br. s., 4H), 2.01-2.16 (m, 2H), 1.82-1.97 (m, 2H), 1.55-1.78 (m, 2H)

Mass Spec: ES+ 393

### 2.51 Example 51

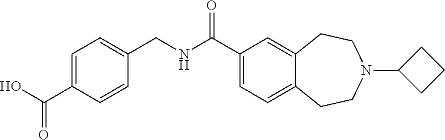

### 4-((3-Cyclobutyl-2,3,4,5-tetrahydro-1H-benzo[d]azepine-7-carboxamido)methyl)benzoic acid

¹H NMR (400 MHz, MeOD) δ 7.89-7.96 (m, 2H), 7.65-7.71 (m, 2H), 7.32-7.39 (m, 2H), 7.23-7.31 (m, 1H), 4.60 (s, 2H), 3.11 (br. s., 4H), 2.91 (br. s., 4H), 2.09-2.32 (m, 4H), 1.67-1.88 (m, 2H)

Mass Spec: ES⁺ 379

### 2.52 Example 52

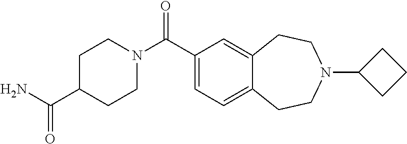

### 1-(3-Cyclobutyl-2,3,4,5-tetrahydro-1H-benzo[d]azepine-7-carbonyl)piperidine-4-carboxamide

¹H NMR (400 MHz, MeOD) δ 7.03-7.31 (m, 7H), 4.49-4.75 (m, 2H), 3.78 (br. s., 2H), 2.77-3.03 (m, 5H), 2.34-2.62 (m, 4H), 2.04-2.19 (m, 2H), 1.84-2.02 (m, 2H), 1.57-1.82 (m, 2H)

Mass Spec: ES⁺ 356

### 2.53 Example 53

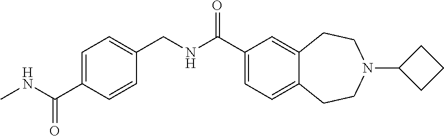

### 3-Cyclobutyl-N-(4-(methylcarbamoyl)benzyl)-2,3,4,5-tetrahydro-1H-benzo[d]azepine-7-carboxamide

¹H NMR (400 MHz, DMSO-d6) δ 8.85-9.13 (m, 1H), 8.28-8.53 (m, 1H), 7.68-7.89 (m, 2H), 7.54-7.71 (m, 2H), 7.29-7.47 (m, 2H), 7.08-7.28 (m, 1H), 4.42-4.59 (m, 2H), 2.88 (br. s., 4H), 2.71-2.83 (m, 4H), 2.36 (br. s., 4H), 1.94-2.06 (m, 2H), 1.72-1.87 (m, 2H), 1.50-1.68 (m, 2H)

Mass Spec: ES⁺ 392

### 2.54 Example 54

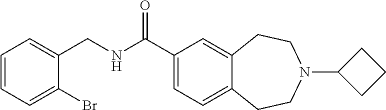

### N-(2-Bromobenzyl)-3-cyclobutyl-2,3,4,5-tetrahydro-1H-benzo[d]azepine-7-carboxamide

¹H NMR (400 MHz, CDCl₃) δ 7.45-7.62 (m, 4H), 7.28-7.34 (m, 1H), 7.12-7.21 (m, 2H), 6.57 (br. s., 1H), 4.65-4.79 (m, 2H), 2.90-3.07 (m, 4H), 2.74-2.86 (m, 1H), 2.47 (br. s., 4H), 2.03-2.15 (m, 2H), 1.87-2.00 (m, 2H), 1.55-1.78 (m, 2H)

Mass Spec: ES⁺ 413, 415

### 2.55 Example 55

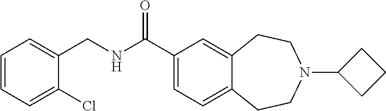

### N-(2-Cyanobenzyl)-3-cyclobutyl-2,3,4,5-tetrahydro-1H-benzo[d]azepine-7-carboxamide

¹H NMR (400 MHz, CDCl₃) δ 7.51-7.74 (m, 5H), 7.33-7.43 (m, 1H), 7.11-7.19 (m, 1H), 6.72-6.90 (m, 1H), 4.73-4.89 (m, 2H), 2.96 (br. s., 4H), 2.70-2.84 (m, 1H), 2.44 (br. s., 4H), 2.01-2.14 (m, 2H), 1.82-1.99 (m, 2H), 1.56-1.79 (m, 2H)

Mass Spec: ES⁺ 360

### 2.56 Example 56

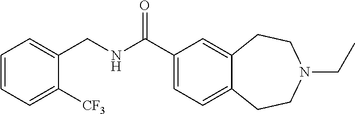

### 3-Ethyl-N-(2-(trifluoromethyl)benzyl)-2,3,4,5-tetrahydro-1H-benzo[d]azepine-7-carboxamide

¹H NMR (400 MHz, MeOD) δ 7.68-7.74 (m, 1H), 7.62-7.68 (m, 2H), 7.50-7.62 (m, 2H), 7.39-7.48 (m, 1H), 7.19-7.27 (m, 1H), 4.75-4.81 (m, 2H), 3.01 (br. s., 4H), 2.54-2.75 (m, 6H), 1.09-1.17 (m, 3H)

Mass Spec: ES⁺ 377

### 2.57 Example 57

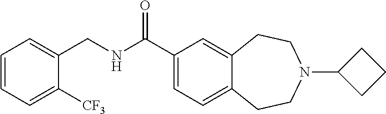

### 3-Cyclobutyl-N-(2-(trifluoromethyl)benzyl)-2,3,4,5-tetrahydro-1H-benzo[d]azepine-7-carboxamide

¹H NMR (400 MHz, MeOD) δ 7.67-7.73 (m, 1H), 7.62-7.67 (m, 2H), 7.50-7.61 (m, 2H), 7.38-7.47 (m, 1H), 7.18-7.25 (m, 1H), 4.74-4.81 (m, 2H), 2.98 (d, J=4.04 Hz, 4H), 2.76-2.90 (m, 1H), 2.48 (br. s., 4H), 2.05-2.16 (m, 2H), 1.86-2.00 (m, 2H), 1.59-1.78 (m, 2H)

Mass Spec: ES⁺ 403

### 2.58 Example 58

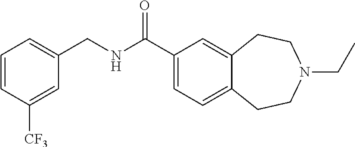

### 3-Ethyl-N-(3-(trifluoromethyl)benzyl)-2,3,4,5-tetrahydro-1H-benzo[d]azepine-7-carboxamide

¹H NMR (400 MHz, MeOD) δ 7.58-7.68 (m, 4H), 7.49-7.58 (m, 2H), 7.18-7.26 (m, 1H), 4.63 (s, 2H), 3.00 (br. s., 4H), 2.54-2.75 (m, 6H), 1.06-1.19 (m, 3H)

Mass Spec: ES⁺ 377

### 2.59 Example 59

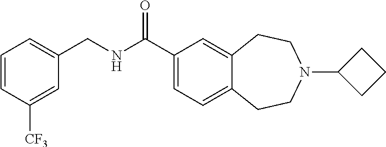

### 3-Cyclobutyl-N-(3-(trifluoromethyl)benzyl)-2,3,4,5-tetrahydro-1H-benzo[d]azepine-7-carboxamide

¹H NMR (400 MHz, MeOD) δ 7.58-7.68 (m, 4H), 7.49-7.58 (m, 2H), 7.17-7.25 (m, 1H), 4.62 (s, 2H), 2.98 (br. s., 4H), 2.77-2.91 (m, 1H), 2.49 (br. s., 4H), 2.03-2.18 (m, 2H), 1.85-2.01 (m, 2H), 1.60-1.80 (m, 2H)

Mass Spec: ES⁺ 403

### 2.60 Example 60

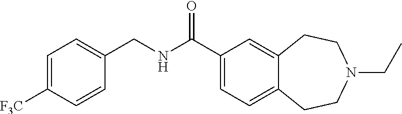

### 3-Ethyl-N-(4-(trifluoromethyl)benzyl)-2,3,4,5-tetrahydro-1H-benzo[d]azepine-7-carboxamide

¹H NMR (400 MHz, DMSO-d6) δ 10.13 (s, 1H), 7.74-7.81 (m, 2H), 7.68-7.73 (m, 2H), 7.31-7.38 (m, 2H), 7.23-7.29 (m, 1H), 7.06-7.13 (m, 1H), 2.86-2.98 (m, 4H), 2.72-2.83 (m, 1H), 2.38 (br. s., 4H), 1.96-2.07 (m, 2H), 1.73-1.87 (m, 2H), 1.51-1.68 (m, 2H)

Mass Spec: ES⁺ 377

### 2.61 Example 61

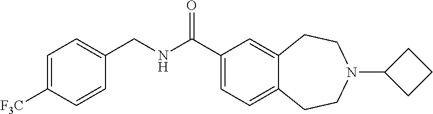

### 3-Cyclobutyl-N-(4-(trifluoromethyl)benzyl)-2,3,4,5-tetrahydro-1H-benzo[d]azepine-7-carboxamide

¹H NMR (400 MHz, MeOD) δ 7.58-7.66 (m, 4H), 7.48-7.57 (m, 2H), 7.17-7.25 (m, 2H), 4.63 (s, 2H), 2.98 (br. s., 4H), 2.76-2.89 (m, 1H), 2.48 (br. s., 4H), 2.04-2.17 (m, 2H), 1.86-2.01 (m, 2H), 1.60-1.78 (m, 2H)

Mass Spec: ES⁺ 403

### 2.62 Example 62

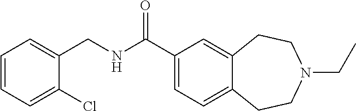

### N-(2-Chlorobenzyl)-3-ethyl-2,3,4,5-tetrahydro-1H-benzo[d]azepine-7-carboxamide

¹H NMR (400 MHz, MeOD) δ 7.58-7.69 (m, 2H), 7.34-7.44 (m, 2H), 7.19-7.32 (m, 3H), 4.65 (s, 2H), 3.01 (br. s., 4H), 2.54-2.77 (m, 6H), 1.08-1.19 (m, 3H)

Mass Spec: ES⁺ 343

### 2.63 Example 63

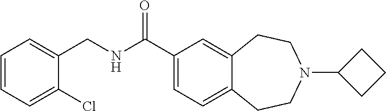

### N-(2-Chlorobenzyl)-3-cyclobutyl-2,3,4,5-tetrahydro-1H-benzo[d]azepine-7-carboxamide

¹H NMR (400 MHz, MeOD) δ 7.59-7.67 (m, 2H), 7.34-7.43 (m, 2H), 7.17-7.31 (m, 3H), 4.65 (s, 2H), 2.90-3.05 (m, 4H), 2.76-2.88 (m, 1H), 2.47 (br. s., 4H), 2.04-2.16 (m, 2H), 1.86-2.00 (m, 2H), 1.61-1.78 (m, 2H)

Mass Spec: ES⁺ 369

### 2.64 Example 64

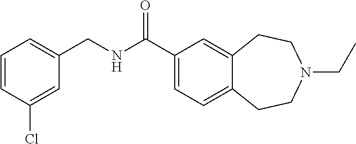

### N-(3-Chlorobenzyl)-3-ethyl-2,3,4,5-tetrahydro-1H-benzo[d]azepine-7-carboxamide

¹H NMR (400 MHz, MeOD) δ 7.58-7.66 (m, 2H), 7.18-7.40 (m, 5H), 4.54 (s, 2H), 3.01 (br. s., 4H), 2.54-2.75 (m, 6H), 1.09-1.18 (m, 3H)

Mass Spec: ES⁺ 343

### 2.65 Example 65

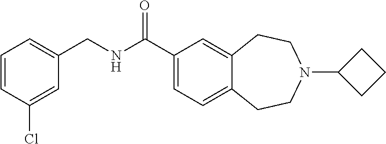

### N-(3-Chlorobenzyl)-3-cyclobutyl-2,3,4,5-tetrahydro-1H-benzo[d]azepine-7-carboxamide

¹H NMR (400 MHz, MeOD) δ 7.57-7.65 (m, 2H), 7.15-7.37 (m, 5H), 4.56 (s, 2H), 2.89-3.04 (m, 4H), 2.75-2.87 (m, 1H), 2.46 (br. s., 4H), 2.02-2.15 (m, 2H), 1.84-2.00 (m, 2H), 1.58-1.78 (m, 2H)

Mass Spec: ES⁺ 369

### 2.66 Example 66

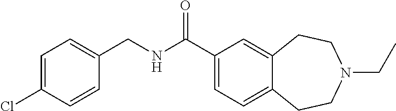

### N-(4-Chlorobenzyl)-3-ethyl-2,3,4,5-tetrahydro-1H-benzo[d]azepine-7-carboxamide

¹H NMR (400 MHz, MeOD) δ 7.57-7.64 (m, 2H), 7.27-7.36 (m, 4H), 7.16-7.23 (m, 1H), 4.52 (s, 2H), 2.90-3.04 (m, 4H), 2.53-2.72 (m, 6H), 1.06-1.16 (m, 3H)

Mass Spec: ES⁺ 343

### 2.67 Example 67

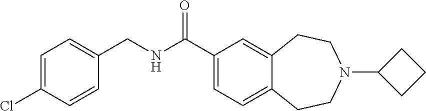

### N-(4-Chlorobenzyl)-3-cyclobutyl-2,3,4,5-tetrahydro-1H-benzo[d]azepine-7-carboxamide

¹H NMR (400 MHz, MeOD) δ 7.55-7.64 (m, 2H), 7.27-7.37 (m, 4H), 7.17-7.24 (m, 1H), 4.53 (s, 2H), 2.98 (br. s., 4H), 2.76-2.89 (m, 1H), 2.48 (br. s., 4H), 2.02-2.17 (m, 2H), 1.86-2.00 (m, 2H), 1.58-1.80 (m, 2H)

Mass Spec: ES⁺ 369

### 2.68 Example 68

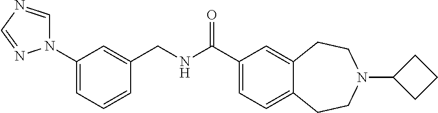

### N-(3-(1H-1,2,4-Triazol-1-yl)benzyl)-3-cyclobutyl-2,3,4,5-tetrahydro-1H-benzo[d]azepine-7-carboxamide

¹H NMR (400 MHz, DMSO-d6) δ 9.28 (s, 1H), 8.95-9.07 (m, 1H), 8.17-8.28 (m, 1H), 7.82 (s, 1H), 7.70-7.78 (m, 1H), 7.62-7.70 (m, 2H), 7.47-7.56 (m, 1H), 7.32-7.42 (m, 1H), 7.16-7.26 (m, 1H), 4.50-4.60 (m, 2H), 2.89 (br. s., 4H), 2.71-2.82 (m, 1H), 2.36 (br. s., 4H), 2.01 (d, J=6.57 Hz, 2H), 1.71-1.89 (m, 2H), 1.50-1.69 (m, 2H)

Mass Spec: ES⁺ 402

### 2.69 Example 69

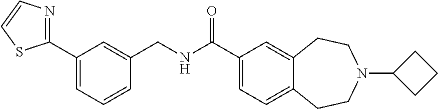

### 3-Cyclobutyl-N-(3-(thiazol-2-yl)benzyl)-2,3,4,5-tetrahydro-1H-benzo[d]azepine-7-carboxamide

¹H NMR (400 MHz, MeOD) δ 7.95 (s, 1H), 7.81-7.88 (m, 2H), 7.61-7.66 (m, 2H), 7.58-7.61 (m, 1H), 7.43-7.50 (m, 2H), 7.18-7.25 (m, 1H), 4.64 (s, 2H), 2.98 (br. s., 4H), 2.77-2.89 (m, 1H), 2.48 (br. s., 4H), 2.05-2.16 (m, 2H), 1.87-2.00 (m, 2H), 1.62-1.78 (m, 2H)

Mass Spec: ES⁺ 418

### 2.70 Example 70

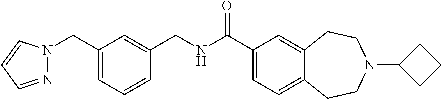

### N-(3-((1H-Pyrazol-1-yl)methyl)benzyl)-3-cyclobutyl-2,3,4,5-tetrahydro-1H-benzo[d]azepine-7-carboxamide

¹H NMR (400 MHz, DMSO-d6) δ 8.85-8.97 (m, 1H), 7.76-7.85 (m, 1H), 7.58-7.68 (m, 2H), 7.39-7.49 (m, 1H), 7.13-7.32 (m, 4H), 7.03-7.10 (m, 1H), 6.21-6.34 (m, 1H), 5.31 (s, 2H), 4.35-4.51 (m, 2H), 2.68-3.04 (m, 5H), 2.37 (br. s., 4H), 1.94-2.10 (m, 2H), 1.71-1.89 (m, 2H), 1.49-1.69 (m, 2H)

Mass Spec: ES⁺ 415

### 2.71 Example 71

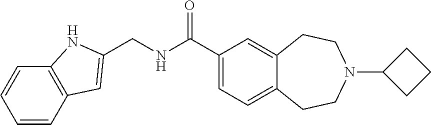

### N-((1H-Indol-2-yl)methyl)-3-cyclobutyl-2,3,4,5-tetrahydro-1H-benzo[d]azepine-7-carboxamide

¹H NMR (400 MHz, MeOD) δ 7.63 (br. s., 3H), 7.41-7.48 (m, 1H), 7.28-7.34 (m, 1H), 7.16-7.24 (m, 1H), 7.01-7.08 (m, 1H), 6.92-6.99 (m, 1H), 6.34 (s, 1H), 4.70 (s, 2H), 2.98 (br. s., 4H), 2.80-2.92 (m, 1H), 2.51 (br. s., 4H), 2.05-2.17 (m, 2H), 1.88-2.01 (m, 2H), 1.60-1.79 (m, 2H)

Mass Spec: ES⁺ 374

### 2.72 Example 72

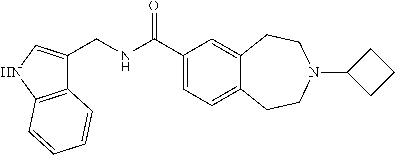

### N-((1H-Indol-3-yl)methyl)-3-cyclobutyl-2,3,4,5-tetrahydro-1H-benzo[d]azepine-7-carboxamide

¹H NMR (400 MHz, MeOD) δ 7.61-7.67 (m, 1H), 7.53-7.58 (m, 2H), 7.32-7.36 (m, 1H), 7.24 (s, 1H), 7.13-7.17 (m, 1H), 7.07-7.12 (m, 1H), 6.97-7.04 (m, 1H), 4.73 (s, 2H), 2.93 (br. s., 4H), 2.75-2.85 (m, 1H), 2.44 (br. s., 4H), 2.02-2.13 (m, 2H), 1.85-1.98 (m, 2H), 1.60-1.76 (m, 2H)

Mass Spec: ES⁺ 374

### 2.73 Example 73

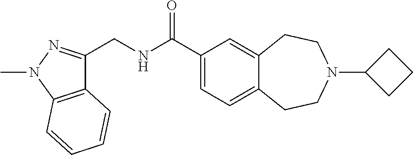

### 3-Cyclobutyl-N-((1-methyl-1H-indazol-3-yl)methyl)-2,3,4,5-tetrahydro-1H-benzo[d]azepine-7-carboxamide

¹H NMR (400 MHz, MeOD) δ 7.79-7.85 (m, 1H), 7.57-7.62 (m, 2H), 7.45-7.51 (m, 1H), 7.36-7.44 (m, 1H), 7.15-7.21 (m, 1H), 7.08-7.15 (m, 1H), 4.02 (s, 3H), 2.89-3.01 (m, 4H), 2.76-2.87 (m, 1H), 2.46 (br. s., 4H), 2.03-2.15 (m, 2H), 1.84-2.01 (m, 2H), 1.61-1.78 (m, 2H)

Mass Spec: ES⁺ 389

### 2.74 Example 74

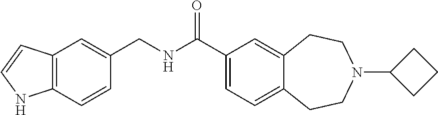

### N-((1H-Indol-5-yl)methyl)-3-cyclobutyl-2,3,4,5-tetrahydro-1H-benzo[d]azepine-7-carboxamide

¹H NMR (400 MHz, MeOD) δ 7.58-7.64 (m, 2H), 7.53 (s, 1H), 7.31-7.37 (m, 1H), 7.17-7.24 (m, 2H), 7.09-7.15 (m, 1H), 6.38-6.43 (m, 1H), 4.63 (s, 3H), 2.97 (br. s., 4H), 2.78-2.88 (m, 1H), 2.47 (br. s., 4H), 2.05-2.15 (m, 2H), 1.86-2.00 (m, 2H), 1.62-1.77 (m, 2H)

Mass Spec: ES⁺ 374

### 2.75 Example 75

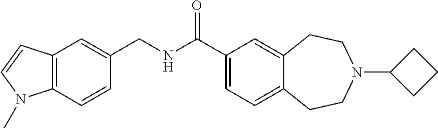

### 3-Cyclobutyl-N-((1-methyl-1H-indol-5-yl)methyl)-2,3,4,5-tetrahydro-1H-benzo[d]azepine-7-carboxamide

¹H NMR (400 MHz, MeOD) δ 7.56-7.64 (m, 2H), 7.52 (br. s., 1H), 7.29-7.36 (m, 1H), 7.19 (m, 2H), 7.09-7.14 (m, 1H), 6.34-6.41 (m, 1H), 4.64 (s, 2H), 3.79 (s, 3H), 2.91-3.03 (m, 4H), 2.76-2.89 (m, 1H), 2.47 (br. s., 4H), 2.04-2.15 (m, 2H), 1.86-1.99 (m, 2H), 1.60-1.77 (m, 2H)

Mass Spec: ES^{F} 388

### 2.76 Example 76

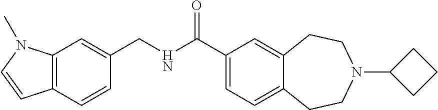

### 3-Cyclobutyl-N-((1-methyl-1H-indol-6-yl)methyl)-2,3,4,5-tetrahydro-1H-benzo[d]azepine-7-carboxamide

¹H NMR (400 MHz, DMSO-d6) δ 8.85-8.98 (m, 1H), 7.60-7.70 (m, 2H), 7.44-7.53 (m, 1H), 7.36 (s, 1H), 7.25-7.31 (m, 1H), 7.15-7.23 (m, 1H), 6.98-7.08 (m, 1H), 6.34-6.42 (m, 1H), 4.52-4.64 (m, 2H), 3.78 (s, 3H), 2.87 (br. s., 4H), 2.67-2.80 (m, 1H), 2.34 (br. s., 4H), 1.96-2.10 (m, 2H), 1.69-1.86 (m, 2H), 1.48-1.69 (m, 2H)

Mass Spec: ES⁺ 388

### 2.77 Example 77

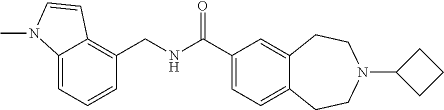

### 3-Cyclobutyl-N-((1-methyl-1H-indol-4-yl)methyl)-2,3,4,5-tetrahydro-1H-benzo[d]azepine-7-carboxamide

1H NMR (400 MHz, DMSO-d6) δ 8.80-8.94 (m, 1H), 7.58-7.74 (m, 2H), 7.27-7.37 (m, 2H), 7.15-7.23 (m, 1H), 7.06-7.14 (m, 1H), 6.91-6.99 (m, 1H), 6.52-6.60 (m, 1H), 4.66-4.77 (m, 2H), 3.35 (s, 3H), 2.87 (br. s., 4H), 2.75 (br. s., 1H), 2.26-2.42 (m, 4H), 1.94-2.07 (m, 2H), 1.71-1.86 (m, 2H), 1.50-1.66 (m, 2H)

Mass Spec: ES⁺ 388

### 2.78 Example 78

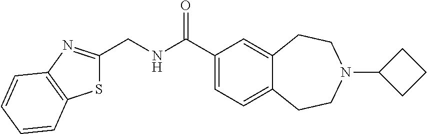

### N-(Benzo[d]thiazol-2-ylmethyl)-3-cyclobutyl-2,3,4,5-tetrahydro-1H-benzo[d]azepine-7-carboxamide

¹H NMR (400 MHz, MeOD) δ 7.92-7.98 (m, 2H), 7.65-7.70 (m, 2H), 7.47-7.54 (m, 1H), 7.38-7.45 (m, 1H), 7.22-7.28 (m, 1H), 4.96 (s, 2H), 2.96-3.06 (m, 4H), 2.81-2.91 (m, 1H), 2.51 (br. s., 4H), 2.07-2.17 (m, 2H), 1.88-2.02 (m, 2H), 1.62-1.79 (m, 2H)

Mass Spec: ES⁺ 392

### 2.79 Example 79

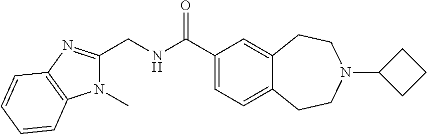

### 3-Cyclobutyl-N-((1-methyl-1H-benzo[d]imidazol-2-yl)methyl)-2,3,4,5-tetrahydro-1H-benzo[d]azepine-7-carboxamide

¹H NMR (400 MHz, DMSO-d6) δ 8.93-9.05 (m, 1H), 7.62-7.71 (m, 2H), 7.55-7.61 (m, 1H), 7.49-7.55 (m, 1H), 7.13-7.29 (m, 3H), 4.69-4.82 (m, 2H), 3.32 (s, 3H), 2.88 (br. s., 4H), 2.70-2.82 (m, 1H), 2.35 (br. s., 4H), 1.93-2.07 (m, 2H), 1.70-1.86 (m, 2H), 1.48-1.68 (m, 2H)

Mass Spec: ES⁺ 389

### 2.80 Example 80

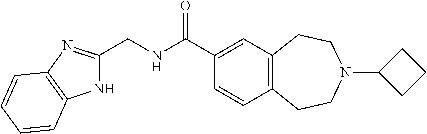

### N-((1H-Benzo[d]imidazol-2-yl)methyl)-3-cyclobutyl-2,3,4,5-tetrahydro-1H-benzo[d]azepine-7-carboxamide

¹H NMR (400 MHz, MeOD) δ 7.63-7.78 (m, 2H), 7.52 (br. s., 2H), 7.13-7.31 (m, 4H), 4.81 (s, 2H), 2.99 (br. s., 4H), 2.77-2.91 (m, 1H), 2.50 (br. s., 4H), 2.05-2.19 (m, 2H), 1.84-2.03 (m, 2H), 1.58-1.81 (m, 2H)

Mass Spec: ES⁺ 375

### 2.81 Example 81

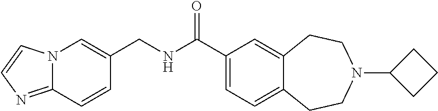

### 3-Cyclobutyl-N-(imidazo[1,2-a]pyridin-6-ylmethyl)-2,3,4,5-tetrahydro-1H-benzo[d]azepine-7-carboxamide

¹H NMR (400 MHz, DMSO-d6) δ 8.89-9.00 (m, 1H), 8.46 (s, 1H), 7.95 (s, 1H), 7.59-7.70 (m, 2H), 7.49-7.57 (m, 2H), 7.16-7.26 (m, 2H), 4.38-4.52 (m, 2H), 2.88 (br. s., 4H), 2.70-2.81 (m, 1H), 2.35 (br. s., 4H), 1.93-2.07 (m, 2H), 1.70-1.86 (m, 2H), 1.47-1.68 (m, 2H)

Mass Spec: ES⁺ 375

### 2.82 Example 82

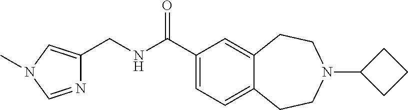

### 3-Cyclobutyl-N-((1-methyl-1H-imidazol-4-yl)methyl)-2,3,4,5-tetrahydro-1H-benzo[d]azepine-7-carboxamide

¹H NMR (400 MHz, MeOD) δ 7.57-7.62 (m, 2H), 7.53 (s, 1H), 7.17-7.22 (m, 1H), 6.99 (s, 1H), 4.45 (s, 2H), 3.68 (s, 3H), 2.98 (br. s., 4H), 2.78-2.89 (m, 1H), 2.48 (br. s., 4H), 2.05-2.18 (m, 2H), 1.87-2.01 (m, 2H), 1.62-1.78 (m, 2H)

Mass Spec: ES⁺ 338

### 2.83 Example 83

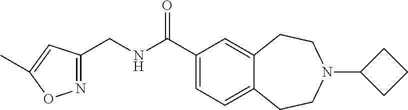

### 3-Cyclobutyl-N-((5-methylisoxazol-3-yl)methyl)-2,3,4,5-tetrahydro-1H-benzo[d]azepine-7-carboxamide

¹H NMR (400 MHz, MeOD) δ 7.58-7.64 (m, 2H), 7.18-7.25 (m, 1H), 6.12 (s, 1H), 4.55 (s, 2H), 2.99 (br. s., 4H), 2.81-2.93 (m, 1H), 2.44-2.61 (m, 4H), 2.39 (s, 3H), 2.06-2.18 (m, 2H), 1.88-2.02 (m, 2H), 1.63-1.79 (m, 2H)

Mass Spec: ES⁺ 340

### 2.84 Example 84

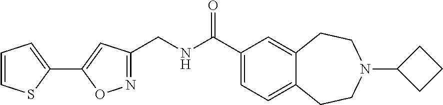

### 3-Cyclobutyl-N-((5-(thiophen-2-yl)isoxazol-3-yl)methyl)-2,3,4,5-tetrahydro-1H-benzo[d]azepine-7-carboxamide

¹H NMR (400 MHz, MeOD) δ 7.60-7.66 (m, 3H), 7.56-7.59 (m, 1H), 7.20-7.25 (m, 1H), 7.14-7.19 (m, 1H), 6.60 (s, 1H), 4.63 (s, 2H), 2.99 (br. s., 4H), 2.81-2.92 (m, 1H), 2.51 (br. s., 4H), 2.07-2.17 (m, 2H), 1.88-2.01 (m, 2H), 1.60-1.79 (m, 2H)

Mass Spec: ES⁺ 408

### 2.85 Example 85

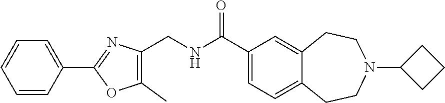

### 3-Cyclobutyl-N-((5-methyl-2-phenyloxazol-4-yl)methyl)-2,3,4,5-tetrahydro-1H-benzo[d]azepine-7-carboxamide

¹H NMR (400 MHz, MeOD) δ 7.94-8.01 (m, 2H), 7.61 (br. s., 2H), 7.41-7.52 (m, 3H), 7.15-7.24 (m, 1H), 4.46 (s, 2H), 2.98 (br. s., 4H), 2.78-2.91 (m, 1H), 2.47 (s, 7H), 2.05-2.17 (m, 2H), 1.86-2.01 (m, 2H), 1.60-1.79 (m, 2H)

Mass Spec: ES⁺ 416

### 2.86 Example 86

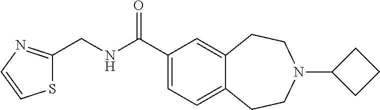

### 3-Cyclobutyl-N-(thiazol-2-ylmethyl)-2,3,4,5-tetrahydro-1H-benzo[d]azepine-7-carboxamide

¹H NMR (400 MHz, DMSO-d6) δ 9.21-9.33 (m, 1H), 7.69-7.78 (m, 1H), 7.58-7.70 (m, 3H), 7.18-7.28 (m, 1H), 4.67-4.79 (m, 2H), 2.89 (br. s., 4H), 2.78 (br. s., 1H), 2.37 (br. s., 4H), 1.94-2.08 (m, 2H), 1.70-1.87 (m, 2H), 1.46-1.69 (m, 2H)

Mass Spec: ES⁺ 342

### 2.87 Example 87

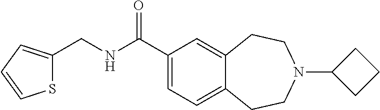

### 3-Cyclobutyl-N-(thiophen-2-ylmethyl)-2,3,4,5-tetrahydro-1H-benzo[d]azepine-7-carboxamide

¹H NMR (400 MHz, MeOD) δ 7.55-7.62 (m, 2H), 7.25-7.29 (m, 1H), 7.16-7.22 (m, 1H), 7.01-7.04 (m, 1H), 6.91-6.96 (m, 1H), 4.71 (s, 2H), 2.92-3.02 (m, 4H), 2.77-2.88 (m, 1H), 2.47 (br. s., 4H), 2.05-2.15 (m, 2H), 1.86-2.00 (m, 2H), 1.61-1.77 (m, 2H)

Mass Spec: ES⁺ 341

### 2.88 Example 88

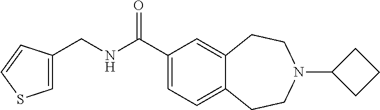

### 3-Cyclobutyl-N-(thiophen-3-ylmethyl)-2,3,4,5-tetrahydro-1H-benzo[d]azepine-7-carboxamide

¹H NMR (400 MHz, MeOD) δ 7.55-7.63 (m, 2H), 7.33-7.37 (m, 1H), 7.22-7.27 (m, 1H), 7.17-7.22 (m, 1H), 7.06-7.11 (m, 1H), 4.55 (s, 2H), 2.97 (br. s., 4H), 2.77-2.87 (m, 1H), 2.38-2.58 (m, 4H), 2.02-2.16 (m, 2H), 1.86-2.00 (m, 2H), 1.59-1.78 (m, 2H)

Mass Spec: ES⁺ 341

### 2.89 Example 89

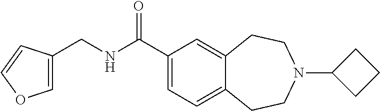

### 3-Cyclobutyl-N-(furan-3-ylmethyl)-2,3,4,5-tetrahydro-1H-benzo[d]azepine-7-carboxamide

¹H NMR (400 MHz, MeOD) δ 7.54-7.61 (m, 2H), 7.47 (s, 1H), 7.43 (s, 1H), 7.15-7.24 (m, 1H), 6.45 (s, 1H), 4.39 (s, 2H), 2.97 (br. s., 4H), 2.77-2.88 (m, 1H), 2.47 (br. s., 4H), 2.04-2.15 (m, 2H), 1.87-2.00 (m, 2H), 1.61-1.78 (m, 2H)

Mass Spec: ES⁺ 325

### 2.90 Example 90

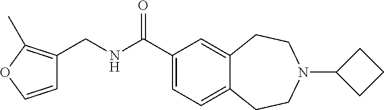

### 3-cyclobutyl-N-((2-methylfuran-3-yl)methyl)-2,3,4,5-tetrahydro-1H-benzo[d]azepine-7-carboxamide

¹H NMR (400 MHz, DMSO-d6) δ 8.60-8.73 (m, 1H), 7.55-7.64 (m, 2H), 7.37-7.44 (m, 1H), 7.15-7.23 (m, 1H), 6.31-6.38 (m, 1H), 4.15-4.25 (m, 2H), 2.87 (br. s., 5H), 2.22-2.45 (m, 7H), 1.94-2.07 (m, 2H), 1.72-1.87 (m, 2H), 1.48-1.67 (m, 2H)

Mass Spec: ES⁺ 339

### 2.91 Example 91

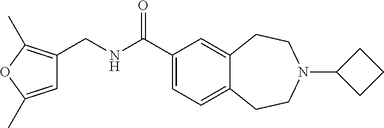

### 3-cyclobutyl-N-((2,5-dimethylfuran-3-yl)methyl)-2,3,4,5-tetrahydro-1H-benzo[d]azepine-7-carboxamide

¹H NMR (400 MHz, DMSO-d₆) δ 8.58-8.65 (m, 1H), 7.54-7.62 (m, 2H), 7.15-7.20 (m, 1H), 5.93 (s, 1H), 4.10-4.15 (m, 2H), 2.82-2.90 (m, 4H), 2.72-2.79 (m, 1H), 2.34 (br. s., 4H), 2.21 (s, 3H), 2.16 (s, 3H), 1.96-2.05 (m, 2H), 1.72-1.84 (m, 2H), 1.51-1.66 (m, 2H) Mass Spec: ES⁺ 353

### 2.92 Example 92

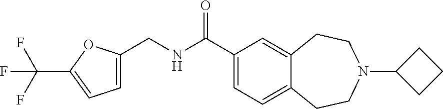

### 3-cyclobutyl-N-((5-(trifluoromethyl)furan-2-yl)methyl)-2,3,4,5-tetrahydro-1H-benzo[d]azepine-7-carboxamide

¹H NMR (400 MHz, DMSO-d₆) δ 8.95-9.04 (m, 1H), 7.57-7.67 (m, 2H), 7.18-7.25 (m, 1H), 7.13-7.18 (m, 1H), 6.48-6.53 (m, 1H), 4.46-4.56 (m, 2H), 2.88 (br. s., 4H), 2.70-2.82 (m, 1H), 2.26-2.43 (m, 4H), 2.00 (br. s., 2H), 1.70-1.88 (m, 2H), 1.49-1.67 (m, 2H)

Mass Spec: ES⁺ 393

### 2.93 Example 93

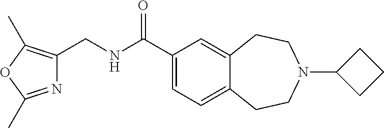

### 3-cyclobutyl-N-((2,5-dimethyloxazol-4-yl)methyl)-2,3,4,5-tetrahydro-1H-benzo[d]azepine-7-carboxamide

¹H NMR (400 MHz, DMSO-d₆) δ 8.65-8.75 (m, 1H), 7.54-7.66 (m, 2H), 7.12-7.22 (m, 1H), 4.15-4.25 (m, 2H), 2.81-2.93 (m, 4H), 2.69-2.80 (m, 1H), 2.22-2.43 (m, 10H), 1.94-2.07 (m, 2H), 1.71-1.86 (m, 2H), 1.50-1.67 (m, 2H)

Mass Spec: ES⁺ 354

### 2.94 Example 94

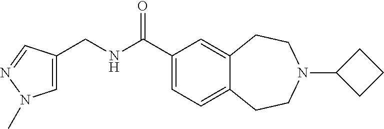

### 3-Cyclobutyl-N-((1-methyl-1H-pyrazol-4-yl)methyl)-2,3,4,5-tetrahydro-1H-benzo[d]azepine-7-carboxamide

¹H NMR (400 MHz, MeOD) δ 7.54-7.59 (m, 3H), 7.45 (s, 1H), 7.16-7.22 (m, 1H), 4.39 (s, 2H), 3.84 (s, 3H), 2.97 (br. s., 4H), 2.77-2.89 (m, 1H), 2.47 (br. s., 4H), 2.05-2.15 (m, 2H), 1.87-2.00 (m, 2H), 1.62-1.78 (m, 2H)

Mass Spec: ES⁺ 339

### 2.95 Example 95

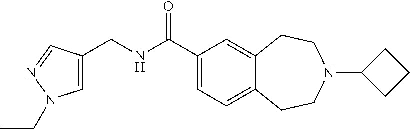

### 3-Cyclobutyl-N-((1-ethyl-1H-pyrazol-4-yl)methyl)-2,3,4,5-tetrahydro-1H-benzo[d]azepine-7-carboxamide

¹H NMR (400 MHz, DMSO-d6) δ 8.62-8.72 (m, 1H), 7.55-7.65 (m, 3H), 7.34 (s, 1H), 7.14-7.20 (m, 1H), 4.21-4.31 (m, 2H), 4.00-4.12 (m, 2H), 2.80-2.91 (m, 4H), 2.68-2.79 (m, 1H), 2.33 (br. s., 4H), 1.94-2.05 (m, 2H), 1.70-1.84 (m, 2H), 1.49-1.66 (m, 2H), 1.32 (t, 3H)

Mass Spec: ES⁺ 353

### 2.96 Example 96

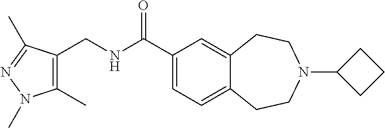

### 3-Cyclobutyl-N-((1,3,5-trimethyl-1H-pyrazol-4-yl)methyl)-2,3,4,5-tetrahydro-1H-benzo[d]azepine-7-carboxamide

¹H NMR (400 MHz, DMSO-d6) δ 8.39-8.50 (m, 1H), 7.53-7.63 (m, 2H), 7.12-7.21 (m, 1H), 4.12-4.22 (m, 2H), 3.35 (s, 3H), 2.80-2.92 (m, 4H), 2.67-2.79 (m, 1H), 2.33 (br. s., 4H), 2.20 (s, 3H), 2.10 (s, 3H), 1.94-2.05 (m, 2H), 1.70-1.84 (m, 2H), 1.47-1.67 (m, 2H)

Mass Spec: ES⁺ 367

### 2.97 Example 97

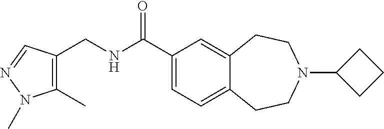

### 3-Cyclobutyl-N-((1,5-dimethyl-1H-pyrazol-4-yl)methyl)-2,3,4,5-tetrahydro-1H-benzo[d]azepine-7-carboxamide

¹H NMR (400 MHz, DMSO-d6) δ 8.54-8.65 (m, 1H), 7.52-7.65 (m, 2H), 7.25 (s, 1H), 7.12-7.21 (m, 1H), 4.16-4.26 (m, 2H), 3.34 (s, 3H), 2.86 (br. s., 4H), 2.68-2.79 (m, 1H), 2.39 (br. s., 4H), 2.19-2.25 (m, 3H), 1.94-2.07 (m, 2H), 1.69-1.85 (m, 2H), 1.50-1.67 (m, 2H)

Mass Spec: ES⁺ 353

### 2.98 Example 98

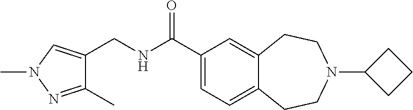

### 3-Cyclobutyl-N-((1,3-dimethyl-1H-pyrazol-4-yl)methyl)-2,3,4,5-tetrahydro-1H-benzo[d]azepine-7-carboxamide

¹H NMR (400 MHz, DMSO-d6) δ 8.54-8.63 (m, 1H), 7.54-7.66 (m, 2H), 7.46 (s, 1H), 7.13-7.20 (m, 1H), 4.16-4.26 (m, 2H), 3.69 (s, 3H), 2.81-2.95 (m, 4H), 2.68-2.80 (m, 1H), 2.24-2.43 (m, 4H), 2.09 (s, 3H), 1.94-2.06 (m, 2H), 1.71-1.85 (m, 2H), 1.49-1.67 (m, 2H)

Mass Spec: ES⁺ 353

### 2.99 Example 99

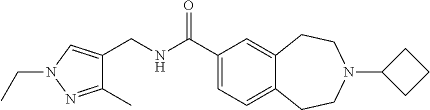

### 3-Cyclobutyl-N-((1-ethyl-3-methyl-1H-pyrazol-4-yl)methyl)-2,3,4,5-tetrahydro-1H-benzo[d]azepine-7-carboxamide

¹H NMR (400 MHz, DMSO-d₆) δ 8.53-8.64 (m, 1H), 7.57-7.66 (m, 2H), 7.51 (s, 1H), 7.17 (d, J=7.58 Hz, 1H), 4.18-4.26 (m, 2H), 3.92-4.03 (m, 2H), 2.87 (br. s., 4H), 2.70-2.81 (m, 1H), 2.27-2.43 (m, 4H), 2.13 (s, 3H), 1.95-2.07 (m, 2H), 1.78 (br. s., 2H), 1.49-1.68 (m, 2H), 1.26-1.34 (m, 3H)

Mass Spec: ES⁺ 367

### 2.100 Example 100

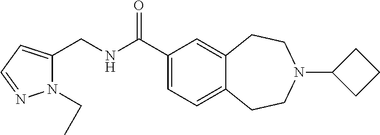

### 3-cyclobutyl-N-((1-ethyl-1H-pyrazol-5-yl)methyl)-2,3,4,5-tetrahydro-1H-benzo[d]azepine-7-carboxamide

¹H NMR (400 MHz, DMSO-d₆) δ 8.82-8.93 (m, 1H), 7.55-7.70 (m, 2H), 7.29-7.38 (m, 1H), 7.15-7.23 (m, 1H), 6.09-6.18 (m, 1H), 4.46-4.55 (m, 2H), 4.09-4.19 (m, 2H), 2.81-2.95 (m, 4H), 2.68-2.80 (m, 1H), 2.34 (br. s., 4H), 1.93-2.10 (m, 2H), 1.70-1.85 (m, 2H), 1.49-1.67 (m, 2H), 1.24-1.34 (m, 3H)

Mass Spec: ES⁺ 353

### 2.101 Example 101

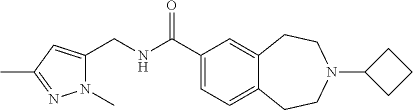

### 3-Cyclobutyl-N-((1,3-dimethyl-1H-pyrazol-5-yl)methyl)-2,3,4,5-tetrahydro-1H-benzo[d]azepine-7-carboxamide

¹H NMR (400 MHz, DMSO-d6) δ 8.77-8.89 (m, 1H), 7.54-7.71 (m, 2H), 7.17-7.27 (m, 1H), 5.92 (s, 1H), 4.36-4.49 (m, 2H), 3.71 (s, 3H), 2.89 (br. s., 4H), 2.70-2.83 (m, 1H), 2.34 (br. s., 4H), 1.96-2.11 (m, 5H), 1.80 (br. s., 2H), 1.49-1.69 (m, 2H)

Mass Spec: ES⁺ 353

### 2.102 Example 102

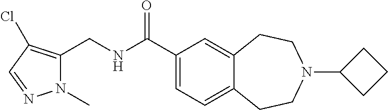

### N-((4-chloro-1-methyl-1H-pyrazol-5-yl)methyl)-3-cyclobutyl-2,3,4,5-tetrahydro-1H-benzo[d]azepine-7-carboxamide

¹H NMR (400 MHz, DMSO-d₆) δ 8.76-8.92 (m, 1H), 7.58-7.65 (m, 2H), 7.49 (s, 1H), 7.17-7.22 (m, 1H), 4.47-4.54 (m, 2H), 3.84 (s, 3H), 2.88 (br. s., 4H), 2.70-2.81 (m, 1H), 2.34 (br. s., 4H), 1.95-2.07 (m, 2H), 1.78 (br. s., 2H), 1.51-1.67 (m, 2H)

Mass Spec: ES⁺ 373

### 2.103 Example 103

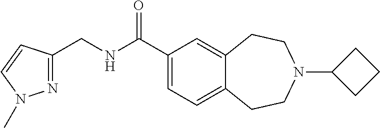

### 3-Cyclobutyl-N-((1-methyl-1H-pyrazol-3-yl)methyl)-2,3,4,5-tetrahydro-1H-benzo[d]azepine-7-carboxamide

¹H NMR (400 MHz, DMSO-d₆) δ 8.70-8.82 (m, 1H), 7.50-7.70 (m, 3H), 7.12-7.24 (m, 1H), 6.07-6.14 (m, 1H), 4.32-4.44 (m, 2H), 3.78 (s, 3H), 2.66-2.98 (m, 5H), 2.24-2.44 (m, 4H), 2.01 (br. s., 2H), 1.79 (br. s., 2H), 1.48-1.69 (m, 2H)

Mass Spec: ES⁺ 339

### 2.104 Example 104

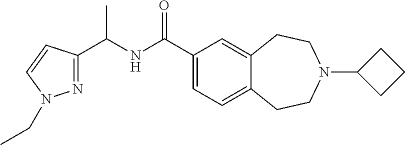

### 3-cyclobutyl-N-(1-(1-ethyl-1H-pyrazol-3-yl)ethyl)-2,3,4,5-tetrahydro-1H-benzo[d]azepine-7-carboxamide

¹H NMR (400 MHz, DMSO-d₆) δ 8.45-8.61 (m, 1H), 7.53-7.67 (m, 3H), 7.11-7.21 (m, 1H), 6.08-6.19 (m, 1H), 5.13-5.26 (m, 1H), 3.99-4.10 (m, 2H), 2.86 (br. s., 4H), 2.66-2.78 (m, 1H), 2.33 (br. s., 4H), 1.98 (d, J=7.33 Hz, 2H), 1.70-1.84 (m, 2H), 1.48-1.66 (m, 2H), 1.39-1.47 (m, 3H), 1.33 (m, 3H)

Mass Spec: ES⁺ 367

### 2.105 Example 105

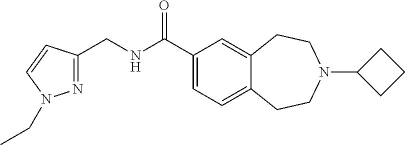

### 3-Cyclobutyl-N-((1-ethyl-1H-pyrazol-3-yl)methyl)-2,3,4,5-tetrahydro-1H-benzo[d]azepine-7-carboxamide

¹H NMR (400 MHz, DMSO-d6) δ 8.69-8.84 (m, 1H), 7.55-7.70 (m, 3H), 7.13-7.23 (m, 1H), 6.06-6.16 (m, 1H), 4.33-4.46 (m, 2H), 4.02-4.11 (m, 2H), 2.87 (br. s., 4H), 2.69-2.82 (m, 1H), 2.35 (br. s., 4H), 1.93-2.09 (m, 2H), 1.70-1.87 (m, 2H), 1.48-1.68 (m, 2H), 1.28-1.39 (m, 3H)

Mass Spec: ES⁺ 353

### 2.106 Example 106

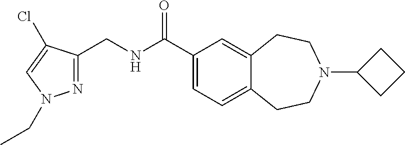

### N-((4-chloro-1-ethyl-1H-pyrazol-3-yl)methyl)-3-cyclobutyl-2,3,4,5-tetrahydro-1H-benzo[d]azepine-7-carboxamide

¹H NMR (400 MHz, DMSO-d₆) δ 8.64-8.75 (m, 1H), 7.95 (s, 1H), 7.58-7.69 (m, 2H), 7.14-7.22 (m, 1H), 4.37-4.48 (m, 2H), 3.99-4.12 (m, 2H), 2.87 (br. s., 5H), 2.36 (br. s., 4H), 1.95-2.07 (m, 2H), 1.79 (br. s., 2H), 1.48-1.68 (m, 2H), 1.28-1.39 (m, 3H)

Mass Spec: ES⁺ 387

### 2.107 Example 107

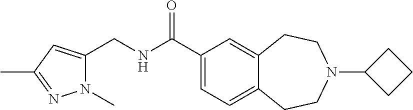

### 3-Cyclobutyl-N-((1,5-dimethyl-1H-pyrazol-3-yl)methyl)-2,3,4,5-tetrahydro-1H-benzo[d]azepine-7-carboxamide

¹H NMR (400 MHz, DMSO-d6) δ 8.67-8.80 (m, 1H), 7.55-7.68 (m, 2H), 7.13-7.25 (m, 1H), 5.90 (s, 1H), 4.24-4.36 (m, 2H), 3.63 (s, 3H), 2.70-2.81 (m, 1H), 2.88 (br. s., 4H), 2.34 (br. s., 4H), 2.19 (s, 3H), 2.02 (br. s., 2H), 1.79 (br. s., 2H), 1.60 (br. s., 2H)

Mass Spec: ES⁺ 353

### 2.108 Example 108

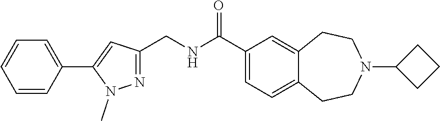

### 3-Cyclobutyl-N-((1-methyl-5-phenyl-1H-pyrazol-3-yl)methyl)-2,3,4,5-tetrahydro-1H-benzo[d]azepine-7-carboxamide

¹H NMR (400 MHz, DMSO-d6) δ 8.77-8.86 (m, 1H), 7.58-7.70 (m, 2H), 7.36-7.55 (m, 5H), 7.14-7.23 (m, 1H), 6.26-6.33 (m, 1H), 4.36-4.49 (m, 2H), 3.80 (s, 3H), 2.87 (br. s., 4H), 2.68-2.79 (m, 1H), 2.34 (br. s., 4H), 1.93-2.08 (m, 2H), 1.70-1.86 (m, 2H), 1.49-1.67 (m, 2H)

Mass Spec: ES⁺ 415

### 2.109 Example 109

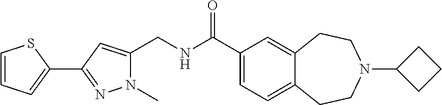

### 3-Cyclobutyl-N-((1-methyl-3-(thiophen-2-yl)-1H-pyrazol-5-yl)methyl)-2,3,4,5-tetrahydro-1H-benzo[d]azepine-7-carboxamide

¹H NMR (400 MHz, DMSO-d6) δ 8.87-8.97 (m, 1H), 7.58-7.69 (m, 2H), 7.37-7.44 (m, 1H), 7.30-7.37 (m, 1H), 7.17-7.26 (m, 1H), 7.00-7.08 (m, 1H), 6.49 (s, 1H), 4.45-4.57 (m, 2H), 3.83 (s, 3H), 2.88 (br. s., 4H), 2.70-2.82 (m, 1H), 2.35 (br. s., 4H), 1.93-2.08 (m, 2H), 1.70-1.88 (m, 2H), 1.49-1.68 (m, 2H)

Mass Spec: ES⁺ 421

### 2.110 Example 110

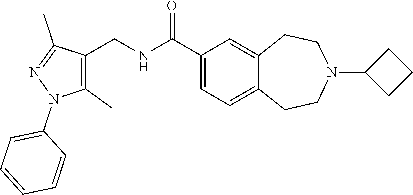

### 3-Cyclobutyl-N-((3,5-dimethyl-1-phenyl-1H-pyrazol-4-yl)methyl)-2,3,4,5-tetrahydro-1H-benzo[d]azepine-7-carboxamide

¹H NMR (400 MHz, MeOD) δ 7.55-7.60 (m, 2H), 7.47-7.55 (m, 2H), 7.37-7.47 (m, 3H), 7.16-7.22 (m, 1H), 4.43 (s, 2H), 2.91-3.03 (m, 4H), 2.77-2.88 (m, 1H), 2.38-2.56 (m, 4H), 2.27-2.35 (m, 6H), 2.05-2.16 (m, 2H), 1.86-1.99 (m, 2H), 1.60-1.78 (m, 2H)

Mass Spec: ES⁺ 429

### 2.111 Example 111

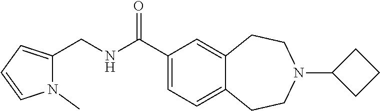

### 3-Cyclobutyl-N-((1-methyl-1H-pyrrol-2-yl)methyl)-2,3,4,5-tetrahydro-1H-benzo[d]azepine-7-carboxamide

¹H NMR (400 MHz, MeOD) δ 7.51-7.60 (m, 2H), 7.11-7.25 (m, 1H), 6.56-6.66 (m, 1H), 6.02-6.09 (m, 1H), 5.91-5.99 (m, 1H), 4.53 (s, 2H), 3.61 (s, 3H), 2.89-3.04 (m, 4H), 2.76-2.88 (m, 1H), 2.47 (br. s., 4H), 2.04-2.16 (m, 2H), 1.84-2.00 (m, 2H), 1.59-1.78 (m, 2H)

Mass Spec: ES⁺ 338

### 2.112 Example 112

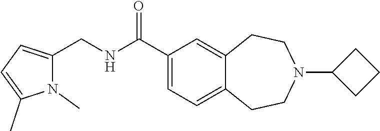

### 3-cyclobutyl-N-((1,5-dimethyl-1H-pyrrol-2-yl)methyl)-2,3,4,5-tetrahydro-1H-benzo[d]azepine-7-carboxamide

¹H NMR (400 MHz, DMSO-d₆) δ 8.52-8.62 (m, 1H), 7.56-7.68 (m, 2H), 7.14-7.21 (m, 1H), 5.81-5.88 (m, 1H), 5.64-5.70 (m, 1H), 4.34-4.45 (m, 2H), 3.41 (s, 3H), 2.81-2.94 (m, 4H), 2.70-2.81 (m, 1H), 2.34 (br. s., 4H), 2.14 (s, 3H), 1.95-2.07 (m, 2H), 1.70-1.85 (m, 2H), 1.51-1.67 (m, 2H)

Mass Spec: ES⁺ 352

### 2.113 Example 113

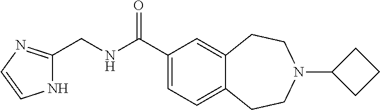

### N-((1H-Imidazol-2-yl)methyl)-3-cyclobutyl-2,3,4,5-tetrahydro-1H-benzo[d]azepine-7-carboxamide

¹H NMR (400 MHz, MeOD) δ 7.58-7.69 (m, 2H), 7.17-7.24 (m, 1H), 6.97 (s, 2H), 4.60 (s, 2H), 2.98 (br. s., 4H), 2.77-2.89 (m, 1H), 2.48 (br. s., 4H), 2.06-2.16 (m, 2H), 1.87-2.00 (m, 2H), 1.62-1.78 (m, 2H)

Mass Spec: ES⁺ 325

### 2.114 Example 114

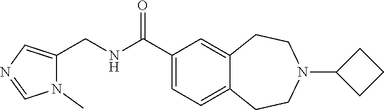

### 3-Cyclobutyl-N-((1-methyl-1H-imidazol-5-yl)methyl)-2,3,4,5-tetrahydro-1H-benzo[d]azepine-7-carboxamide

¹H NMR (400 MHz, MeOD) δ 7.57-7.63 (m, 3H), 7.18-7.25 (m, 1H), 6.96 (s, 1H), 4.60 (s, 2H), 3.72 (s, 3H), 2.94-3.04 (m, 4H), 2.79-2.91 (m, 1H), 2.50 (br. s., 4H), 2.07-2.18 (m, 2H), 1.88-2.02 (m, 2H), 1.62-1.80 (m, 2H)

Mass Spec: ES⁺ 338

### 2.115 Example 115

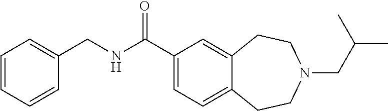

### N-benzyl-3-isobutyl-2,3,4,5-tetrahydro-1H-benzo[d]azepine-7-carboxamide

MS ES⁺ : 337 (M+H)

¹H NMR (400 MHz, CDCl₃): δ 7.49-7.57 (m, 2H), 7.28-7.40 (m, 5H), 7.11-7.16 (m, 1H), 4.63-4.68 (m, 2H), 2.91-2.98 (m, 4H), 2.55-2.64 (m, 4H), 2.16-2.22 (m, 2H), 1.74-1.86 (m, 1H), 0.89-0.96 (m, 6H)

### 2.116 Example 116

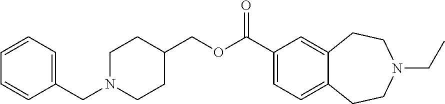

### (1-Benzylpiperidin-4-yl)methyl 3-ethyl-2,3,4,5-tetrahydro-1H-benzo[d]azepine-7-carboxylate

¹H NMR (400 MHz, DMSO-d6) δ 7.60-7.67 (m, 2H), 7.14-7.29 (m, 6H), 4.01-4.08 (m, 2H), 3.38 (s, 2H), 2.81-2.89 (m, 4H), 2.71-2.79 (m, 2H), 2.37-2.49 (m, 6H), 1.81-1.91 (m, 2H), 1.58-1.69 (m, 3H), 1.15-1.29 (m, 2H), 0.89-0.97 (m, 3H)

Mass Spec: ES⁺ 407

### 2.117 Example 117

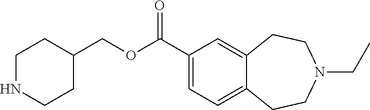

### Piperidin-4-ylmethyl 3-ethyl-2,3,4,5-tetrahydro-1H-benzo[d]azepine-7-carboxylate

¹H NMR (400 MHz, D₂O) δ 7.84-7.92 (m, 2H), 7.34-7.42 (m, 1H), 4.21-4.30 (m, 2H), 3.70-3.82 (m, 2H), 3.43-3.51 (m, 2H), 3.13-3.34 (m, 6H), 2.98-3.12 (m, 4H), 2.13-2.26 (m, 1H), 2.02-2.11 (m, 2H), 1.52-1.66 (m, 2H), 1.28-1.37 (m, 3H)

Mass Spec: ES⁺ 316

### 2.118 Example 118

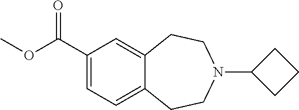

### Methyl 3-cyclobutyl-2,3,4,5-tetrahydro-1H-benzo[d]azepine-7-carboxylate

¹H NMR (400 MHz, MeOD) δ 7.71-7.81 (m, 2H), 7.17-7.25 (m, 1H), 3.88 (s, 3H), 2.98 (br. s., 4H), 2.78-2.89 (m, 1H), 2.39-2.59 (m, 4H), 2.05-2.16 (m, 2H), 1.87-2.00 (m, 2H), 1.60-1.78 (m, 2H)

Mass Spec: ES⁺ 260

### 2.119 Example 119

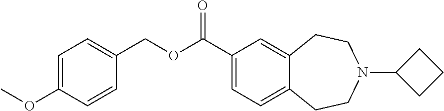

### 4-Methoxybenzyl 3-cyclobutyl-2,3,4,5-tetrahydro-1H-benzo[d]azepine-7-carboxylate

1H NMR (400 MHz, CDCl₃) δ 7.81-7.87 (m, 1H), 7.80 (s, 1H), 7.35-7.41 (m, 2H), 7.13-7.19 (m, 1H), 6.88-6.94 (tn, 2H), 5.28 (s, 2H), 3.82 (s, 3H), 3.08 (br. s., 4H), 2.90-3.01 (m, 1H), 2.67 (br. s., 4H), 2.07-2.24 (m, 4H), 1.56-1.81 (m, 2H)

Mass Spec: ES⁺ 366

### 2.120 Example 120

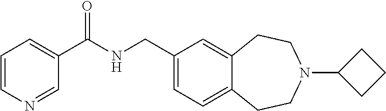

### N-((3-Cyclobutyl-2,3,4,5-tetrahydro-1H-benzo[d]azepin-7-yl)methyl)nicotinamide

MS ES⁺ : 336 (M+H)

¹H NMR (400 MHz, MeOD): δ 8.96-9.02 (m, 1H), 8.65-8.71 (m, 1H), 8.21-8.30 (m, 1H), 7.50-7.58 (m, 1H), 7.02-7.14 (m, 3H), 4.53 (s, 2H), 2.87-2.96 (m, 4H), 2.76-2.86 (m, 1H), 2.36-2.53 (m, 4H), 2.05-2.14 (m, 2H), 1.86-1.99 (m, 2H), 1.61-1.78 (m, 2H)

### 2.121 Example 121

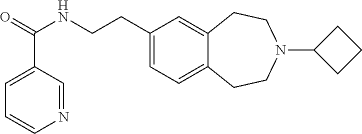

### N-(2-(3-Cyclobutyl-2,3,4,5-tetrahydro-1H-benzo[d]azepin-7-yl)ethyl)nicotinamide

¹H NMR (400 MHz, MeOD) δ 8.78-8.98 (m, 1H), 8.58-8.71 (m, 1H), 8.08-8.27 (m, 1H), 7.39-7.64 ((m, 1H), 6.92-7.14 (m, 3H), 3.49-3.72 (m, 2H), 2.71-3.01 (m, 7H), 2.44 (br. s., 4H), 2.01-2.19 (m, 2H), 1.82-2.01 (m, 2H), 1.54-1.82 (m, 2H)

Mass Spec: ES⁺ 350

### 2.122 Example 122

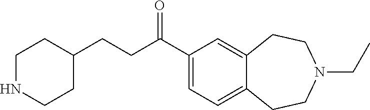

### 1-(3-ethyl-2,3,4,5-tetrahydro-1H-benzo[d]azepin-7-yl)-3-(piperidin-4-yl)propan-1-one Hydrochloride

NMR: ¹H NMR (400 MHz, D₂O) δ 7.74-7.86 (m, 2H), 7.31-7.39 (m, 1H), 3.65-3.78 (m, 2H), 3.31-3.41 (m, 2H), 2.85-3.31 (m, 12H), 1.86-1.99 (m, 2H), 1.55-1.70 (m, 3H), 1.33 (none, 6H), 1.22-1.44 (m, 5H)

Mass Spec: ES⁺ 315

### 2.123 Example 123

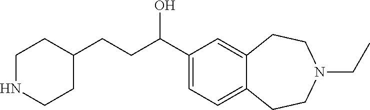

### 1-(3-ethyl-2,3,4,5-tetrahydro-1H-benzo[d]azepin-7-yl)-3-(piperidin-4-yl)propan-1-ol Formate

NMR: ¹H NMR (400 MHz, MeOD) δ 8.47 (br. s., 2H), 7.11-7.29 (m, 3H), 4.50-4.65 (m, 1H), 3.43-3.55 (m, 2H), 3.06-3.41 (m, 10H), 2.89-2.99 (m, 2H), 1.83-2.01 (m, 2H), 1.63-1.83 (m, 2H), 1.51-1.66 (m, 1H), 1.12-1.50 (m, 9H)

Mass Spec: ES⁺ 317

### 2.124 Example 124

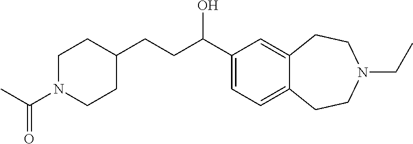

### 1-(4-(3-(3-ethyl-2,3,4,5-tetrahydro-1H-benzo[d]azepin-7-yl)-3-hydroxypropyl)piperidin-1-yl)ethanone

NMR: ¹H NMR (400 MHz, MeOD) δ 6.98-7.15 (m, 3H), 4.34-4.63 (m, 2H), 3.76-3.95 (m, 1H), 3.00-3.13 (m, 1H), 2.85-2.99 (m, 4H), 2.48-2.75 (m, 7H), 2.05 (s, 3H), 1.61-1.89 (m, 4H), 1.43-1.58 (m, 1H), 1.29-1.44 (m, 1H), 0.91-1.26 (m, 6H)

Mass Spec: ES⁺ 359

### 2.125 Example 125

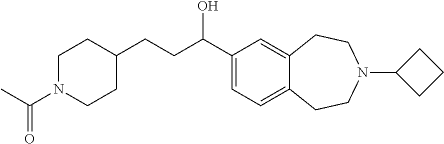

### 1-(4-(3-(3-cyclobutyl-2,3,4,5-tetrahydro-1H-benzo[d]azepin-7-yl)-3-hydroxypropyl)piperidin-1-yl)ethanone

NMR: ¹H NMR (400 MHz, DMSO-d₆) δ 6.95-7.08 (m, 3H), 4.99-5.10 (m, 1H), 4.22-4.47 (m, 2H), 3.64-3.82 (m, 1H), 2.85-3.02 (m, 1H), 2.65-2.86 (m, 5H), 2.21-2.47 (m, 5H), 1.88-2.06 (m, 5H), 1.69-1.86 (m, 2H), 1.47-1.68 (m, 6H), 1.35-1.45 (m, 1H), 1.20-1.34 (m, 1H), 1.05-1.19 (m, 1H), 0.90-1.04 (m, 1H), 0.74-0.90 (m, 1H)

Mass Spec: ES⁺ 385

### 2.126 Example 126

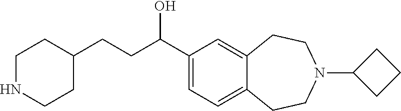

### 1-(3-cyclobutyl-2,3,4,5-tetrahydro-1H-benzo[d]azepin-7-yl)-3-(piperidin-4-yl)propan-1-ol

NMR: ¹H NMR (400 MHz, DMSO-d₆) δ 6.86-7.13 (m, 3H), 4.99 (br. s., 1H), 4.28-4.45 (m, 1H), 2.59-2.94 (m, 7H), 2.16-2.44 (m, 5H), 1.88-2.12 (m, 2H), 1.68-1.86 (m, 2H), 1.40-1.66 (m, 7H), 1.14-1.36 (m, 2H), 0.98-1.13 (m, 2H), 0.76-0.98 (m, 2H)

Mass Spec: ES⁺ 343

### 2.127 Example 127

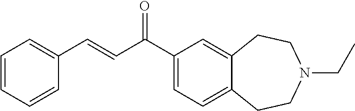

### (E)-1-(3-ethyl-2,3,4,5-tetrahydro-1H-benzo[d]azepin-7-yl)-3-phenylprop-2-en-1-one

NMR: ¹H NMR (400 MHz, MeOD) δ 7.83-7.93 (m, 2H), 7.70-7.81 (m, 4H), 7.40-7.50 (m, 3H), 7.31 (d, J=7.83 Hz, 1H), 2.99-3.16 (m, 4H), 2.53-2.86 (m, 6H), 1.07-1.22 (m, 3H)

Mass Spec: ES⁺ 306

### 2.128 Example 128

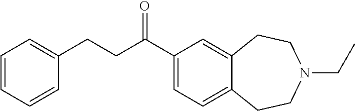

### 1-(3-ethyl-2,3,4,5-tetrahydro-1H-benzo[d]azepin-7-yl)-3-phenylpropan-1-one Hydrochloride

NMR: ¹H NMR (400 MHz, D₂O) δ 7.65-7.73 (m, 1H), 7.62 (s, 1H), 7.13-7.33 (m, 6H), 3.60-3.76 (m, 4H), 2.83-3.33 (m, 10H), 1.23-1.39 (m, 3H)

Mass Spec: ES⁺ 308

### 2.129 Example 129

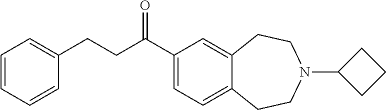

### 1-(3-cyclobutyl-2,3,4,5-tetrahydro-1H-benzo[d]azepin-7-yl)-3-phenylpropan-1-one

NMR: ¹H NMR (400 MHz, CDCl₃) δ ppm 1.55-1.79 (m, 2H) 1.82-2.01 (m, 2H) 2.01-2.15 (m, 2H) 2.46 (br. s., 4H) 2.72-2.86 (m, 1H) 2.92-3.03 (m, 4H) 3.01-3.14 (m, 2H) 3.20-3.36 (m, 2H) 7.09-7.42 (m, 6H) 7.64-7.80 (m, 2H)

Mass Spec: ES⁺ 334

### 2.130 Example 130

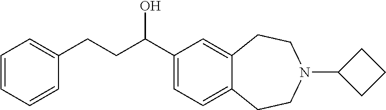

### 1-(3-cyclobutyl-2,3,4,5-tetrahydro-1H-benzo[d]azepin-7-yl)-3-phenylpropan-1-ol Formate

NMR: ¹H NMR (400 MHz, MeOD) δ 8.52 (s, 1H), 6.98-7.42 (m, 8H), 4.49-4.67 (m, 1H), 3.51-3.69 (m, 1H), 2.92-3.26 (m, 8H), 2.51-2.80 (m, 2H), 2.19-2.42 (m, 4H), 1.66-2.13 (m, 4H)

Mass Spec: ES⁺ 336

### 2.131 Example 131

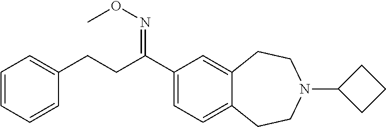

### 1-(3-cyclobutyl-2,3,4,5-tetrahydro-1H-benzo[d]azepin-7-yl)-3-phenylpropan-1-one O-methyl oxime

NMR: ¹H NMR (400 MHz, CDCl₃) δ ppm 1.54-1.81 (m, 4H) 1.93 (br. s., 2H) 2.01-2.18 (m, 2H) 2.45 (br. s., 3H) 2.73-3.06 (m, 6H) 3.07-3.22 (m, 2H) 3.61 (s, 3H) 6.69 (s, 1H) 6.76-6.90 (m, 1H) 7.04-7.13 (m, 1H) 7.13-7.21 (m, 3H) 7.21-7.34 (m, 2H)

Mass Spec: ES⁺ 363

### 2.132 Example 132

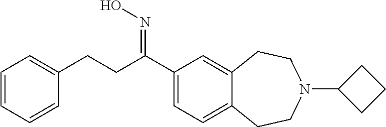

### 1-(3-cyclobutyl-2,3,4,5-tetrahydro-1H-benzo[d]azepin-7-yl)-3-phenylpropan-1-one oxime

NMR: ¹H NMR (400 MHz, CDCl₃) δ 7.03-7.61 (m, 9H), 3.02-3.12 (m, 2H), 2.85-3.01 (m, 6H), 2.73-2.85 (m, 1H), 2.47 (br. s., 4H), 2.02-2.17 (m, 2H), 1.84-2.02 (m, 2H), 1.48-1.79 (m, 2H)

Mass Spec: ES⁺ 349

### 2.133 Example 133

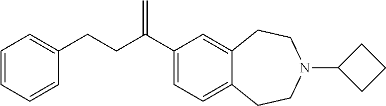

### 3-cyclobutyl-7-(4-phenylbut-1-en-2-yl)-2,3,4,5-tetrahydro-1H-benzo[d]azepine

NMR: ¹H NMR (400 MHz, CDCl₃) δ 7.13-7.40 (m, 7H), 7.02-7.13 (m, 1H), 5.22-5.38 (m, 1H), 5.04 (s, 1H), 2.93 (br. s., 4H), 2.68-2.86 (m, 5H), 2.47 (br. s., 4H), 1.99-2.17 (m, 2H), 1.82-2.00 (m, 2H), 1.51-1.79 (m, 2H)

Mass Spec: ES⁺ 332

### 2.134 Example 134

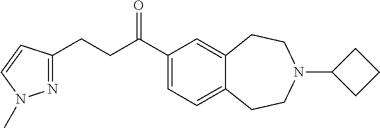

### 1-(3-cyclobutyl-2,3,4,5-tetrahydro-1H-benzo[d]azepin-7-yl)-3-(1-methyl-1H-pyrazol-3-yl)propan-1-one

NMR: ¹H NMR (400 MHz, CDCl₃) δ 7.67-7.82 (m, 2H), 7.22-7.31 (m, 1H), 7.12-7.22 (m, 1H), 6.04-6.14 (m, 1H), 3.85 (s, 3H), 3.29-3.41 (m, 2H), 3.07 (t, J=7.58 Hz, 2H), 2.97 (br. s., 4H), 2.71-2.84 (m, 1H), 2.45 (br. s., 4H), 2.02-2.16 (m, 2H), 1.82-2.01 (m, 2H), 1.53-1.79 (m, 2H)

Mass Spec: ES⁺ 338

### 2.135 Example 135

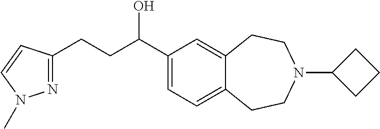

### 1-(3-cyclobutyl-2,3,4,5-tetrahydro-1H-benzo[d]azepin-7-yl)-3-(1-methyl-1H-pyrazol-3-yl)propan-1-ol

NMR: ¹H NMR (400 MHz, MeOD) δ 7.35-7.54 (m, 1H), 6.94-7.24 (m, 3H), 5.94-6.21 (m, 1H), 4.46-4.71 (m, 1H), 3.69-3.90 (m, 3H), 2.75-3.04 (m, 5H), 2.29-2.73 (m, 6H), 1.84-2.28 (m, 5H), 1.55-1.82 (m, 3H)

Mass Spec: ES⁺ 323 (M−16)

### 2.136 Example 136

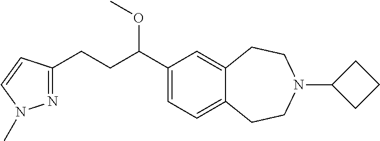

### 3-cyclobutyl-7-(1-methoxy-3-(1-methyl-1H-pyrazol-3-yl)propyl)-2,3,4,5-tetrahydro-1H-benzo[d]azepine Hydrochloride

NMR: ¹H NMR (400 MHz, MeOD) δ 7.74-8.08 (m, 1H), 6.98-7.38 (m, 3H), 6.31-6.53 (m, 1H), 4.10-4.26 (m, 1H), 3.93-4.09 (m, 3H), 3.60-3.85 (m, 4H), 3.33-3.50 (m, 2H), 2.98-3.24 (m, 4H), 2.65-2.96 (m, 4H), 2.26-2.62 (m, 4H), 1.68-2.22 (m, 4H)

Mass Spec: ES⁺ 322 (M−32)

### 2.137 Example 137

### 1-(3-cyclobutyl-2,3,4,5-tetrahydro-1H-benzo[d]azepin-7-yl)-3-(1-methyl-1H-pyrazol-5-yl)propan-1-ol

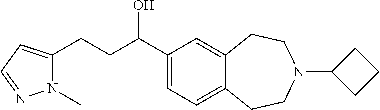

¹H NMR: (400 MHz, MeOD) δ 7.34 (s, 1H), 7.06-7.18 (m, 3H), 6.09 (s, 1H), 4.52-4.70 (m, 1H), 3.66-3.79 (m, 3H), 2.79-3.01 (m, 5H), 2.61-2.79 (m, 2H), 2.49 (br. s., 4H), 1.87-2.19 (m, 6H), 1.59-1.82 (m, 2H)

MS ES⁺ : 340

### 2.138 Example 138

### 1-(4-((3-cyclobutyl-2,3,4,5-tetrahydro-1H-benzo[d]azepin-7-yl)(hydroxy)methyl)piperidin-1-yl)ethanone

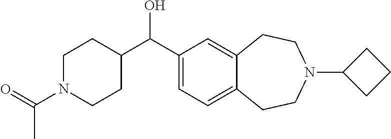

¹H NMR: (400 MHz, MeOD) δ 7.00-7.16 (m, 3H), 4.42-4.64 (m, 1H), 4.27-4.34 (m, 1H), 3.80-4.01 (m, 1H), 2.78-3.12 (m, 6H), 2.36-2.63 (m, 5H), 1.61-2.21 (m, 10H), 1.05-1.44 (m, 4H)

MS ES⁺ : 357

### 2.139 Example 139

### 1-(4-(2-(3-cyclobutyl-2,3,4,5-tetrahydro-1H-benzo[d]azepin-7-yl)-2-hydroxyethyl)piperidin-1-yl)ethanone

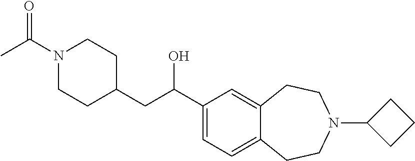

¹H NMR: (400 MHz, MeOD) δ 7.00-7.18 (m, 3H), 4.66-4.74 (m, 1H), 4.42-4.55 (m, 1H), 3.84-3.96 (m, 1H), 3.01-3.16 (m, 1H), 2.86-3.01 (m, 5H), 2.46-2.69 (m, 5H), 2.05-2.20 (m, 5H), 1.91-2.05 (m, 2H), 1.62-1.91 (m, 6H), 1.48-1.62 (m, 1H), 1.03-1.31 (m, 2H)

MS ES⁺: 371

### 2.140 Example 140

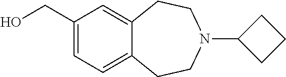

### (3-cyclobutyl-2,3,4,5-tetrahydro-1H-benzo[d]azepin-7-yl)methanol

¹H NMR: ¹H NMR (400 MHz, MeOD) δ 7.01-7.15 (m, 3H), 4.55 (s, 2H), 2.72-3.03 (m, 5H), 2.30-2.61 (m, 4H), 2.05-2.19 (m, 2H), 1.86-2.04 (m, 2H), 1.59-1.81 (m, 2H)

MS ES⁺ : 232

### 2.141 Example 141

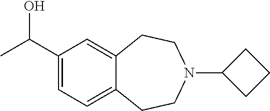

### 1-(3-cyclobutyl-2,3,4,5-tetrahydro-1H-benzo[d]azepin-7-yl)ethanol

¹H NMR: ¹H NMR (400 MHz, MeOD)*7.04-7.16 (m, 3H), 4.75-4.81 (m, 1H), 2.81-2.98 (m, 5H), 2.41-2.59 (m, 4H), 2.07-2.17 (m, 2H), 1.89-2.03 (m, 2H), 1.63-1.79 (m, 2H), 1.39-1.46 (m, 3H)

MS ES⁺ : 246

### 2.142 Example 142 Racemic

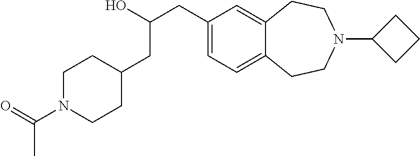

### 1-(4-(3-(3-cyclobutyl-2,3,4,5-tetrahydro-1H-benzo[d]azepin-7-yl)-2-hydroxypropyl)piperidin-1-yl)ethanone

¹H NMR (400 MHz, MeOD) δ 6.92-7.09 (m, 3H), 4.38-4.54 (m, 1H), 3.81-3.96 (m, 2H), 3.01-3.17 (m, 1H), 2.81-2.98 (m, 5H), 2.38-2.76 (m, 7H), 2.05-2.18 (m, 5H), 1.90-2.04 (m, 2H), 1.61-1.89 (m, 5H), 1.28-1.48 (m, 2H), 0.83-1.27 (m, 2H)

MS ES+: 385

### 2.143 Example 143

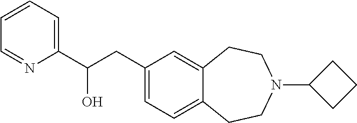

### 2-(3-cyclobutyl-2,3,4,5-tetrahydro-1H-benzo[d]azepin-7-yl)-1-(pyridin-2-yl)ethanol

¹H NMR (400 MHz, MeOD) δ 8.41-8.53 (m, 1H), 7.75-7.87 (m, 1H), 7.41-7.57 (m, 1H), 7.26-7.37 (m, 1H), 6.85-7.02 (m, 3H), 4.86-4.97 (m, 1H), 3.04-3.14 (m, 1H), 2.77-2.98 (m, 6H), 2.46 (br. s., 4H), 2.04-2.20 (m, 2H), 1.87-2.03 (m, 2H), 1.57-1.82 (m, 2H)

MS ES+: 323

### 2.144 Example 144 Racemic

### 1-(3-cyclobutyl-2,3,4,5-tetrahydro-1H-benzo[d]azepin-7-yl)-3-(pyridin-4-yl)propan-2-ol

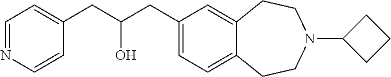

¹H NMR (400 MHz, MeOD) δ 8.29-8.36 (m, 2H), 7.19-7.28 (m, 2H), 6.88-7.02 (m, 3H), 3.94-4.03 (m, 1H), 2.75-2.91 (m, 6H), 2.59-2.73 (m, 3H), 2.43 (br. s., 4H), 1.99-2.12 (m, 2H), 1.82-1.98 (m, 2H), 1.55-1.75 (m, 2H)

MS ES+: 337

### 2.145 Example 145

### 2-(3-cyclobutyl-2,3,4,5-tetrahydro-1H-benzo[d]azepin-7-yl)-1-(pyridin-3-yl)ethanol

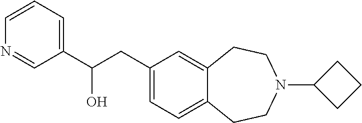

¹H NMR: (400 MHz, CDCl₃) δ 8.61-8.62 (m, 1H), 8.53-8.55 (m, 1H), 7.73-7.75 (m, 1H), 7.28-7.31 (m, 1H), 7.03-7.05 (m, 1H), 6.96-6.98 (m, 2H), 4.92-4.95 (m, 1H), 2.88-3.04 (m, 2H), 2.86 (s, 4H), 2.78 (s, 1H), 2.53 (s, 1H), 2.44 (s, 4H), 2.04-2.10 (m, 2H), 1.63 (s, 2H), 1.58-1.72 (m, 2H).

MS ES⁺ : 323

### 2.146 Example 146 racemic

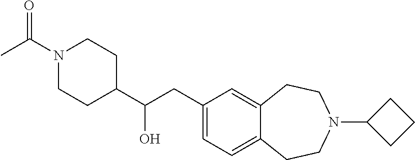

### 1-(4-(2-(3-cyclobutyl-2,3,4,5-tetrahydro-1H-benzo[d]azepin-7-yl)-1-hydroxyethyl)piperidin-1-yl)ethanone

¹H NMR (400 MHz, MeOD) δ 6.95-7.09 (m, 3H), 4.51-4.66 (m, 1H), 3.97 (br. s., 1H), 3.54-3.69 (m, 1H), 3.01-3.13 (m, 1H), 2.73-2.99 (m, 6H), 2.34-2.69 (m, 6H), 2.05-2.20 (m, 5H), 1.84-2.05 (m, 3H), 1.56-1.82 (m, 4H), 1.16-1.56 (m, 2H)

MS ES+: 371

### 2.147 Example 147

### 1-(4-(3-(3-cyclobutyl-2,3,4,5-tetrahydro-1H-benzo[d]azepin-7-yl)-2-hydroxypropyl)piperidin-1-yl)propan-1-one-racemate

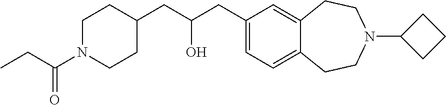

¹H NMR: (400 MHz, MeOD) δ 6.92-7.06 (m, 3H), 4.42-4.53 (m, 1H), 3.80-3.98 (m, 2H), 2.98-3.17 (m, 1H), 2.79-2.97 (m, 5H), 2.34-2.76 (m, 9H), 2.06-2.18 (m, 2H), 1.89-2.04 (m, 2H), 1.59-1.89 (m, 5H), 1.26-1.48 (m, 2H), 1.11 (m, 5H)

MS ES⁺ : 399

### 2.148 Example 148

### 1-(4-(3-(3-cyclobutyl-2,3,4,5-tetrahydro-1H-benzo[d]azepin-7-yl)-2-hydroxypropyl)piperidin-1-yl)ethanone-single enantiomer

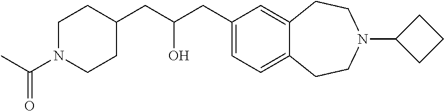

¹H NMR: (400 MHz, MeOD) δ

MS ES⁺:

### 2.149 Example 149

### 1-(4-(3-(3-cyclobutyl-2,3,4,5-tetrahydro-1H-benzo[d]azepin-7-yl)-2-hydroxypropyl)piperidin-1-yl)ethanone-single enantiomer

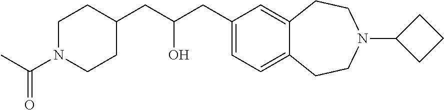

¹H NMR: (400 MHz, MeOD) δ

MS ES⁺:

### 2.150 Example 150 (Racemic)

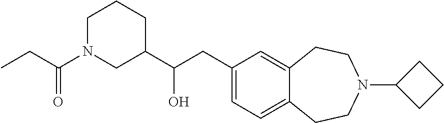

### 1-(4-(2-(3-cyclobutyl-2,3,4,5-tetrahydro-1H-benzo[d]azepin-7-yl)-1-hydroxyethyl)piperidin-1-yl)propan-1-one

¹H NMR (400 MHz, MeOD) δ 6.72-6.88 (m, 3H), 4.38 (br. s., 1H), 3.79 (br. s., 1H), 3.32-3.44 (m, 1H), 2.76-2.90 (m, 1H), 2.52-2.76 (m, 6H), 2.12-2.48 (m, 8H), 1.90 (br. s., 2H), 1.63-1.84 (m, 3H), 1.33-1.59 (m, 4H), 1.13 (br. s., 2H), 0.85-0.96 (m, 3H)

MS ES+: 385

### 2.151 Example 151 racemic

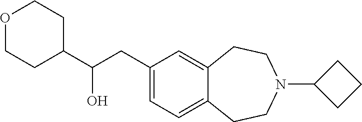

### 2-(3-cyclobutyl-2,3,4,5-tetrahydro-1H-benzo[d]azepin-7-yl)-1-(tetrahydro-2H-pyran-4-yl)ethanol

¹H NMR (400 MHz, MeOD) δ 6.92-7.08 (m, 3H), 3.90-4.06 (m, 2H), 3.49-3.62 (m, 1H), 3.37-3.45 (m, 1H), 2.75-2.99 (m, 7H), 2.32-2.66 (m, 5H), 2.05-2.19 (m, 2H), 1.87-2.04 (m, 2H), 1.38-1.85 (m, 7H)

MS ES+: 330

### 2.152 Example 152

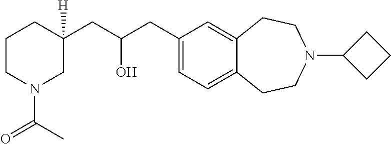

### 1-((3S)-3-(3-(3-cyclobutyl-2,3,4,5-tetrahydro-1H-benzo[d]azepin-7-yl)-2-hydroxypropyl)piperidin-1-yl)ethanone

¹H NMR (400 MHz, CDCl₃) δ ppm 1.11-1.31 (m, 1H) 1.58 (br. s., 13H) 2.01-2.25 (m, 5H) 2.77 (d, J=2.02 Hz, 11H) 3.61-4.04 (m, 2H) 4.25-4.49 (m, 1H) 6.96 (br. s., 2H) 7.05 (d, J=7.83 Hz, 1H)

MS ES+: 385

### 2.153 Example 153

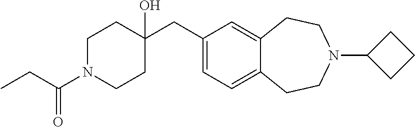

### 1-(4-((3-cyclobutyl-2,3,4,5-tetrahydro-1H-benzo[d]azepin-7-yl)methyl)-4-hydroxypiperidin-1-yl)propan-1-one

¹H NMR (400 MHz, DMSO-d₆) δ ppm 0.81-0.96 (m, 3H) 1.15-1.40 (m, 4H) 1.42-1.61 (m, 2H) 1.62-1.80 (m, 2H) 1.86-2.03 (m, 2H) 2.11-2.35 (m, 6H) 2.54 (s, 2H) 2.60-2.87 (m, 6H) 3.11-3.22 (m, 1H) 3.44-3.57 (m, 1H) 3.93-4.08 (m, 1H) 4.29 (s, 1H) 6.80-6.96 (m, 3H)

MS ES+: 371

### 2.154 Example 154

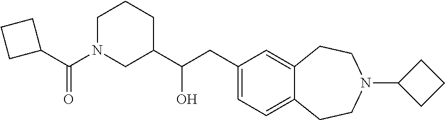

### cyclobutyl(3-(2-(3-cyclobutyl-2,3,4,5-tetrahydro-1H-benzo[d]azepin-7-yl)-1-hydroxyethyl)piperidin-1-yl)methanone

¹H NMR (400 MHz, MeOD) δ 6.84-7.09 (m, 3H) 4.43-4.66 (m, 1H) 3.76-3.94 (m, 1H) 3.52-3.66 (m, 1H) 3.35-3.47 (m, 1H) 2.93-3.02 (m, 1H) 2.85-2.93 (m, 4H) 2.76-2.83 (m, 1H) 2.57-2.65 (m, 1H) 2.52-2.59 (m, 1H) 2.47 (s, 4H) 2.06-2.35 (m, 6H) 1.78-2.05 (m, 5H) 1.52-1.78 (m, 4H) 1.16-1.43 (m, 3H).

MS ES⁺ 411

### 2.155 Example 155

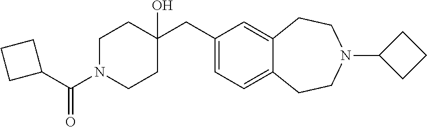

### cyclobutyl(4-((3-cyclobutyl-2,3,4,5-tetrahydro-1H-benzo[d]azepin-7-yl)methyl)-4-hydroxypiperidin-1-yl)methanone

¹H NMR (400 MHz, DMSO-d₆) δ ppm 1.12-1.36 (m, 4H) 1.42-1.85 (m, 6H) 1.87-2.10 (m, 6H) 2.15-2.39 (m, 4H) 2.53 (s, 2H) 2.59-2.89 (m, 6H) 3.03-3.23 (m, 2H) 3.28-3.39 (m, 1H) 3.87-4.03 (m, 1H) 4.27 (s, 1H) 6.75-6.99 (m, 3H)

MS ES+: 397

### 2.156 Example 156

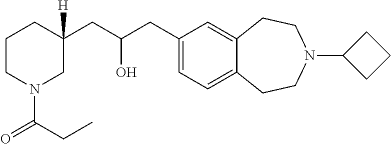

### 1-((3R)-3-(3-(3-cyclobutyl-2,3,4,5-tetrahydro-1H-benzo[d]azepin-7-yl)-2-hydroxypropyl)piperidin-1-yl)ethanone

¹H NMR (400 MHz, MeOD) δ 6.85-7.11 (m, 3H) 4.19-4.43 (m, 1H) 3.68-3.98 (m, 2H) 2.58-3.19 (m, 9H) 2.28-2.57 (m, 5H) 1.55-2.18 (m, 9H) 1.01-1.55 (m, 8H)

MS ES⁺ 399

### 2.157 Example 157

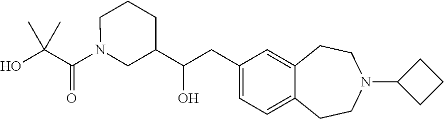

### 1-(4-((3-cyclobutyl-2,3,4,5-tetrahydro-1H-benzo[d]azepin-7-yl)methyl)-4-hydroxypiperidin-1-yl)-2-hydroxy-2-methylpropan-1-one

¹H NMR (400 MHz, DMSO-d₆) d ppm 1.28 (s, 6H) 1.31-1.47 (m, 4H) 1.49-1.68 (m, 2H) 1.68-1.86 (m, 2H) 1.91-2.07 (m, 2H) 2.23-2.43 (m, 4H) 2.60 (s, 2H) 2.67-2.86 (m, 5H) 4.33 (s, 1H) 5.27 (s, 1H) 6.84-7.06 (m, 3H) 4H were not assigned.

MS ES+: 401

### 2.158 Example 158

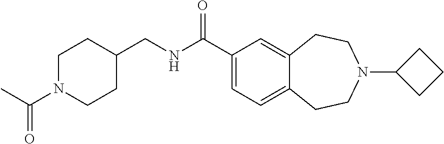

### N-((1-acetylpiperidin-4-yl)methyl)-3-cyclobutyl-2,3,4,5-tetrahydro-1H-benzo[d]azepine-7-carboxamide

¹H NMR (400 MHz, CHLOROFORM-d) δ 7.36-7.49 (m, 2H), 7.04-7.14 (m, 1H), 6.06-6.18 (m, 1H), 4.48-4.64 (m, 1H), 3.69-3.83 (m, 1H), 3.30-3.41 (m, 1H), 3.17-3.29 (m, 1H), 2.81-3.04 (m, 5H), 2.64-2.79 (m, 1H), 2.23-2.56 (m, 5H), 1.94-2.08 (m, 6H), 1.43-1.90 (m, 4H), 0.98-1.27 (m, 4H)

MS ES⁺ 384

### 2.159 Example 159

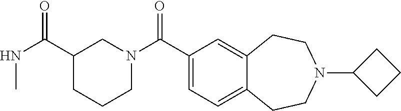

### 1-(3-cyclobutyl-2,3,4,5-tetrahydro-1H-benzo[d]azepine-7-carbonyl)-N-methylpiperidine-3-carboxamide

NMR: ¹H NMR (400 MHz, METHANOL-d₄) δ 7.00-7.47 (m, 3H), 4.38-4.65 (m, 1H), 3.61-3.86 (m, 1H), 2.25-3.21 (m, 15H), 2.06-2.22 (m, 2H), 1.40-2.04 (m, 8H)

MS ES⁺ 370

### 2.160 Example 160

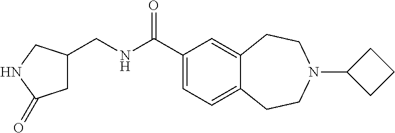

### 3-cyclobutyl-N-((5-oxopyrrolidin-3-yl)methyl)-2,3,4,5-tetrahydro-1H-benzo[d]azepine-7-carboxamide

¹H NMR (400 MHz, MeOD) δ 7.52-7.68 (m, 2H), 7.17-7.29 (m, 1H), 3.39-3.59 (m, 3H), 3.18-3.28 (m, 1H), 2.93-3.08 (m, 4H), 2.76-2.92 (m, 2H), 2.36-2.63 (m, 5H), 2.06-2.26 (m, 3H), 1.87-2.04 (m, 2H), 1.61-1.82 (m, 2H)

MS ES⁺ 342

### 2.161 Example 161

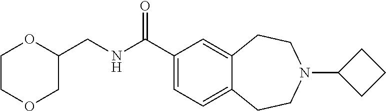

### N-((1,4-dioxan-2-yl)methyl)-3-cyclobutyl-2,3,4,5-tetrahydro-1H-benzo[d]azepine-7-carboxamide

¹H NMR (400 MHz, MeOD) δ 7.38-7.54 (m, 2H), 7.02-7.16 (m, 1H), 3.53-3.76 (m, 5H), 3.41-3.52 (m, 1H), 3.23-3.34 (m, 3H), 2.81-2.96 (m, 4H), 2.67-2.80 (m, 1H), 2.25-2.51 (m, 4H), 1.93-2.07 (m, 2H), 1.76-1.90 (m, 2H), 1.50-1.69 (m, 2H)

MS ES⁺ 345

### 2.162 Example 162

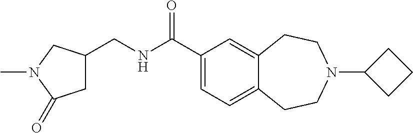

### 3-cyclobutyl-N-((1-methyl-5-oxopyrrolidin-3-yl)methyl)-2,3,4,5-tetrahydro-1H-benzo[d]azepine-7-carboxamide

¹H NMR (400 MHz, MeOD) δ 7.43-7.57 (m, 2H), 7.12-7.21 (m, 1H), 6.24-6.39 (m, 1H), 3.40-3.65 (m, 3H), 3.19-3.28 (m, 1H), 2.90-3.03 (m, 4H), 2.67-2.89 (m, 5H), 2.35-2.64 (m, 5H), 2.15-2.28 (m, 1H), 2.03-2.15 (m, 2H), 1.85-1.99 (m, 2H), 1.58-1.77 (m, 2H)

MS ES⁺ 356

### 2.163 Example 163

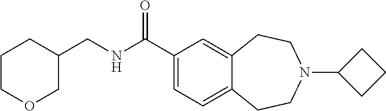

### 3-cyclobutyl-N-((tetrahydro-2H-pyran-3-yl)methyl)-2,3,4,5-tetrahydro-1H-benzo[d]azepine-7-carboxamide

¹H NMR (400 MHz, MeOD) δ 7.52-7.64 (m, 2H), 7.16-7.26 (m, 1H), 3.77-3.94 (m, 2H), 3.37-3.52 (m, 2H), 3.21-3.29 (m, 2H), 2.94-3.05 (m, 4H), 2.78-2.92 (m, 1H), 2.36-2.61 (m, 4H), 2.06-2.19 (m, 2H), 1.84-2.01 (m, 4H), 1.54-1.80 (m, 5H)

MS ES⁺ 343

### 2.164 Example 164

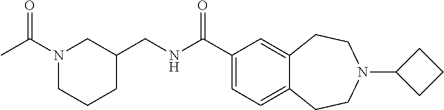

### N-((1-acetylpiperidin-3-yl)methyl)-3-cyclobutyl-2,3,4,5-tetrahydro-1H-benzo[d]azepine-7-carboxamide

¹H NMR (400 MHz, MeOD) δ 7.53-7.69 (m, 2H), 7.16-7.28 (m, 1H), 4.23-4.41 (m, 1H), 3.74-3.92 (m, 1H), 3.13-3.27 (m, 2H), 2.74-3.09 (m, 6H), 2.39-2.73 (m, 4H), 2.05-2.18 (m, 5H), 1.64-2.05 (m, 7H), 1.28-1.61 (m, 2H), 0.83-0.95 (m, 1H)

MS ES⁺ 384

### 2.165 Example 165

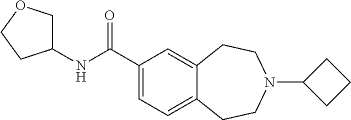

### 3-cyclobutyl-N-(tetrahydrofuran-3-yl)-2,3,4,5-tetrahydro-1H-benzo[d]azepine-7-carboxamide

¹H NMR (400 MHz, MeOD) δ 7.53-7.67 (m, 2H), 7.16-7.26 (m, 1H), 4.52-4.65 (m, 1H), 3.92-4.07 (m, 2H), 3.78-3.91 (m, 1H), 3.65-3.77 (m, 1H), 2.93-3.07 (m, 4H), 2.79-2.93 (m, 1H), 2.38-2.67 (m, 4H), 2.24-2.38 (m, 1H), 2.06-2.20 (m, 2H), 1.87-2.07 (m, 3H), 1.59-1.82 (m, 2H)

MS ES⁺ 315

### 2.166 Example 166

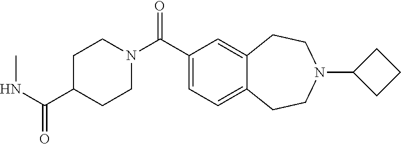

### 1-(3-cyclobutyl-2,3,4,5-tetrahydro-1H-benzo[d]azepine-7-carbonyl)-N-methylpiperidine-4-carboxamide

¹H NMR (400 MHz, MeOD) δ 7.14-7.25 (m, 3H), 4.53-4.76 (m, 1H), 3.74-3.91 (m, 1H), 2.79-3.21 (m, 7H), 2.67-2.78 (m, 4H), 2.36-2.63 (m, 4H), 2.06-2.21 (m, 2H), 1.83-2.03 (m, 3H), 1.55-1.82 (m, 5H)

MS ES⁺ 370

### 2.167 Example 167

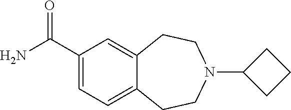

### 3-cyclobutyl-2,3,4,5-tetrahydro-1H-benzo[d]azepine-7-carboxamide

¹H NMR (400 MHz, MeOD) δ 7.64 (br. s., 2H), 7.22 (d, J=7.83 Hz, 1H), 2.76-3.09 (m, 5H), 2.50 (br. s., 4H), 2.12 (br. s., 2H), 1.86-2.04 (m, 2H), 1.57-1.86 (m, 2H)

MS ES⁺ 245

### 2.168 Example 168

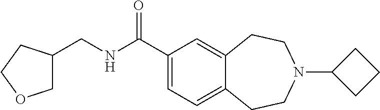

### 3-cyclobutyl-N-((tetrahydrofuran-3-yl)methyl)-2,3,4,5-tetrahydro-1H-benzo[d]azepine-7-carboxamide

¹H NMR (400 MHz, MeOD) δ 7.48-7.67 (m, 2H), 7.12-7.29 (m, 1H), 3.66-3.96 (m, 3H), 3.53-3.66 (m, 1H), 3.35-3.45 (m, 2H), 2.91-3.06 (m, 4H), 2.74-2.92 (m, 1H), 2.28-2.68 (m, 5H), 1.84-2.19 (m, 5H), 1.57-1.81 (m, 3H)

MS ES⁺ 329

### 2.169 Example 169

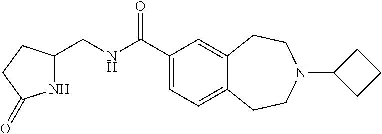

### 3-cyclobutyl-N-((5-oxopyrrolidin-2-yl)methyl)-2,3,4,5-tetrahydro-1H-benzo[d]azepine-7-carboxamide

¹H NMR (400 MHz, MeOD) δ 7.42-7.54 (m, 2H), 7.02-7.16 (m, 1H), 3.74-3.86 (m, 1H), 3.26-3.43 (m, 2H), 2.81-2.93 (m, 4H), 2.64-2.80 (m, 1H), 2.28-2.51 (m, 4H), 2.10-2.29 (m, 3H), 1.92-2.05 (m, 2H), 1.74-1.90 (m, 3H), 1.50-1.68 (m, 2H)

MS ES⁺ 342

### 2.170 Example 170

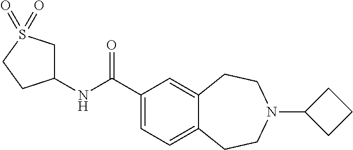

### 3-Cyclobutyl-2,3,4,5-tetrahydro-1H-benzo[d]azepine-7-carboxylic acid (1,1-dioxo-tetrahydro-1lambda*6*-thiophen-3-yl)-amide

¹H NMR (400 MHz, CDCl₃) δ 7.46-7.58 (m, 2H), 7.09-7.22 (m, 1H), 6.64-6.80 (m, 1H), 4.95-5.09 (m, 1H), 3.39-3.53 (m, 1H), 3.05-3.36 (m, 3H), 2.87-3.03 (m, 4H), 2.72-2.87 (m, 1H), 2.56-2.69 (m, 1H), 2.31-2.54 (m, 4H), 2.02-2.15 (m, 2H), 1.81-1.99 (m, 2H), 1.65-1.78 (m, 2H)

MS ES⁺ 363

### 2.171 Example 171

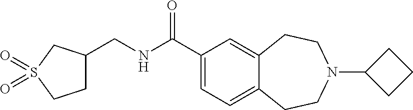

### 3-Cyclobutyl-2,3,4,5-tetrahydro-1H-benzo[d]azepine-7-carboxylic acid (1,1-dioxo-tetrahydro-1lambda*6*-thiophen-3-ylmethyl)-amide

¹H NMR (400 MHz, MeOD) δ 7.55-7.66 (m, 2H), 7.19-7.26 (m, 1H), 3.45-3.60 (m, 2H), 3.18-3.29 (m, 2H), 3.05-3.17 (m, 1H), 2.95-3.05 (m, 4H), 2.74-2.95 (m, 3H), 2.31-2.62 (m, 5H), 2.06-2.19 (m, 2H), 1.88-2.04 (m, 3H), 1.62-1.80 (m, 2H)

MS ES⁺ 377

### 2.172 Example 172

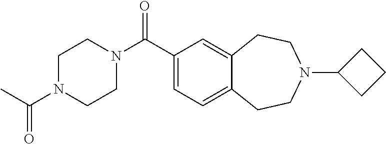

### 1-(4-(3-cyclobutyl-2,3,4,5-tetrahydro-1H-benzo[d]azepine-7-carbonyl)piperazin-1-yl)ethanone

¹H NMR (400 MHz, MeOD) δ ppm 1.67-2.01 (m, 2H) 2.07-2.28 (m, 3H) 2.40 (br. s., 4H) 2.70-2.95 (m, 2H) 3.04-3.27 (m, 2H) 3.41-4.00 (m, 13H) 7.17-7.45 (m, 3H)

MS ES+ 356

### 2.173 Example 173

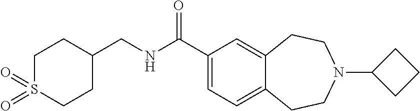

### 1,1-Dioxo-tetrahydro-1lambda*6*-thiopyran-4-carboxylic acid (3-cyclobutyl-2,3,4,5-tetrahydro-1H-benzo[d]azepin-7-ylmethyl)-amide

¹H NMR (400 MHz, MeOD) δ 7.00-7.11 (m, 3H), 4.27-4.35 (m, 2H), 3.06-3.23 (m, 5H), 2.86-3.00 (m, 5H), 2.42-2.62 (m, 4H), 2.08-2.36 (m, 6H), 1.90-2.06 (m, 2H), 1.63-1.81 (m, 2H)

MS ES⁺ 391

### 2.174 Example 174

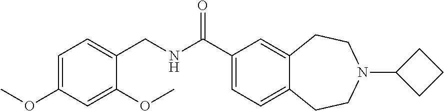

### 3-cyclobutyl-N-(2,4-dimethoxybenzyl)-2,3,4,5-tetrahydro-1H-benzo[d]azepine-7-carboxamide

¹H NMR (400 MHz, DMSO) δ 8.63 (t, 1H), 7.64 (d, 1H), 7.62 (d, 1H), 7.19 (d, 1H), 7.07 (d, 1H), 6.55 (d, 1H), 6.47 (dd, 1H), 4.35 (d, 2H), 3.81 (s, 3H), 3.74 (s, 3H), 2.81-2.91 (m, 4H), 2.71-2.81 (m, 1H), 2.28-2.43 (bs, 4H), 1.96-2.05 (m, 2H), 1.71-1.84 (m, 2H), 1.51-1.66 (m, 2H)

MS ES⁺ 395

### 2.175 Example 175

### N-((3-cyclobutyl-2,3,4,5-tetrahydro-1H-benzo[d]azepin-7-yl)methyl)tetrahydrofuran-3-carboxamide

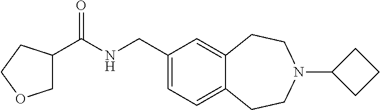

¹H NMR: (400 MHz, MeOD) δ 6.96-7.13 (m, 3H), 4.25-4.39 (m, 2H), 3.85-4.02 (m, 2H), 3.74-3.85 (m, 2H), 3.00-3.13 (m, 1H), 2.79-2.99 (m, 5H), 2.49 (br. s., 4H), 2.05-2.21 (m, 4H), 1.87-2.04 (m, 2H), 1.59-1.84 (m, 2H)

MS ES⁺ 329

### 2.176 Example 176

### N-((3-cyclobutyl-2,3,4,5-tetrahydro-1H-benzo[d]azepin-7-yl)methyl)tetrahydro-2H-pyran-4-carboxamide

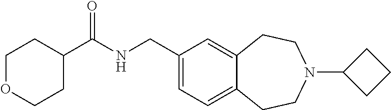

¹H NMR: (400 MHz, MeOD) δ 6.99-7.12 (m, 3H), 4.31 (s, 2H), 3.95-4.02 (m, 2H), 3.39-3.53 (m, 2H), 2.77-3.01 (m, 5H), 2.33-2.59 (m, 4H), 2.06-2.20 (m, 2H), 1.88-2.04 (m, 2H), 1.64-1.87 (m, 6H)

MS ES⁺ 343

### 2.177 Example 177

### N-((3-cyclobutyl-2,3,4,5-tetrahydro-1H-benzo[d]azepin-7-yl)methyl)-1-methyl-1H-pyrazole-5-carboxamide

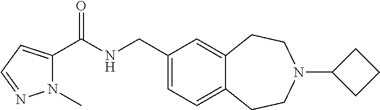

¹H NMR: (400 MHz, MeOD) δ 7.39-7.55 (m, 1H), 7.04-7.19 (m, 3H), 6.76-6.86 (m, 1H), 4.48 (s, 2H), 4.09-4.21 (m, 3H), 2.79-3.00 (m, 5H), 2.50 (br. s., 4H), 2.06-2.22 (m, 2H), 1.86-2.04 (m, 2H), 1.58-1.84 (m, 2H)

MS ES⁺ 339

### 2.178 Example 178

### 1-(4-(2-(3-cyclobutyl-2,3,4,5-tetrahydro-1H-benzo[d]azepin-7-yl)-2-hydroxyethyl)piperidin-1-yl)ethanone

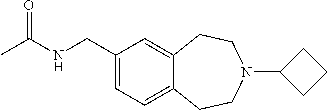

¹H NMR: (400 MHz, MeOD) δ 6.93-7.24 (m, 3H), 4.30 (br. s., 2H), 2.77-3.03 (m, 5H), 2.48 (br. s., 4H), 2.06-2.21 (m, 2H), 1.88-2.05 (m, 5H), 1.61-1.82 (m, 2H)

MS ES⁺ 273

### 2.179 Example 2.179

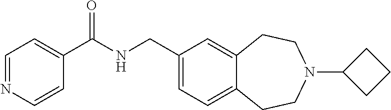

### N-((3-cyclobutyl-2,3,4,5-tetrahydro-1H-benzo[d]azepin-7-yl)methyl)isonicotinamide

¹H NMR: ¹H NMR (400 MHz, MeOD) δ 8.55-8.80 (m, 2H), 7.74-7.90 (m, 2H), 7.01-7.22 (m, 3H), 4.40-4.65 (m, 2H), 2.71-3.03 (m, 5H), 2.25-2.68 (m, 4H), 2.03-2.18 (m, 2H), 1.85-2.02 (m, 2H), 1.59-1.80 (m, 2H)

MS ES⁺ 336

### 2.180 Example 180

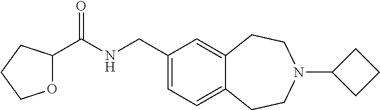

### N-((3-cyclobutyl-2,3,4,5-tetrahydro-1H-benzo[d]azepin-7-yl)methyl)tetrahydrofuran-2-carboxamide

¹H NMR: ¹H NMR (400 MHz, MeOD) δ 6.93-7.12 (m, 3H), 4.24-4.41 (m, 3H), 3.93-4.07 (m, 1H), 3.78-3.93 (m, 1H), 2.70-3.00 (m, 5H), 2.19-2.59 (m, 5H), 2.03-2.18 (m, 2H), 1.79-2.04 (m, 5H), 1.58-1.80 (m, 2H)

MS ES⁺ 329

### 2.181 Example 181

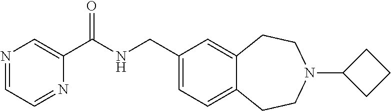

### N-((3-cyclobutyl-2,3,4,5-tetrahydro-1H-benzo[d]azepin-7-yl)methyl)pyrazine-2-carboxamide

¹H NMR: NMR (400 MHz, MeOD) δ 9.20-9.34 (m, 1H), 8.76-8.87 (m, 1H), 8.61-8.74 (m, 1H), 7.12-7.27 (m, 3H), 4.50-4.69 (m, 2H), 3.22-3.61 (m, 5H), 3.02-3.18 (m, 4H), 2.09-2.43 (m, 4H), 1.71-1.96 (m, 2H)

MS ES⁺ 337

### 2.182 Example 182

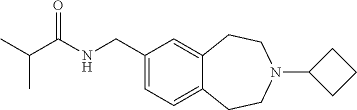

### N-((3-cyclobutyl-2,3,4,5-tetrahydro-1H-benzo[d]azepin-7-yl)methyl)isobutyramide

¹H NMR: NMR (400 MHz, CDCl₃) δ 6.84-6.99 (m, 3H), 5.43-5.62 (m, 1H), 4.21-4.34 (m, 2H), 2.73-2.88 (m, 4H), 2.60-2.73 (m, 1H), 2.19-2.45 (m, 4H), 1.89-2.02 (m, 2H), 1.73-1.88 (m, 2H), 1.44-1.65 (m, 3H), 1.01-1.12 (m, 6H)

MS ES⁺ 301

### 2.183 Example 183

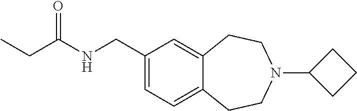

### N-((3-cyclobutyl-2,3,4,5-tetrahydro-1H-benzo[d]azepin-7-yl)methyl)propionamide

¹H NMR: ¹H NMR (400 MHz, MeOD) δ 6.98-7.11 (m, 3H), 4.28-4.36 (m, 2H), 2.77-2.98 (m, 5H), 2.37-2.57 (m, 4H), 2.21-2.30 (m, 2H), 2.05-2.18 (m, 2H), 1.87-2.02 (m, 2H), 1.63-1.80 (m, 2H), 1.10-1.21 (m, 3H)

MS ES⁺ 287

### 2.184 Example 184

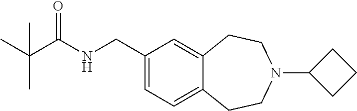

### N-((3-cyclobutyl-2,3,4,5-tetrahydro-1H-benzo[d]azepin-7-yl)methyl)pivalamide

¹H NMR: ¹H NMR (400 MHz, MeOD) δ 6.95-7.11 (m, 3H), 4.26-4.35 (m, 2H), 2.80-2.96 (m, 5H), 2.39-2.57 (m, 4H), 2.06-2.18 (m, 2H), 1.89-2.02 (m, 2H), 1.61-1.81 (m, 2H), 1.22 (s, 9H)

MS ES⁺ 315

### 2.185 Example 185

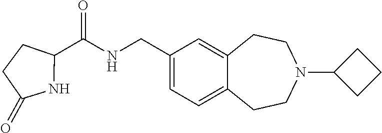

### N-((3-cyclobutyl-2,3,4,5-tetrahydro-1H-benzo[d]azepin-7-yl)methyl)-5-oxopyrrolidine-2-carboxamide

¹H NMR (400 MHz, MeOD) δ ppm 1.59-1.81 (m, 2H) 1.85-2.03 (m, 2H) 2.05-2.20 (m, 3H) 2.24-2.62 (m, 7H) 2.76-3.05 (m, 5H) 4.21 (s, 1H) 4.28-4.41 (m, 2H) 7.07 (d, 3H)

MS ES+ 342

### 2.186 Example 186

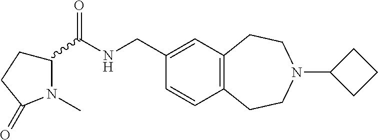

### N-((3-cyclobutyl-2,3,4,5-tetrahydro-1H-benzo[d]azepin-7-yl)methyl)-1-methyl-5-oxopyrrolidine-2-carboxamide

¹H NMR (400 MHz, MeOD) δ ppm 1.50-1.85 (m, 2H) 1.87-2.21 (m, 4H) 2.49 (br. s., 6H) 2.78 (s, 3H) 2.93 (br. s., 5H) 4.05-4.25 (m, 1H) 4.37 (s, 2H) 7.01-7.20 (m, 3H)

MS ES⁺ 326

### 2.187 Example 187

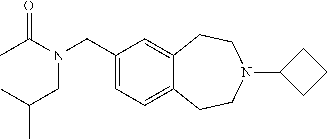

### N-((3-cyclobutyl-2,3,4,5-tetrahydro-1H-benzo[d]azepin-7-yl)methyl)-N-isobutylacetamide

¹H NMR (400 MHz, MeOD) δ 7.04-7.15 (m, 1H), 6.92-7.02 (m, 2H), 4.56-4.62 (m, 2H), 3.19-3.23 (m, 1H), 3.10-3.16 (m, 1H), 2.81-2.99 (m, 5H), 2.49 (br. s., 4H), 2.19 (s, 2H), 2.07-2.17 (m, 6H), 1.88-2.07 (m, 5H), 1.61-1.79 (m, 3H)

MS ES⁺ 329

### 2.188 Example 188

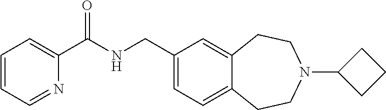

### N-((3-cyclobutyl-2,3,4,5-tetrahydro-1H-benzo[d]azepin-7-yl)methyl)picolinamide

¹H NMR (400 MHz, DMSO) d ppm 1.41-1.83 (m, 2H) 1.86-2.27 (m, 3H) 2.53-2.76 (m, 4H) 2.75-3.13 (m, 3H) 4.33-4.56 (m, 2H) 6.97-7.24 (m, 3H) 7.51-7.69 (m, 1H) 7.89-8.14 (m, 2H) 8.56-8.72 (m, 1H) 9.09-9.33 (m, 1H)

MS ES+ 336

### 2.189 Example 189

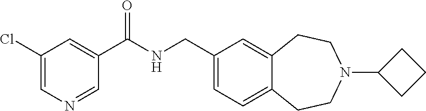

### 5-chloro-N-((3-cyclobutyl-2,3,4,5-tetrahydro-1H-benzo[d]azepin-7-yl)methyl)nicotinamide

¹H NMR (400 MHz, DMSO)₅ ppm 1.44-1.68 (m, 2H) 1.69-1.87 (m, 2H) 1.93-2.08 (m, 2H) 2.18-2.45 (m, 4H) 2.82 (br. s., 5H) 4.44 (d, 2H) 6.98-7.17 (m, 3H) 8.34 (t, 1H) 8.79 (d, 1H) 8.99 (d, 1H) 9.17-9.30 (m, 1H)

MS ES+ 370

### 2.190 Example 190

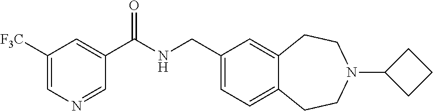

### N-((3-cyclobutyl-2,3,4,5-tetrahydro-1H-benzo[d]azepin-7-yl)methyl)-5-(trifluoromethyl)nicotinamide

¹H NMR (400 MHz, DMSO δ ppm 1.47-1.70 (m, 2H) 1.70-1.93 (m, 2H) 1.93-2.12 (m, 2H) 2.18-2.46 (m, 4H) 2.63-3.03 (m, 5H) 4.39-4.54 (m, 2H) 7.10 (br. s., 3H) 8.61 (br. s., 1H) 9.14 (s, 1H) 9.32 (s, 2H)

MS ES+ 404

### 2.191 Example 191

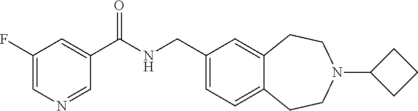

### N-((3-cyclobutyl-2,3,4,5-tetrahydro-1H-benzo[d]azepin-7-yl)methyl)-5-fluoronicotinamide

¹H NMR (400 MHz, DMSO) δ ppm 1.56 (m, 2H) 1.69-1.87 (m, 2H) 1.92-2.08 (m, 2H) 2.17-2.43 (m, 4H) 2.82 (br. s., 5H) 4.41 (s, 2H) 7.00-7.16 (m, 3H) 7.42-7.51 (m, 1H) 8.12-8.26 (m, 1H) 8.29-8.42 (m, 1H) 8.95-9.11 (m, 1H)

MS ES⁺ 354

### 2.192 Example 192

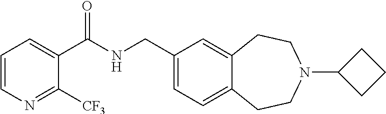

### N-((3-cyclobutyl-2,3,4,5-tetrahydro-1H-benzo[d]azepin-7-yl)methyl)-2-(trifluoromethyl)nicotinamide

¹H NMR (400 MHz, DMSO) δ ppm 1.44-1.68 (m, 2H) 1.69-1.87 (m, 2H) 1.92-2.07 (m, 2H) 2.22-2.43 (m, 4H) 2.66-2.90 (m, 5H) 4.32-4.46 (m, 2H) 6.99-7.15 (m, 3H) 7.72-7.86 (m, 1H) 7.98-8.12 (m, 1H) 8.75-8.85 (m, 1H) 9.06-9.19 (m, 1H)

MS ES⁺ 404

### 2.193 Example 193

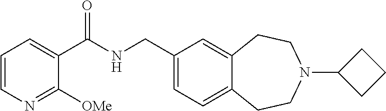

### N-((3-cyclobutyl-2,3,4,5-tetrahydro-1H-benzo[d]azepin-7-yl)methyl)-2-methoxynicotinamide

¹H NMR (400 MHz, DMSO) δ ppm 1.49-1.66 (m, 2H) 1.69-1.86 (m, 2H) 1.93-2.05 (m, 2H) 2.20-2.43 (m, 4H) 2.67-2.86 (m, 5H) 3.97 (s, 3H) 4.38-4.48 (m, 2H) 7.02-7.08 (m, 3H) 7.09-7.16 (m, 1H) 8.09-8.16 (m, 1H) 8.27-8.34 (m, 1H) 8.67-8.81 (m, 1H)

MS ES⁺ 366

### 2.194 Example 194

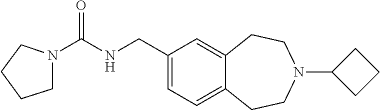

### N-((3-cyclobutyl-2,3,4,5-tetrahydro-1H-benzo[d]azepin-7-yl)methyl)pyrrolidine-1-carboxamide

¹H NMR (400 MHz, DMSO-d6) δ ppm 1.43-1.69 (m, 2H) 1.79 (t, 5H) 1.92-2.10 (m, 2H) 2.13-2.47 (m, 4H) 2.81 (br. s., 5H) 3.22 (t, 4H) 3.28-3.55 (m, 1H) 4.16 (d, 2H) 6.51-6.67 (m, 1H) 7.00 (br. s., 3H)

MS ES+ 328

### 2.195 Example 195

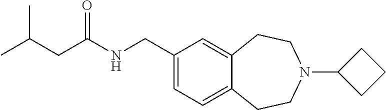

### N-((3-cyclobutyl-2,3,4,5-tetrahydro-1H-benzo[d]azepin-7-yl)methyl)-3-methylbutanamide

¹H NMR (400 MHz, METHANOL-d4) δ ppm 0.96 (d, 6H) 1.55-1.81 (m, 2H) 1.84-2.01 (m, 2H) 2.03-2.19 (m, 5H) 2.27-2.62 (m, 4H) 2.72-3.03 (m, 5H) 4.26-4.38 (m, 2H) 7.05 (s, 3H)

MS ES+ 315

### 2.196 Example 196

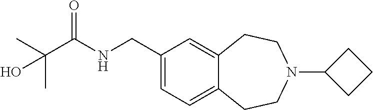

### N-((3-cyclobutyl-2,3,4,5-tetrahydro-1H-benzo[d]azepin-7-yl)methyl)-2-hydroxy-2-methylpropanamide

¹H NMR (400 MHz, METHANOL-d4) δ ppm 1.15 (s, 6H) 1.35-1.57 (m, 2H) 1.61-1.79 (m, 2H) 1.79-1.96 (m, 2H) 2.06-2.38 (m, 4H) 2.50-2.75 (m, 5H) 4.05-4.14 (m, 2H) 6.81 (d, 3H)

MS ES+ 317

### 2.197 Example 197

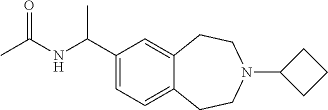

### N-(1-(3-cyclobutyl-2,3,4,5-tetrahydro-1H-benzo[d]azepin-7-yl)ethyl)acetamide

¹H NMR (400 MHz, METHANOL-d₄) δ 7.01-7.11 (m, 3H), 2.79-2.97 (m, 5H), 2.36-2.58 (m, 4H), 2.06-2.18 (m, 3H), 1.89-2.01 (m, 5H), 1.61-1.78 (m, 2H), 1.37-1.46 (m, 3H)

MS ES⁺ 287

### 2.198 Example 198

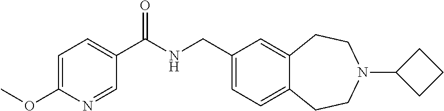

### N-((3-cyclobutyl-2,3,4,5-tetrahydro-1H-benzo[d]azepin-7-yl)methyl)-6-methoxynicotinamide

¹H NMR (400 MHz, METHANOL-d₄) δ ppm 1.72 (none, 2H) 1.87-2.10 (m, 2H) 2.08-2.28 (m, 2H) 2.35-2.76 (m, 4H) 2.95 (br. s., 5H) 3.98 (s, 3H) 4.53 (s, 2H) 6.87 (d, 1H) 7.05-7.21 (m, 3H) 8.06-8.19 (m, 1H) 8.67 (d, 1H)

MS ES⁺ 366

### 2.199 Example 199

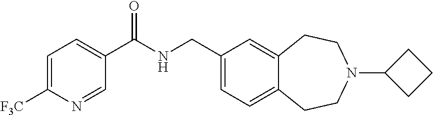

### N-((3-cyclobutyl-2,3,4,5-tetrahydro-1H-benzo[d]azepin-7-yl)methyl)-6-(trifluoromethyl)nicotinamide

¹H NMR (400 MHz, METHANOL-d₄) δ ppm 1.55 (none, 2H) 1.75-1.91 (m, 2H) 1.93-2.12 (m, 2H) 2.18-2.60 (m, 4H) 2.82 (d, 5H) 4.45 (s, 2H) 7.01 (d, 3H) 7.75-7.91 (m, 1H) 8.26-8.44 (m, 1H) 9.02 (s, 1H)

MS ES⁺ 404

### 2.200 Example 200

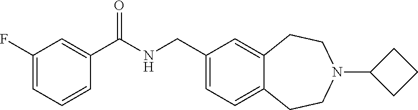

### N-((3-cyclobutyl-2,3,4,5-tetrahydro-1H-benzo[d]azepin-7-yl)methyl)-3-fluorobenzamide

¹H NMR (400 MHz, DMSO) δ ppm 1.49-1.65 (m, 2H) 1.69-1.84 (m, 2H) 1.92-2.05 (m, 2H) 2.17-2.44 (m, 4H) 2.68-2.86 (m, 5H) 4.34-4.49 (m, 2H) 6.98-7.11 (m, 3H) 7.34-7.43 (m, 1H) 7.48-7.57 (m, 1H) 7.64-7.72 (m, 1H) 7.72-7.79 (m, 1H) 9.02-9.15 (m, 1H)

MS ES⁺ 353

### 2.201 Example 201

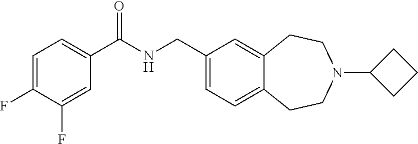

### N-((3-cyclobutyl-2,3,4,5-tetrahydro-1H-benzo[d]azepin-7-yl)methyl)-3,4-difluorobenzamide

¹H NMR (400 MHz, METHANOL-d₄) δ ppm 1.69 (none, 2H) 1.95 (br. s., 2H) 2.11 (br. s., 2H) 2.49 (br. s., 4H) 2.93 (br. s., 5H) 4.52 (s, 2H) 7.11 (br. s., 3H) 7.30-7.52 (m, 1H) 7.65-7.88 (m, 2H)

MS ES⁺ 371

### 2.202 Example 202

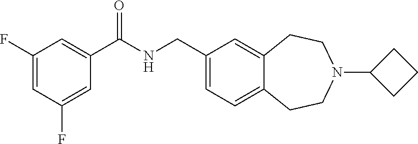

### N-((3-cyclobutyl-2,3,4,5-tetrahydro-1H-benzo[d]azepin-7-yl)methyl)-3,5-difluorobenzamide

¹H NMR (400 MHz, METHANOL-d₄) δ ppm 1.55-1.86 (m, 2H) 1.86-2.07 (m, 2H) 2.07-2.27 (m, 2H) 2.33-2.75 (m, 4H) 2.95 (br. s., 5H) 4.52 (s, 2H) 7.04-7.27 (m, 4H) 7.48 (dd, 2H)

MS ES⁺ 371

### 2.203 Example 203

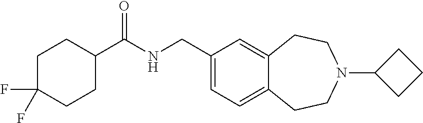

### N-((3-cyclobutyl-2,3,4,5-tetrahydro-1H-benzo[d]azepin-7-yl)methyl)-4,4-difluorocyclohexanecarboxamide

¹H NMR (400 MHz, DMSO-d₆) δ ppm 1.61 (br. s., 4H) 1.79 (t, 6H) 2.01 (br. s., 4H) 2.33 (d, 4H) 2.80 (br. s., 5H) 4.19 (d, 2H) 6.87-7.00 (m, 2H) 7.04 (d, J=7.33 Hz, 1H) 8.30 (br. s., 1H)

MS ES⁺ 377

### 2.204 Example 204

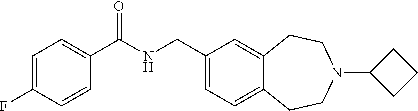

### N-((3-cyclobutyl-2,3,4,5-tetrahydro-1H-benzo[d]azepin-7-yl)methyl)-4-fluorobenzamide

¹H NMR (400 MHz, METHANOL-d₄) δ ppm 1.70 (none, 2H) 1.83-2.04 (m, 2H) 2.05-2.18 (m, 2H) 2.34-2.65 (m, 4H) 2.93 (br. s., 5H) 4.53 (s, 2H) 7.02-7.16 (m, 3H) 7.16-7.26 (m, 2H) 7.92 (d, 2H)

MS ES⁺ 353

### 2.205 Example 205

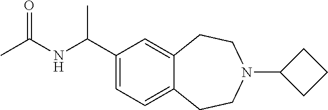

### N-(1-(3-cyclobutyl-2,3,4,5-tetrahydro-1H-benzo[d]azepin-7-yl)ethyl)acetamide

NMR: ¹H NMR (400 MHz, METHANOL-d₄) δ 7.00-7.15 (m, 3H), 4.90-5.03 (m, 1H), 2.81-2.98 (m, 5H), 2.42-2.57 (m, 4H), 2.05-2.19 (m, 2H), 1.90-2.03 (m, 5H), 1.62-1.79 (m, 2H), 1.37-1.48 (m, 3H)

MS ES⁺ 287

### 2.206 Example 206

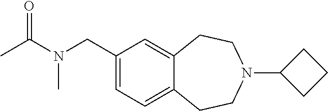

### N-((3-cyclobutyl-2,3,4,5-tetrahydro-1H-benzo[d]azepin-7-yl)methyl)-N-methylacetamide

NMR: ¹H NMR (400 MHz, METHANOL-d₄) δ 7.04-7.17 (m, 1H), 6.92-7.04 (m, 2H), 4.49-4.60 (m, 2H), 2.80-3.01 (m, 8H), 2.38-2.57 (m, 4H), 2.07-2.20 (m, 5H), 1.88-2.03 (m, 2H), 1.60-1.80 (m, 2H)

MS ES⁺ 287

### 2.207 Example 207

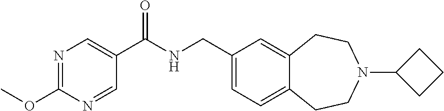

### N-((3-cyclobutyl-2,3,4,5-tetrahydro-1H-benzo[d]azepin-7-yl)methyl)-2-methoxypyrimidine-5-carboxamide

NMR: ¹H NMR (400 MHz, DMSO) δ 9.11 (t, 1H), 9.03 (s, 2H), 7.03-7.10 (m, 3H), 4.43 (d, 2H), 3.98 (s, 3H), 2.70-2.85 (m, 5H), 2.28-2.40 (m, 4H), 1.96-2.04 (m, 2H), 1.72-1.84 (m, 2H), 1.48-1.66 (m, 2H)

MS ES⁺ 367

### 2.208 Example 208

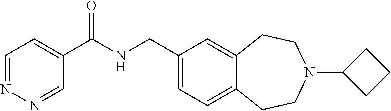

### N-((3-cyclobutyl-2,3,4,5-tetrahydro-1H-benzo[d]azepin-7-yl)methyl)pyridazine-4-carboxamide

¹H NMR (400 MHz, DMSO) δ 9.56-9.60 (m, 1H), 9.41-9.50 (m, 2H), 8.03 (dd, 1H), 7.04-7.11 (m, 3H), 4.46 (d, 2H), 2.70-2.87 (m, 5H), 2.26-2.42 (m, 4H), 1.95-2.05 (m, 2H), 1.71-1.84 (m, 2H), 1.48-1.66 (m, 1H)

MS ES⁺ 337

### 2.209 Example 209

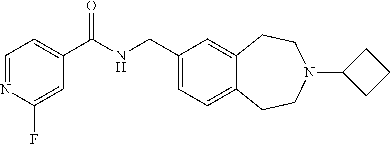

### N-((3-cyclobutyl-2,3,4,5-tetrahydro-1H-benzo[d]azepin-7-yl)methyl)-2-fluoroisonicotinamide

¹H NMR (400 MHz, DMSO) δ 9.40 (s, 1H), 8.47 (d, 1H), 7.84 (d, 1H), 7.63 (s, 1H), 7.09-7.17 (m, 3H), 4.50 (d, 2H), 2.87 (bs, 4H), 2.76-2.86 (m, 1H), 2.32-2.47 (bs, 4H), 2.02-2.12 (m, 2H), 1.77-1.90 (m, 2H), 1.57-1.72 (m, 2H)

MS ES⁺ 354

### 2.210 Example 210

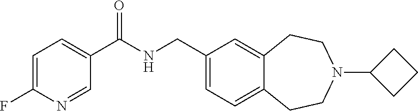

### N-((3-cyclobutyl-2,3,4,5-tetrahydro-1H-benzo[d]azepin-7-yl)methyl)-6-fluoronicotinamide

¹H NMR (400 MHz, DMSO) δ 9.12-9.21 (m, 1H), 8.74 (d, 1H), 8.42 (td, 1H), 7.31 (dd, 1H), 7.03-7.09 (m, 3H), 4.43 (d, 2H), 2.70-2.84 (m, 5H), 2.28-2.39 (bs, 4H), 1.95-2.04 (m, 2H), 1.72-1.83 (m, 2H), 1.50-1.66 (m, 2H)

MS ES⁺ 354

### 2.211 Example 211

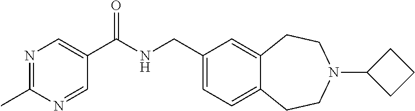

### N-((3-cyclobutyl-2,3,4,5-tetrahydro-1H-benzo[d]azepin-7-yl)methyl)-2-methylpyrimidine-5-carboxamide

¹H NMR (400 MHz, DMSO) δ 9.23 (t, 1H), 9.09 (s, 2H), 7.03-7.11 (m, 3H), 4.44 (d, 2H), 2.71-2.86 (m, 5H), 2.68 (s, 3H), 2.27-2.42 (m, 4H), 1.95-2.05 (m, 2H), 1.72-1.85 (m, 2H), 1.48-1.66 (m, 2H)

MS ES⁺ 351

### 2.212 Example 212

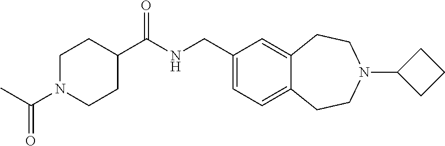

### 1-acetyl-N-((3-cyclobutyl-2,3,4,5-tetrahydro-1H-benzo[d]azepin-7-yl)methyl)piperidine-4-carboxamide

¹H NMR (400 MHz, DMSO) δ 8.10-8.18 (m, 1H), 7.00-7.06 (m, 1H), 6.91-7.00 (m, 2H), 4.28-4.40 (m, 1H), 4.20 (d, 2H), 3.76-3.87 (m, 1H), 2.99-3.09 (m 1H), 2.73-2.86 (m, 5H), 2.53-2.64 (m, 1H), 2.29-2.48 (m, 5H), 1.95-2.07 (m, 5H), 1.67-1.87 (m, 4H), 1.48-1.67 (m, 3H), 1.34-1.49 (m, 1H)

MS ES⁺ 384

### 2.213 Example 213

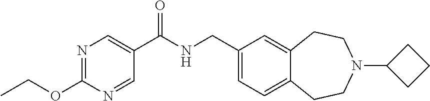

### N-((3-cyclobutyl-2,3,4,5-tetrahydro-1H-benzo[d]azepin-7-yl)methyl)-2-ethoxypyrimidine-5-carboxamide

¹H NMR (400 MHz, DMSO) δ 9.09 (t, 1H), 9.01 (s, 2H), 7.02-7.11 (m, 3H), 4.38-4.47 (m, 4H), 2.66-2.86 (m, 5H), 2.24-2.40 (m, 4H), 1.95-2.04 (m, 2H), 1.71-1.84 (m, 2H), 1.48-1.65 (m, 2H), 1.35 (t, 3H)

MS ES⁺ 381

### 2.214 Example 214

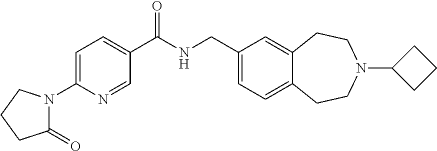

### N-((3-cyclobutyl-2,3,4,5-tetrahydro-1H-benzo[d]azepin-7-yl)methyl)-6-(2-oxopyrrolidin-1-yl)nicotinamide

¹H NMR (400 MHz, DMSO) δ 9.04 (t, 1H), 8.87 (d, 1H), 8.37 (d, 1H), 8.26 (dd, 1H), 7.01-7.10 (m, 3H), 4.43 (d, 2H), 4.03 (t, 2H), 2.66-2.85 (m, 5H), 2.61 (t, 2H), 2.25-2.41 (m, 4H), 1.93-2.12 (m, 4H), 1.70-1.83 (m, 2H), 1.48-1.66 (m, 2H)

MS ES⁺ 419

### 2.215 Example 215

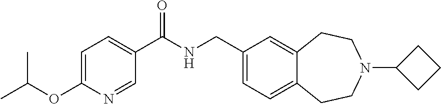

### N-((3-cyclobutyl-2,3,4,5-tetrahydro-1H-benzo[d]azepin-7-yl)methyl)-6-isopropoxynicotinamide

¹H NMR (400 MHz, DMSO) δ 8.94 (t, 1H), 8.68 (d, 1H), 8.13 (dd, 1H), 7.00-7.09 (m, 3H), 6.80 (d, 1H), 5.31 (septet, 1H), 4.41 (d, 2H), 2.72-2.87 (m, 5H), 2.28-2.44 (m, 4H), 1.95-2.05 (m, 2H), 1.73-1.86 (m, 2H), 1.48-1.66 (m, 2H), 1.31 (d, 6H)

MS ES⁺ 394

### 2.216 Example 216

### N-((3-cyclobutyl-2,3,4,5-tetrahydro-1H-benzo[d]azepin-7-yl)methyl)-3-hydroxy-3-methylbutanamide

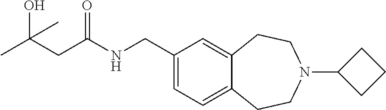

¹H NMR: (400 MHz, MeOD) δ 6.95-7.12 (m, 3H) 4.34 (s, 2H) 3.55 (s, 2H) 2.88-2.94 (m, 4H) 2.78-2.88 (m, 1H) 2.46 (br. s., 4H) 2.04-2.17 (m, 2H) 1.87-2.01 (m, 2H) 1.58-1.79 (m, 2H) 1.14-1.19 (m, 6H)

MS ES⁺: 331

### 2.217 Example 217

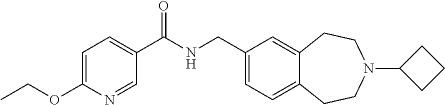

### N-((3-cyclobutyl-2,3,4,5-tetrahydro-1H-benzo[d]azepin-7-yl)methyl)-6-ethoxynicotinamide

¹H NMR (400 MHz, DMSO) δ 8.95 (t, 1H), 8.68 (d, 1H), 8.14 (dd, 1H), 7.01-7.09 (m, 3H), 6.85 (d, 1H), 4.41 (d, 2H), 4.36 (quart, 2H), 2.71-2.86 (m, 5H), 2.26-2.45 (m, 4H), 1.94-2.05 (m, 2H), 1.70-1.86 (m, 2H), 1.47-1.67 (m, 2H), 1.33 (t, 3H)

MS ES⁺ 380

### 2.218 Example 218

### N-((3-cyclobutyl-2,3,4,5-tetrahydro-1H-benzo[d]azepin-7-yl)methyl)-6-(methylamino)nicotinamide

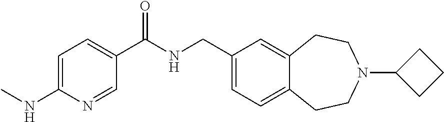

¹H NMR: (400 MHz, DMSO-d₆) δ 8.43-8.58 (m, 2H), 7.77 (m, 1H), 6.85-7.03 (m, 4H), 6.36 (m, 1H), 4.22-4.36 (m, 2H), 2.73 (m, 8H), 2.13-2.34 (m, 4H), 1.85-2.01 (m, 2H), 1.61-1.79 (m, 2H), 1.49 (m, 2H)

MS ES⁺ : 365

### 2.219 Example 219

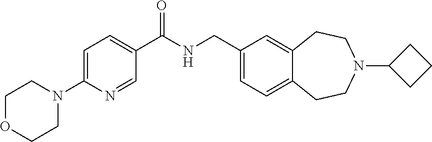

### N-((3-cyclobutyl-2,3,4,5-tetrahydro-1H-benzo[d]azepin-7-yl)methyl)-6-morpholinonicotinamide

¹H NMR (400 MHz, DMSO) δ 8.72-8.85 (m, 1H), 8.67 (s, 1H), 7.92-8.11 (m, 1H), 6.95-7.14 (m, 3H), 6.79-6.92 (m, 1H), 4.17-4.77 (m, 2H), 3.64-3.81 (m, 4H), 3.46-3.62 (m, 4H), 2.67-2.90 (m, 5H), 2.19-2.44 (m, 4H), 1.90-2.09 (m, 2H), 1.68-1.89 (m, 2H), 1.46-1.68 (m, 2H)

MS ES⁺ 421

### 2.220 Example 220

### N-((3-cyclobutyl-2,3,4,5-tetrahydro-1H-benzo[d]azepin-7-yl)methyl)-6-(pyrrolidin-1-yl)nicotinamide

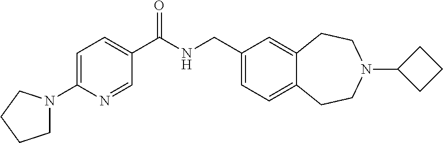

¹H NMR: (400 MHz, DMSO-d₆) δ 8.51-8.75 (m, 2H), 7.95 (m, 1H), 7.03 (br. s., 3H), 6.45 (m, 1H), 4.28-4.44 (m, 2H), 3.38-3.53 (m, 4H), 2.65-2.89 (m, 5H), 2.19-2.42 (m, 4H), 1.95 (br. s., 6H), 1.66-1.85 (m, 2H), 1.57 (m, 2H)

MS ES⁺ : 404

### 2.221 Example 221

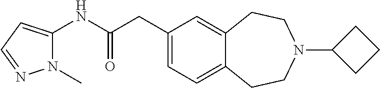

### 2-(3-cyclobutyl-2,3,4,5-tetrahydro-1H-benzo[d]azepin-7-yl)-N-(1-methyl-1H-pyrazol-5-yl)acetamide

¹H NMR (400 MHz, MeOD) δ ppm 1.60-1.81 (m, 2H) 1.87-2.05 (m, 2H) 2.06-2.20 (m, 2H) 2.36-2.63 (m, 4H) 2.81-3.02 (m, 5H) 3.62-3.71 (m, 5H) 6.21 (d, 1H) 7.12 (d, 3H) 7.34-7.44 (m, 1H)

MS ES⁺ 339

### 2.222 Example 222

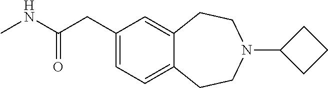

### 2-(3-cyclobutyl-2,3,4,5-tetrahydro-1H-benzo[d]azepin-7-yl)-N-methylacetamide

¹H NMR (400 MHz, MeOD) δ ppm 1.59-1.81 (m, 2H) 1.86-2.03 (m, 2H) 2.05-2.20 (m, 2H) 2.35-2.60 (m, 4H) 2.73 (s, 3H) 2.80-2.99 (m, 5H) 3.44 (s, 2H) 7.04 (s, 3H)

MS ES⁺ 273

### 2.223 Example 223

### 2-(3-cyclobutyl-2,3,4,5-tetrahydro-1H-benzo[d]azepin-7-yl)-N-(tetrahydro-2H-pyran-4-yl)acetamide

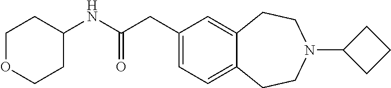

¹H NMR: (400 MHz, MeOD) δ 7.05 (s, 3H), 3.79-4.00 (m, 3H), 3.43 (s, 4H), 2.77-2.98 (m, 5H), 2.34-2.59 (m, 4H), 2.03-2.19 (m, 2H), 1.86-2.04 (m, 2H), 1.59-1.88 (m, 4H), 1.40-1.60 (m, 2H)

MS ES⁺ : 365

### 2.224 Example 224

### 2-(3-cyclobutyl-2,3,4,5-tetrahydro-1H-benzo[d]azepin-7-yl)-N-(pyridin-3-yl)acetamide

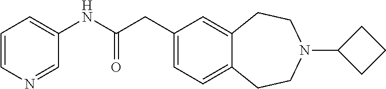

¹H NMR: (400 MHz, MeOD) δ 8.74 (m, 1H), 8.22-8.30 (m, 1H), 8.08-8.17 (m, 1H), 7.35-7.44 (m, 1H), 7.03-7.19 (m, 3H), 3.67 (s, 2H), 2.77-3.00 (m, 5H), 2.30-2.66 (m, 4H), 2.04-2.19 (m, 2H), 1.85-2.04 (m, 2H), 1.71 (m, 2H)

MS ES⁺ : 336

### 2.225 Example 225

### 2-(3-cyclobutyl-2,3,4,5-tetrahydro-1H-benzo[d]azepin-7-yl)-N-isobutylacetamide

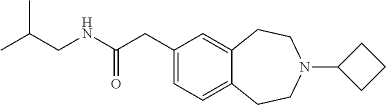

¹H NMR: (400 MHz, MeOD) δ 7.06 (s, 3H), 3.42-3.48 (m, 2H), 2.97-3.04 (m, 2H), 2.79-2.96 (m, 5H), 2.35-2.62 (m, 4H), 2.06-2.19 (m, 2H), 1.89-2.06 (m, 2H), 1.60-1.86 (m, 3H), 0.89 (m, 6H)

MS ES⁺ : 337

### 2.226 Example 226

### 2-(3-cyclobutyl-2,3,4,5-tetrahydro-1H-benzo[d]azepin-7-yl)-1-morpholinoethanone

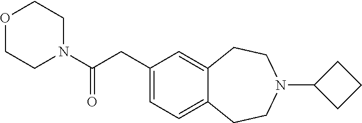

¹H NMR (400 MHz, MeOD) δ 6.95-7.15 (m, 3H), 3.74 (s, 2H), 3.57-3.68 (m, 4H), 3.45-3.56 (m, 4H), 2.77-2.98 (m, 5H), 2.48 (br. s., 4H), 2.06-2.23 (m, 2H), 1.87-2.05 (m, 2H), 1.60-1.85 (m, 2H)

MS ES⁺ : 329

### 2.227 Example 227

### Cyclobutyl(4-(2-(3-cyclobutyl-2,3,4,5-tetrahydro-1H-benzo[d]azepin-7-yl)-1-hydroxyethyl)-4-methylpiperidin-1-yl)methanone

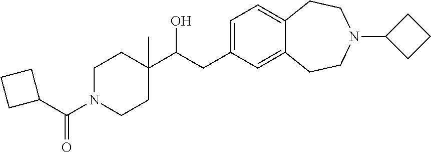

¹H NMR (400 MHz, DICHLOROMETHANE-d₂) δ 6.93 (br. s., 3H), 4.13-4.32 (m, 1H), 3.46-3.59 (m, 1H), 3.36-3.46 (m, 1H), 3.06-3.33 (m, 2H), 2.66-3.02 (m, 7H), 2.21-2.51 (m, 7H), 1.83 (br. s., 8H), 1.42-1.76 (m, 6H), 1.30-1.43 (m, 1H), 1.05 (s, 3H)

MS ES⁺ : 425

### 2.228 Example 228

### Racemic

### 1-(4-(2-(3-cyclobutyl-2,3,4,5-tetrahydro-1H-benzo[d]azepin-7-yl)-1-hydroxyethyl)-4-methylpiperidin-1-yl)propan-1-one

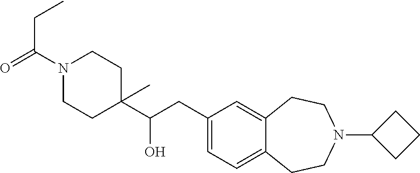

¹H NMR (400 MHz, CHLOROFORM-d) δ ppm 1.09 (s, 3H) 1.12-1.21 (m, 3H) 1.35-1.46 (m, 1H) 1.46-1.52 (m, 1H) 1.52-1.79 (m, 7H) 1.83-2.01 (m, 1H) 2.02-2.18 (m, 2H) 2.29-2.56 (m, 6H) 2.72-3.04 (m, 6H) 3.18-3.33 (m, 1H) 3.39-3.48 (m, 1H) 3.63-3.76 (m, 1H) 4.29-4.42 (m, 1H) 6.95 (br. s., 2H) 7.03-7.09 (m, 1H)

MS ES⁺ : 399

### 2.229 Example 229

### 1-(1-(cyclobutanecarbonyl)-4-methylpiperidin-4-yl)-2-(3-cyclobutyl-2,3,4,5-tetrahydro-1H-benzo[d]azepin-7-yl)ethanone

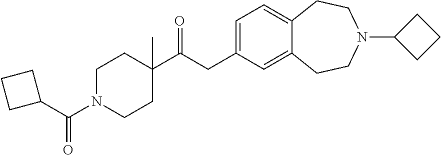

¹H NMR (400 MHz, DICHLOROMETHANE-d₂) δ 7.00-7.05 (m, 1H), 6.85-6.91 (m, 2H), 3.77-3.86 (m, 1H), 3.66-3.76 (m, 2H), 3.31-3.41 (m, 1H), 3.22 (quin, J=8.59 Hz, 1H), 3.06-3.17 (m, 2H), 2.71-2.92 (m, 5H), 2.39 (br. s., 4H), 2.18-2.33 (m, 2H), 2.00-2.16 (m, 6H), 1.74-1.99 (m, 3H), 1.53-1.73 (m, 2H), 1.42-1.53 (m, 1H), 1.31-1.42 (m, 1H), 1.22 (s, 3H)

MS ES⁺ : 423

### 2.230 Example 230

### 1-(cyclobutanecarbonyl)-N-((3-cyclobutyl-2,3,4,5-tetrahydro-1H-benzo[d]azepin-7-yl)methyl)-1,2,3,6-tetrahydropyridine-4-carboxamide

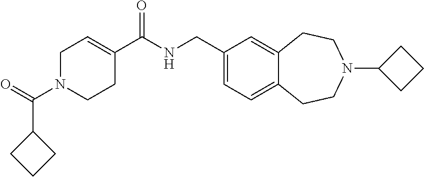

¹H NMR (400 MHz, DMSO-d6) δ ppm 8.24-8.44 (m, 1H), 6.90-7.06 (m, 3H), 6.46-6.58 (m, 1H), 4.20-4.30 (m, 2H), 3.93-4.08 (m, 2H), 3.48-3.58 (m, 1H), 3.33-3.44 (m, 2H), 2.64-2.86 (m, 5H), 2.20-2.40 (m, 6H), 2.04-2.20 (m, 4H), 1.83-2.04 (m, 3H), 1.67-1.83 (m, 3H), 1.49-1.66 (m, 2H)

MS ES⁺ : 422

### 2.231 Example 231

### 4-((3-cyclobutyl-2,3,4,5-tetrahydro-1H-benzo[d]azepin-7-yl)methyl)-N-ethyl-4-hydroxycyclohexanecarboxamide

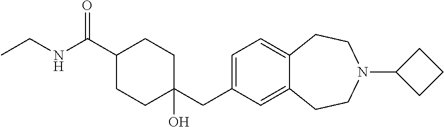

¹H NMR (400 MHz, DMSO-d₆) δ ppm 0.72-0.85 (m, 3H) 0.98-1.08 (m, 2H) 1.11-1.32 (m, 4H) 1.33-1.74 (m, 7H) 1.76-1.89 (m, 2H) 2.05-2.23 (m, 4H) 2.35-2.41 (m, 2H) 2.48-2.68 (m, 5H) 2.75-2.92 (m, 2H) 3.73-3.82 (m, 1H) 6.59-6.88 (m, 3H) 7.29-7.45 (m, 1H)

MS ES⁺ : 385

### 2.232 Example 232

### Enantiomer of Example 142

### 1-(4-(3-(3-cyclobutyl-2,3,4,5-tetrahydro-1H-benzo[d]azepin-7-yl)-2-hydroxypropyl)piperidin-1-yl)propan-1-one

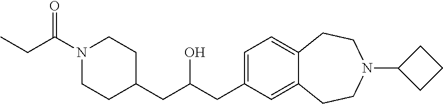

¹H NMR (400 MHz, MeOD) δ 6.92-7.09 (m, 3H), 4.38-4.54 (m, 1H), 3.81-3.96 (m, 2H), 3.01-3.17 (m, 1H), 2.81-2.98 (m, 5H), 2.38-2.76 (m, 7H), 2.05-2.18 (m, 5H), 1.90-2.04 (m, 2H), 1.61-1.89 (m, 5H), 1.28-1.48 (m, 2H), 0.83-1.27 (m, 2H)

MS ES⁺ : 385

### 2.233 Example 233

### Enantiomer of Example 142

### 1-(4-(3-(3-cyclobutyl-2,3,4,5-tetrahydro-1H-benzo[d]azepin-7-yl)-2-hydroxypropyl)piperidin-1-yl)propan-1-one

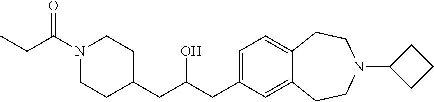

¹H NMR (400 MHz, MeOD) δ 6.92-7.09 (m, 3H), 4.38-4.54 (m, 1H), 3.81-3.96 (m, 2H), 3.01-3.17 (m, 1H), 2.81-2.98 (m, 5H), 2.38-2.76 (m, 7H), 2.05-2.18 (m, 5H), 1.90-2.04 (m, 2H), 1.61-1.89 (m, 5H), 1.28-1.48 (m, 2H), 0.83-1.27 (m, 2H)

MS ES⁺ : 385

### 2.234 Example 234

### N-((3-cyclobutyl-2,3,4,5-tetrahydro-1H-benzo[d]azepin-7-yl)methyl)-6-(ethylamino)nicotinamide

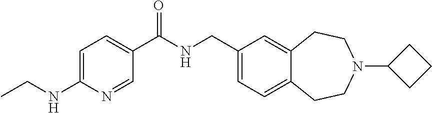

¹H NMR (400 MHz, DMSO-d6) δ ppm 8.56-8.66 (m, 1H), 8.49-8.56 (m, 1H), 7.75-7.87 (m, 1H), 6.92-7.08 (m, 4H), 6.37-6.46 (m, 1H), 4.29-4.42 (m, 2H), 3.23-3.29 (m, 2H), 2.64-2.88 (m, 5H), 2.32 (br. s., 4H), 1.91-2.06 (m, 2H), 1.67-1.84 (m, 2H), 1.45-1.66 (m, 2H), 1.03-1.19 (m, 3H)

MS ES⁺ : 379

### 2.235 Example 235

### N-(4-((3-cyclobutyl-2,3,4,5-tetrahydro-1H-benzo[d]azepin-7-yl)methyl)-4-hydroxycyclohexyl)propionamide

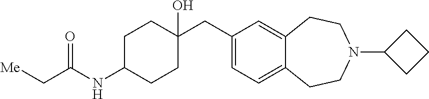

¹H NMR (400 MHz, DMSO-d₆) δ ppm 0.77-0.85 (m, 3H) 1.08-1.20 (m, 2H) 1.23-1.51 (m, 8H) 1.57-1.70 (m, 2H) 1.80-1.92 (m, 4H) 2.12-2.26 (m, 4H) 2.38-2.44 (m, 2H) 2.53-2.70 (m, 5H) 3.18-3.29 (m, 1H) 3.85 (s, 1H) 6.72-6.78 (m, 2H) 6.79-6.87 (m, 1H) 7.36-7.47 (m, 1H)

MS ES⁺ : 385

### 2.236 Example 236

### 6-acetyl-N-((3-cyclobutyl-2,3,4,5-tetrahydro-1H-benzo[d]azepin-7-yl)methyl)nicotinamide

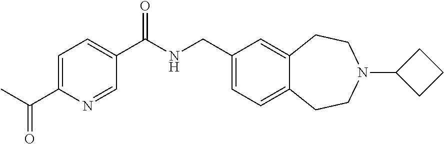

¹H NMR (400 MHz, DMSO-d₆) δ ppm 1.47-1.66 (m, 2H) 1.69-1.88 (m, 2H) 1.93-2.09 (m, 2H) 2.23-2.41 (m, 4H) 2.62-2.70 (m, 3H) 2.71-2.92 (m, 5H) 4.34-4.56 (m, 2H) 6.98-7.17 (m, 3H) 7.98-8.10 (m, 1H) 8.32-8.45 (m, 1H) 9.08-9.20 (m, 1H) 9.26-9.40 (m, 1H)

MS ES⁺: 378

### 2.237 Example 237

### N-((3-cyclobutyl-2,3,4,5-tetrahydro-1H-benzo[d]azepin-7-yl)methyl)-6-(1-hydroxyethyl)nicotinamide

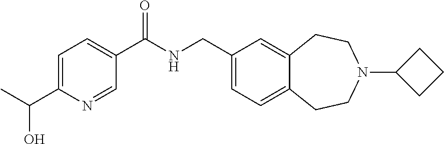

¹H NMR (400 MHz, DMSO-d₆) δ ppm 1.34-1.41 (m, 3H) 1.51-1.65 (m, 2H) 1.71-1.84 (m, 2H) 1.94-2.06 (m, 2H) 2.26-2.39 (m, 4H) 2.71-2.84 (m, 5H) 4.40-4.47 (m, 2H) 4.73-4.83 (m, 1H) 5.44-5.49 (m, 1H) 7.02-7.10 (m, 3H) 7.56-7.64 (m, 1H) 8.19-8.27 (m, 1H) 8.92-8.97 (m, 1H) 9.03-9.16 (m, 1H)

MS ES⁺: 380

### 2.238 Example 238

### N-((3-cyclobutyl-2,3,4,5-tetrahydro-1H-benzo[d]azepin-7-yl)methyl)-6-(1-hydroxycyclobutyl)nicotinamide

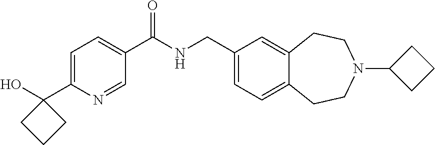

¹H NMR (400 MHz, DMSO-d₆) δ ppm 1.51-1.68 (m, 2H) 1.72-2.06 (m, 6H) 2.18-2.44 (m, 6H) 2.52-2.60 (m, 2H) 2.75-2.90 (m, 5H) 4.39-4.48 (m, 2H) 5.84 (s, 1H) 7.02-7.10 (m, 3H) 7.62-7.69 (m, 1H) 8.15-8.23 (m, 1H) 8.98-9.04 (m, 1H) 9.06-9.15 (m, 1H)

MS ES⁺ : 406

### 2.239 Example 239

### N-((3-cyclobutyl-2,3,4,5-tetrahydro-1H-benzo[d]azepin-7-yl)methyl)-6-(2-hydroxypropan-2-yl)nicotinamide

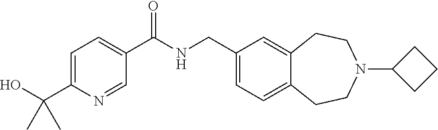

¹H NMR (400 MHz, DMSO-d₆) δ ppm 1.45 (s, 6H) 1.51-1.66 (m, 2H) 1.68-1.84 (m, 2H) 1.93-2.06 (m, 2H) 2.23-2.42 (m, 4H) 2.68-2.87 (m, 5H) 4.36-4.50 (m, 2H) 5.31 (s, 1H) 7.00-7.11 (m, 3H) 7.71-7.79 (m, 1H) 8.15-8.25 (m, 1H) 8.90-8.99 (m, 1H) 9.02-9.15 (m, 1H)

MS ES⁺ : 394

### 2.240 Example 240

### 1-(4-(2-(3-cyclobutyl-2,3,4,5-tetrahydro-1H-benzo[d]azepin-7-yl)acetyl)-4-fluoropiperidin-1-yl)propan-1-one

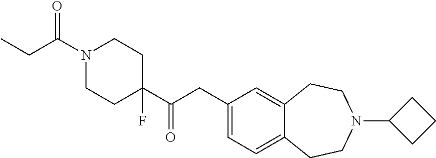

¹H NMR (400 MHz, DICHLOROMETHANE-d₂) δ ppm 0.96-1.07 (m, 3H) 1.43-2.03 (m, 11H) 2.20-2.42 (m, 5H) 2.72-2.90 (m, 6H) 3.17-3.34 (m, 1H) 3.62-3.75 (m, 1H) 3.79-3.89 (m, 2H) 4.35-4.48 (m, 1H) 6.78-6.89 (m, 2H) 6.92-7.02 (m, 1H)

MS ES⁺: 401

### 2.241 Example 241

### 1-(4-(2-(3-cyclobutyl-2,3,4,5-tetrahydro-1H-benzo[d]azepin-7-yl)-1-hydroxyethyl)-4-fluoropiperidin-1-yl)propan-1-one

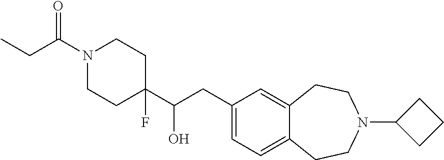

¹H NMR (400 MHz, DICHLOROMETHANE-d₂) δ ppm 1.09-1.19 (m, 3H) 1.53-2.17 (m, 12H) 2.30-2.51 (m, 5H) 2.51-2.62 (m, 1H) 2.72-3.00 (m, 6H) 3.30-3.44 (m, 1H) 3.66-3.90 (m, 2H) 4.52-4.67 (m, 1H) 6.95-7.15 (m, 3H) 111 was not determined

MS ES⁺ : 403

### 2.242 Example 242

### N-((3-(3-fluorocyclobutyl)-2,3,4,5-tetrahydro-1H-benzo[d]azepin-7-yl)methyl)-6-(pyrrolidin-1-yl)nicotinamide

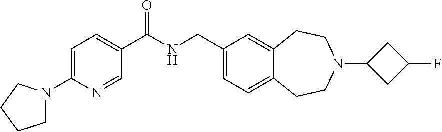

¹H NMR (400 MHz, CD3OD) δ 8.50-8.62 (m, 1H) 7.89-8.02 (m, 1H) 7.00-7.14 (m, 3H) 6.43-6.56 (m, 1H) 4.97-5.23 (m, 1H) 4.49 (s, 2H) 3.41-3.57 (m, 4H) 3.11-3.25 (m, 1H) 2.84-2.99 (m, 4H) 2.48 (br. s., 4H) 2.19-2.41 (m, 4H) 1.97-2.12 (m, 4H).

MS ES⁺ : 423.

### 2.243 Example 243

### (S)—N-((3-cyclobutyl-2,3,4,5-tetrahydro-1H-benzo[d]azepin-7-yl)methyl)-6-(3-fluoropyrrolidin-1-yl)nicotinamide

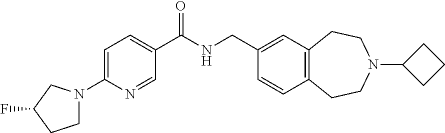

¹H NMR (400 MHz, DMSO-d₆) δ 8.53-8.83 (m, 2H), 7.84-8.07 (m, 1H), 6.88-7.20 (m, 3H), 6.27-6.65 (m, 1H), 5.27-5.58 (m, 1H), 4.24-4.50 (m, 2H), 3.40-3.98 (m, 4H), 2.66-2.90 (m, 5H), 1.91 (none, 12H)

MS ES⁺ : 423

### 2.244 Example 244

### 1-(3-cyclobutyl-2,3,4,5-tetrahydro-1H-benzo[d]azepin-7-yl)-3-(6-methoxypyridin-3-yl)propan-2-one

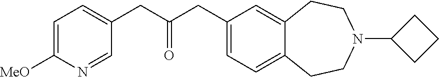

¹H NMR (400 MHz, DICHLOROMETHANE-d₂) δ ppm 1.52-1.82 (m, 2H) 1.77-2.00 (m, 2H) 2.01-2.21 (m, 2H) 2.31-2.61 (m, 4H) 2.74-3.02 (m, 5H) 3.69 (s, 2H) 3.73 (s, 2H) 3.92 (s, 3H) 6.67-6.76 (m, 1H) 6.89-7.01 (m, 2H) 7.04-7.14 (m, 1H) 7.31-7.43 (m, 1H) 7.87-7.96 (m, 1H)

MS ES⁺: 365

### 2.245 Example 245

### N-((3-cyclobutyl-2,3,4,5-tetrahydro-1H-benzo[d]azepin-7-yl)methyl)-6-(3-hydroxypyrrolidin-1-yl)nicotinamide

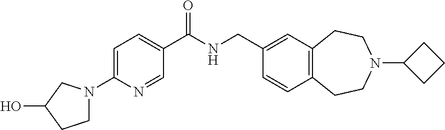

¹H NMR (400 MHz, DMSO-d₆) δ 8.48-8.78 (m, 2H), 7.84-8.12 (m, 1H), 6.83-7.15 (m, 3H), 6.22-6.58 (m, 1H), 4.81-5.08 (m, 1H), 4.17-4.54 (m, 3H), 3.33-3.65 (m, 4H), 2.64-2.90 (m, 5H), 2.16-2.41 (m, 4H), 1.76 (none, 9H)

MS ES⁺: 421

### 2.246 Example 246

### (R)—N-((3-cyclobutyl-2,3,4,5-tetrahydro-1H-benzo[d]azepin-7-yl)methyl)-6-(3-fluoropyrrolidin-1-yl)nicotinamide

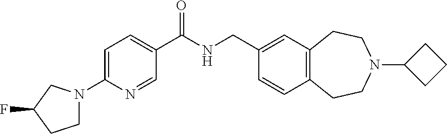

¹H NMR (400 MHz, DMSO-d₆) δ 8.53-8.83 (m, 2H), 7.84-8.07 (m, 1H), 6.88-7.20 (m, 3H), 6.27-6.65 (m, 1H), 5.27-5.58 (m, 1H), 4.24-4.50 (m, 2H), 3.40-3.98 (m, 4H), 2.66-2.90 (m, 5H), 1.91 (none, 12H)

MS ES⁺ : 423

### 2.247 Example 247

### N-((3-cyclobutyl-2,3,4,5-tetrahydro-1H-benzo[d]azepin-7-yl)methyl)-2-(pyrrolidin-1-yl)pyrimidine-5-carboxamide

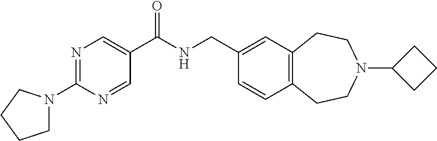

¹H NMR (400 MHz, DMSO-d₆) δ 8.79 (s, 3H), 6.82-7.23 (m, 3H), 4.24-4.59 (m, 2H), 3.41-3.65 (m, 4H), 2.63-3.04 (m, 4H), 2.6-2.4 (6H, overlapped by solvent peak) 2.13-2.42 (m, 2H), 1.77 (none, 7H)

MS ES⁺ : 407

### 2.248 Example 248

### 1-(3-cyclobutyl-2,3,4,5-tetrahydro-1H-benzo[d]azepin-7-yl)-3-(6-methoxypyridin-3-yl)propan-2-ol

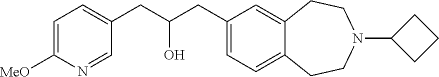

¹H NMR (400 MHz, DICHLOROMETHANE-d₂) δ ppm 1.59-1.78 (m, 5H) 2.00-2.21 (m, 2H) 2.29-2.57 (m, 4H) 2.60-2.95 (m, 9H) 3.92 (s, 3H) 3.95-4.06 (m, 1H) 6.68-6.76 (m, 1H) 6.95-7.11 (m, 3H) 7.47-7.57 (m, 1H) 7.97-8.09 (m, 1H)

MS ES⁺ : 367

### 2.249 Example 249

### N-((3-cyclobutyl-2,3,4,5-tetrahydro-1H-benzo[d]azepin-7-yl)methyl)-6-(pyrrolidin-1-yl)pyridazine-3-carboxamide

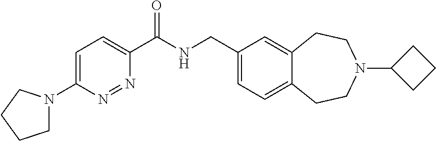

¹H NMR (400 MHz, DMSO-d₆) δ ppm 1.47-1.66 (m, 2H) 1.69-1.85 (m, 2H) 1.91-2.08 (m, 6H) 2.22-2.42 (m, 4H) 2.66-2.85 (m, 5H) 3.42-3.62 (m, 4H) 4.36-4.49 (m, 2H) 6.91-6.98 (m, 1H) 7.00-7.11 (m, 3H) 7.77-7.88 (m, 1H) 9.11-9.26 (m, 1H)

MS ES⁺ : 406

### 2.250 Example 250

### Diastereomer of Example 325

### 1-((3R)-3-(3-(3-cyclobutyl-2,3,4,5-tetrahydro-1H-benzo[d]azepin-7-yl)-2-hydroxypropyl)piperidin-1-yl)propan-1-one

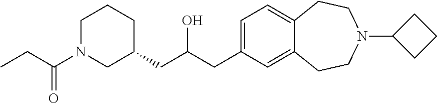

¹H NMR (400 MHz, CD3OD) δ 6.89-7.06 (m, 3H) 4.20-4.32 (m, 1H) 3.69-3.97 (m, 2H) 3.01-3.15 (m, 1H) 2.77-2.97 (m, 5H) 2.56-2.75 (m, 3H) 2.21-2.56 (m, 6H) 2.03-2.19 (m, 2H) 1.88-2.03 (m, 2H) 1.76-1.88 (m, 1H) 1.56-1.76 (m, 4H) 1.18-1.57 (m, 4H) 1.02-1.16 (m, 3H).

MS ES⁺ : 399

### 2.251 Example 251

### Diastereomer of Example 325

### 1-((3R)-3-(3-(3-cyclobutyl-2,3,4,5-tetrahydro-1H-benzo[d]azepin-7-yl)-2-hydroxypropyl)piperidin-1-yl)propan-1-one

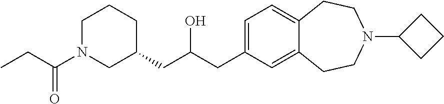

1H NMR (400 MHz, CD3OD) δ 6.87-7.07 (m, 3H) 4.20-4.35 (m, 1H) 3.71-3.91 (m, 2H) 2.98-3.11 (m, 1H) 2.57-2.94 (m, 8H) 2.28-2.54 (m, 6H) 2.01-2.18 (m, 2H) 1.81-2.01 (m, 3H) 1.53-1.78 (m, 4H) 1.25-1.53 (m, 3H) 1.02-1.17 (m, 4H).

MS ES⁺ : 399

### 2.252 Example 252

### N-((3-cyclobutyl-2,3,4,5-tetrahydro-1H-benzo[d]azepin-7-yl)methyl)-6-(2-hydroxypropylamino)nicotinamide

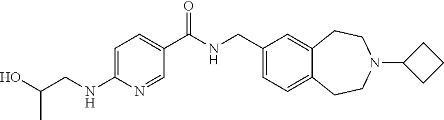

¹H NMR (400 MHz, DMSO-d₆) δ ppm 1.03-1.12 (m, 3H) 1.48-1.67 (m, 2H) 1.69-1.86 (m, 2H) 1.91-2.08 (m, 2H) 2.23-2.42 (m, 4H) 2.67-2.85 (m, 5H) 3.16-3.29 (m, 2H) 3.71-3.86 (m, 1H) 4.32-4.44 (m, 2H) 4.70-4.79 (m, 1H) 6.47-6.58 (m, 1H) 6.97-7.08 (m, 4H) 7.77-7.86 (m, 1H) 8.49-8.55 (m, 1H) 8.56-8.66 (m, 1H)

MS ES⁴: 409

### 2.253 Example 253

### N-((3-cyclobutyl-2,3,4,5-tetrahydro-1H-benzo[d]azepin-7-yl)methyl)-6-(cyclobutylamino)nicotinamide

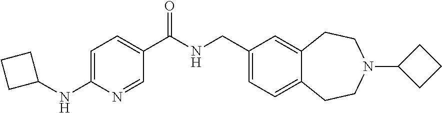

¹H NMR (400 MHz, MeOD-d₄) δ 8.44-8.52 (m, 1H), 7.78-7.89 (m, 1H), 6.99-7.14 (m, 3H), 6.40-6.51 (m, 1H), 4.42-4.53 (m, 2H), 4.24-4.38 (m, 1H), 2.71-2.99 (m, 5H), 2.33-2.55 (m, 6H), 2.04-2.17 (m, 2H), 1.85-2.04 (m, 4H), 1.54-1.85 (m, 4H)

MS ES⁺: ES⁺

### 2.254 Example 254

### N-((3-cyclobutyl-2,3,4,5-tetrahydro-1H-benzo[d]azepin-7-yl)methyl)-6-(cyclopropylamino)nicotinamide

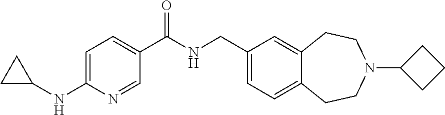

¹H NMR (400 MHz, MeOD-d₄) δ 8.48-8.60 (m, 1H), 7.91-8.01 (m, 1H), 7.03-7.15 (m, 3H), 6.69-6.79 (m, 1H), 4.51 (s, 2H), 2.93 (br. s., 5H), 2.30-2.67 (m, 4H), 2.06-2.18 (m, 2H), 1.86-2.07 (m, 2H), 1.60-1.81 (m, 2H), 0.76-0.91 (m, 2H), 0.47-0.62 (m, 2H)

MS ES⁺: ES⁺

### 2.255 Example 255

### N-((3-cyclobutyl-2,3,4,5-tetrahydro-1H-benzo[d]azepin-7-yl)methyl)morpholine-4-carboxamide

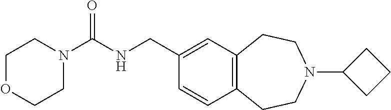

¹H NMR (400 MHz, CHLOROFORM-d) δ 6.85 (s, 1H), 4.33-4.67 (m, 1H), 4.05-4.31 (m, 2H), 3.37-3.62 (m, 4H), 3.03-3.28 (m, 4H), 1.66-3.01 (m, 16H).

MS ES⁺ : 345

### 2.256 Example 256

### N-((3-cyclobutyl-2,3,4,5-tetrahydro-1H-benzo[d]azepin-7-yl)methyl)-2-(pyrrolidin-1-yl)pyrimidine-5-carboxamide

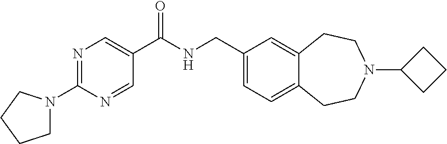

NMR: ¹H NMR (400 MHz, DMSO) δ 8.65 (t, 1H), 8.53 (s, 1H), 7.82 (s, 1H), 6.90-7.01 (m, 3H), 4.30 (d, 2H), 3.37-3.49 (m, 4H), 2.57-2.79 (m, 5H), 2.11-2.33 (m, 4H), 1.82-1.98 (m, 6H), 1.61-1.78 (m, 2H), 1.39-1.57 (m, 2H)

MS ES⁺ : 406

### 2.257 Example 257

### Enantiomer of Example 228

### 1-{4-[2-(3-cyclobutyl-2,3,4,5-tetrahydro-1H-3-benzazepin-7-yl)-1-hydroxyethyl]-4-methylpiperidin-1-yl}propan-1-one

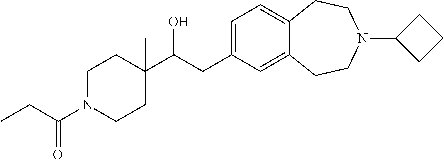

1H NMR (400 MHz, CHLOROFORM-d) d ppm 1.09 (s, 3H) 1.12-1.21 (m, 3H) 1.35-1.46 (m, 1H) 1.46-1.52 (m, 1H) 1.52-1.79 (m, 7H) 1.83-2.01 (m, 1H) 2.02-2.18 (m, 2H) 2.29-2.56 (m, 6H) 2.72-3.04 (m, 6H) 3.18-3.33 (m, 1H) 3.39-3.48 (m, 1H) 3.63-3.76 (m, 1H) 4.29-4.42 (m, 1H) 6.95 (br. s., 2H) 7.03-7.09 (m, 1H)

MS ES⁺: 399

### 2.258 Example 258

### Enantiomer of Example 228

### 1-{4-[2-(3-cyclobutyl-2,3,4,5-tetrahydro-1H-3-benzazepin-7-yl)-1-hydroxyethyl]-4-methylpiperidin-1-yl}propan-1-one

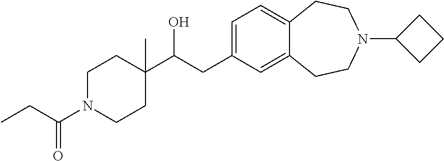

1H NMR (400 MHz, CHLOROFORM-d) d ppm 1.09 (s, 3H) 1.12-1.21 (m, 3H) 1.35-1.46 (m, 1H) 1.46-1.52 (m, 1H) 1.52-1.79 (m, 7H) 1.83-2.01 (m, 1H) 2.02-2.18 (m, 2H) 2.29-2.56 (m, 6H) 2.72-3.04 (m, 6H) 3.18-3.33 (m, 1H) 3.39-3.48 (m, 1H) 3.63-3.76 (m, 1H) 4.29-4.42 (m, 1H) 6.95 (br. s., 2H) 7.03-7.09 (m, 1H)

MS ES⁺: 399

### 2.259 Example 259

### (S)—N-((3-cyclobutyl-2,3,4,5-tetrahydro-1H-benzo[d]azepin-7-yl)methyl)tetrahydrofuran-2-carboxamide hydrochloride

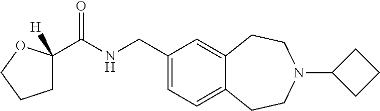

¹H NMR (400 MHz, DMSO-d₆) δ 10.60-10.88 (m, 1H), 8.20-8.38 (m, 1H), 7.07 (s, 3H), 4.23 (m, 3H), 3.84-3.99 (m, 1H), 3.71-3.84 (m, 1H), 3.55-3.72 (m, 1H), 3.43-3.56 (m, 2H), 3.19-3.30 (m, 2H), 2.87-3.02 (m, 2H), 2.64-2.83 (m, 2H), 2.28-2.45 (m, 2H), 2.04-2.26 (m, 3H), 1.73 (m, 5H)

MS ES⁻: 327

### 2.260 Example 260

### (R)—N-((3-cyclobutyl-2,3,4,5-tetrahydro-1H-benzo[d]azepin-7-yl)methyl)tetrahydrofuran-2-carboxamide hydrochloride

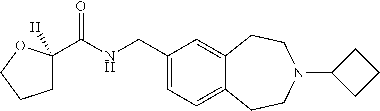

¹H NMR (400 MHz, DMSO-d₆) δ 10.59-10.90 (m, 1H), 8.21-8.38 (m, 1H), 7.07 (s, 3H), 4.23 (s, 3H), 3.83-3.98 (m, 1H), 3.72-3.84 (m, 1H), 3.55-3.72 (m, 1H), 3.43-3.55 (m, 2H), 3.20-3.29 (m, 2H), 2.85-3.03 (m, 2H), 2.62-2.84 (m, 2H), 2.29-2.45 (m, 2H), 2.03-2.27 (m, 3H), 1.51-1.95 (m, 5H)

MS ES⁺: 329

### 2.261 Example 261

### (S)—N-((3-cyclobutyl-2,3,4,5-tetrahydro-1H-benzo[d]azepin-7-yl)methyl)-6-(2-methylpyrrolidin-1-yl)nicotinamide

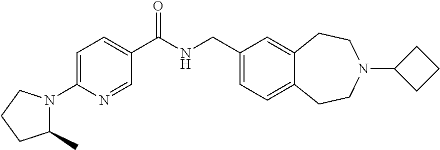

NMR: NMR (400 MHz, DMSO) δ 8.60-8.72 (m, 2H), 7.95 (d, 1H), 6.96-7.09 (m, 3H), 6.45 (d, 1H), 4.39 (d, 2H), 4.13-4.24 (m, 1H), 3.45-3.56 (m, 1H), 3.25-3.35 (m, 1H), 2.61-2.87 (m, 5H), 2.20-2.40 (m, 4H), 1.86-2.11 (m, 5H), 1.39-1.85 (m, 5H), 1.15 (d, 3H)

MS ES⁺: 419

### 2.262 Example 262

### N-((3-(3-hydroxycyclobutyl)-2,3,4,5-tetrahydro-1H-benzo[d]azepin-7-yl)methyl)-6-methoxynicotinamide

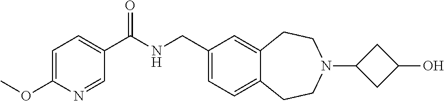

1H NMR (400 MHz, CD3OD) δ 8.60-8.69 (m, 1H) 8.04-8.14 (m, 1H) 7.02-7.19 (m, 3H) 6.78-6.89 (m, 1H) 4.50 (s, 2H) 3.94 (s, 3H) 3.86-3.95 (m, 1H) 2.94 (br. s., 4H) 2.41-2.73 (m, 7H) 1.76-1.92 (m, 2H).

MS ES⁺ : 382.

### 2.263 Example 263

### N-[(3-cyclobutyl-2,3,4,5-tetrahydro-1H-3-benzazepin-7-yl)methyl]-3,5-dimethyl-1,2-oxazole-4-carboxamide

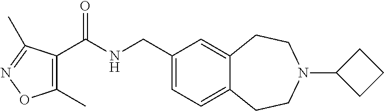

NMR: ¹H NMR (400 MHz, CHLOROFORM-d) δ 7.03-7.18 (m, 3H), 5.69-5.89 (m, 1H), 4.48-4.65 (m, 2H), 2.86-3.02 (m, 4H), 2.71-2.86 (m, 1H), 2.55-2.68 (m, 4H), 2.33-2.55 (m, 6H), 2.02-2.17 (m, 2H), 1.83-2.00 (m, 2H), 1.59-1.79 (m, 2H)

MS ES⁺ : 353

### 2.264 Example 264

### (R)—N-((3-cyclobutyl-2,3,4,5-tetrahydro-1H-benzo[d]azepin-7-yl)methyl)-6-(2-methylpyrrolidin-1-yl)nicotinamide

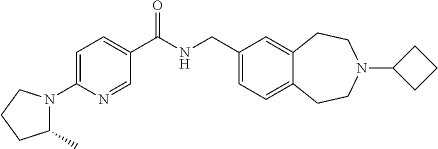

NMR: ¹H NMR (400 MHz, DMSO) δ 8.57-8.69 (m, 2H), 7.94 (dd, 1H), 6.98-7.07 (m, 3H), 6.46 (d, 1H), 4.38 (d, 2H), 4.15-4.24 (m, 1H), 3.47-3.55 (m, 1H), 3.25-3.35 (m, 1H), 2.63-2.85 (m, 5H), 2.24-2.40 (m, 4H), 1.89-2.11 (m, 5H), 1.48-1.84 (m, 5H), 1.16 (d, 3H)

MS ES⁺ : 419

### 2.265 Example 265

### (R)—N-((3-cyclobutyl-2,3,4,5-tetrahydro-1H-benzo[d]azepin-7-yl)methyl)-2-methylpyrrolidine-1-carboxamide

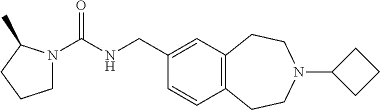

¹H NMR (400 MHz, METHANOL-d₄) δ 6.97-7.22 (m, 3H), 6.34-6.72 (m, 1H), 4.11-4.44 (m, 2H), 3.85-4.04 (m, 1H), 3.47-3.78 (m, 1H), 3.31-3.45 (m, 1H), 3.25 (1H, under solvent peak) 2.84-3.19 (m, 4H), 2.09-2.42 (m, 5H), 1.45-2.09 (m, 8H), 1.14 (none, 4H)

MS ES⁺ : 342

### 2.266 Example 266

### N-((3-cyclobutyl-2,3,4,5-tetrahydro-1H-benzo[d]azepin-7-yl)methyl)-2-methyltetrahydrofuran-2-carboxamide hydrochloride

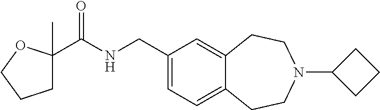

¹H NMR (400 MHz, DMSO-d₆) δ 10.69-10.97 (m, 1H), 8.06-8.26 (m, 1H), 7.07 (s, 3H), 4.23 (m, 2H), 3.80-3.97 (m, 2H), 3.58-3.79 (m, 1H), 3.42-3.58 (m, 2H), 3.27-3.42 (m, 2H), 2.87-3.03 (m, 2H), 2.63-2.85 (m, 2H), 2.30-2.46 (m, 2H), 2.08-2.30 (m, 3H), 1.83-1.97 (m, 1H), 1.56-1.83 (m, 4H), 1.33 (s, 3H)

MS ES⁺: ES⁺

### 2.267 Example 267

### N-((3-cyclobutyl-2,3,4,5-tetrahydro-1H-benzo[d]azepin-7-yl)methyl)-2,2-dimethyltetrahydro-2H-pyran-4-carboxamide

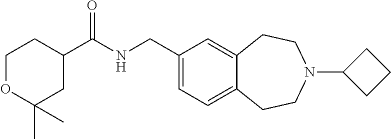

¹H NMR (400 MHz, MeOD-d₄) δ 6.80-6.94 (m, 3H), 4.13 (s, 2H), 3.49-3.62 (m, 2H), 2.60-2.80 (m, 5H), 2.43-2.59 (m, 1H), 2.31 (br. s., 4H), 1.94 (m, 2H), 1.78 (br. s., 2H), 1.48 (m, 6H), 1.07 (m, 6H)

MS ES⁺ : 371

### 2.268 Example 268

### N-((3-cyclobutyl-2,3,4,5-tetrahydro-1H-benzo[d]azepin-7-yl)methyl)-2,4-dimethylthiazole-5-carboxamide

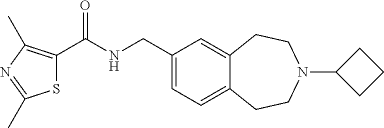

¹H NMR (400 MHz, MeOD-d₄) δ 6.87-6.99 (m, 3H), 4.30 (s, 2H), 2.62-2.83 (m, 5H), 2.51 (s, 3H), 2.41 (s, 3H), 2.21-2.39 (m, 4H), 1.90-2.01 (m, 2H), 1.71-1.85 (m, 2H), 1.54 (m, 2H)

MS ES⁺ : 370

### 2.269 Example 269

### 6-(pyrrolidin-1-yl)-N-((3-(tetrahydrofuran-3-yl)-2,3,4,5-tetrahydro-1H-benzo[d]azepin-7-yl)methyl)nicotinamide

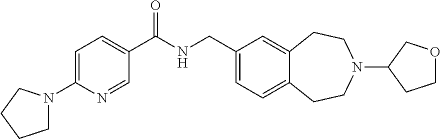

¹H NMR (400 MHz, DMSO-d₆) δ ppm 1.66-1.82 (m, 1H) 1.86-2.03 (m, 5H) 2.53-2.64 (m, 2H) 2.75-2.89 (m, 4H) 3.13-3.25 (m, 1H) 3.38-3.47 (m, 4H) 3.47-3.55 (m, 1H) 3.56-3.66 (m, 1H) 3.69-3.86 (m, 2H) 4.31-4.45 (m, 2H) 6.41-6.50 (m, 1H) 6.97-7.09 (m, 3H) 7.90-8.02 (m, 1H) 8.58-8.72 (m, 2H) 2H were overlapping with residual DMSO

MS ES⁺: 421

### 2.270 Example 270

### N-((3-cyclobutyl-2,3,4,5-tetrahydro-1H-benzo[d]azepin-7-yl)methyl)-6-((2-hydroxypropyl)(methyl)amino)nicotinamide

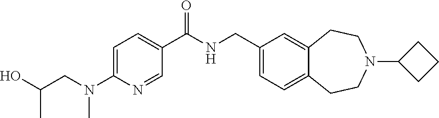

¹H NMR (400 MHz, DMSO-d₆) δ ppm 1.05-1.10 (m, 3H) 1.50-1.68 (m, 2H) 1.71-1.85 (m, 2H) 1.94-2.09 (m, 2H) 2.24-2.43 (m, 4H) 2.68-2.86 (m, 5H) 3.11 (s, 3H) 3.37-3.48 (m, 1H) 3.51-3.62 (m, 1H) 3.85-4.00 (m, 1H) 4.34-4.45 (m, 2H) 4.69-4.76 (m, 1H) 6.60-6.70 (m, 1H) 6.97-7.11 (m, 3H) 7.91-8.00 (m, 1H) 8.59-8.63 (m, 1H) 8.64-8.73 (m, 1H)

MS ES⁺: 423

### 2.271 Example 271

### N-((3-cyclobutyl-2,3,4,5-tetrahydro-1H-benzo[d]azepin-7-yl)methyl)-5-methylisoxazole-3-carboxamide

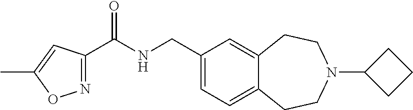

¹H NMR (400 MHz, DMSO-d₆) δ ppm 1.49-1.66 (m, 2H) 1.69-1.84 (m, 2H) 1.93-2.07 (m, 2H) 2.21-2.41 (m, 4H) 2.43-2.48 (m, 3H) 2.69-2.85 (m, 5H) 4.27-4.43 (m, 2H) 6.50-6.58 (m, 1H) 6.96-7.10 (m, 3H) 9.08-9.22 (m, 1H)

MS ES⁺ : 340

### 2.272 Example 272

### N-((3-cyclobutyl-2,3,4,5-tetrahydro-1H-benzo[d]azepin-7-yl)methyl)isoxazole-5-carboxamide

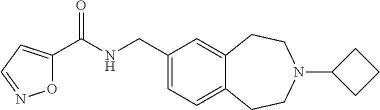

¹H NMR (400 MHz, DMSO-d₆) δ ppm 1.40-1.61 (m, 2H) 1.62-1.79 (m, 2H) 1.85-2.01 (m, 2H) 2.26 (br. s., 4H) 2.62-2.80 (m, 5H) 4.24-4.39 (m, 2H) 6.91-7.08 (m, 4H) 8.61-8.74 (m, 1H) 9.24-9.46 (m, 1H)

MS ES⁺ : 326

### 2.273 Example 273

### N-[(3-cyclobutyl-2,3,4,5-tetrahydro-1H-3-benzazepin-7-yl)methyl]-6-(dimethylamino)pyridine-3-carboxamide

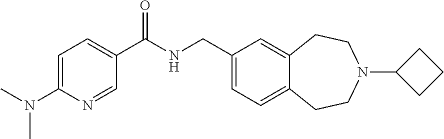

NMR: ¹H NMR (400 MHz, CHLOROFORM-d) δ 8.40-8.54 (m, 1H), 7.72-7.86 (m, 1H), 6.92-7.02 (m, 3H), 6.34-6.44 (m, 1H), 5.95-6.11 (m, 1H), 4.47 (d, 2H), 2.97-3.11 (m, 6H), 2.74-2.87 (m, 4H), 2.61-2.73 (m, 1H), 2.23-2.45 (m, 4H), 1.89-2.03 (m, 2H), 1.73-1.86 (m, 2H), 1.50-1.65 (m, 2H)

MS ES⁺ : 379, ES⁻: 377

### 2.274 Example 274

### 2-(3-cyclobutyl-2,3,4,5-tetrahydro-1H-benzo[d]azepin-7-yl)-N-(6-methoxypyridin-3-yl)acetamide

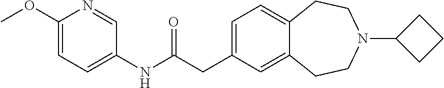

¹H NMR (400 MHz, DMSO-d₆) δ 10.14 (s, 1H), 8.31-8.39 (m, 1H), 7.84-7.95 (m, 1H), 7.05 (br. s., 3H), 6.74-6.82 (m, 1H), 3.81 (s, 3H), 3.55 (s, 2H), 2.82 (br. s., 5H), 2.34 (br. s., 4H), 2.00 (d, J=5.81 Hz, 2H), 1.79 (br. s., 2H), 1.48-1.69 (m, 2H)

MS ES⁺ : 366

### 2.275 Example 275

### N-((3-cyclobutyl-2,3,4,5-tetrahydro-1H-benzo[d]azepin-7-yl)methyl)-2-methoxy-4-methylthiazole-5-carboxamide

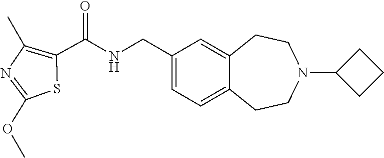

¹H NMR (400 MHz, MeOD) δ 6.94 (s, 3H), 4.30 (s, 2H), 3.94 (s, 3H), 2.79 (br. s., 5H), 2.35 (s, 7H), 1.91-2.06 (m, 2H), 1.71-1.91 (m, 2H), 1.44-1.66 (m, 2H)

MS ES⁺ : 386

### 2.276 Example 276

### N-((3-cyclobutyl-2,3,4,5-tetrahydro-1H-benzo[d]azepin-7-yl)methyl)-3-methylisoxazole-4-carboxamide

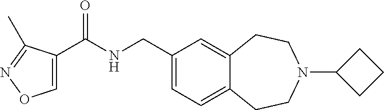

¹H NMR (400 MHz, MeOD) δ 9.14 (s, 1H), 7.23 (s, 3H), 4.59 (s, 2H), 2.90-3.14 (m, 5H), 2.60 (s, 7H), 2.25 (m, 2H), 2.09 (br. s., 2H), 1.70-1.96 (m, 2H)

MS ES⁺ : 340

### 2.277 Example 277

### N-((3-cyclobutyl-2,3,4,5-tetrahydro-1H-benzo[d]azepin-7-yl)methyl)-3,3-difluoropyrrolidine-1-carboxamide

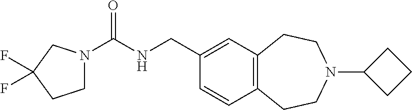

¹H NMR (400 MHz, METHANOL-d₄) δ 7.07 (s, 3H), 4.21 (s, 2H), 3.42-3.67 (m, 7H), 3.06-3.17 (m, 2H), 2.90-3.04 (m, 2H), 2.61-2.76 (m, 2H), 2.12-2.39 (m, 6H), 1.60-1.91 (m, 2H)

MS ES⁺ : 364

### 2.278 Example 278

### (S)—N-((3-cyclobutyl-2,3,4,5-tetrahydro-1H-benzo[d]azepin-7-yl)methyl)-2-methylpyrrolidine-1-carboxamide

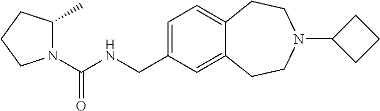

¹H NMR (400 MHz, METHANOL-d₄) δ 6.97-7.22 (m, 3H), 6.34-6.72 (m, 1H), 4.11-4.44 (m, 2H), 3.85-4.04 (m, 1H), 3.47-3.78 (m, 1H), 3.31-3.45 (m, 1H), 3.25 (1H, under solvent peak) 2.84-3.19 (m, 4H), 2.09-2.42 (m, 5H), 1.45-2.09 (m, 8H), 1.14 (none, 4H)

MS ES⁺ : 342

### 2.279 Example 279

### N-((3-cyclobutyl-2,3,4,5-tetrahydro-1H-benzo[d]azepin-7-yl)methyl)-3-methylisoxazole-5-carboxamide

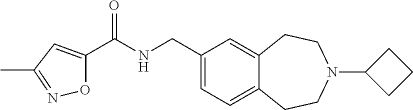

¹H NMR (400 MHz, DMSO-d₆) δ ppm 1.43-1.61 (m, 2H) 1.64-1.79 (m, 2H) 1.89-2.01 (m, 2H) 2.19-2.34 (m, 7H) 2.62-2.81 (m, 5H) 4.27-4.36 (m, 2H) 6.86-6.91 (m, 1H) 6.93-7.05 (m, 3H) 9.24-9.37 (m, 1H)

MS ES⁺ : 340

### 2.280 Example 280

### N-[(3-cyclobutyl-2,3,4,5-tetrahydro-1H-3-benzazepin-7-yl)methyl]-6-[(cyclopropylmethyl)amino]pyridine-3-carboxamide

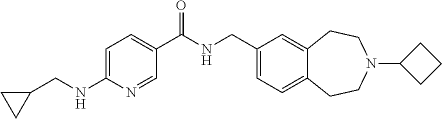

NMR: ¹H NMR (400 MHz, CHLOROFORM-d) δ 8.25-8.31 (m, 1H), 7.61-7.69 (m, 1H), 6.82-6.91 (m, 3H), 6.10-6.19 (m, 1H), 5.83-5.94 (m, 1H), 4.70-4.80 (m, 1H), 4.30-4.38 (m, 2H), 2.91-2.99 (m, 2H), 2.61-2.75 (m, 4H), 2.48-2.62 (m, 1H), 2.11-2.30 (m, 4H), 1.79-1.90 (m, 2H), 1.59-1.77 (m, 2H), 1.37-1.52 (m, 2H), 0.81-0.93 (m, 1H), 0.29-0.38 (m, 2H), −0.01-0.10 (m, 2H)

MS ES⁺ : 405, ES⁻: 403

### 2.281 Example 281

### N-[(3-cyclobutyl-2,3,4,5-tetrahydro-1H-3-benzazepin-7-yl)methyl]-6-[(3S)-tetrahydrofuran-3-yloxy]pyridine-3-carboxamide

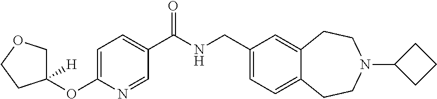

NMR: ¹H NMR (400 MHz, CHLOROFORM-d) δ 8.55-8.61 (m, 1H), 7.95-8.06 (m, 1H), 7.08-7.15 (m, 3H), 6.74-6.83 (m, 1H), 6.16-6.26 (m, 1H), 5.58-5.66 (m, 1H), 4.55-4.64 (m, 2H), 3.97-4.10 (m, 2H), 3.86-3.97 (m, 2H), 2.88-3.01 (m, 4H), 2.73-2.85 (m, 1H), 2.37-2.55 (m, 4H), 2.21-2.36 (m, 1H), 2.04-2.20 (m, 3H), 1.85-2.00 (m, 2H), 1.62-1.79 (m, 2H)

MS ES⁺ : 422, ES⁻: 420

### 2.282 Example 282

### N-[(3-cyclobutyl-2,3,4,5-tetrahydro-1H-3-benzazepin-7-yl)methyl]-6-(tetrahydrofuran-3-ylamino)pyridine-3-carboxamide

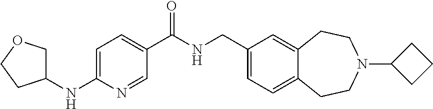

NMR: ¹H NMR (400 MHz, CHLOROFORM-d) δ 8.39-8.49 (m, 1H), 7.74-7.86 (m, 1H), 6.94-7.09 (m, 3H), 6.24-6.36 (m, 1H), 5.96-6.12 (m, 1H), 4.76-4.88 (m, 1H), 4.44-4.56 (m, 2H), 4.33-4.44 (m, 1H), 3.84-3.94 (m, 2H), 3.70-3.82 (m, 1H), 3.58-3.68 (m, 1H), 2.76-2.87 (m, 4H), 2.59-2.76 (m, 1H), 2.18-2.46 (m, 5H), 1.90-2.06 (m, 2H), 1.70-1.91 (m, 3H), 1.52-1.68 (m, 2H)

MS ES⁺ : 421, ES⁺: 419

### 2.283 Example 283

### N-((3-cyclobutyl-2,3,4,5-tetrahydro-1H-benzo[d]azepin-7-yl)methyl)piperidine-1-carboxamide

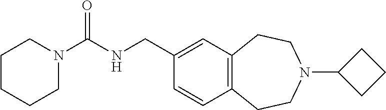

¹H NMR (400 MHz, METHANOL-d₄) δ 7.11-7.24 (m, 3H), 4.32 (s, 2H), 3.59-3.81 (m, 3H), 3.37-3.46 (m, 4H), 3.18-3.30 (m, 2H), 3.02-3.13 (m, 2H), 2.73-2.86 (m, 2H), 2.27-2.47 (m, 4H), 1.75-2.00 (m, 2H), 1.62-1.72 (m, 2H), 1.51-1.60 (m, 4H)

MS ES⁺ : 342

### 2.284 Example 284

### N-((3-cyclobutyl-2,3,4,5-tetrahydro-1H-benzo[d]azepin-7-yl)methyl)-2,5-dimethyloxazole-4-carboxamide

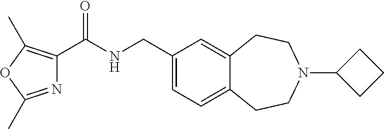

¹H NMR (400 MHz, MeOD) δ 7.09 (m 3H), 4.47 (s, 2H), 2.93 (br. s., 5H), 2.34-2.64 (m, 10H), 2.05-2.20 (m, 2H), 1.86-2.04 (m, 2H), 1.58-1.84 (m, 2H)

MS ES⁺ : 354

### 2.285 Example 285

### N-[(3-cyclobutyl-2,3,4,5-tetrahydro-1H-3-benzazepin-7-yl)methyl]-6-(tetrahydro-2H-pyran-4-ylamino)pyridine-3-carboxamide

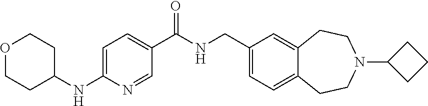

NMR: ¹H NMR (400 MHz, METHANOL-d₄) δ 8.36-8.44 (m, 1H), 7.70-7.78 (m, 1H), 6.94-7.04 (m, 3H), 6.38-6.47 (m, 1H), 4.38 (s, 2H), 3.81-3.95 (m, 3H), 3.40-3.50 (m, 2H), 2.75-2.92 (m, 5H), 2.31-2.59 (m, 4H), 1.97-2.09 (m, 2H), 1.78-1.93 (m, 4H), 1.52-1.71 (m, 2H), 1.37-1.52 (m, 2H)

MS ES⁺ : 435

### 2.286 Example 286

### N-[(3-cyclobutyl-2,3,4,5-tetrahydro-1H-3-benzazepin-7-yl)methyl]-4,4-difluoropiperidine-1-carboxamide

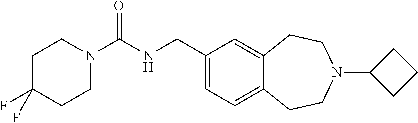

NMR: ¹H NMR (400 MHz, METHANOL-d₄) δ 7.17-7.30 (m, 3H), 4.41-4.50 (m, 2H), 3.65-3.76 (m, 4H), 3.05-3.33 (m, 5H), 2.67-2.97 (m, 4H), 2.28-2.41 (m, 2H), 2.03-2.26 (m, 6H), 1.81-1.99 (m, 2H)

MS ES⁺ : 378

### 2.287 Example 287

### N-((3-(3-hydroxycyclobutyl)-2,3,4,5-tetrahydro-1H-benzo[d]azepin-7-yl)methyl)-6-(pyrrolidin-1-yl)nicotinamide

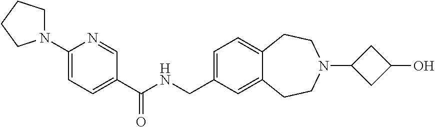

¹H NMR (400 MHz, CD3OD) δ 8.55-8.59 (m, 1H) 7.91-7.98 (m, 1H) 7.03-7.12 (m, 3H) 6.48-6.53 (m, 1H) 4.49 (s, 2H) 3.85-3.96 (m, 1H) 3.44-3.55 (m, 4H) 2.87-2.97 (m, 4H) 2.30-2.60 (m, 7H) 2.01-2.08 (m, 4H) 1.75-1.87 (m, 2H).

MS ES⁺: 421.

### 2.288 Example 288

### N-((3-cyclobutyl-2,3,4,5-tetrahydro-1H-benzo[d]azepin-7-yl)methyl)-3-methoxycyclobutanecarboxamide

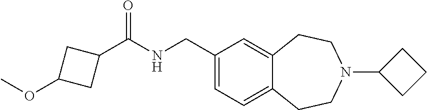

¹H NMR (400 MHz, MeOD) δ 6.96-7.15 (m, 3H), 4.31 (m, 2H), 4.05-4.19 (m, 0.3H), 3.73-3.89 (m, 0.7H), 3.25 (s, 3H), 2.97-3.10 (m, 0.3H), 2.92 (m, 5H), 2.56-2.70 (m, 0.7H), 2.33-2.56 (m, 6H), 2.04-2.25 (m, 4H), 1.84-2.05 (m, 2H), 1.55-1.82 (m, 2H)

MS ES⁺: 343 (Two peaks cis and trans isomers)

### 2.289 Example 289

### N-((3-cyclobutyl-2,3,4,5-tetrahydro-1H-benzo[d]azepin-7-yl)methyl)-6-(tetrahydro-2H-pyran-4-yloxy)nicotinamide

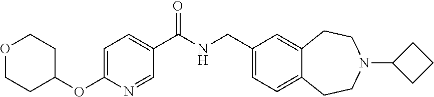

¹H NMR (400 MHz, DMSO-d₆) δ 8.95 (s, 1H), 8.62-8.72 (m, 1H), 8.08-8.21 (m, 1H), 7.05 (br. s., 3H), 6.80-6.93 (m, 1H), 5.18-5.32 (m, 1H), 4.41 (m, 2H), 3.79-3.88 (m, 2H), 3.45-3.61 (m, 2H), 2.81 (br. s., 5H), 2.34 (m, 4H), 1.92-2.09 (m, 4H), 1.71-1.91 (m, 2H), 1.46-1.71 (m, 4H)

MS ES⁺: 436

### 2.290 Example 290

### (S)—N-((3-cyclobutyl-2,3,4,5-tetrahydro-1H-benzo[d]azepin-7-yl)methyl)-6-(2-hydroxypropylamino)nicotinamide

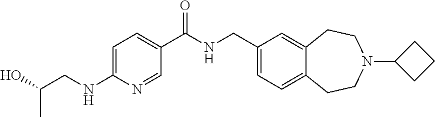

¹H NMR (400 MHz, DMSO-d₆) δ ppm 0.81-0.92 (m, 3H) 1.25-1.47 (m, 2H) 1.47-1.67 (m, 2H) 1.70-1.89 (m, 2H) 1.99-2.23 (m, 4H) 2.45-2.75 (m, 5H) 2.95-3.09 (m, 2H) 3.50-3.65 (m, 1H) 4.11-4.22 (m, 2H) 4.49-4.59 (m, 1H) 6.27-6.36 (m, 1H) 6.71-6.97 (m, 4H) 7.54-7.68 (m, 1H) 8.28-8.34 (m, 1H) 8.35-8.49 (m, 1H)

MS ES⁺: 409

### 2.291 Example 291

### N-[(3-cyclobutyl-2,3,4,5-tetrahydro-1H-3-benzazepin-7-yl)methyl]-6-[(3R)-tetrahydrofuran-3-yloxy]pyridine-3-carboxamide

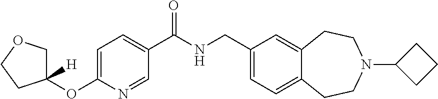

NMR: NMR: ¹H NMR (400 MHz, METHANOL-d₄) δ 8.80-8.87 (m, 1H), 8.27-8.33 (m, 1H), 7.24-7.34 (m, 3H), 6.99-7.08 (m, 1H), 5.77-5.84 (m, 1H), 4.66-4.75 (m, 2H), 4.03-4.25 (m, 4H), 2.97-3.16 (m, 5H), 2.57-2.74 (m, 4H), 2.43-2.57 (m, 1H), 2.24-2.37 (m, 3H), 2.06-2.22 (m, 2H), 1.81-1.97 (m, 2H)

MS ES⁺ : 422, ES⁻: 420

### 2.292 Example 292

### N-[(3-cyclobutyl-2,3,4,5-tetrahydro-1H-3-benzazepin-7-yl)methyl]-6-[(3S)-tetrahydrofuran-3-ylamino]pyridine-3-carboxamide

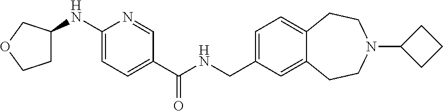

1H NMR (400 MHz, METHANOL-d4) d 8.51-8.60 (m, 1H), 7.84-7.92 (m, 1H), 7.05-7.18 (m, 3H), 6.53-6.60 (m, 1H), 4.45-4.56 (m, 3H), 3.94-4.03 (m, 2H), 3.79-3.91 (m, 1H), 3.64-3.72 (m, 1H), 2.86-3.04 (m, 5H), 2.44-2.72 (m, 4H), 2.25-2.37 (m, 1H), 2.10-2.21 (m, 2H), 1.88-2.07 (m, 3H), 1.64-1.81 (m, 2H)

MS ES⁺ : 421

### 2.293 Example 293

### 3-chloro-N-[(3-cyclobutyl-2,3,4,5-tetrahydro-1H-3-benzazepin-7-yl)methyl]cyclobutanecarboxamide

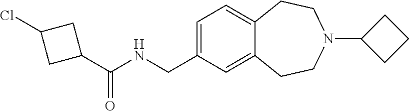

1H NMR (400 MHz, MeOD) d 6.97-7.11 (m, 3H), 4.57-4.69 (m, 0.3H), 4.34-4.46 (m, 0.7H), 4.31 (m, 2H), 3.23-3.30 (m, 0.3H), 2.62-3.00 (m, 7.7H), 2.31-2.65 (m, 6H), 2.05-2.19 (m, 2H), 1.86-2.04 (m, 2H), 1.57-1.83 (m, 2H)

MS ES⁺ : 347

### 2.294 Example 294

### 2-(3-cyclobutyl-2,3,4,5-tetrahydro-1H-3-benzazepin-7-yl)-N-[6-(pyrrolidin-1-yl)pyridin-3-yl]acetamide

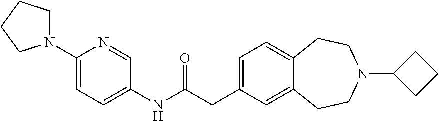

1H NMR (400 MHz, MeOD) d 8.11-8.20 (m, 1H), 7.65-7.76 (m, 1H), 7.05-7.18 (m, 3H), 6.42-6.53 (m, 1H), 3.61 (s, 2H), 3.42 (m, 4H), 2.96 (m, 5H), 2.57 (br. s., 4H), 2.15 (m, 2H), 1.90-2.10 (m, 6H), 1.75 (br. s., 2H)

MS ES⁺ : 405

### 2.295 Example 295

### N-[(3-cyclobutyl-2,3,4,5-tetrahydro-1H-3-benzazepin-7-yl)methyl]-6-{[(2S)-2-hydroxypropyl](methyl)amino}pyridine-3-carboxamide

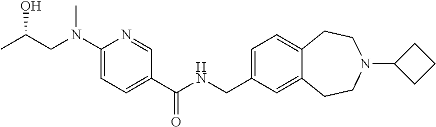

1H NMR (400 MHz, DMSO-d6) d ppm 1.03-1.17 (m, 3H) 1.52-1.70 (m, 2H) 1.73-1.90 (m, 2H) 1.96-2.13 (m, 2H) 2.21-2.48 (m, 4H) 2.71-2.89 (m, 5H) 3.15 (s, 3H) 3.41-3.50 (m, 1H) 3.54-3.66 (m, 1H) 3.89-4.05 (m, 1H) 4.36-4.50 (m, 2H) 4.71-4.81 (m, 1H) 6.64-6.74 (m, 1H) 6.98-7.15 (m, 3H) 7.93-8.06 (m, 1H) 8.62-8.67 (m, 1H) 8.67-8.76 (m, 1H)

MS ES⁺ : 423

### 2.296 Example 296

### 6-methoxy-N-{[3-(tetrahydrofuran-3-yl)-2,3,4,5-tetrahydro-1H-3-benzazepin-7-yl]methyl}pyridine-3-carboxamide

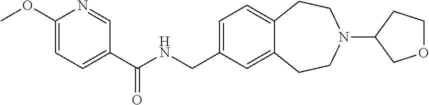

1H NMR (400 MHz, CHLOROFORM-d) d 8.56-8.63 (m, 1H), 7.97-8.05 (m, 1H), 7.06-7.15 (m, 3H), 6.74-6.82 (m, 1H), 6.14-6.28 (m, 1H), 4.55-4.65 (m, 2H), 3.91-4.04 (m, 4H), 3.83-3.91 (m, 1H), 3.67-3.81 (m, 2H), 3.23-3.33 (m, 1H), 2.86-2.99 (m, 4H), 2.66-2.78 (m, 2H), 2.50-2.65 (m, 2H), 1.99-2.11 (m, 1H), 1.82-1.96 (m, 1H)

MS ES⁺ : 382

### 2.297 Example 297

### N-{[3-(3-methoxycyclobutyl)-2,3,4,5-tetrahydro-1H-3-benzazepin-7-yl]methyl}-6-(pyrrolidin-1-yl)pyridine-3-carboxamide

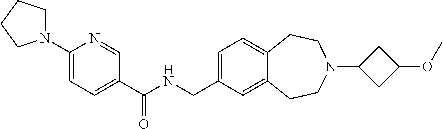

1H NMR (400 MHz, CD3OD) δ 1H NMR (400 MHz, METHANOL-d4) d ppm 8.52-8.63 (m, 1H) 7.88-8.00 (m, 1H) 6.99-7.13 (m, 3H) 6.48-6.55 (m, 1H) 4.49 (s, 2H) 3.54-3.67 (m, 1H) 3.45-3.54 (m, 4H) 3.24 (s, 3H) 2.85-2.96 (m, 4H) 2.37-2.58 (m, 7H) 1.97-2.10 (m, 4H) 1.70-1.86 (m, 2H)

MS ES⁺ : 435

### 2.298 Example 298

### N-[(3-cyclobutyl-2,3,4,5-tetrahydro-1H-3-benzazepin-7-yl)methyl]-3,3-dimethylcyclobutanecarboxamide

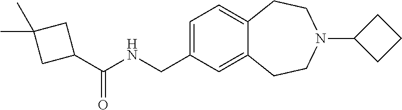

1H NMR (400 MHz, MeOD) d 6.96-7.13 (m, 3H), 4.30 (s, 2H), 2.99-3.14 (m, 1H), 2.92 (m, 5H), 2.23-2.66 (m, 4H), 1.84-2.21 (m, 8H), 1.58-1.81 (m, 2H), 1.20 (s, 3H), 1.10 (s, 3H)

MS ES⁺: 341

### 2.299 Example 299

### N-[(3-cyclobutyl-2,3,4,5-tetrahydro-1H-3-benzazepin-7-yl)methyl]-6-[(3R)-tetrahydrofuran-3-ylamino]pyridine-3-carboxamide

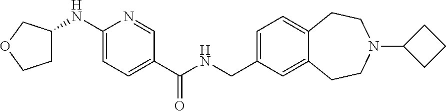

1H NMR (400 MHz, METHANOL-d4) d 8.49-8.59 (m, 1H), 7.81-7.92 (m, 1H), 7.05-7.15 (m, 3H), 6.51-6.61 (m, 1H), 4.44-4.56 (m, 3H), 3.94-4.03 (m, 2H), 3.81-3.91 (m, 1H), 3.65-3.74 (m, 1H), 2.80-2.99 (m, 5H), 2.40-2.60 (m, 4H), 2.26-2.37 (m, 1H), 2.06-2.17 (m, 2H), 1.88-2.03 (m, 3H), 1.63-1.79 (m, 2H)

MS ES⁺: 421

### 2.300 Example 300

### 6-((S)-3-fluoropyrrolidin-1-yl)-N-((3-(tetrahydrofuran-3-yl)-2,3,4,5-tetrahydro-1H-benzo[d]azepin-7-yl)methyl)nicotinamide

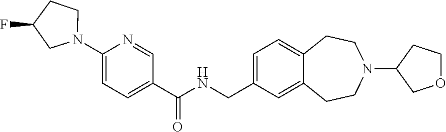

1H NMR (400 MHz, CD3OD-d4) δ 8.49-8.59 (m, 1H), 7.81-7.92 (m, 1H), 7.05-7.15 (m, 3H), 6.51-6.61 (m, 1H), 4.44-4.56 (m, 3H), 3.94-4.03 (m, 2H), 3.81-3.91 (m, 1H), 3.65-3.74 (m, 1H), 2.80-2.99 (m, 5H), 2.40-2.60 (m, 4H), 2.26-2.37 (m, 1H), 2.06-2.17 (m, 2H), 1.88-2.03 (m, 3H), 1.63-1.79 (m, 2H)

MS ES⁺ : 439

### 2.301 Example 301

### N-((3-cyclobutyl-2,3,4,5-tetrahydro-1H-benzo[d]azepin-7-yl)methyl)-2-(ethylamino)pyrimidine-5-carboxamide

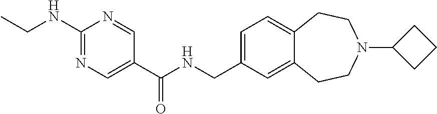

1H NMR (400 MHz, CD3OD) δ 8.34-8.94 (m, 2H), 6.73-7.32 (m, 3H), 4.12-4.45 (m, 2H), 3.23-3.48 (m, 2H), 2.53-2.88 (m, 5H), 1.34-2.49 (m, 10H), 1.12 (m, 3H)

MS ES⁺ : 380

### 2.302 Example 302

### N-((3-cyclobutyl-2,3,4,5-tetrahydro-1H-benzo[d]azepin-7-yl)methyl)-2-(methylamino)pyrimidine-5-carboxamide

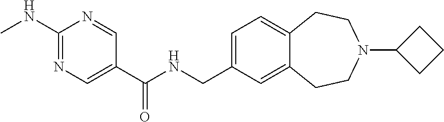

1H NMR (400 MHz, CD3OD) δ 8.28-8.88 (m, 2H), 6.73-7.26 (m, 3H), 4.10-4.45 (m, 2H), 2.52-2.93 (m, 8H), 1.88 (m, 10H)

MS ES⁺ : 365

### 2.303 Example 303

### 3-cyclobutyl-7-((2-(trifluoromethyl)pyrrolidin-1-yl)methyl)-2,3,4,5-tetrahydro-1H-benzo[d]azepine

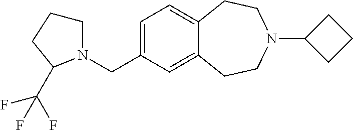

1H NMR (400 MHz, CDCl₃-d) δ 7.07 (s, 3H), 4.01-4.28 (m, 1H), 3.49-3.71 (m, 1H), 3.17-3.41 (m, 1H), 2.69-3.11 (m, 6H), 2.25-2.66 (m, 5H), 1.88 (none, 10H)

MS ES⁺ : 354

### 2.304 Example 304

### 1-((3-cyclobutyl-2,3,4,5-tetrahydro-1H-benzo[d]azepin-7-yl)methyl)pyrrolidin-2-one

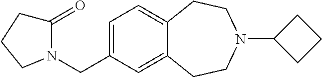

1H NMR (400 MHz, CDCl₃-d) δ 6.94-7.15 (m, 3H), 4.31-4.52 (m, 2H), 3.19-3.40 (m, 2H), 2.84-3.00 (m, 4H), 2.69-2.84 (m, 1H), 2.33-2.58 (m, 5H), 1.81-2.19 (m, 6H), 1.68 (none, 3H)

MS ES⁺ : 299

### 2.305 Example 305

### 6-cyclobutoxy-N-((3-cyclobutyl-2,3,4,5-tetrahydro-1H-benzo[d]azepin-7-yl)methyl)nicotinamide

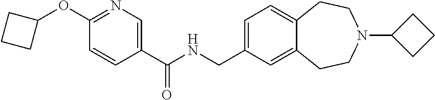

NMR: 1H NMR (400 MHz, DMSO) d 8.94 (t, 1H), 8.66 (d, 1H), 8.14 (dd, 1H), 7.00-7.07 (m, 3H), 6.84 (dd, 1H), 5.18 (quint, 1H), 4.41 (d, 2H), 2.68-2.84 (m, 5H), 2.23-2.46 (m, 6H), 1.94-2.13 (m, 4H), 1.70-1.84 (m, 3H), 1.48-1.70 (m, 3H)

MS ES⁺: 406

### 2.306 Example 306

### N-((3-cyclobutyl-2,3,4,5-tetrahydro-1H-benzo[d]azepin-7-yl)methyl)-6-propoxynicotinamide

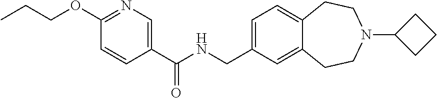

NMR: 1H NMR (400 MHz, DMSO) d 8.84 (s, 1H), 8.69 (s, 1H), 8.15 (dd, 1H), 7.05 (s, 3H), 6.85 (dd, 1H), 4.35-4.51 (m, 2H), 4.16-4.35 (m, 2H), 2.70-2.92 (m, 5H), 2.25-2.45 (m, 4H), 1.91-2.10 (m, 2H), 1.68-1.90 (m, 4H), 1.48-1.68 (m, 2H), 0.78-1.16 (m, 3H)

MS ES⁺ : 394

### 2.307 Example 307

### N-((3-cyclobutyl-2,3,4,5-tetrahydro-1H-benzo[d]azepin-7-yl)methyl)-6-(hydroxymethyl)nicotinamide

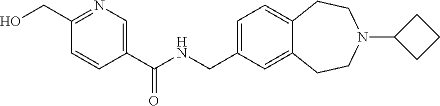

1H NMR (400 MHz, CHLOROFORM-d) d 8.72-9.13 (m, 1H), 7.90-8.28 (m, 1H), 7.31-7.48 (m, 1H), 7.01-7.20 (m, 3H), 6.19-6.51 (m, 1H), 4.73-5.03 (m, 2H), 4.46-4.73 (m, 2H), 3.41-3.65 (m, 1H), 2.69-3.07 (m, 5H), 2.25-2.68 (m, 3H), 1.87 (none, 6H)

MS ES⁴: 366

### 2.308 Example 308

### 2-(pyrrolidin-1-yl)-N-((3-(tetrahydrofuran-3-yl)-2,3,4,5-tetrahydro-1H-benzo[d]azepin-7-yl)methyl)pyrimidine-5-carboxamide

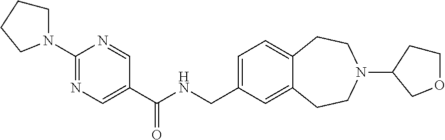

NMR: 1H NMR (400 MHz, CHLOROFORM-d) d 8.64-8.82 (m, 2H), 7.03-7.17 (m, 3H), 6.05-6.19 (m, 1H), 4.51-4.67 (m, 2H), 3.93-4.03 (m, 1H), 3.84-3.92 (m, 1H), 3.69-3.82 (m, 2H), 3.56-3.69 (m, 4H), 3.22-3.35 (m, 1H), 2.85-3.02 (m, 4H), 2.66-2.79 (m, 2H), 2.51-2.65 (m, 2H), 1.97-2.12 (m, 5H), 1.83-1.97 (m, 1H)

MS ES⁺ : 422

### 2.309 Example 309

### 6-methoxy-N-((3-(3-methoxycyclobutyl)-2,3,4,5-tetrahydro-1H-benzo[d]azepin-7-yl)methyl)nicotinamide

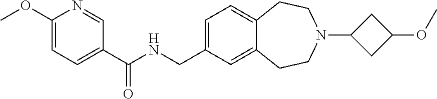

1H NMR (400 MHz, CD3OD) δ 8.61-8.70 (m, 1H) 8.05-8.13 (m, 1H) 7.02-7.12 (m, 3H) 6.79-6.87 (m, 1H) 4.50 (s, 2H) 3.96 (s, 3H) 3.62 (m, 1H) 3.24 (s, 3H) 2.87-2.96 (m, 4H) 2.34-2.62 (m, 7H) 1.72-1.86 (m, 2H).

MS ES⁺ : 396

### 2.310 Example 310

### 6-((R)-3-fluoropyrrolidin-1-yl)-N-((3-(tetrahydrofuran-3-yl)-2,3,4,5-tetrahydro-1H-benzo[d]azepin-7-yl)methyl)nicotinamide

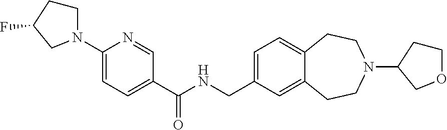

1H NMR (400 MHz, METHANOL-d4) δ 8.57-8.69 (m, 1H), 7.94-8.05 (m, 1H), 7.03-7.16 (m, 3H), 6.54-6.62 (m, 1H), 5.31-5.52 (m, 1H), 4.45-4.56 (m, 2H), 3.92-4.00 (m, 1H), 3.82-3.91 (m, 2H), 3.65-3.80 (m, 5H), 3.52-3.63 (m, 1H), 2.87-3.00 (m, 4H), 2.54-2.79 (m, 4H), 2.06-2.45 (m, 3H), 1.83-1.96 (m, 1H)

MS ES⁺ : 439

### 2.311 Example 311

### N-((3-(2-methyltetrahydrofuran-3-yl)-2,3,4,5-tetrahydro-1H-benzo[d]azepin-7-yl)methyl)-6-(pyrrolidin-1-yl)nicotinamide

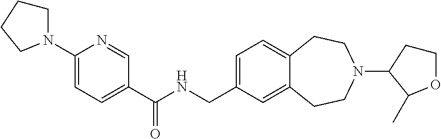

1H NMR (400 MHz, DMSO-d6) δ ppm 1.06-1.19 (m, 3H) 1.79-2.03 (m, 6H) 2.54-2.67 (m, 2H) 2.77-2.89 (m, 4H) 2.93-3.03 (m, 1H) 3.37-3.49 (m, 4H) 3.54-3.69 (m, 1H) 3.79-3.89 (m, 1H) 3.93-4.05 (m, 1H) 4.34-4.44 (m, 2H) 6.40-6.52 (m, 1H) 6.97-7.10 (m, 3H) 7.89-8.01 (m, 1H) 8.57-8.73 (m, 2H), 2H was hidden with the residue of DMSO.

MS ES⁺ : 435

### 2.312 Example 312

### N-[(3-cyclobutyl-2,3,4,5-tetrahydro-1H-3-benzazepin-7-yl)methyl]-2-[(cyclopropylmethyl)amino]pyrimidine-5-carboxamide

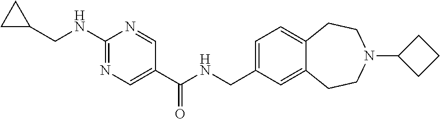

1H NMR (400 MHz, CHLOROFORM-d) δ 8.70 (br. s., 2H), 7.02-7.17 (m, 3H), 6.12 (br. s., 1H), 5.58 (br. s., 1H), 4.57 (d, J=5.31 Hz, 2H), 3.33 (t, J=6.32 Hz, 2H), 1.43-3.11 (m, 15H), 0.98-1.19 (m, 1H), 0.49-0.67 (m, 2H), 0.27 (q, J=4.88 Hz, 2H)

MS ES⁺: 406

### 2.313 Example 313

### N-((3-cyclobutyl-2,3,4,5-tetrahydro-1H-benzo[d]azepin-7-yl)methyl)-N-methyl-2-(pyrrolidin-1-yl)pyrimidine-5-carboxamide

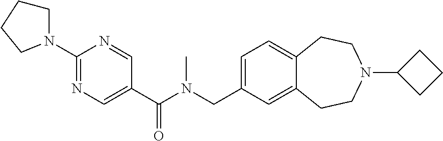

1H NMR (400 MHz, DICHLOROMETHANE-d2) δ 8.38 (s, 2H), 6.83-7.11 (m, 3H), 4.51 (s, 2H), 3.30-3.59 (m, 5H), 2.90 (s, 8H), 2.34 (br. s., 3H), 1.36-2.14 (m, 10H)

MS ES⁺: 421

### 2.314 Example 314

### N-[(3-cyclobutyl-2,3,4,5-tetrahydro-1H-3-benzazepin-7-yl)methyl]-2-ethyl-4-methyl-1,3-oxazole-5-carboxamide

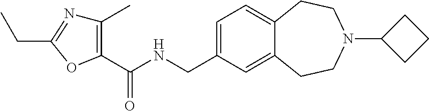

1H NMR (400 MHz, METHANOL-d4) δ 6.98-7.16 (m, 3H), 4.45 (s, 2H), 2.74-3.00 (m, 7H), 2.30-2.59 (m, 7H), 2.03-2.15 (m, 2H), 1.85-2.01 (m, 2H), 1.58-1.84 (m, 2H), 1.25-1.48 (m, 3H)

MS ES⁺: 368

### 2.315 Example 315

### N-[(3-cyclobutyl-2,3,4,5-tetrahydro-1H-3-benzazepin-7-yl)methyl]-6-[(1-methoxy-2-methylpropan-2-yl)oxy]pyridine-3-carboxamide

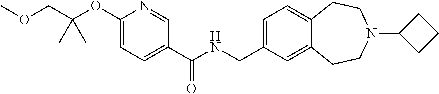

1H NMR (400 MHz, CD3OD) δ 8.55-8.67 (m, 1H) 7.98-8.07 (m, 1H) 7.00-7.13 (m, 3H) 6.69-6.78 (m, 1H) 4.50 (s, 2H) 3.72 (s, 2H) 3.36 (s, 3H) 2.88-2.95 (m, 4H) 2.76-2.87 (m, 1H) 2.46 (br. s., 4H) 2.04-2.15 (m, 2H) 1.86-2.00 (m, 2H) 1.61-1.77 (m, 2H) 1.57 (s, 6H).

MS ES⁺ : 438

### 2.316 Example 316

### (R)—N-((3-cyclobutyl-2,3,4,5-tetrahydro-1H-benzo[d]azepin-7-yl)methyl)-6-((2-hydroxypropyl)(methyl)amino)nicotinamide

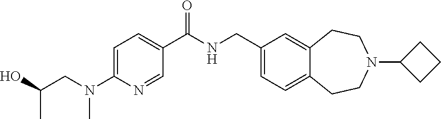

1H NMR (400 MHz, DMSO-d6) δ ppm 0.98-1.12 (m, 3H) 1.47-1.66 (m, 2H) 1.69-1.85 (m, 2H) 1.93-2.08 (m, 2H) 2.21-2.42 (m, 4H) 2.66-2.86 (m, 5H) 3.10 (s, 3H) 3.36-3.46 (m, 1H) 3.49-3.61 (m, 1H) 3.85-3.98 (m, 1H) 4.32-4.45 (m, 2H) 4.67-4.76 (m, 1H) 6.58-6.70 (m, 1H) 6.96-7.09 (m, 3H) 7.90-7.99 (m, 1H) 8.58-8.62 (m, 1H) 8.62-8.72 (m, 1H)

MS ES⁺ : 423

### 2.317 Example 317

### (S)—N-((3-cyclobutyl-2,3,4,5-tetrahydro-1H-benzo[d]azepin-7-yl)methyl)-6-(1-methoxypropan-2-yloxy)nicotinamide

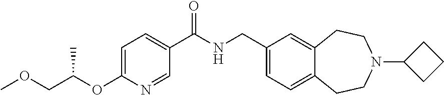

1H NMR (400 MHz, CD3OD) δ 8.61-8.64 (m, 1H) 8.06-8.10 (m, 1H) 7.04-7.12 (m, 3H) 6.77-6.82 (m, 1H) 5.38-5.48 (m, 1H) 4.50 (s, 2H) 3.50-3.64 (m, 2H) 3.37 (s, 3H) 2.87-2.95 (m, 4H) 2.77-2.87 (m, 1H) 2.46 (br. s., 4H) 2.04-2.15 (m, 2H) 1.87-1.99 (m, 2H) 1.60-1.77 (m, 2H) 1.29-1.34 (m, 3H).

MS ES⁺ : 424

### 2.318 Example 318

### (S)—N-((3-cyclobutyl-2,3,4,5-tetrahydro-1H-benzo[d]azepin-7-yl)methyl)-6-(2-methoxypropoxy)nicotinamide

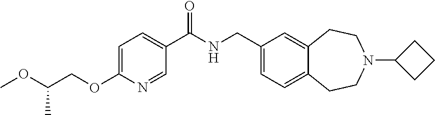

1H NMR (400 MHz, CD3OD) δ 8.61-8.67 (m, 1H) 8.07-8.14 (m, 1H) 7.02-7.13 (m, 3H) 6.83-6.89 (m, 1H) 4.50 (s, 2H) 4.27-4.39 (m, 2H) 3.71-3.80 (m, 1H) 3.41 (s, 3H) 2.88-2.96 (m, 4H) 2.78-2.88 (m, 1H) 2.46 (br. s., 4H) 2.05-2.15 (m, 2H) 1.87-2.00 (m, 2H) 1.60-1.77 (m, 2H) 1.20-1.27 (m, 3H).

MS ES⁺ : 424

### 2.319 Example 319

### cis-N-((3-cyclobutyl-2,3,4,5-tetrahydro-1H-benzo[d]azepin-7-yl)methyl)-3-(tetrahydro-2H-pyran-4-yloxy)cyclobutanecarboxamide

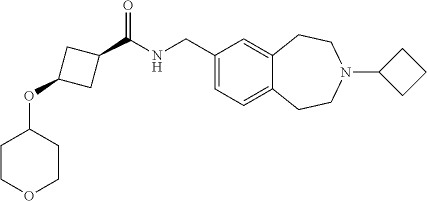

1H NMR (400 MHz, DICHLOROMETHANE-d2) Shift 6.84-7.06 (m, 3H), 5.68 (br. s., 1H), 4.19-4.31 (m, 2H), 3.72-3.94 (m, 3H), 3.39 (tt, J=4.23, 8.91 Hz, 1H), 3.22-3.34 (m, 2H), 2.79 (br. s., 4H), 1.32-2.52 (m, 20H)

MS ES⁺ : 413

### 2.320 Example 320 trans-N-((3-cyclobutyl-2,3,4,5-tetrahydro-1H-benzo[d]azepin-7-yl)methyl)-3-(tetrahydro-2H-pyran-4-yloxy)cyclobutanecarboxamide

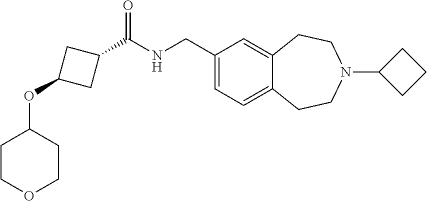

1H NMR (400 MHz, DICHLOROMETHANE-d2) Shift 6.94-7.18 (m, 3H), 5.77 (br. s., 1H), 4.28-4.49 (m, 3H), 3.81-3.99 (m, 2H), 3.44-3.61 (m, 1H), 3.32-3.46 (m, 2H), 2.65-3.11 (m, 5H), 2.28-2.59 (m, 5H), 2.15-2.28 (m, 2H), 2.01-2.15 (m, 2H), 1.31-2.01 (m, 10H)

MS ES⁺ : 413

### 3.321 Example 321

### N-((3-cyclobutyl-2,3,4,5-tetrahydro-1H-benzo[d]azepin-7-yl)methyl)-2-methyloxazole-4-carboxamide

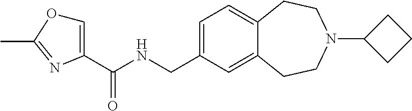

1H NMR (400 MHz, MeOD) d 8.21-8.31 (m, 1H), 7.04-7.13 (m, 3H), 4.49 (s, 2H), 2.76-2.99 (m, 5H), 2.48 (s, 7H), 2.05-2.19 (m, 2H), 1.87-2.03 (m, 2H), 1.58-1.82 (m, 2H)

MS ES⁺ : 340

### 3.322 Example 322

### (R)—N-((3-cyclobutyl-2,3,4,5-tetrahydro-1H-benzo[d]azepin-7-yl)methyl)-2-(3-fluoropyrrolidin-1-yl)pyrimidine-5-carboxamide

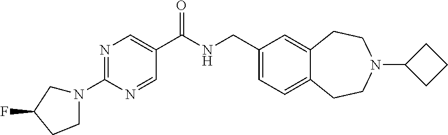

1H NMR (400 MHz, CD3OD/CD2Cl2) δ 8.79 (s, 2H) 7.00-7.14 (m, 3H) 5.26-5.48 (m, 1H) 4.49 (s, 2H) 3.58-4.05 (m, 4H) 2.75-2.98 (m, 5H) 2.17-2.60 (m, 6H) 2.03-2.17 (m, 2H) 1.84-2.01 (m, 2H) 1.57-1.77 (m, 2H).

MS ES⁺ : 424

### 3.323 Example 323

### (S)—N-((3-cyclobutyl-2,3,4,5-tetrahydro-1H-benzo[d]azepin-7-yl)methyl)-2-(3-fluoropyrrolidin-1-yl)pyrimidine-5-carboxamide

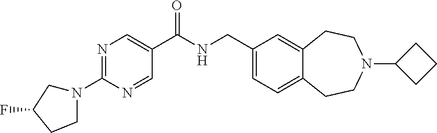

1H NMR (400 MHz, CD3OD/CD2Cl2) δ 8.79 (s, 2H) 7.01-7.13 (m, 3H) 5.29-5.48 (m, 1H) 4.49 (s, 2H) 3.58-4.03 (m, 4H) 2.75-3.01 (m, 5H) 2.16-2.67 (m, 6H) 2.03-2.14 (m, 2H) 1.86-2.03 (m, 2H) 1.58-1.78 (m, 2H).

MS ES⁺ : 424

### 3.324 Example 324

### N-((3-cyclobutyl-2,3,4,5-tetrahydro-1H-benzo[d]azepin-7-yl)methyl)-2-(dimethylamino)pyrimidine-5-carboxamide

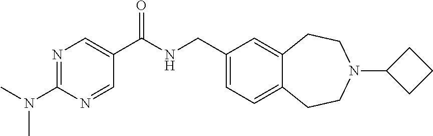

1H NMR (400 MHz, CD3OD) δ 8.76 (s, 2H) 7.02-7.15 (m, 3H) 4.48 (s, 2H) 3.23 (s, 6H) 2.76-2.98 (m, 5H) 2.48 (br. s., 4H) 2.03-2.18 (m, 2H) 1.85-2.01 (m, 2H) 1.56-1.80 (m, 2H).

MS ES⁺ : 380

### 3.325 Example 325

### 1-((3R)-3-(3-(3-cyclobutyl-2,3,4,5-tetrahydro-1H-benzo[d]azepin-7-yl)-2-hydroxypropyl)piperidin-1-yl)propan-1-one

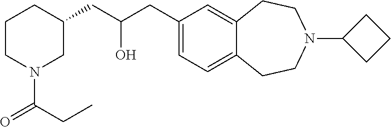

1H NMR (400 MHz, CD3OD) δ 6.85-7.11 (m, 3H) 4.19-4.43 (m, 1H) 3.68-3.98 (m, 2H) 2.58-3.19 (m, 9H) 2.28-2.57 (m, 5H) 1.55-2.18 (m, 9H) 1.01-1.55 (m, 8H)

MS ES⁺ : 399

### 3. BIOLOGICAL EFFICACY OF COMPOUNDS OF THE INVENTION

### 3.1 In Vitro H₃ Binding Assay

The ability of compounds to bind to the H3 receptor was determined by measuring the reduction in tritiated N-α-methyl-histamine (³H—NαMH) binding in a competition binding assay. Changes in the levels of bound radio-label were monitored by scintillation counting with a Trilux Microbeta (Perkin Elmer).

Membranes were prepared from CHO-K1 cells stably expressing human H3 receptor; routinely grown as monolayers in Ham's F12 medium (Invitrogen) supplemented with 10% Foetal Clone 111 (Hyclone), 500 μg/ml G418 (Invitrogen), 5 μg/ml blasticidine S (Invivogen) and 50 mg/ml Gentamicin (Sigma) in 5% CO₂ at 37° C. Cells were grown to 80-95% confluency, rinsed once with 1×PBS (Invitrogen) and detached by incubating with 1×PBS containing 0.02% EDTA (Sigma) for 10 minutes at room temperature. Cells were collected by centrifugation at 900×g, 4° C. for 10 minutes. Cells were rinsed once with 1×PBS and re-suspended in ice cold homogenisation buffer (50 mM Tris-HCl (pH 7.4), 2.5 mM EDTA, 5 mM MgCl₂, 200 mM Sucrose) at 1×10⁷ cells/ml and kept on ice. Cells were homogenised on ice and debris removed by centrifugation at 500×g, 4° C. for 5 minutes. The resulting supernatant was centrifuged at 75,600×g, 4° C. for 60 minutes. Membranes were suspended in homogenisation buffer, protein concentration was determined (BCA Protein Assay kit (Pierce)), diluted to 2.2 mg/ml, dispensed into 1 ml aliquots and stored at −80° C.

Membranes were thawed on ice, sonicated with 4 cycles of 20 pulses (50% amplitude, 0.5 pulse) (UP200S Hielscher) on ice, diluted in assay buffer (50 mM Tris-HCl (pH7.4), 5 mM MgCl₂) to 62.5 μg/ml. Compound was serially diluted in DMSO before being diluted 1:10 with assay buffer. 5 μg of membrane in 80 μl of assay buffer was added per well of a 96 well polystyrene plate (Corning). 10 pd of compound was added per well. The assay was initiated by the addition of 10 μl of 20 nM ³H—NαMH per well and incubated for one hour at room temperature with shaking. Total binding was determined in the presence of 1% DMSO and non-specific binding was determined by the inclusion of 1 μM R-α-methyl-histamine (RαMH). Incubations were then filtered through filtermat A (Perkin Elmer) and washed three times with assay buffer. Filtermats were dried at 42° C. for two hours, scintillant added and the level of bound radioactivity determined.

IC50 values for compounds were determined from seven point log scale dose-response studies and represent the concentration of compound required to inhibit 50% of the specific binding of 2 nM ³H—NαMH (difference between total and non-specific binding). Curves were generated using the average of duplicate wells for each data point and analyzed using nonlinear regression of sigmoidal dose response (variable slope).

### 3.2 In Vitro H₃Functional Assay

The functional activity of compounds at the H3 receptor was determined by measuring changes in the level of intracellular cAMP using a cAMP response element driven luciferase reporter assay. The changes in luciferase expression were monitored by a luminescence plate reader, Analyst HT (MDS Analytical). Increases in intracellular cAMP were readily detected upon activation of protein kinase A by forskolin (Sigma) and suppression of this response observed with the application of the H3 receptor agonist RαMH (Sigma).

CHO(dhfr⁺)-cre-luc cells stably expressing human H3 receptor were routinely grown as monolayers in Minimal Essential Medium α (MEMα) (Invitrogen) supplemented with 10% dialysed FBS (Hyclone), in 5% CO₂ at 37° C. 48 hours prior to assay, cells were seeded in clear-base white walled 384-well plates (Corning) at a density of 5000 cells/well. On the day of assay, growth media was removed and replaced with 15 μA of assay buffer (MEMα, 5 mg/ml fatty acid free BSA (Sigma)) per well. Cells were then incubated for 30 minutes at 37° C., 5% CO₂. Compound was serially diluted in DMSO before being diluted 1:10 with assay buffer. 2.5 of compound diluted in assay buffer was added and cells incubated for 5 minutes at 37° C., 5% CO₂. 2.5 μl of each reagent was then added in the following order: RαMH (10 nM), isobutylmethylxanthine (1-methyl-3-(2-methylpropyl)-7H-purine-2,6-dione; IBMX) (500 μM) (Sigma) and forskolin (1 μM). Cells were then incubated for 90 minutes at 37° C., 5% CO₂, followed by 30 minutes at room temperature. At the end of incubation 25 μl of Steadylite reagent (Perkin Elmer) was added, plates were sealed and placed on a shaker for 5 minutes. The level of light output to determine the level of luciferase expression was then measured.

IC50 values for compounds were determined from ten point half log scale dose-response studies and represent the concentration of compound required to prevent 50% inhibition of forskolin stimulated cells in the presence of RαMH alone. Curves were generated using the average of duplicate wells for each data point and analyzed using nonlinear regression of four parameter dose response.

### 3.3 Results

The results of the biological assays carried out in 3.1 and 3.2 above were as follows:

| Example | hH3 binding | hH3 functional |
|---|---|---|
| Compound | IC₅₀/nM | IC₅₀/nM |
| Example 1 | 1 | 0.7 |
| Example 5 | 22 | 31 |
| Example 11 | 30 | |
| Example 19 | 20 | |
| Example 20 | 11 | 3 |
| Example 21 | 13 | |
| Example 23 | 11 | 2 |
| Example 24 | 44 | 16 |
| Example 25 | 37 | |
| Example 26 | 36 | |
| Example 27 | 21 | |
| Example 29 | 13 | 8 |
| Example 30 | 5 | |
| Example 34 | 26 | 5 |
| Example 35 | 11 | 1 |
| Example 36 | 12 | 2 |
| Example 38 | 9 | |
| Example 40 | 3 | 2 |
| Example 42 | 9 | 30 |
| Example 44 | 14 | 3 |
| Example 45 | 13 | |
| Example 49 | 34 | |
| Example 50 | 24 | |
| Example 53 | 51 | 6 |
| Example 54 | 10 | |
| Example 55 | 12 | |
| Example 57 | 12 | |
| Example 59 | 6 | 3 |
| Example 63 | 6 | |
| Example 65 | 4 | |
| Example 67 | 11 | |
| Example 68 | 1 | 3 |
| Example 69 | 2 | 0.5 |
| Example 70 | 5 | 3 |
| Example 72 | 1 | 2 |
| Example 73 | 12 | 1 |
| Example 74 | 3 | 1 |
| Example 75 | 15 | |
| Example 77 | 3 | 1 |
| Example 78 | 4 | 2 |
| Example 79 | 6 | 3 |
| Example 81 | 0.4 | 0.8 |
| Example 84 | 10 | |
| Example 85 | 14 | |
| Example 86 | 3 | 1 |
| Example 87 | 4 | |
| Example 88 | 5 | 2 |
| Example 89 | 18 | |
| Example 90 | 10 | |
| Example 91 | 6 | |
| Example 92 | 4 | 1 |
| Example 93 | 29 | |
| Example 95 | 18 | |
| Example 96 | 18 | |
| Example 97 | 9 | 2 |
| Example 98 | 15 | |
| Example 99 | 18 | |
| Example 100 | 2 | 0.5 |
| Example 102 | 18 | |
| Example 103 | 11 | 1 |
| Example 106 | 13 | |
| Example 107 | 9 | 4 |
| Example 108 | 1 | 4 |
| Example 109 | 2 | |
| Example 110 | 17 | |
| Example 111 | 7 | 7 |
| Example 113 | 13 | |
| Example 114 | 12 | 19 |
| Example 120 | 1 | |
| Example 131 | 30 | |
| Example 132 | 6 | |
| Example 134 | 13 | 1 |
| Example 137 | 26 | |
| Example 143 | 24 | 7 |
| Example 146 | 10 | 10 |
| Example 155 | 24 | |
| Example 164 | 10 | 5 |
| Example 167 | 22 | 71 |
| Example 171 | 14 | 4 |
| Example 181 | 21 | 4 |
| Example 191 | 13 | 2 |
| Example 194 | 26 | 7 |
| Example 211 | 6 | 9 |
| Example 216 | 41 | 20 |
| Example 221 | 11 | 3 |
| Example 224 | 2 | 4 |
| Example 231 | 23 | 15 |
| Example 233 | 2 | 16 |
| Example 239 | 16 | 1 |
| Example 242 | 0.6 | 3 |
| Example 244 | 10 | 6 |
| Example 251 | 11 | 6 |
| Example 253 | 1 | 4 |
| Example 256 | 0.3 | 3 |
| Example 257 | 25 | 7 |
| Example 262 | 25 | 10 |
| Example 263 | 18 | 6 |
| Example 265 | 6 | 4 |
| Example 268 | 4 | |
| Example 269 | 2.5 | |
| Example 274 | 9 | |
| Example 275 | 7 | |
| Example 276 | 19 | |
| Example 280 | 0.3 | |
| Example 281 | 0.8 | |
| Example 282 | 1 | |
| Example 287 | 1 | |
| Example 290 | 3 | |
| Example 294 | 8 | |
| Example 300 | 5 | |
| Example 302 | 3 | |
| Example 304 | 61 | 34 |
| Example 306 | 18 | 2 |
| Example 313 | 1 | |
| Example 315 | 2 | |
| Example 316 | 0.9 | |
| Example 319 | 62 | |

These results indicate that compounds of the invention have potent antagonist or inverse agonist activity at the H3 receptor, both in terms of binding and in terms of inhibition of the functional response caused by receptor activation. The compounds tested above exhibit IC₅₀ values of less than 1 μM, and several compounds show low nanomolar affinity at the H3 receptor. Accordingly, the compounds of the invention are expected to have usefulness in the prevention or treatment of conditions, such as those discussed above, in which H3 receptor activity is implicated.

### REFERENCES

1. J.-M. Arrang, M. Garbarg and J.-C. Schwartz. Nature, 1983, 302, 832
2. T. W. Lovenberg, B. L. Roland, S. J. Wilson, X. Jiang, J. Pyati, A. Huvar, M. R. Jackson and M. G. Erlander. Mol. Pharmacol., 1999, 55, 1101.
3. S. J. Hill, C. Ganellin, H. Timmermans, J. C. Schwartz, N. Shankley, J. M. Young, W. Schunack, R. Levi and H. L. Haas. Pharmacol. Rev., 1997, 49, 253.
4. Passani M B, Lin J-S, Hancock A, Crochet S, Blandina P. The histamine H3 receptor as a novel therapeutic target for cognitive and sleep disorders. Trends Pharmacol. Sci. 2004; 25:618-25.
5. Witkin J M, Nelson D L. Selective histamine H3 receptor antagonists for treatment of cognitive deficiencies and other disorders of the central nervous system. Pharmacol. Ther. 2004; 103:1-20
6. Monti J. M et al. Effect of Selective activation or blockade of the hitamine H3 receptor on sleep and wakefulness. 1991 Eur. J. Pharmacol. 205, 283-287.
7. Esbenshade T. A. et al. Biochemical Pharmacology 68 (2004) 933-945.
8. Morimoto T, Yamamoto Y, Yamatodani A. Leptin facilitates histamine release from the hypothalamus in rats. Brain Res. 2000; 868:367-9
9. A. A. Hancock. Biochem. Pharmacol., 2006, 71, 1103.
10. A. A. Hancock and M. E. Brune. Expert Opin. Investig. Drugs, 2005, 14, 223
11. D. Farzin, L. Asghari and M. Nowrouzi. Pharmacol. Biochem. Behay., 2002, 72, 751.
12. WO 04/089410
13. Medhurst A. D. et al. Biochemical Pharmacology 73 (2007) 1182-94
14. Esbenshade T. A et al. J. Pharmacol. Exp. Ther. 2005 313(1) 165-75

1. A compound having the formula: 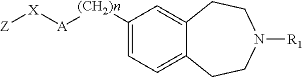
wherein:
R1 is a group selected from C₃₋₈ cycloalkyl, C₁₋₆ alkyl, C₁₋₆ alkylene-C₃₋₈ cycloalkyl, each of which groups may optionally be substituted with C₁₋₆ alkyl, halogen, haloC₁₋₆ alkyl or OR15, or R1 is heterocyclyl, optionally substituted with C₁₋₆ alkyl, haloC₁₋₆ alkyl or OR15;
n is 0, 1, 2, 3 or 4, the alkylene group —(CH₂)ₙ— formed thereby being optionally substituted with a group selected from C₁₋₄ alkyl, C₃₋₈ cycloalkyl and arylsulfonyl;
A is a group selected from —N(R2)CO—, —CON(R2)-, —OC(O)—, —C(O)O—, —CO—, —C(R2)(OR3)-, —C(═N—O—R3)-, —C(═CR2R3)-, —C₃₋₈ cycloalkylene-, —C(R2)(haloC₁₋₆ alkyl)-, C₁₋₄ alkylene and —C(OR3)(haloC₁₋₆ alkyl)-;
R2 and R3 are each independently selected from H, C₁₋₆ alkyl, and C₃₋₈ cycloalkyl, or, when A is —N(R2)CO— and X is absent, R2 may form, together with the adjacent nitrogen atom and Z, an N-containing heterocyclyl group, which may optionally be substituted;
X is absent or is C₁₋₄ alkylene or C₂₋₄ alkenylene, each of which may optionally be substituted with one or more C₁₋₄ alkyl groups, OR16, halogen or haloC₁₋₆ alkyl;
Z is selected from aryl, heteroaryl, C₃₋₈ cycloalkyl, and heterocyclyl, each of which may optionally be substituted by a group selected from —Y-aryl, —Y-heteroaryl, cycloalkyl and —Y-heterocyclyl, or, when X is present, Z may be H, or, when X is absent and A is —C(R2)(OR3)- or —N(R2)CO—, Z may be H, or, when A is —N(R2)CO— and X is absent, Z may form, together with the adjacent nitrogen atom and R2, an N-containing heterocyclyl group which may optionally be substituted, wherein, when A is —CO—, Z is linked to X or A via a carbon atom and wherein, when A is —N(R2)CO— and Z is H, R1 is C₃₋₈ cycloalkyl; and
Y represents a bond, C₁₋₆ alkylene, CO, NR14, COC₂₋₆ alkenylene, O, SO₂ or NHCOC₁₋₆ alkylene;
wherein said cycloalkyl, aryl, heteroaryl and heterocyclyl groups Z may be optionally substituted by one or more substituents which may be the same or different, and which are selected from halogen, haloC₁₋₆ alkyl, hydroxy, cyano, nitro, ═O, —R4, —CO₂R4, —COR4, —NR5R6, —C₁₋₆ alkyl-NR5R6, —C₃₋₈ cyclo alkyl-NR5R6, —CONR12R13, —NR12COR13, —NR5SO₂R6, —OCONR5R6, —NR5CO₂R6, —NR4CONR5R6 or —SO₂NR5R6-SHR8, -alkyl-OR8, —SOR8, —OR9, —SO₂R9, —OSO₂R9, -alkyl-SO₂R9, -alkyl-CONHR9, -alkyl-SONHR9, -alkyl-COR10, —CO-alkyl-R10, —O-alkyl-R11 (wherein R4, R5 and R6 independently represent hydrogen, C₁₋₆ alkyl, —C₃₋₈ cycloalkyl, —C₁₋₆ alkylene-C₃₋₈ cycloalkyl, aryl, heterocyclyl or heteroaryl, wherein R8 represents C₁₋₆ alkyl, wherein R9 represents C₁₋₆ alkyl or aryl, wherein R10 represents aryl, wherein R11 represents C₃₋₈ cycloalkyl or aryl, R12, R13, R14, R15 and R16 each independently represent H or C₁₋₆ alkyl, and wherein —NR5R6 and —NR12R13 may represent a nitrogen containing heterocyclyl group); wherein said R4, R5, R6R8, R9, R10 and R11 groups may be optionally substituted by one or more substituents which may be the same or different, and which are selected from the group consisting of halogen, hydroxy, C₁₋₆ alkyl, C₁₋₆ alkoxy, cyano, amino, ═O or trifluoromethyl;
and wherein substituents of Z selected from —Y-aryl, —Y-heteroaryl, —Y—C₃₋₈ cycloalkyl and —Y-heterocyclyl may be optionally substituted by one or more substituents selected from ═O, hydroxy, cyano, nitro, halogen, haloC₁₋₆ alkyl and C₁₋₆ alkyl;
and wherein, when A is C₁₋₄ alkylene, said cycloalkyl, aryl, heteroaryl or heterocyclyl group Z (such as a heterocyclyl group Z) is substituted at least with hydroxy, CF₃, or ═O;
and wherein, when A is CON(R2) n is 1;
or a pharmaceutically acceptable salt or ester thereof,
provided that: when A is —CO—, R1 is CH₃, C₃₋₈ cycloalkyl-substituted C₁₋₆ alkylene or n-butyl, n is 0 and X is —CH₂CH₂—, Z is not N-benzyl substituted 4-piperidinyl, N-(3-fluorobenzyl)-substituted 4-piperidinyl or N-acetyl substituted 4-piperidinyl;
when A is —OC(O)—, R1 is cyclobutyl, n is 0 and X is —CH₂CH₂—, Z is not H;
when A is —OC(O)—, R1 is n-propyl, n is 0 and X is —CH₂—, Z is not H; and
when A is —CO—, R1 is CH₃, n is 0 and X is CH₂, Z is not H.

2. A compound according to claim 1, wherein: R1 is C₁₋₆ alkyl, C₃₋₈ cycloalkyl-C₁₋₆ alkylene, or C₃₋₈ cycloalkyl, each of which may optionally be substituted by one or two halogens, hydroxy or C₁₋₆ alkoxy (such as methoxy), or R1 is heterocyclyl, optionally substituted by hydroxy, C₁₋₆ alkoxy or C₁₋₆ alkyl;
n is 0, 1 or 2;
A is —N(R2)CO—, —OC(O)—, —CON(R2)-, —CO—, —C(R2)(OR3)-, C₁₋₄alkylene, —C(═N—O—R3)- or —C(═CHR3)-;
R2 and R3 are each independently H or C₁₋₆ alkyl;
or, when A is —N(R2)CO— and X is absent, R2 may form, together with the adjacent nitrogen atom and Z, an N-containing heterocyclyl group which may optionally be substituted by one to three halogen atoms or carbamoyl groups;
X is absent or is C₁₋₄ alkylene or C₂₋₄ alkenylene, each of which may optionally be substituted with a C₁₋₄ alkyl group; and
Z is aryl, heteroaryl, C₃₋₈ cycloalkyl or heterocyclyl,
each of which may optionally be substituted by
(1) a group selected from —Y-aryl, —Y-heteroaryl, —Y-heterocyclyl, and —Y—C₃₋₈ cycloalkyl,
wherein Y represents a bond, O, NR14, or C₁₋₆ alkylene, and said aryl is selected from phenyl, said heteroaryl is selected from triazolyl, thiazolyl, thienyl and pyrazolyl, said heterocyclyl is selected from morpholinyl, tetrahydrofuranyl, tetrahydropyranyl and pyrrolidinyl, and said C₃₋₈ cycloalkyl is selected from cyclobutyl or cyclopropyl; or
(2) one to three substituents selected from
C₁₋₆ alkyl, halogen, haloC₁₋₆ alkyl, cyano, amino, C₁₋₆ alkoxy, C₁₋₆ alkyl-carbonyl, hydroxy-substituted C₁₋₆ alkyl-carbonyl, C₃₋₈ cycloalkyl-carbonyl, carboxyl, C₁₋₆ alkoxy-carbonyl, carbamoyl, C₁₋₆ alkyl-carbamoyl; C₁₋₆ alkylamino, and ═O; or
Z may be H when X is present, or Z may be H when X is absent and A is —C(R2)(OR3)- or —N(R2)CO—,
wherein substituents of Z selected from —Y-aryl, —Y-heteroaryl, —Y—C₃₋₈cycloalkyl and —Y-heterocyclyl may be optionally substituted by one or more substituents selected from ═O, hydroxy, cyano, nitro, halogen, haloC₁₋₆ alkyl and C₁₋₆ alkyl;
wherein, when A is C₁₋₄ alkylene, said cycloalkyl, aryl, heteroaryl or heterocyclyl group Z (such as a heterocyclyl group Z) is substituted at least with hydroxy, CF₃ or ═O;
and wherein, when A is CON(R2), n is 1.

3. A compound according to claim 1, wherein: R1 is C₁₋₆ alkyl or C₃₋₈ cycloalkyl, optionally substituted with halogen or C₁₋₆ alkoxy, or R1 is heterocyclyl, optionally substituted with C₁₋₆ alkyl;
n is 1;
A is —CON(R2)- or —N(R2)CO—;
R2 is selected from H and C₁₋₆ alkyl;
X is absent or is C₁₋₄ alkylene, which may be optionally substituted with one or more C₁₋₄ alkyl or hydroxy groups;
Z is aryl, heteroaryl, C₃₋₈ cycloalkyl or heterocyclyl,
each of which may optionally be substituted by
(1) a group selected from —Y-aryl, —Y-heteroaryl, Y-heterocyclyl, and —Y—C₃₋₈ cycloalkyl,
wherein Y represents a bond, O, NR14, or C₁₋₆ alkylene, and said aryl is selected from phenyl, said heteroaryl is selected from triazolyl, thiazolyl, thienyl and pyrazolyl, said heterocyclyl is selected from morpholinyl, tetrahydrofuranyl, tetrahydropyranyl, and pyrrolidinyl, and said C₃₋₈ cycloalkyl is selected from cyclopropyl and cyclobutyl; or
(2) one to three substituents selected from
C₁₋₆ alkyl, halogen, haloC₁₋₆ alkyl), cyano, amino, C₁₋₆ alkylamino, N,N—C₁₋₆ dialkylamino, C₁₋₆ alkoxy, C₁₋₆ alkyl-carbonyl, carboxyl, C₁₋₆ alkoxy-carbonyl, carbamoyl, C₁₋₆ alkyl-carbamoyl, hydroxy C₁₋₆ alkyl and ═O; or
Z may be H when X is present,
wherein substituents of Z selected from —Y-aryl, —Y-heteroaryl, —Y—C₃₋₈cycloalkyl and —Y-heterocyclyl may be optionally substituted by one or more substituents selected from ═O, hydroxy, cyano, nitro, halogen, haloC₁₋₆ alkyl and C₁₋₆ alkyl.

4. A compound according to claim 1, wherein: R1 is C₁₋₆ alkyl or C₃₋₈ cycloalkyl;
n is 1;
A is —C(R2)(OR3)-;
R2 and R3 are each independently H or C₁₋₆ alkyl;
X is absent or is C₁₋₄ alkylene;
Z is heteroaryl, or heterocyclyl,
each of which may optionally be substituted by one to three substituents selected from
C₁₋₆ alkyl, halogen, haloC₁₋₆ alkyl, cyano, hydroxy, amino, C₁₋₆ alkoxy, C₁₋₆ alkyl-carbonyl, hydroxy-substituted C₁₋₆ alkyl-carbonyl, carboxyl, C₁₋₆ alkoxy-carbonyl, C₃₋₈ cycloalkyl-carbonyl, carbamoyl, C₁₋₆ alkyl-carbamoyl.

5. A compound according to claim 1, wherein: R1 is C₁₋₆ alkyl or C₃₋₈ cycloalkyl;
n is 0;
A is C₁₋₄ alkylene;
R2 and R3 are each independently H or C₁₋₆ alkyl;
X is absent or is C₁₋₄ alkylene;
Z is heteroaryl, or heterocyclyl, each of which may optionally be substituted by one to three substituents selected from C₁₋₆ alkyl, halogen), haloC₁₋₆ alkyl, cyano, hydroxy, amino, C₁₋₆ alkoxy, C₁₋₆ alkyl-carbonyl, hydroxy-substituted C₁₋₆ alkyl-carbonyl, carboxyl, C₁₋₆ alkoxy-carbonyl, C₃₋₈ cycloalkyl-carbonyl, carbamoyl, C₁₋₆ alkyl-carbamoyl,
wherein said heteroaryl or heterocyclyl group Z is substituted at least with hydroxy, CF₃ or ═O.

6. A compound according to claim 1, wherein R1 is C₁₋₃ alkyl, heterocyclyl or C₃₋₈ cycloalkyl.

7. A compound according to claim 1, wherein R1 is C₁₋₃ alkyl substituted with C₃₋₈ cycloalkyl.

8. A compound according to claim 1, wherein R1 is cyclopropylethyl or cyclopropylmethyl.

9. A compound according to claim 1, wherein R1 is selected from methyl, ethyl, isopropyl, cyclopropyl, cyclobutyl, tetrahydrofuranyl and cyclopentyl.

10. A compound according to claim 1, wherein R1 is further substituted with a group selected from F, methyl, hydroxy, C₁₋₆ alkoxy and CH₂F.

11. A compound according to claim 1, wherein R2 is H.

12. A compound according to claim 1, wherein R3 is H.

13. A compound according to claim 1, wherein X is a straight chain C₁₋₄ alkylene group, optionally having one or more methyl or ethyl substituents.

14. A compound according to claim 13, wherein X is methylene or ethylene.

15. A compound according to claim 1, wherein Z is aryl, heteroaryl, C₃₋₈ cycloalkyl or heterocyclyl, each of which may be substituted with one or more substituents selected from C₁₋₆ alkyl, halogen, haloC₁₋₆ alkyl, cyano, amino, C₁₋₆ alkoxy, —COR4, —CONR12R13, aryl, and heteroaryl.

16. A compound according to claim 1, wherein Z is heteroaryl, said heteroaryl being selected from thienyl, furyl, furazanyl, pyrrolyl, triazolyl, tetrazolyl, imidazolyl, imidazopyridyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl, quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, fluoropyridinyl, benzofuranyl, isobenzofuranyl, benzothienyl, benzimidazolyl, benzoxazolyl, benzoisoxazolyl, benzothiazolyl, benzoisothiazolyl, benzoxadiazolyl, and benzothiadiazolyl groups.

17. A compound according to claim 1, wherein Z is aryl, said aryl being selected from phenyl, naphthyl and tetrahydronaphthalenyl groups.

18. A compound according claim 1, wherein Z is heterocyclyl, said heterocyclyl being selected from pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl, indolinyl, isoindolinyl, benzopyranyl, quinuclidinyl, 2,3,4,5-tetrahydro-1H-3-benzazepine, and tetrahydroisoquinolinyl groups.

19. A compound according to claim 1, wherein Z is cycloalkyl, said cycloalkyl being selected from cyclopropyl, cyclobutyl, cyclopentyl, cyclohexyl, cycloheptyl and cyclooctyl groups.

20. A compound according to claim 1, wherein A is —CON(R2)-, and n is 0, 1 or 2.

21. A compound according to claim 1, wherein A is —OC(O)— or —C(O)O—, and n is 0, 1 or 2.

22. A compound according to claim 1, wherein A is —C(R2)(OR3)- or —CO—, and n is 0, 1 or 2.

23. A compound according to claim 20, wherein R2 is H.

24. A compound according to claim 22, wherein R3 is H or C₁₋₄ alkyl.

25. A pharmaceutical composition comprising a compound according to claim 1, together with one or more pharmaceutically acceptable excipients.

26. (canceled)

27. (canceled)

28. A method of treatment or prevention of a condition whose development or symptoms are linked to histamine H3 receptor activity, the method comprising the administration, to a subject in need of such treatment or prevention, of a therapeutically effective amount of a compound according to claim 1, wherein the provisos to claim 1 do not apply.

29. A method according to claim 28, wherein the condition is a disorder of the central nervous system.

30. A method according to claim 29, wherein the disorder is selected from schizophrenia, neurodegenerative disorders (such as Alzheimer's Disease), cognitive disorders (such as dementia), sleep disorders, pain, obesity, attentional disorders and epilepsy.

31. An intermediate compound having the formula: 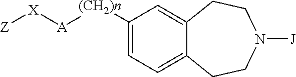
wherein n, A, X and Z have the same meaning as in claim 1, or Z—X-A- together represents C₁₋₆ alkylsulfonyloxy, nitro, halogen (such as Br), carbaldehyde O—C₁₋₆ alkyl oxime, amino, amino attached to an amino protecting group or arylsulfonyl, and wherein J is an amino protecting group or H, provided that Z is joined to X or A via a carbon atom when Z contains a piperazinyl moiety, and provided that:
when A is —OC(O)—, J is H, n is 0 and X is —CH₂CH₂—, Z is not H;
when A is —OC(O)—, J is tert-butoxycarbonyl, n is 0 and X is —CH₂—, Z is not H;
when A is —NHCO—, J is tert-butoxycarbonyl, n is 0 and X is -isopropyl, Z is not H; and
when A is —NHCO—, J is tert-butoxycarbonyl, aminoiminomethyl or H, n is 0 and X is —CH₂— or —CH₂CH₂—, Z is not pyrrolidin-2-yl substituted with oxo, phenylpropyl and acetic acid substituents.

32. An intermediate compound having the formula: 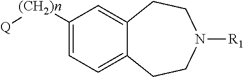
wherein n and R1 have the same meaning as in claim 1, and wherein Q is selected from cyano, amino, amino attached to an amino protecting group, arylsulfonyl and halogen (such as Br).

33. (canceled)

34. A method of synthesis of a compound according to claim 1, wherein A is —N(R2)CO—, the method comprising the reaction of an intermediate having the formula: 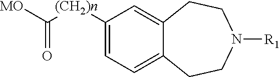
with an amine (Z—X)(R2)NH in the presence of a catalyst, wherein n, Z, X, R1 and R2 have the same meaning as in claim 1, and wherein M represents H or a monovalent metal cation.

35. A method of synthesis of a compound according to claim 1, wherein A is —CO— or —C(R2)(OR3)- and X is present, the method comprising the reaction of a protected intermediate: 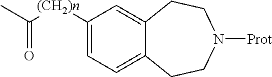
with an aldehyde Z—CHO in the presence of a catalyst, followed by deprotection of the protected amine and substitution thereof with R1 and, optionally, by catalytic hydrogenation, wherein n, Z, X, R1, R2 and R3 have the same meaning as in claim 1, and wherein Prot represents an amine protecting group.

36. A compound according to claim 22, wherein R2 is H.

37. A compound according to claim 23, wherein R3 is H or C₁₋₄ alkyl.
